(19) United States
(12) Patent Application Publication
(10) Pub. No.: US 2025/0177533 A1
(43) Pub. Date: Jun. 5, 2025

(54) PHOTODYNAMIC THERAPY AND DIAGNOSIS

(71) Applicant: RMW CHO Group Limited, Hong Kong (CN)

(72) Inventors: Sebastian M. Marcuccio, Melbourne (AU); Andrew N. Stephens, Melbourne (AU); Christopher D. Donner, Melbourne (AU); Honsue Cho, Melbourne (AU); Sacha Novakovic, Melbourne (AU); Ramesh Ailuri, Melbourne (AU)

(21) Appl. No.: 18/713,550

(22) PCT Filed: Nov. 28, 2022
(86) PCT No.: PCT/EP2022/083551

§ 371 Date: May 24, 2024

(30) Foreign Application Priority Data
Nov. 26, 2021 (GB) 2117133.5
Jun. 1, 2022 (GB) 2208157.4

(51) Int. Cl. A61K 41/00; A61K 39/395; A61K 47/32; C07D 487/22; C07H 15/26

(52) U.S. Cl. CPC A61K 41/0071; A61K 39/3955; A61K 47/32; C07D 487/22; C07H 15/26

(57) ABSTRACT

The present invention relates to phyllochlorin analogues and their pharmaceutically acceptable salts, and compositions comprising phyllochlorin analogues and their pharmaceutically acceptable salts. Phyllochlorin analogues and pharmaceutically acceptable salts thereof are suitable for use in photodynamic therapy, cytoluminescent therapy and photodynamic diagnosis, for example, for treating or detecting a tumour, or for antiviral treatment. The present invention also relates to the use of phyllochlorin analogues and pharmaceutically acceptable salts thereof in the manufacture of a phototherapeutic or photodiagnostic agent, and to a method of photodynamic therapy, cytoluminescent therapy or photodynamic diagnosis, for example, for treating or detecting a tumour, or for antiviral treatment.

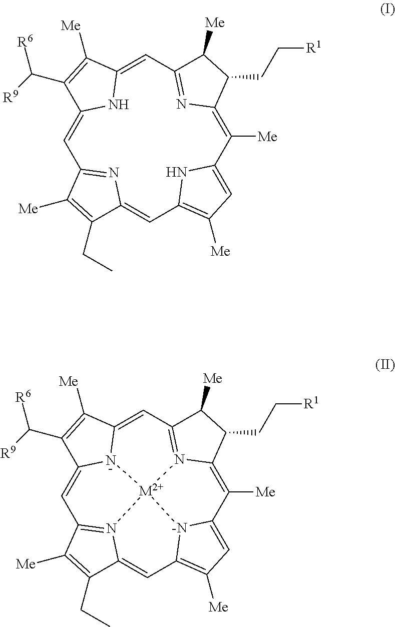

### TECHNICAL FIELD

The present invention relates to phyllochlorin analogues and their pharmaceutically acceptable salts, and compositions comprising phyllochlorin analogues and their pharmaceutically acceptable salts. Phyllochlorin analogues and pharmaceutically acceptable salts thereof are suitable for use in photodynamic therapy, cytoluminescent therapy and photodynamic diagnosis, for example, for treating or detecting a tumour, or for antiviral treatment. The present invention also relates to the use of phyllochlorin analogues and pharmaceutically acceptable salts thereof in the manufacture of a phototherapeutic or photodiagnostic agent, and to a method of photodynamic therapy, cytoluminescent therapy or photodynamic diagnosis, for example, for treating or detecting a tumour, or for antiviral treatment.

The structure of ‘phyllochlorin’ is shown below:

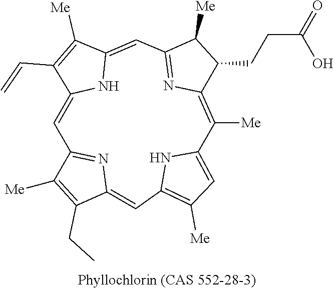 [SMILES: C=Cc1c(C)c2cc3nc(c(C)c4cc(C)c(cc5nc(cc1[nH]2)C(C)=C5CC)[nH]4)[C@@H](CCC(=O)O)[C@@H]3C]

### 3-((7S,8S)-18-ethyl-2,5,8,12,17-pentamethyl-13-vinyl-7H,8H-porphyrin-7-yl) propanoic acid

### BACKGROUND ART

Porphyrins and their analogues are known photosensitive chemical compounds, which can absorb light photons and emit them at higher wavelengths. There are many applications for such unique properties and PDT (photodynamic therapy) is one of them.

Presently, there are two generations of photosensitizers for PDT. The first generation comprises heme porphyrins (blood derivatives), and the second for the most part are chlorophyll analogues. The later compounds are known as chlorins and bacteriochlorins.

Chlorin e4 has been shown to display good photosensitive activity. It was indicated that chlorin e4 has a protective effect against indomethacin-induced gastric lesions in rats and TAA- or CCl4-induced acute liver injuries in mice. It was therefore suggested that chlorin e4 may be a promising new drug candidate for anti-gastrelcosis and liver injury protection. WO 2009/040411 suggests the use of a chlorin e4 zinc complex in photodynamic therapy and WO 2014/091241 suggests the use of chlorin e4 disodium in photodynamic therapy.

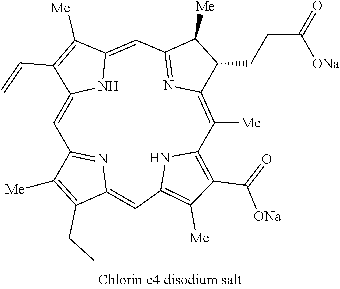

However, there is an ongoing need for better photosensitizers. There is a need for compounds that have a high singlet oxygen quantum yield and for compounds that have a strong photosensitizing ability, preferably in organic and aqueous media. There is also a need for compounds that have a high fluorescence quantum yield. In addition, there is a need for compounds and/or compositions which have a higher phototoxicity, a lower dark toxicity, good stability, good solubility, and/or are easily purified.

### SUMMARY OF THE INVENTION

A first aspect of the present invention provides a compound of formula (I) or a complex of formula (II):

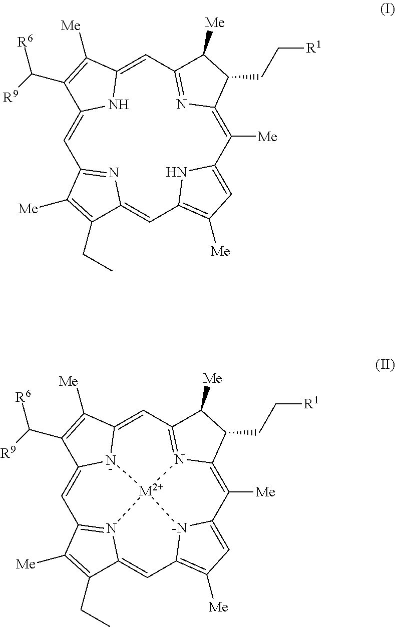

or a pharmaceutically acceptable salt thereof, wherein:
—R¹ is selected from —CH₂OR², —CH₂SR², —CH₂S(O)R², —CH₂S(O)₂R², —CH₂N(R²)₂, —R², —C(O)—OR³, —C(O)—SR³, —C(O)—N(R³)₂, —C(S)—OR³, —C(S)—SR³ or —C(S)—N(R³)₂;
—R², each independently, is selected from —H, —C(O)R⁴, —C(O)—OR⁴, —C(O)—SR⁴, —C(O)—N(R⁴)₂, —C(S)—OR⁴, —C(S)—SR⁴, —C(S)—N(R⁴)₂, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁷]Y, —R^{α}—[N(R⁵)₂ (R^{5′})], —R^{α}—[P(R⁵)₂ (R^{5′})] or —R^{α}—[R⁷];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁷]Y, —R^{α}—[N(R⁵)₂ (R^{5′})], —R^{α}—[P(R⁵)₂ (R^{5′})] or —R^{α}—[R⁷];
—R^{α}—, each independently, is selected from a C₁-C₄₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R⁵, each independently, is selected from C₁-C₄ alkyl, C₁-C₄ haloalkyl, (CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O), —H or —O—(CH₂CH₂O)ₙ—CH₃ groups;
—R^{5′} is selected from C₁-C₄ alkyl, C₁-C₄ haloalkyl, —(CH₂CH₂O)ₙ—H, (CH₂CH₂O)ₙ—CH₃, phenyl or C₅-C₆ heteroaryl, each substituted with —CO₂, wherein the phenyl or C₅-C₆ heteroaryl may optionally be further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from —OR², —N(R²)₂, —SR², —S(O)R², —S(O)₂R², or —X;
—R⁷ is —[NC₅H₅] optionally substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O), —H or —O—(CH₂CH₂O)ₙ—CH₃ groups;
—R^{7′} is —[NC₅H₅] substituted with —CO₂ and optionally further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O), —H or —O—(CH₂CH₂O)ₙ—CH₃ groups;
—R⁹ is hydrogen or methyl; or —R⁶ and —R⁹ together form an oxo (═O) group;
n is 1, 2, 3, 4, 5 or 6;
X is a halo group;
Y is a counter anion;
Z is a counter cation; and
M²⁺ is a metal cation.

The first aspect of the present invention also provides a compound of formula (I) or a complex of formula (II):

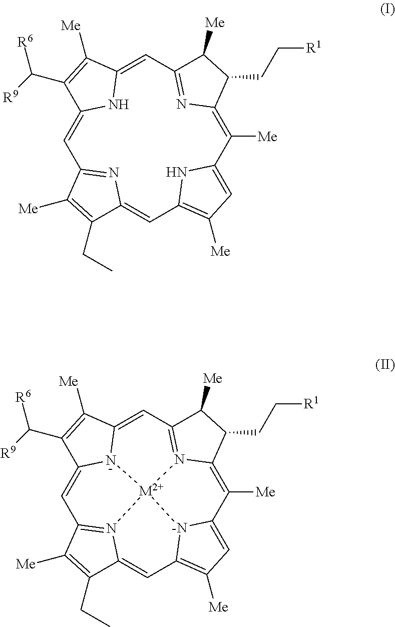

or a pharmaceutically acceptable salt thereof, wherein:
—R¹ is selected from —CH₂OR², —CH₂SR², —CH₂S(O)R², —CH₂S(O)₂R², —CH₂N(R²)₂, —R², —C(O)—OR³, —C(O)—SR³, —C(O)—N(R³)₂, —C(S)—OR³, —C(S)—SR³ or —C(S)—N(R³)₂;
—R², each independently, is selected from —H, —C(O)R⁴, —C(O)—OR⁴, —C(O)—SR⁴, —C(O)—N(R⁴)₂, —C(S)—OR⁴, —C(S)—SR⁴, —C(S)—N(R⁴)₂, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁷]Y, —R^{α}—[N(R⁵)₂ (R^{5′})], —R^{α}—[P(R⁵)₂ (R^{5′})] or —R^{α}—[R⁷];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁷]Y, —R^{α}—[N(R⁵)₂ (R^{5′})], —R^{α}—[P(R⁵)₂ (R^{5′})] or —R^{α}—[R⁷];
—R^{α}—, each independently, is selected from a C₁-C₄₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R⁵, each independently, is selected from C₁-C₄ alkyl, C₁-C₄ haloalkyl, (CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O), —H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂, wherein the phenyl or C₅-C₆ heteroaryl may optionally be further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from —OR², —SR², —S(O)R², —S(O)₂R², or —X;
—R⁷ is —[NC₅H₅] optionally substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O), —H or —O—(CH₂CH₂O)ₙ—CH₃ groups;
—R^{7′} is —[NC₃H₅] substituted with —CO₂ and optionally further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁹ is hydrogen or methyl;
n is 1, 2, 3, 4, 5 or 6;
X is a halo group;
Y is a counter anion;
Z is a counter cation; and
M²⁺ is a metal cation.

The first aspect of the present invention also provides a compound of formula (I) or a complex of formula (II):

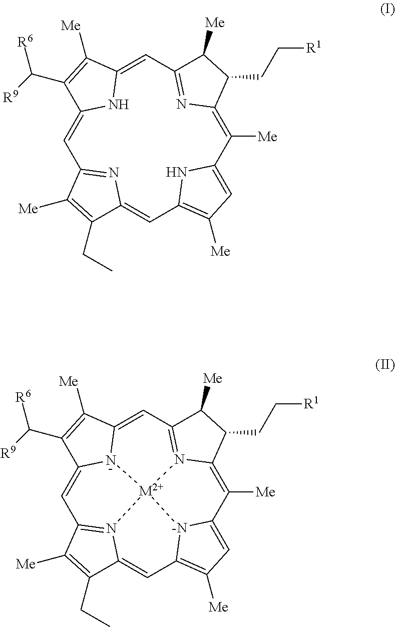

wherein
—R¹ is selected from —CH₂OR², —CH₂SR², —CH₂S(O)R², —CH₂S(O)₂R², —CH₂N(R²)₂, —R², —C(O)—OR³, —C(O)—SR³, —C(O)—N(R³)₂, —C(S)—OR³, —C(S)—SR³ or —C(S)—N(R³)₂;
—R², each independently, is selected from —H, —C(O)R⁴, —C(O)—OR⁴, —C(O)—SR⁴, —C(O)—N(R⁴)₂, —C(S)—OR⁴, —C(S)—SR⁴, —C(S)—N(R⁴)₂, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R) 2, —R^{α}—X, —R^{α}—[N(R⁵)₃]Y, —R^{α}—[P(R⁵)₃]Y or —R^{α}—[R⁷]Y;
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O) R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y or —R^{α}—[R⁷]Y;
—R^{α}—, each independently, is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O, S, P or Se in its carbon skeleton;
—R⁵, each independently, is selected from C₁-C₄ alkyl, C₁-C₄ haloalkyl, halo, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
—R⁶ is selected from —OR², —SR², —S(O)R², —S(O)₂R², or —X;
—R⁷ is —[NC₃H₅] optionally substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
n is 1, 2, 3 or 4;
Y is a counter ion;
X is a halo group;
M²⁺ is a metal ion;
   or a pharmaceutically acceptable salt thereof.

A second aspect of the present invention provides a compound of formula (I) or a complex of formula (II) according to the first aspect of the invention, for use in medicine.

In one embodiment of the first or second aspect of the present invention, the compound of formula (I) or (II) is not:
(i) methyl 3-(18-ethyl-13-(hydroxymethyl)-2,5,8,12,17-pentamethyl-7H,8H-porphyrin-7-yl) propanoate;
(ii) methyl 3-(18-ethyl-13-(hydroxymethyl)-2,5,8,12,17-pentamethyl-7H,8H-porphyrin-7-yl) propanoate copper complex;
(iii) methyl 3-(18-ethyl-13-(hydroxymethyl)-2,5,8,12,17-pentamethyl-7H,8H-porphyrin-7-yl) propanoate zinc complex;
(iv) methyl 3-(18-ethyl-13-formyl-2,5,8,12,17-pentamethyl-7H,8H-porphyrin-7-yl) propanoate;
   or an enantiomer of any thereof;
   or a racemic mixture of any thereof;
   or a salt of any thereof.

In the context of the present specification, a “hydrocarbyl” substituent group or a hydrocarbyl moiety in a substituent group only includes carbon and hydrogen atoms but, unless stated otherwise, does not include any heteroatoms, such as N, O, S, P or Se in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O, S, P or Se in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₆₀ hydrocarbyl group, more typically a C₁-C₄₀ hydrocarbyl group, more typically a C₁-C₂₀ hydrocarbyl group. More typically a hydrocarbyl group is a C₁-C₁₂ hydrocarbyl group. More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear (i.e. straight-chained) or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds. Examples of alkynyl groups/moieties include ethynyl, propargyl, but-1-ynyl and but-2-ynyl. Typically an alkynyl group is a C₂-C₁₂ alkynyl group. More typically an alkynyl group is a C₂-C₆ alkynyl group. An “alkynylene” group is similarly defined as a divalent alkynyl group.

A “cyclic” substituent group or a cyclic moiety in a substituent group refers to any hydrocarbyl ring, wherein the hydrocarbyl ring may be saturated or unsaturated (including aromatic) and may include one or more heteroatoms, e.g. N, O, S, P or Se in its carbon skeleton. Examples of cyclic groups include cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms. More typically, a cyclic group is a 3- to 7-membered monocyclic group, which means it contains from 3 to 7 ring atoms.

A “heterocyclic” substituent group or a heterocyclic moiety in a substituent group refers to a cyclic group or moiety including one or more carbon atoms and one or more (such as one, two, three or four) heteroatoms, e.g. N, O, S, P or Se in the ring structure. Examples of heterocyclic groups include heteroaryl groups as discussed below and non-aromatic heterocyclic groups such as azetidinyl, azetinyl, tetrahydrofuranyl, pyrrolidinyl, tetrahydrothiophenyl, tetrahydropyranyl, piperidinyl, piperazinyl, morpholinyl, thiomorpholinyl, oxetanyl, thietanyl, pyrazolidinyl, imidazolidinyl, dioxolanyl, oxathiolanyl, thianyl and dioxanyl groups.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

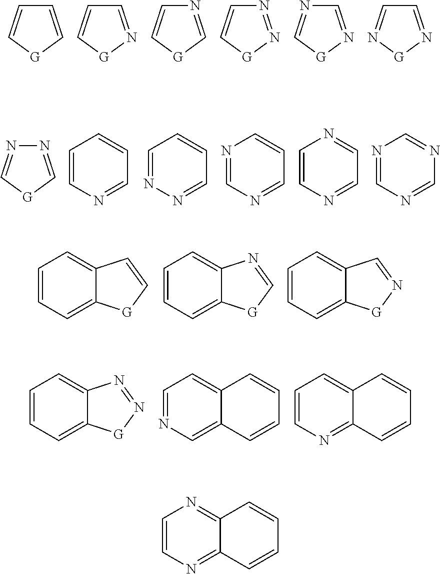

wherein G=O, S or NH.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

For the purposes of the present specification, in an optionally substituted group or moiety (such as —R^{β}):
(i) each hydrogen atom may optionally be replaced by a monovalent substituent independently selected from halo; —CN; —NO₂; —N₃; —R^{x}; —OH; —OR^{x}; —R^{y}-halo; —R^{y}—CN; —R^{y}—NO₂; —R^{y}—N₃; —R^{y}—R^{x}; —R^{y}—OH; —R^{y}—OR^{x}; —SH; —SR^{x}; —SOR^{x}; —SO₂H; —SO₂R^{x}; —SO₂NH₂; —SO₂NHR^{x}; —SO₂N(R^{x})₂; —R^{y}—SH; —R^{y}—SR^{x}; —R^{y}—SOR^{x}; —R^{y}—SO₂H; —R^{y}—SO₂R^{x}; —R^{y}—SO₂NH₂;
—R^{y}—SO₂NHR^{x}; —R^{y}—SO₂N(R^{x})₂; —NH₂; —NHR^{x}; —N(R^{x})₂; —N⁺(R^{x})₃; —R^{y}—NH₂; —R^{y}—NHR^{x}; —R^{y}—N(R^{x})₂; —R^{y}—N⁺(R^{x})₃; —CHO; —COR^{x}; —COOH; —COOR^{x}; —OCOR^{x}; —R^{y}—CHO; —R^{y}—COR^{x}; —R^{y}—COOH; —R^{y}—COOR^{x}; or —R^{y}—OCOR^{x}; and/or
(ii) any two hydrogen atoms attached to the same carbon atom may optionally be replaced by a T-bonded substituent independently selected from oxo (═O), ═S, ═NH, or ═NR^{x}; and/or
(iii) any two hydrogen atoms attached to the same or different atoms, within the same optionally substituted group or moiety, may optionally be replaced by a bridging substituent independently selected from —O—, —S—, —NH—, —N(R^{x})—, —N⁺(R^{x})₂— or —R^{y}—;
   wherein each —R^{y}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted with one or more halo and/or —R^{x} groups; and
   wherein each —R^{x} is independently selected from a C₁-C₆ alkyl, C₂-C₆ alkenyl, C₂-C₆ alkynyl or C₂-C₆ cyclic group, or wherein any two or three-R^{x} attached to the same nitrogen atom may, together with the nitrogen atom to which they are attached, form a C₂-C₇ cyclic group, and wherein any-R^{x} may optionally be substituted with one or more C₁-C₄ alkyl, C₁-C₄ haloalkyl, —O(C₁-C₄ alkyl), —O(C₁-C₄ haloalkyl), halo, —OH, —NH₂, —CN, or oxo (═O) groups.

Typically a substituted group comprises 1, 2, 3 or 4 substituents, more typically 1, 2 or 3 substituents, more typically 1 or 2 substituents, and more typically 1 substituent.

Unless stated otherwise, any divalent bridging substituent (e.g. —O—, —S—, —NH—, —N(R^{x})—, —N⁺(R^{x})₂— or —R^{y}—) of an optionally substituted group or moiety must only be attached to the specified group or moiety and may not be attached to a second group or moiety, even if the second group or moiety can itself be optionally substituted.

The term “halo” includes fluoro, chloro, bromo and iodo.

Unless stated otherwise, where a group is prefixed by the term “halo”, such as a haloalkyl or halomethyl group, it is to be understood that the group in question is substituted with one or more halo groups independently selected from fluoro, chloro, bromo and iodo. Typically, the maximum number of halo substituents is limited only by the number of hydrogen atoms available for substitution on the corresponding group without the halo prefix. For example, a halomethyl group may contain one, two or three halo substituents. A haloethyl or halophenyl group may contain one, two, three, four or five halo substituents. Similarly, unless stated otherwise, where a group is prefixed by a specific halo group, it is to be understood that the group in question is substituted with one or more of the specific halo groups. For example, the term “fluoromethyl” refers to a methyl group substituted with one, two or three fluoro groups.

Unless stated otherwise, where a group is said to be “halo-substituted”, it is to be understood that the group in question is substituted with one or more halo groups independently selected from fluoro, chloro, bromo and iodo. Typically, the maximum number of halo substituents is limited only by the number of hydrogen atoms available for substitution on the group said to be halo-substituted. For example, a halo-substituted methyl group may contain one, two or three halo substituents. A halo-substituted ethyl or halo-substituted phenyl group may contain one, two, three, four or five halo substituents.

Unless stated otherwise, any reference to an element is to be considered a reference to all isotopes of that element. Thus, for example, unless stated otherwise any reference to hydrogen is considered to encompass all isotopes of hydrogen including deuterium and tritium.

Unless stated otherwise, any reference to a compound or group is to be considered a reference to all tautomers of that compound or group.

Where reference is made to a hydrocarbyl or other group including one or more heteroatoms N, O, S, P or Se in its carbon skeleton, or where reference is made to a carbon atom of a hydrocarbyl or other group being replaced by an N, O, S, P or Se atom, what is intended is that:

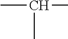

is replaced by

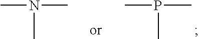

—CH₂— is replaced by —NH—, —PH—, —O—, —S— or —Se—; —CH₃ is replaced by —NH₂, —PH2, —OH, —SH or —SeH;
—CH═ is replaced by —N═ or —P═;
CH₂═ is replaced by NH═, PH═, O═, S═ or Se═; or
CH≡ is replaced by N═ or P═;
provided that the resultant group comprises at least one carbon atom. For example, methoxy, dimethylamino and aminoethyl groups are considered to be hydrocarbyl groups including one or more heteroatoms N, O, S, P or Se in their carbon skeleton.

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O, S, P or Se, are to be counted as carbon atoms when calculating the number of carbon atoms in a Cₓ-C_{y} group. For example, a morpholinyl group is to be considered a C₆ heterocyclic group, not a C₄ heterocyclic group.

The π electrons of the chlorin ring are delocalised and therefore the chlorin ring can be depicted by more than one resonance structure. Resonance structures are different ways of drawing the same compound. Two of the resonance structures of the chlorin ring are depicted directly below:

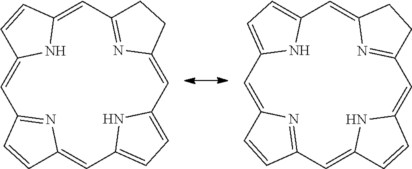 [SMILES: C1=Cc2cc3ccc(cc4nc(cc5ccc(cc1n2)[nH]5)CC4)[nH]3 ; C1=Cc2cc3ccc(cc4nc(cc5ccc(cc1n2)[nH]5)CC4)[nH]3]

Typically a complex comprises a central metal atom or ion known as the coordination centre and a bound molecule or ion which is known as a ligand. In the present specification, the bond between the coordination centre and the ligand is depicted as shown in the complex on the below left (where the attraction between an anionic ligand and a central metal cation is represented by four dashed lines), but equivalently it could be depicted as shown in the complex on the below right (where the attraction between a ligand molecule and a central metal atom is represented by two covalent bonds and two dashed lines):

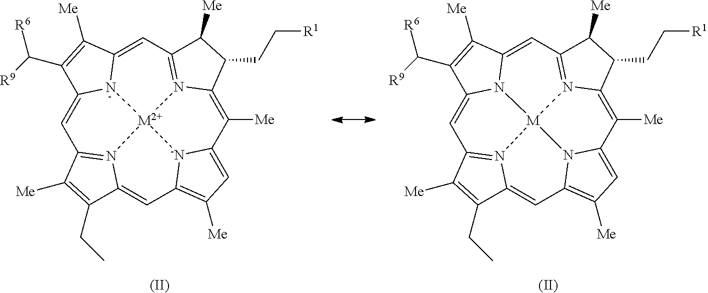

As used herein —[NC₅H₅]Y refers to:

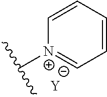

In one embodiment of the first or second aspect of the present invention, X is a halo group selected from fluoro, chloro, bromo, or iodo. In one embodiment, X is chloro or bromo.

In one embodiment of the first or second aspect of the present invention, there is provided a compound of formula (I).

In one embodiment of the first or second aspect of the present invention, Y is a counter anion selected from halides (for example fluoride, chloride, bromide, or iodide) or other inorganic anions (for example bisulfate, hexafluorophosphate (PF6), nitrate, perchlorate, phosphate, or sulfate) or organic anions (for example acetate, ascorbate, aspartate, benzoate, besylate (benzenesulfonate), bicarbonate, bis(trifluoromethanesulfonyl)imide (TFSI), bitartrate, butyrate, camsylate (camphorsulfonate), carbonate, citrate, decanoate, edetate, esylate (ethanesulfonate), fumarate, galactarate, gluceptate, gluconate, glutamate, glycolate, hexanoate, β-hydroxybutyrate, 2-hydroxyethanesulfonate, hydroxymaleate, hydroxynaphthoate, isethionate, lactate, lactobionate, malate, maleate, mandelate, mesylate (methanesulfonate), methylsulfate, mucate, napsylate (naphthalene-2-sulfonate), octanoate, oleate, ornithinate, pamoate, pantothenate, polygalacturonate, propanoate, propionate, salicylate, stearate, succinate, tartrate, teoclate, tetrakis[3,5-bis(trifluoromethyl)phenyl]borate (BARF), tetrakis(pentafluorophenyl)borate (F5-TPB), tetraphenylborate (TPB), tosylate (toluene-p-sulfonate), or triflate (trifluoromethanesulfonate)).

In another embodiment of the first or second aspect of the present invention, Y is a counter anion selected from halides (for example fluoride, chloride, bromide, or iodide) or other inorganic anions (for example bisulfate, nitrate, perchlorate, phosphate, or sulfate) or organic anions (for example acetate, aspartate, benzoate, besylate (benzenesulfonate), butyrate, camsylate (camphorsulfonate), citrate, esylate (ethanesulfonate), fumarate, galactarate, gluconate, glutamate, glycolate, 2-hydroxyethanesulfonate, hydroxymaleate, lactate, malate, maleate, mandelate, mesylate (methanesulfonate), napsylate (naphthalene-2-sulfonate), ornithinate, pamoate, pantothenate, propanoate, salicylate, succinate, tartrate, tosylate (toluene-p-sulfonate), or triflate (trifluoromethanesulfonate)). In one embodiment, Y is fluoride, chloride, bromide or iodide. In one embodiment, Y is chloride or bromide.

In one embodiment of the first or second aspect of the present invention, Z is a counter cation selected from inorganic cations (for example lithium, sodium, potassium, magnesium, calcium or ammonium cation) or organic cations (for example amine cations (for example choline or meglumine cation) or amino acid cations (for example arginine cation).

In one embodiment of the first or second aspect of the present invention, M²⁺ is a metal cation selected from Zn²⁺, Cu²⁺, Fe²⁺, Pd²⁺ or Pt²⁺. In one embodiment, M²⁺ is Zn²⁺.

In one embodiment of the first or second aspect of the present invention, —R¹ is selected from —C(O)—OR³, —C(O)—SR³, —C(O)—N(R³)₂, —C(S)—OR³, —C(S)—SR³ or —C(S)—N(R³)₂. In one embodiment, —R¹ is selected from —C(O)—OR³, —C(O)—SR³, —C(O)—N(R³)₂ or —C(S)—N(R³)₂. In one embodiment, —R¹ is selected from —C(O)—OR³, —C(O)—SR³ or —C(O)—N(R³)₂.

In one embodiment of the first or second aspect of the present invention, —R¹ is selected from —C(O)—OR³, —C(O)—SR³, —C(O)—N(R³)₂, —C(S)—OR³, —C(S)—SR³ or —C(S)—N(R³)₂, and each —R³ is C₁-C₄ alkyl (preferably methyl). In one embodiment, —R¹ is selected from —C(O)—OR³, —C(O)—SR³, —C(O)—N(R³)₂ or —C(S)—N(R³)₂, and each —R³ is C₁-C₄ alkyl (preferably methyl). In one embodiment, —R¹ is selected from —C(O)—OR³, —C(O)—SR³ or —C(O)—N(R³)₂, and each —R³ is C₁-C₄ alkyl (preferably methyl). In one embodiment, —R¹ is —C(O)—OR³ and —R³ is C₁-C₄ alkyl (preferably methyl).

In one embodiment of the first or second aspect of the present invention, —R¹ is selected from —C(O)—OR³, —C(O)—SR³, —C(O)—N(R³)₂, —C(S)—OR³, —C(S)—SR³ or —C(S)—N(R³)₂, and each —R³ is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R¹ is selected from —C(O)—OR³, —C(O)—SR³ or —C(O)—N(R³)₂, and each —R³ is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R¹ is selected from —C(O)—OR³ or —C(O)—SR³, and —R³ is selected from —R^{α}—OR^{β} or —R^{α}—SR^{β}, and —R^{β} is a saccharidyl group. Typically in these embodiments, —R^{α}— is a C₁-C₁₂ alkylene group (preferably a C₁-C₈alkylene group, or a C₁-C₆ alkylene group), a —(CH₂CH₂O)ₘ—CH₂CH₂— group or a —(CH₂CH₂S)ₘ—CH₂CH₂— group, all optionally substituted, wherein m is 1, 2, 3 or 4.

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}), wherein —R² or —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}), wherein —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R³ is H or C₁-C₄ alkyl (preferably methyl). In one embodiment, —R¹ is —C(O)—N(R³)(R^{3′}), wherein —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl (preferably methyl). In one embodiment, —R¹ is —C(O)—N(R³)(R^{3′}), wherein —R³ is selected from —R^{α}—OR^{β} or —R^{α}—SR^{β}, and —R^{β} is a saccharidyl group, and —R^{3′} is H or C₁-C₄ alkyl (preferably methyl). Typically in these embodiments, —R^{α}— is selected from a C₁-C₁₂ alkylene group, wherein one, two, three or four carbon atoms in the backbone of the alkylene group may optionally be replaced by a heteroatom or group independently selected from O, S, NH or NMe. Alternatively, in these embodiments, —R^{α}— is a C₁-C₁₂ alkylene group (preferably a C₁-C₈ alkylene group, or a C₁-C₆ alkylene group), a —(CH₂CH₂O)ₘ—CH₂CH₂— group or a —(CH₂CH₂S)ₘ—CH₂CH₂— group, all optionally substituted, wherein m is 1, 2, 3 or 4.

An —R^{3′} group refers to an —R³ group attached to the same atom as another —R³ group. —R³ and —R^{3′} may be the same or different. Preferably-R³ and —R^{3′} are different.

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}), wherein —R² or —R³ is selected from —R^{α}—R^{β} or —R^{β}, and —R^{β} is a saccharidyl group, and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}), wherein —R³ is selected from —R^{α}—R^{β} or —R^{β}, and —R^{β} is a saccharidyl group, and —R^{3′} is H or C₁-C₄ alkyl (preferably methyl). In one embodiment, —R¹ is —C(O)—N(R³)(R^{3′}), wherein —R³ is selected from —R^{α}—R^{β} or —R^{β}, and —R^{β} is a saccharidyl group, and —R^{3′} is H or C₁-C₄ alkyl (preferably methyl). Typically in these embodiments, —R^{α}— is a C₁-C₁₂ alkylene group (preferably a C₁-C₈alkylene group, or a C₁-C₆ alkylene group), a —(CH₂CH₂O)ₘ— group or a —(CH₂CH₂S)ₘ— group, all optionally substituted, wherein m is 1, 2, 3 or 4.

In any of the embodiments in the four preceding paragraphs, the saccharidyl group may optionally be substituted, for example, with a protecting group such as acetyl or a natural amino acid such as valine. Amino acids can be attached to saccharidyl groups, for example, by forming an ester between a carboxylic acid group of the amino acid and a hydroxyl group of the saccharidyl group.

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}), wherein —R² or —R³ is selected from —R^{α}—R^{β} or —R^{β}, and —R^{β} is a C₁-C₈alkyl group optionally substituted with one or more (such as one, two, three, four, five, six, seven or eight) —OH or —OAc groups, and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}), wherein —R³ is selected from —R^{α}—R^{β} or —R^{β}, and —R^{β} is a C₁-C₈alkyl group optionally substituted with one or more (such as one, two, three, four, five, six, seven or eight) hydroxyl groups, and —R^{3′} is H or C₁-C₄ alkyl (preferably methyl). In one embodiment, —R¹ is —C(O)—N(R³)(R^{3′}), wherein —R³ is selected from —R^{α}—R^{β} or —R^{β}, and —R^{β} is a C₁-C₈ alkyl group optionally substituted with one or more (such as one, two, three, four, five, six, seven or eight) hydroxyl groups, and —R^{3′} is H or C₁-C₄ alkyl (preferably methyl). Typically in these embodiments, —R^{α}— is an unsubstituted C₁-C₆ alkylene group, or an unsubstituted C₁-C₄ alkylene group, or an unsubstituted C₁-C₂ alkylene group.

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); wherein —R² or —R³ is selected from —R^{α}—H or —R^{α}—OH; —R^{α}— is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S; and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}); wherein —R³ is selected from —R^{α}—H or —R^{α}—OH; —R^{α}— is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S; and —R^{3′} is H or C₁-C₄ alkyl (preferably methyl). In one embodiment, —R¹ is —C(O)—N(R³)(R^{3′}); wherein —R³ is selected from —R^{α}—H or —R^{α}—OH; —R^{α}— is selected from a C₁-C₁₂ alkylene group, wherein one or more carbon atoms in the backbone of the alkylene group may optionally be replaced by one or more heteroatoms O or S; and —R^{3′} is H or C₁-C₄ alkyl (preferably methyl).

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); wherein —R² or —R³ is —R^{β}; —R^{β} is a C₁-C₁₂ alkyl or C₂-C₁₂ alkenyl group optionally substituted with one or more (such as one, two, three, four or five) substituents independently selected from halo, —CN, —NO₂, —N₃, —OH, —OR^{x}, —SH, —SR^{x}, —SOR^{x}, —SO₂H, —SO₂R^{x}, —SO₂NH₂, —SO₂NHR^{x}, —SO₂N(R^{x})₂, —NH₂, —NHR^{x}, —N(R^{x})₂, —N⁺(R^{x})₃, —CHO, —COR^{x}, —COOH, —COOR^{x}, —OCOR^{x}, or —NH—CO—CR^{z}—NH₂; each —R^{x} is independently selected from C₁-C₄ alkyl; —R^{z} is the side chain of a natural amino acid; and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}); wherein —R³ is —R^{β}; —R^{β} is a C₁-C₁₂ alkyl group optionally substituted with one or more (such as one, two, three, four or five) substituents independently selected from halo, —CN, —NO₂, —N₃, —OH, —OR^{x}, —SH, —SR^{x}, —SOR^{x}, —SO₂H, —SO₂R^{x}, —SO₂NH₂, —SO₂NHR^{x}, —SO₂N(R^{x})₂, —NH₂, —NHR^{x}, —N(R^{x})₂, —N⁺(R^{x})₃, —CHO, —COR^{x}, —COOH, —COOR^{x}, —OCOR^{x}, or —NH—CO—CR^{z}—NH₂; each —R^{x} is independently selected from C₁-C₄ alkyl; —R^{z} is the side chain of a natural amino acid; and —R^{3′} is H or C₁-C₄ alkyl (preferably methyl). In one embodiment, —R¹ is —C(O)—N(R³)(R^{3′}); wherein —R³ is —R^{β}; —R^{β} is a C₁-C₈ alkyl group optionally substituted with one or more (such as one, two or three) substituents independently selected from halo, —CN, —NO₂, —N₃, —OH, —OR^{x}, —SH, —SR^{x}, —SOR^{x}, —SO₂H, —SO₂R^{x}, —SO₂NH₂, —SO₂NHR^{x}, —SO₂N(R^{x})₂, —NH₂, —NHR^{x}, —N(R^{x})₂, —N⁺(R^{x})₃, —CHO, —COR^{x}, —COOH, —COOR^{x}, —OCOR^{x}, or —NH—CO—CR^{z}—NH₂; each —R^{x} is independently selected from C₁-C₄ alkyl; —R^{z} is the side chain of a natural amino acid; and —R^{3′} is H or C₁-C₄ alkyl (preferably methyl).

In one embodiment of the first or second aspect of the present invention, —R¹ is selected from —CO—(NR^{zz}—CHR^{z}—CO)ᵥ—N(R^{zz})₂ and —CO—(NR^{zz}—CHR^{z}—CO)ᵥ—OR^{zz}; wherein each —R^{z} is independently selected from the side chains of natural amino acids; each —R^{zz} is independently selected from hydrogen and C₁-C₄ alkyl (preferably methyl); and v is 1, 2, 3, 4, 5, 6, 7 or 8.

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); wherein —R² or —R³ is —R^{β}; —R^{β} is selected from a C₁-C₂₀ alkyl group, wherein the alkyl group may optionally be substituted with one, two, three or four halo groups, and wherein one, two, three, four, five or six carbon atoms in the backbone of the alkyl group may optionally be replaced by a heteroatom or group independently selected from O, S, NH or NMe; and —R^{3′} is H or C₁-C₄ alkyl (preferably methyl).

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); —R^{3′} is H or C₁-C₄ alkyl (preferably methyl); and —R² or —R³ is selected from —R^{α}—[N(R⁵)₃]Y, —R^{α}—[P(R⁵)₃]Y, or —R^{α}—[R⁷]Y. In one embodiment, —R¹ is selected from —R², —C(O)—OR³, —C(O)—SR³, —C(O)—N(R³)(R^{3′}) or —C(S)—N(R³)(R^{3′}); —R^{3′} is H or C₁-C₄ alkyl (preferably methyl); —R² or —R³ is selected from —R^{α}—[N(R⁵)₃]Y, —R^{α}—[P(R⁵)₃]Y, or —R^{α}—[R⁷]Y; each —R⁵ is independently selected from C₁-C₄ alkyl or phenyl wherein the phenyl is optionally substituted with one, two or three C₁-C₄ alkyl or C₁-C₄ alkoxy groups; —R⁷ is —[NC₅H₅] optionally substituted with one, two or three C₁-C₄ alkyl or C₁-C₄ alkoxy groups; —R^{α}— is selected from a C₁-C₁₂ alkylene group, wherein one, two, three or four carbon atoms in the backbone of the alkylene group may optionally be replaced by a heteroatom or group independently selected from O, S, NH or NMe; and Y is a counter ion (preferably a halide).

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); wherein —R² or —R³ is —R^{α}—[P(R⁵)₃]Y; each —R⁵ is independently selected from phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O), —H or —O—(CH₂CH₂O)ₙ—CH₃ groups; n is 1, 2, 3 or 4; Y is fluoride, chloride, bromide or iodide; and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}); wherein —R³ is —R^{α}—[P(R⁵)₃]Y; each —R⁵ is independently selected from phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O), —H or —O—(CH₂CH₂O)ₙ—CH₃ groups; n is 1, 2, 3 or 4; Y is fluoride, chloride, bromide or iodide; and —R^{3′} is H or C₁-C₄ alkyl (preferably methyl). In one embodiment, —R¹ is —C(O)—N(R³)(R^{3′}); wherein —R³ is —R^{α}—[P(R⁵)₃]Y; each —R⁵ is independently selected from phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups; n is 1, 2, 3 or 4; Y is fluoride, chloride, bromide or iodide; and —R^{3′} is H or C₁-C₄ alkyl (preferably methyl). Typically in these embodiments, —R^{α}— is a C₁-C₁₂ alkylene group (preferably a C₁-C₈alkylene group, or a C₁-C₆ alkylene group), a —(CH₂CH₂O)ₘ—CH₂CH₂— group or a —(CH₂CH₂S)ₘ—CH₂CH₂— group, all optionally substituted, wherein m is 1, 2, 3 or 4.

In one embodiment of the first or second aspect of the present invention, —R¹ is —C(O)—OR³, wherein —R³ is selected from hydrogen, C₁-C₄ alkyl (preferably methyl) or a cation (such as a lithium, sodium, potassium, magnesium, calcium, ammonium, amine (such as choline or meglumine), or amino acid (such as arginine) cation). In one embodiment, —R¹ is —C(O)—OR³, wherein —R³ is selected from C₁-C₄ alkyl (preferably methyl) or a cation (such as a lithium, sodium, potassium, magnesium, calcium, ammonium, amine (such as choline or meglumine), or amino acid (such as arginine) cation).

In one embodiment of the first or second aspect of the present invention, —R¹ is —C(O)—N(R³)₂. In one embodiment, —R¹ is —C(O)—N(C₁-C₄ alkyl) (R³) or —C(O)—NHR³. In one embodiment, —R¹ is —C(O)—N(CH₃)(R³) or —C(O)—NHR³. In one embodiment, —R¹ is —C(O)—N(C₁-C₄ alkyl) (R³). In one embodiment, —R¹ is —C(O)—N(CH₃)(R³).

In one embodiment of the first or second aspect of the present invention, —R¹ is selected from —CH₂OR², —CH₂SR², —CH₂S(O)R², —CH₂S(O)₂R², —CH₂N(R²)₂, or —R². In one embodiment, —R¹ is selected from —CH₂OR², —CH₂SR², —CH₂N(R²)₂, or —R². In one embodiment, —R¹ is selected from —CH₂OR², —CH₂SR², or —CH₂N(R²)₂. In one embodiment, —R¹ is selected from —CH₂OR² or —CH₂SR². In one embodiment, —R¹ is —CH₂OR². In one embodiment, —R¹ is —R², and —R² is —R^{α}—X.

In one embodiment of the first or second aspect of the present invention, —Re is selected from —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, or —R^{α}—[NC₅H₅]Y. In one embodiment, —R² is selected from —R^{α}—OR^{β}, —R^{α}—SR^{β}, —R^{α}—S(O)R^{β} or —R^{α}—S(O)₂R^{β}. In one embodiment, —R² is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R² is selected from —R^{α}—OR^{β} or —R^{α}—SR^{β}. In one embodiment, —R² is selected from —R^{α}—OR^{β} or —R^{α}—SR^{β}, and —R^{β} is a saccharidyl group.

In one embodiment of the first or second aspect of the present invention, —R² is selected from —C(O)R⁴, —C(O)—OR⁴, —C(O)—SR⁴, —C(O)—N(R⁴)₂, —C(S)—OR⁴, —C(S)—SR⁴ or —C(S)—N(R⁴)₂. In one embodiment, —R² is selected from —C(O)R⁴, —C(O)—OR⁴, —C(O)—SR⁴, —C(O)—N(R⁴)₂ or —C(S)—N(R⁴)₂. In one embodiment, —R² is selected from —C(O)R⁴, —C(O)—OR⁴, —C(O)—SR⁴ or —C(O)—N(R⁴)₂.

In one embodiment of the first or second aspect of the present invention, —R² is —C(O)—N(R⁴)(R^{4′}), wherein —R⁴ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{4′} is H or C₁-C₄ alkyl (preferably methyl). In one embodiment, —R² is —C(O)—N(R⁴)(R^{4′}), wherein —R⁴ is selected from —R^{α}—OR^{β} or —R^{α}—SR^{β}, and —R^{β} is a saccharidyl group, and —R^{4′} is H or C₁-C₄ alkyl (preferably methyl).

An —R^{4′} group refers to an —R⁴ group attached to the same atom as another —R⁴ group. —R⁴ and —R^{4′} may be the same or different. Preferably-R⁴ and —R^{4′} are different.

In one embodiment of the first or second aspect of the present invention, —R² is —C(O)—N(R⁴)₂. In one embodiment, —R² is —C(O)—N(C₁-C₄ alkyl) (R⁴). In one embodiment, —R² is —C(O)—N(CH₃)(R⁴).

In one embodiment of the first or second aspect of the present invention, —R⁶ is selected from —OR², —N(R²)₂, —SR², —S(O)R² or —S(O)₂R². In one embodiment, —R⁶ is selected from —OR², —SR², —S(O)R² or —S(O)₂R². In one embodiment, —R⁶ is selected from —OR² or —SR². In one embodiment, —R⁶ is —OR².

In one embodiment of the first or second aspect of the present invention, —R⁶ is selected from —OR², —N(R²)₂, —SR², —S(O)R² or —S(O)₂R², and —R² is selected from —H, —C(O)R⁴, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, or —R^{α}—[NC₅H₅]Y. In one embodiment, —R⁶ is selected from —OR², —SR², —S(O)R² or —S(O)₂R², and —R² is selected from —H, —C(O)R⁴, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, or —R^{α}—[NC₅H₅]Y. In one embodiment, —R⁶ is selected from —OR² or —SR², and —R² is selected from —H, —C(O)R⁴, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, or —R^{α}—[NC₅H₅]Y.

In one embodiment of the first or second aspect of the present invention, —R⁶ is selected from —OR², —N(R²)₂, —N(R²)(R^{2′}), —SR², —S(O)R² or —S(O)₂R²; —R^{2′} is selected from hydrogen or C₁-C₄ alkyl (preferably hydrogen or methyl); —R² is selected from —R^{α}—OR^{β}, —R^{α}—SR^{β}, —R^{α}—S(O)R^{β} or —R^{α}—S(O)₂R^{β}; and optionally-R^{β} is a saccharidyl group. In one embodiment, —R⁶ is selected from —OR², —SR², —S(O)R² or —S(O)₂R², and —R² is selected from —R^{α}—OR^{β}, —R^{α}—SR^{β}, —R^{α}—S(O)R^{β} or —R^{α}—S(O)₂R^{β}, and optionally-R^{β} is a saccharidyl group. In one embodiment, —R⁶ is selected from —OR², —SR², —S(O)R² or —S(O)₂R², and —R² is selected from —R^{α}—OR^{β} or —R^{α}—SR^{β}, and optionally-R^{β} is a saccharidyl group. In one embodiment, —R⁶ is selected from —OR² or —SR², and —R² is selected from —R^{α}—OR^{β}, —R^{α}—SR^{β}, —R^{α}—S(O)R^{β} or —R^{α}—S(O)₂R^{β}, and optionally-R^{β} is a saccharidyl group. In one embodiment, —R⁶ is selected from —OR² or —SR², and —R² is selected from —R^{α}—OR^{β} or —R^{α}—SR^{β}, and optionally-R^{β} is a saccharidyl group.

In one embodiment of the first or second aspect of the present invention, —R⁶ is selected from —OR², —N(R²)₂, —N(R²)(R^{2′}), —SR², —S(O)R² or —S(O)₂R²; —R^{2′} is selected from hydrogen or C₁-C₄ alkyl (preferably hydrogen or methyl); and —R² is —C(O)R⁴. In one embodiment, —R⁶ is selected from —OR², —N(R²)₂, —N(R²)(R^{2′}), —SR², —S(O)R² or —S(O)₂R²; —R^{2′} is selected from hydrogen or C₁-C₄ alkyl (preferably hydrogen or methyl); —R² is —C(O)R⁴; —R⁴ is selected from —R^{α}—OR^{β}, —R^{α}—SR^{β}, —R^{α}—S(O)R^{β} or —R^{α}—S(O)₂R^{β}; and —R^{β} is a saccharidyl group. In one embodiment, —R⁶ is selected from —OR², —N(R²)₂, —N(R²)(R^{2′}), —SR², —S(O)R² or —S(O)₂R²; —R^{2′} is selected from hydrogen or C₁-C₄ alkyl (preferably hydrogen or methyl); —R² is —C(O)R⁴; —R⁴ is selected from —R^{α}—OR^{β} or —R^{α}—SR^{β}; and —R^{β} is a saccharidyl group.

In one embodiment of the first or second aspect of the present invention, —R⁶ is selected from —OR², —SR², —S(O)R² or —S(O)₂R², and —R² is —C(O)R⁴. In one embodiment, —R⁶ is selected from —OR², —SR², —S(O)R² or —S(O)₂R², and —R² is —C(O)R⁴, and —R⁴ is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R⁶ is selected from —OR², —SR², —S(O)R² or —S(O)₂R², and —R² is —C(O)R⁴, and —R⁴ is selected from —R^{α}—OR^{β} or —R^{α}—SR^{β}, and —R^{β} is a saccharidyl group.

In one embodiment of the first or second aspect of the present invention, —R⁶ is selected from —OR² or —SR², and —R² is —C(O)R⁴. In one embodiment, —R⁶ is selected from —OR² or —SR², and —R² is —C(O)R⁴, and —R⁴ is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R⁶ is selected from —OR² or —SR², and —R² is —C(O)R⁴, and —R⁴ is selected from —R^{α}—OR^{β} or —R^{α}—SR^{β}, and —R^{β} is a saccharidyl group.

In one embodiment of the first or second aspect of the present invention, —R⁶ is selected from —OR², —N(R²)₂, —N(R²)(R^{2′}), —SR², —S(O)R² or —S(O)₂R²; —R^{2′} is selected from hydrogen or C₁-C₄ alkyl (preferably hydrogen or methyl); —R² is selected from —R^{β}—R^{α}—OR^{β}, —R^{α}—SR^{β}, —R^{α}—S(O)R^{β} or —R^{α}—S(O)₂R^{β}; —R^{β} is a saccharidyl group; and —R^{α}— is selected from a C₁-C₁₂ alkylene group, wherein one, two, three or four carbon atoms in the backbone of the alkylene group may optionally be replaced by a heteroatom or group independently selected from O, S, NH or NMe. In one embodiment, —R⁶ is selected from —OR², —N(R²)(R^{2′}) or —SR²; —R^{2′} is selected from hydrogen or C₁-C₄ alkyl (preferably hydrogen or methyl); —R² is selected from —R^{β}, —R^{α}—OR^{β} or —R^{α}—SR^{β}; —RP is a saccharidyl group; and —R^{α}— is selected from a C₁-C₁₂ alkylene group, wherein one, two, three or four carbon atoms in the backbone of the alkylene group may optionally be replaced by a heteroatom or group independently selected from O, S, NH or NMe.

In any of the embodiments in the five preceding paragraphs, the saccharidyl group may optionally be substituted, for example, with a protecting group such as acetyl or a natural amino acid such as valine. Amino acids can be attached to saccharidyl groups, for example, by forming an ester between a carboxylic acid group of the amino acid and a hydroxyl group of the saccharidyl group.

In one embodiment of the first or second aspect of the present invention, —R⁶ is selected from —OR², —N(R²)₂, —N(R²)(R^{2′}), —SR², —S(O)R² or —S(O)₂R²; —R^{2′} is selected from hydrogen, C₁-C₄ alkyl or —CO₂ (C₁-C₄ alkyl); —R² is selected from —C(O)R⁴, —C(O)—OR⁴, —C(O)—N(R⁴)(R^{4′}), —R^{α}—[N(R⁵)₃]Y, —R^{α}—[P(R⁵)₃]Y, or —R^{α}—[R⁷]Y; —R^{4′} is selected from hydrogen or C₁-C₄ alkyl; and —R⁴ is selected from —R^{α}—[N(R⁵)₃]Y, —R^{α}—[P(R⁵)₃]Y, or —R^{α}—[R⁷]Y. In one embodiment, —R⁶ is selected from —OR², —N(R²)(R^{2′}), —SR², —S(O)R² or —S(O)₂R²; —R^{2′} is selected from hydrogen, C₁-C₄ alkyl or —CO₂ (C₁-C₄ alkyl); —R² is selected from —C(O)R⁴, —C(O)—OR⁴, —C(O)—N(R⁴)(R^{4′}), —R^{α}—[N(R⁵)₃]Y, —R^{α}—[P(R⁵)₃]Y, or —R^{α}—[R⁷]Y; —R^{4′} is selected from hydrogen or C₁-C₄ alkyl; —R⁴ is selected from —R^{α}—[N(R⁵)₃]Y, —R^{α}—[P(R⁵)₃]Y, or —R^{α}—[R⁷]Y; each —R⁵ is independently selected from C₁-C₄ alkyl or phenyl wherein the phenyl is optionally substituted with one, two or three C₁-C₄ alkyl or C₁-C₄ alkoxy groups; —R⁷ is —[NC₅H₅] optionally substituted with one, two or three C₁-C₄ alkyl or C₁-C₄ alkoxy groups; —R^{α}— is selected from a C₁-C₁₂ alkylene group, wherein one, two, three or four carbon atoms in the backbone of the alkylene group may optionally be replaced by a heteroatom or group independently selected from O, S, NH or NMe; and Y is a counter ion (preferably a halide). In one embodiment, —R⁶ is selected from —OR² or —N(R²)(R^{2′}); —R^{2′} is selected from hydrogen, C₁-C₄ alkyl or —CO₂ (C₁-C₄ alkyl); —R² is selected from —C(O)R⁴, —C(O)—OR⁴, —C(O)—N(R⁴)(R^{4′}), —R^{α}—[N(R⁵)₃]Y, —R^{α}—[P(R⁵)₃]Y, or —R^{α}—[R⁷]Y; —R^{4′} is selected from hydrogen or C₁-C₄ alkyl; —R⁴ is selected from —R^{α}—[N(R⁵)₃]Y, —R^{α}—[P(R⁵)₃]Y, or —R^{α}—[R⁷]Y; each —R⁵ is independently selected from C₁-C₄ alkyl or phenyl wherein the phenyl is optionally substituted with one, two or three C₁-C₄ alkyl or C₁-C₄ alkoxy groups; —R⁷ is —[NC₅H₅] optionally substituted with one, two or three C₁-C₄ alkyl or C₁-C₄ alkoxy groups; —R^{α}— is selected from a C₁-C₁₂ alkylene group, wherein one, two, three or four carbon atoms in the backbone of the alkylene group may optionally be replaced by a heteroatom or group independently selected from O, S, NH or NMe; and Y is a counter ion (preferably a halide).

In one embodiment of the first or second aspect of the present invention, —R⁶ is selected from —OR², —N(R²)₂, —SR², —S(O)R² or —S(O)₂R²; and —R² is selected from hydrogen, C₁-C₄ alkyl, —CO(C₁-C₄ alkyl) or —CO₂ (C₁-C₄ alkyl). In one embodiment, —R⁶ is selected from —OR² or —N(R²)₂; and —R² is selected from hydrogen, C₁-C₄ alkyl, —CO(C₁-C₄ alkyl) or —CO₂ (C₁-C₄ alkyl).

In one embodiment of the first or second aspect of the present invention, —R⁶ is selected from —OR², —N(R²)₂, —N(R²)(R^{2′}), —SR², —S(O)R² or —S(O)₂R²; —R^{2′} is selected from hydrogen or C₁-C₄ alkyl; —R² is selected from —R⁴, —C(O)R⁴, —C(O)—OR⁴ or —C(O)—N(R⁴)(R^{4′}); —R^{4′} is selected from hydrogen or C₁-C₄ alkyl; and —R⁴ is selected from a C₁-C₁₂ alkyl group, wherein the alkyl group may optionally be substituted with one, two, three or four halo groups, and wherein one, two, three or four carbon atoms in the backbone of the alkyl group may optionally be replaced by a heteroatom or group independently selected from O, S, NH or NMe. In one embodiment, —R⁶ is selected from —OR² or —N(R²)(R^{2′}); —R^{2′} is selected from hydrogen or C₁-C₄ alkyl; —R² is selected from —R⁴, —C(O)R⁴, —C(O)—OR⁴ or —C(O)—N(R⁴)(R^{4′}); —R^{4′} is selected from hydrogen or C₁-C₄ alkyl; and —R⁴ is selected from a C₁-C₁₂ alkyl group, wherein the alkyl group may optionally be substituted with one, two, three or four halo groups, and wherein one, two, three or four carbon atoms in the backbone of the alkyl group may optionally be replaced by a heteroatom or group independently selected from O, S, NH or NMe.

In one embodiment of the first or second aspect of the present invention, —R⁹ is hydrogen or methyl. In one embodiment, —R⁹ is methyl. In a preferred embodiment, —R⁹ is hydrogen.

In one embodiment of the first or second aspect of the present invention, —R⁶ and —R⁹ together form an oxo (═O) group.

In one embodiment of the first or second aspect of the present invention, each —R^{α}— is independently a C₁-C₁₂ alkylene group, a —(CH₂CH₂O)ₘ— group, a —(CH₂CH₂S)ₘ— group, a —(CH₂CH₂O)ₘ—CH₂CH₂— group or a —(CH₂CH₂S)ₘ—CH₂CH₂— group, all optionally substituted, wherein m is 1, 2, 3 or 4. In one embodiment, each —R^{α}— is independently a C₁-C₁₂ alkylene group, a —(CH₂CH₂O)ₘ— group or a —(CH₂CH₂S)ₘ— group, all optionally substituted, wherein m is 1, 2, 3 or 4. In one embodiment, each —R^{α}— is independently a C₁-C₁₂ alkylene group or a —(CH₂CH₂O)ₘ— group, both optionally substituted, wherein m is 1, 2, 3 or 4. In one embodiment, each —R^{α}— is independently an optionally substituted-(CH₂CH₂O)ₘ— group, wherein m is 1, 2, 3 or 4.

In one embodiment of the first or second aspect of the present invention, each —R^{α}— is independently a C₁-C₈alkylene group, or a C₁-C₆ alkylene group, or a C₂-C₄ alkylene group, all optionally substituted.

In one embodiment of the first or second aspect of the present invention, each —R^{α}— is independently unsubstituted or substituted with one or more substituents independently selected from halo, C₁-C₄ alkyl, or C₁-C₄ haloalkyl. In one embodiment, each —R^{α}— is independently unsubstituted or substituted with one or two substituents independently selected from halo, C₁-C₄ alkyl, or C₁-C₄ haloalkyl. In one embodiment, each —R^{α}— is unsubstituted.

In one embodiment of the first or second aspect of the present invention, each —R^{β} is independently a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton.

In one embodiment of the first or second aspect of the present invention, at least one —R^{β} is independently a C₁-C₆ alkyl group, or a C₁-C₄ alkyl group, or a methyl group, all optionally substituted. In one embodiment, each —R^{β} is independently a C₁-C₆ alkyl group, or a C₁-C₄ alkyl group, or a methyl group, all optionally substituted.

In one embodiment of the first or second aspect of the present invention, at least one —RP is independently a saccharidyl group. In one embodiment, each —R^{β} is independently a saccharidyl group.

In one embodiment of the first or second aspect of the present invention, each —R^{β} is independently unsubstituted or substituted with one or more substituents independently selected from halo, C₁-C₄ alkyl, or C₁-C₄ haloalkyl. In one embodiment, each —R^{β} is independently unsubstituted or substituted with one or two substituents independently selected from halo, C₁-C₄ alkyl, or C₁-C₄ haloalkyl. In one embodiment, each —R^{β} is unsubstituted.

In one embodiment of the first or second aspect of the present invention, each —R³ is independently selected from —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, or —R^{α}—[NC₅H₅]Y. In one embodiment, each —R³ is independently selected from —R^{α}—OR^{β}, —R^{α}—SR^{β}, —R^{α}—S(O)R^{β} or —R^{α}—S(O)₂R^{β}. In one embodiment, each —R³ is independently selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, each —R³ is independently selected from —R^{α}—OR^{β} or —R^{α}—SR^{β}. In one embodiment, each —R³ is independently selected from —R^{α}—OR^{β} or —R^{α}—SR^{β}, and —R^{β} is a saccharidyl group.

In one embodiment of the first or second aspect of the present invention, each —R⁴ is independently selected from —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, or —R^{α}—[NC₅H₅]Y. In one embodiment, each —R⁴ is independently selected from —R^{α}—OR^{β}, —R^{α}—SR^{β}, —R^{α}—S(O)R^{β} or —R^{α}—S(O)₂R^{β}. In one embodiment, each —R⁴ is independently selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, each —R⁴ is independently selected from —R^{α}—OR^{β} or —R^{α}—SR^{β}. In one embodiment, each —R⁴ is independently selected from —R^{α}—OR^{β} or —R^{α}—SR^{β}, and —R^{β} is a saccharidyl group.

In one embodiment of the first or second aspect of the present invention, at least one of —R², —R³ or —R⁴ is independently selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, at least one of —R², —R³ or —R⁴ is independently selected from —R^{α}—OR^{β} or —R^{α}—SR^{β}, and —R^{β} is a saccharidyl group.

For the purposes of the present invention, a “saccharidyl group” is any group comprising at least one monosaccharide subunit, wherein each monosaccharide subunit may optionally be substituted and/or modified. Typically, a saccharidyl group consist of one or more monosaccharide subunits, wherein each monosaccharide subunit may optionally be substituted and/or modified.

Typically, a carbon atom of a single monosaccharide subunit of each saccharidyl group is directly attached to the remainder of the compound, most typically via a single bond.

For the purposes of the present specification, where it is stated that a first atom or group is “directly attached” to a second atom or group it is to be understood that the first atom or group is covalently bonded to the second atom or group with no intervening atom(s) or group(s) being present. For example, for the group —(C═O)N(CH₃)₂, the carbon atom of each methyl group is directly attached to the nitrogen atom and the carbon atom of the carbonyl group is directly attached to the nitrogen atom, but the carbon atom of the carbonyl group is not directly attached to the carbon atom of either methyl group.

Typically, each saccharidyl group is derived from the corresponding saccharide by substitution of a hydroxyl group of the saccharide with the group defined by the remainder of the compound.

A single bond between an anomeric carbon of a monosaccharide subunit and a substituent is called a glycosidic bond. A glycosidic group is linked to the anomeric carbon of a monosaccharide subunit by a glycosidic bond. The bond between the saccharidyl group and the remainder of the compound may be a glycosidic or a non-glycosidic bond. Typically, the bond between the saccharidyl group and the remainder of the compound is a glycosidic bond, such that the saccharidyl group is a glycosyl group. Where the bond between the saccharidyl group and the remainder of the compound is a glycosidic bond, the glycosidic bond may be in the α or β configuration. Typically, such a glycosidic bond is in the β configuration.

For the purposes of the present invention, where a saccharidyl group “contains x monosaccharide subunits”, this means that the saccharidyl group has x monosaccharide subunits and no more. In contrast, where a saccharidyl group “comprises x monosaccharide subunits”, this means that the saccharidyl group has x or more monosaccharide subunits.

Each saccharidyl group may be independently selected from a monosaccharidel, disaccharidyl, oligosaccharidyl or polysaccharidyl group. As will be understood, a monosaccharidel group contains a single monosaccharide subunit. Similarly, a disaccharidyl group contains two monosaccharide subunits. As used herein, an “oligosaccharidyl group” contains from 2 to 9 monosaccharide subunits. Examples of oligosaccharidyl groups include trisaccharidyl, tetrasaccharidyl, pentasaccharidyl, hexasaccharidyl, heptasaccharidyl, octasaccharidyl and nonasaccharidyl groups. As used herein, a “polysaccharidyl group” contains 10 or more monosaccharide subunits (such as 10-50, or 10-30, or 10-20, or 10-15 monosaccharide subunits).

Each monosaccharide subunit within a disaccharidyl, oligosaccharidyl or polysaccharidyl group may be the same or different. Each monosaccharide subunit within a disaccharidyl, oligosaccharidyl or polysaccharidyl group may be connected to another monosaccharide subunit within the group via a glycosidic or a non-glycosidic bond. Typically each monosaccharide subunit within a disaccharidyl, oligosaccharidyl or polysaccharidyl group is connected to another monosaccharide subunit within the group via a glycosidic bond, which may be in the a or β configuration.

Each oligosaccharidyl or polysaccharidyl group may be a linear, branched or macrocyclic oligosaccharidyl or polysaccharidyl group. Typically, each oligosaccharidyl or polysaccharidyl group is a linear or branched oligosaccharidyl or polysaccharidyl group.

In one embodiment, at least one —R^{β} is a monosaccharidel or disaccharidyl group.

In a further embodiment, at least one —R^{β} is a monosaccharidel group. For example, at least one —R^{β} may be a glycosyl group containing a single monosaccharide subunit, wherein the monosaccharide subunit may optionally be substituted and/or modified. Typically at least one —R^{β} is a glycosyl group containing a single monosaccharide subunit, wherein the monosaccharide subunit may optionally be substituted. More typically, at least one —R^{β} is a glycosyl group containing a single monosaccharide subunit, wherein the monosaccharide subunit is unsubstituted.

In one embodiment, at least one —R^{β} is an aldosyl group, wherein the aldosyl group may optionally be substituted and/or modified. For example, at least one —R^{β} may be selected from a glycerosyl, aldotetrosyl (such as erythrosyl or threosyl), aldopentosyl (such as ribosyl, arabinosyl, xylosyl or lyxosyl) or aldohexosyl (such as allosyl, altrosyl, glucosyl, mannosyl, gulosyl, idosyl, galactosyl or talosyl) group, any of which may optionally be substituted and/or modified.

In another embodiment, at least one —R^{β} is a ketosyl group, wherein the ketosyl group may optionally be substituted and/or modified. For example, at least one —R^{β} may be selected from an erythrulosyl, ketopentosyl (such as ribulosyl or xylulosyl) or ketohexosyl (such as psicosyl, fructosyl, sorbosyl or tagatosyl) group, any of which may optionally be substituted and/or modified.

Each monosaccharide subunit may be present in a ring-closed (cyclic) or open-chain (acyclic) form. Typically, each monosaccharide subunit in at least one —R^{β} is present in a ring-closed (cyclic) form. For example, at least one —R^{β} may be a glycosyl group containing a single ring-closed monosaccharide subunit, wherein the monosaccharide subunit may optionally be substituted and/or modified. Typically in such a scenario, at least one —R^{β} is a pyranosyl or furanosyl group, such as an aldopyranosyl, aldofuranosyl, ketopyranosyl or ketofuranosyl group, any of which may optionally be substituted and/or modified. More typically, at least one —R^{β} is a pyranosyl group, such as an aldopyranosyl or ketopyranosyl group, any of which may optionally be substituted and/or modified.

In one embodiment, at least one —R^{β} is selected from a ribopyranosyl, arabinopyranosyl, xylopyranosyl, lyxopyranosyl, allopyranosyl, altropyranosyl, glucopyranosyl, mannopyranosyl, gulopyranosyl, idopyranosyl, galactopyranosyl or talopyranosyl group, any of which may optionally be substituted and/or modified.

In a further embodiment, at least one —R^{β} is a glucosyl group, such as a glucopyranosyl group, wherein the glucosyl or the glucopyranosyl group may optionally be substituted and/or modified. Typically, at least one —R^{β} is a glucosyl group, wherein the glucosyl group is optionally substituted. More typically, at least one —R^{β} is an unsubstituted glucosyl group.

Each monosaccharide subunit may be present in the D- or L-configuration. Typically, each monosaccharide subunit is present in the configuration in which it most commonly occurs in nature.

In one embodiment, at least one —R^{β} is a D-glucosyl group, such as a D-glucopyranosyl group, wherein the D-glucosyl or the D-glucopyranosyl group may optionally be substituted and/or modified. Typically, at least one —R^{β} is a D-glucosyl group, wherein the D-glucosyl group is optionally substituted. More typically, at least one —R^{β} is an unsubstituted D-glucosyl group.

For the purposes of the present invention, in a substituted monosaccharidel group or monosaccharide subunit:
(a) one or more of the hydroxyl groups of the monosaccharidel group or monosaccharide subunit are each independently replaced with —H, —F, —Cl, —Br, —I, —CF₃, —CCl₃, —CBr₃, —Cl₃, —SH, —NH₂, —N₃, —NH═NH₂, —CN, —NO₂, —COOH, —R^{b}, —O—R^{b}, —S—R^{b}, —R^{a}—O—R^{b}, —R^{a}—S—R^{b}, —SO—R^{b}, —SO₂—R^{b}, —SO₂—OR^{b}, —O—SO—R^{b}, —O—SO₂—R^{b}, —O—SO₂—OR^{b}, —NR^{b}—SO—R^{b}, —NR^{b}—SO₂—R^{b}, —NR^{b}—SO₂—OR^{b}, —R^{a}—SO—R^{b}, —R^{a}—SO₂—R^{b}, —R^{a}—SO₂—OR^{b}, —SO—N(R^{b})₂, —SO₂—N(R^{b})₂, —O—SO—N(R^{b})₂, —O—SO₂—N(R^{b})₂, —NR^{b}—SO—N(R^{b})₂, —NR^{b}—SO₂—N(R^{b})₂, —R^{a}—SO—N(R^{b})₂, —R^{a}—SO₂—N(R^{b})₂, —N(R^{b})₂, —N(R^{b})₃⁺, —R^{a}—N(R^{b})₂, —R^{a}—N(R^{b})₃⁺, —P(R^{b})₂, —PO(R^{b})₂, —OP(R^{b})₂, —OPO(R^{b})₂, —R^{a}—P(R^{b})₂, —R^{a}—PO(R^{b})₂, —OSi(R^{b})₃, —R^{a}—Si(R^{b})₃, —CO—R^{b}, —CO—OR^{b}, —CO—N(R^{b})₂, —O—CO—R^{b}, —O—CO—OR^{b}, —O—CO—N(R^{b})₂, —NR^{b}—CO—R^{b}, —NR^{b}—CO—OR^{b}, —NR^{b}—CO—N(R^{b})₂, —R^{a}—CO—R^{b}, —R^{a}—CO—OR^{b}, or —R^{a}—CO—N(R^{b})₂; and/or
(b) one, two or three hydrogen atoms directly attached to a carbon atom of the monosaccharidel group or monosaccharide subunit are each independently replaced with —F, —Cl, —Br, —I, —CF₃, —CCl₃, —CBr₃, —Cl₃, —OH, —SH, —NH₂, —N₃, —NH═NH₂, —CN, —NO₂, —COOH, —R^{b}, —O—R^{b}, —S—R^{b}, —R^{α}—O—R^{b}, —R^{α}—S—R^{b}, —SO—R^{b}, —SO₂—R^{b}, —SO₂—OR^{b}, —O—SO—R^{b}, —O—SO₂—R^{b}, —O—SO₂—OR^{b}, —NR^{b}—SO—R^{b}, —NR^{b}—SO₂—R^{b}, —NR^{b}—SO₂—OR^{b}, —R^{a}—SO—R^{b}, —R^{a}—SO₂—R^{b}, —R^{a}—SO₂—OR^{b}, —SO—N(R^{b})₂, —SO₂—N(R^{b})₂, —O—SO—N(R^{b})₂, —O—SO₂—N(R^{b})₂, —NR^{b}—SO—N(R^{b})₂, —NR^{b}—SO₂—N(R^{b})₂, —R^{a}—SO—N(R^{b})₂, —R^{a}—SO₂—N(R^{b})₂, —N(R^{b})₂, —N(R^{b})₃⁺, —R^{a}—N(R^{b})₂, —R^{a}—N(R^{b})₃⁺, —P(R^{b})₂, —PO(R^{b})₂, —OP(R^{b})₂, —OPO(R^{b})₂, —R^{a}—P(R^{b})₂, —R^{a}—PO(R^{b})₂, —OSi(R^{b})₃, —R^{a}—Si(R^{b})₃, —CO—R^{b}, —CO—OR^{b}, —CO—N(R^{b})₂, —O—CO—R^{b}, —O—CO—OR^{b}, —O—CO—N(R^{b})₂, —NR^{b}—CO—R^{b}, —NR^{b}—CO—OR^{b}, —NR^{b}—CO—N(R^{b})₂, —R^{a}—CO—R^{b}, —R^{a}—CO—OR^{b}, or —R^{a}—CO—N(R^{b})₂; and/or
(c) one or more of the hydroxyl groups of the monosaccharidel group or monosaccharide subunit, together with the hydrogen attached to the same carbon atom as the hydroxyl group, are each independently replaced with ═O, ═S, ═NR^{b}, or ═N(R^{b})₂⁺; and/or
(d) any two hydroxyl groups of the monosaccharidel group or monosaccharide subunit are together replaced with —O—R^{c}—, —S—R^{c}—, —SO—R^{c}—, —SO₂—R^{c}—, or —NR^{b}—R^{c}—;
wherein:
   each —R^{a} is independently a substituted or unsubstituted alkylene, alkenylene or alkynylene group which optionally includes one or more heteroatoms each independently selected from O, N and S in its carbon skeleton and preferably comprises 1-10 carbon atoms;
   each —R^{b} is independently hydrogen, or a substituted or unsubstituted, straight-chained, branched or cyclic alkyl, alkenyl, alkynyl, aryl, arylalkyl, arylalkenyl, arylalkynyl, alkylaryl, alkenylaryl or alkynylaryl group which optionally includes one or more heteroatoms each independently selected from O, N and S in its carbon skeleton and preferably comprises 1-15 carbon atoms; and
   each —R^{c}— is independently a chemical bond, or a substituted or unsubstituted alkylene, alkenylene or alkynylene group which optionally includes one or more heteroatoms each independently selected from O, N and S in its carbon skeleton and preferably comprises 1-10 carbon atoms;
   provided that the monosaccharidel group or monosaccharide subunit comprises at least one, preferably at least two or at least three, —OH, —O—R^{b}, —O—SO—R^{b}, —O—SO₂—R^{b}, —O—SO₂—OR^{b}, —O—SO—N(R^{b})₂, —O—SO₂—N(R^{b})₂, —OP(R^{b})₂, —OPO(R^{b})₂, —OSi(R^{b})₃, —O—CO—R^{b}, —O—CO—OR^{b}, —O—CO—N(R^{b})₂, or —O—R^{c}—.

Typically, in a substituted monosaccharidel group or monosaccharide subunit:
(a) one or more of the hydroxyl groups of the monosaccharidel group or monosaccharide subunit are each independently replaced with —H, —F, —CF₃, —SH, —NH₂, —N₃, —CN, —NO₂, —COOH, —R^{b}, —O—R^{b}, —S—R^{b}, —N(R^{b})₂, —OPO(R^{b})₂, —OSi(R^{b})₃, —O—CO—R^{b}, —O—CO—OR^{b}, —O—CO—N(R^{b})₂, —NR^{b}—CO—R^{b}, —NR^{b}—CO—OR^{b}, or —NR^{b}—CO—N(R^{b})₂; and/or
(b) one or two of the hydrogen atoms directly attached to a carbon atom of the monosaccharidel group or monosaccharide subunit are each independently replaced with —F, —CF₃, —OH, —SH, —NH₂, —N₃, —CN, —NO₂, —COOH, —R^{b}, —O—R^{b}, —S—R^{b}, —N(R^{b})₂, —OPO(R^{b})₂, —OSi(R^{b})₃, —O—CO—R^{b}, —O—CO—OR^{b}, —O—CO—N(R^{b})₂, —NR^{b}—CO—R^{b}, —NR^{b}—CO—OR^{b}, or —NR^{b}—CO—N(R^{b})₂; and/or
(c) one hydroxyl group of the monosaccharidel group or monosaccharide subunit, together with the hydrogen attached to the same carbon atom as the hydroxyl group, is replaced with ═O; and/or
(d) any two hydroxyl groups of the monosaccharidel group or monosaccharide subunit are together replaced with —O—R^{c}— or —NR^{b}—R^{c}—;
wherein:
   each —R^{b} is independently hydrogen, or a substituted or unsubstituted, straight-chained, branched or cyclic alkyl, alkenyl, alkynyl, aryl, arylalkyl, arylalkenyl, arylalkynyl, alkylaryl, alkenylaryl or alkynylaryl group which optionally includes one, two or three heteroatoms each independently selected from O and N in its carbon skeleton and comprises 1-8 carbon atoms; and
   each —R^{c}— is independently a substituted or unsubstituted alkylene, alkenylene or alkynylene group which optionally includes one, two or three heteroatoms each independently selected from O and N in its carbon skeleton and comprises 1-8 carbon atoms;
   provided that the monosaccharidel group or monosaccharide subunit comprises at least two, preferably at least three, —OH, —O—R^{b}, —OPO(R^{b})₂, —OSi(R^{b})₃, —O—CO—R^{b}, —O—CO—OR^{b}, —O—CO—N(R^{b})₂, or —O—R^{c}—.

In one embodiment, —R^{β} is a saccharidyl group and one or more of the hydroxyl groups of the saccharidyl group are each independently replaced with —O—CO—R^{b}, wherein each —R^{b} is independently C₁-C₄ alkyl, preferably methyl. In one embodiment, —R^{β} is a saccharidyl group and all of the hydroxyl groups of the saccharidyl group are each independently replaced with —O—CO—R^{b}, wherein each —R^{b} is independently C₁-C₄ alkyl, preferably methyl.

In a modified monosaccharidel group or monosaccharide subunit:
(a) the ring of the modified monosaccharidel group or monosaccharide subunit, or what would be the ring in the ring-closed form of the modified monosaccharidel group or monosaccharide subunit, is partially unsaturated; and/or
(b) the ring oxygen of the modified monosaccharidel group or monosaccharide subunit, or what would be the ring oxygen in the ring-closed form of the modified monosaccharidel group or monosaccharide subunit, is replaced with —S— or —NR^{d}—, wherein —R^{d} is independently hydrogen, or a substituted or unsubstituted, straight-chained, branched or cyclic alkyl, alkenyl, alkynyl, aryl, arylalkyl, arylalkenyl, arylalkynyl, alkylaryl, alkenylaryl or alkynylaryl group which optionally includes one or more heteroatoms each independently selected from O, N and S in its carbon skeleton and preferably comprises 1-15 carbon atoms.

Alternately, where the modified monosaccharide subunit forms part of a disaccharidyl, oligosaccharidyl or polysaccharidyl group, —R^{d} may be a further monosaccharide subunit or subunits forming part of the disaccharidyl, oligosaccharidyl or polysaccharidyl group, wherein any such further monosaccharide subunit or subunits may optionally be substituted and/or modified.

Typically, in a modified monosaccharidel group or monosaccharide subunit:
(a) the ring of the modified monosaccharidel group or monosaccharide subunit, or what would be the ring in the ring-closed form of the modified monosaccharidyl group or monosaccharide subunit, contains a single C═C; and/or
(b) the ring oxygen of the modified monosaccharidel group or monosaccharide subunit, or what would be the ring oxygen in the ring-closed form of the modified monosaccharidel group or monosaccharide subunit, is replaced with —NR^{d}—, wherein —R^{d} is independently hydrogen, or a substituted or unsubstituted, straight-chained, branched or cyclic alkyl, alkenyl, alkynyl, aryl, arylalkyl, arylalkenyl, arylalkynyl, alkylaryl, alkenylaryl or alkynylaryl group which optionally includes one, two or three heteroatoms each independently selected from O and N in its carbon skeleton and comprises 1-8 carbon atoms.

Typical examples of substituted and/or modified monosaccharide subunits include those corresponding to:
(i) deoxy sugars, such as deoxyribose, fucose, fuculose and rhamnose, wherein a hydroxyl group of the monosaccharidel group or monosaccharide subunit has been replaced by —H;
(ii) amino sugars, such as glucosamine and galactosamine, wherein a hydroxyl group of the monosaccharidel group or monosaccharide subunit has been replaced by —NH₂, most typically at the 2-position; and
(iii) sugar acids, containing a —COOH group, such as aldonic acids (e.g. gluconic acid), ulosonic acids, uronic acids (e.g. glucuronic acid) and aldaric acids (e.g. gularic or galactaric acid).

In one embodiment of the first or second aspect of the present invention, at least one —R^{β} is a monosaccharidel group selected from:

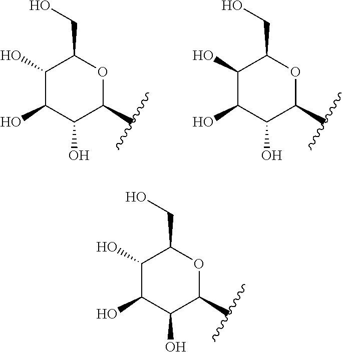 [SMILES: *[C@@H]1O[C@H](CO)[C@@H](O)[C@H](O)[C@H]1O ; *[C@@H]1O[C@H](CO)[C@H](O)[C@H](O)[C@H]1O ; *[C@@H]1O[C@H](CO)[C@@H](O)[C@H](O)[C@@H]1O]

Preferably in the compound or complex according to the first or second aspect of the present invention, at least one —R^{β} is:

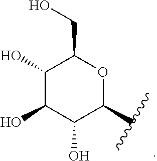 [SMILES: *[C@@H]1O[C@H](CO)[C@@H](O)[C@H](O)[C@H]1O]

In one embodiment of the first or second aspect of the present invention, at least one of —R², —R³ or —R⁴ is independently selected from —R^{α}—OR^{β}, —R^{α}—SR^{β}, —R^{α}—S(O)R^{β} or —R^{α}—S(O)₂R^{β} (preferably from —R^{α}—OR^{β} or —R^{α}—SR^{β}), and —R^{β} is selected from:

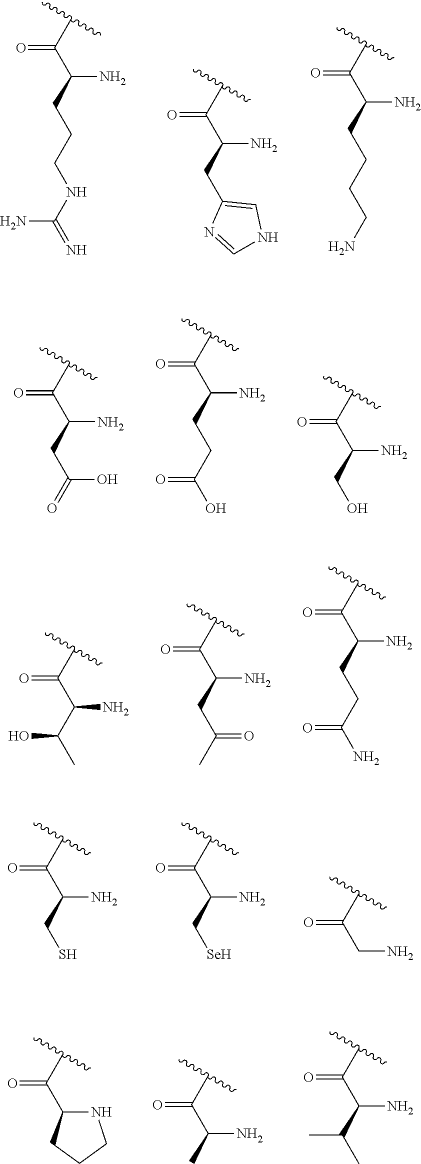

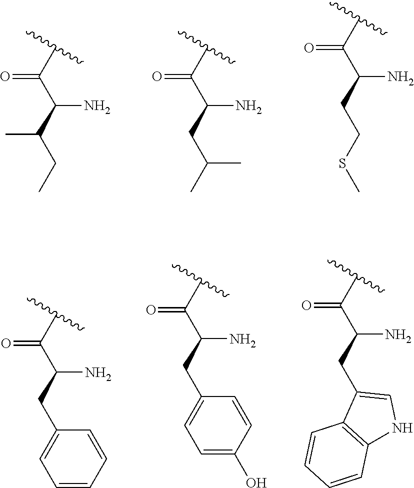

In one embodiment of the first or second aspect of the present invention, at least one of —R², —R³ or —R⁴ is independently selected from —R^{α}—[N(R⁵)₃]Y, —R^{α}—[P(R⁵)₃]Y, —R^{α}—[R⁷]Y, —R^{α}—[N(R⁵)₂ (R^{5′})], —R^{α}—[P(R⁵)₂ (R^{5′})], or —R^{α}—[R⁷]. In one embodiment, at least one of —R², —R³ or —R⁴ is independently selected from —R^{α}—[N(R⁵)₃]Y, —R^{α}—[P(R⁵)₃]Y, or —R^{α}—[R⁷]Y. In one embodiment, at least one of —R², —R³ or —R⁴ is independently selected from:

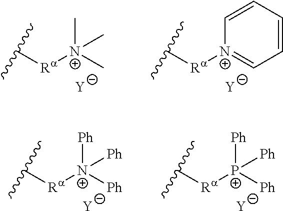

In the first or second aspect of the present invention, each —R⁵ may be the same or different. In a preferred embodiment, each —R⁵ is the same.

In one embodiment of the first or second aspect of the present invention, each —R⁵ is independently unsubstituted or substituted with one or two substituents. In one embodiment, each —R⁵ is unsubstituted.

In one embodiment of the first or second aspect of the present invention, —R⁷ is unsubstituted or substituted with one or two substituents. In one embodiment, —R⁷ is unsubstituted.

In one embodiment, —R⁷ is not substituted at the 4-position of the pyridine ring with a halo group. In one embodiment, —R⁷ is unsubstituted at the 4-position of the pyridine ring. In one embodiment, —R⁷ is unsubstituted.

In one embodiment of the first or second aspect of the present invention, each of —R¹ and —R⁶ independently comprises from 1 to 100 atoms other than hydrogen, preferably from 1 to 80 atoms other than hydrogen, preferably from 1 to 60 atoms other than hydrogen, preferably from 1 to 50 atoms other than hydrogen, and preferably from 1 to 45 atoms other than hydrogen.

In a particularly preferred embodiment, the first or second aspect of the present invention provides a compound of formula (I) or a complex of formula (II):

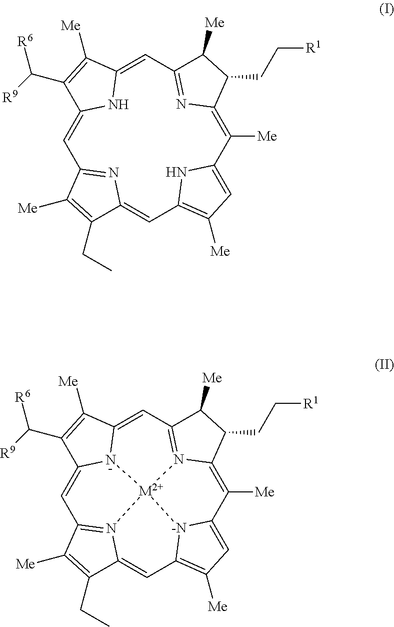

or a pharmaceutically acceptable salt thereof, wherein:
—R¹ is selected from:
(a) —C(O)—OR³, —C(O)—SR³ or —C(O)—N(R³)₂, and R³, each independently, is —R^{β}, and —R^{β} is a C₁-C₄ alkyl group, more preferably, R¹ is —C(O)—OR³ and R³ is —R^{β}, and —R^{β} is a C₁-C₄ alkyl group; or
(b) —C(O)—OR³, —C(O)—SR³ or —C(O)—N(R³)(R^{3′}), R³ is selected from —R^{α}—OR^{β} or —R^{α}—SR^{β} and —R^{β} is a saccharidyl group, and R^{3′} is H or C₁-C₄ alkyl;
—R⁶ is selected from —OR² or —SR², and —R² is selected from —R^{α}—OR^{β} or —R^{α}—SR^{β}, and —R^{β} is a saccharidyl group;
—R^{α}—, each independently, is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S;
R⁹ is hydrogen or methyl (preferably —R⁹ is hydrogen); and
M²⁺ is a metal cation.

In a particularly preferred embodiment, the first or second aspect of the present invention provides a compound of formula (I) or a complex of formula (II):

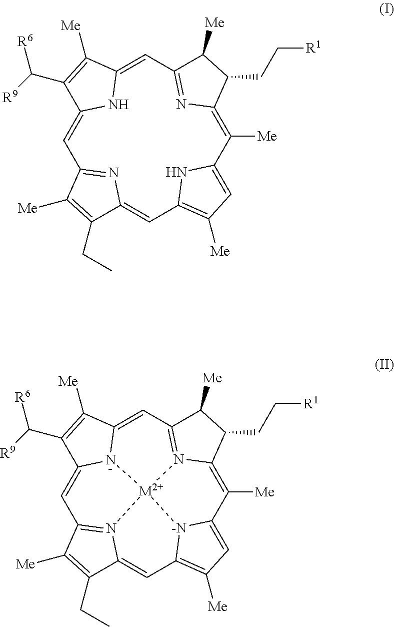

or a pharmaceutically acceptable salt thereof, wherein:
—R¹ is selected from —C(O)—OR³, —C(O)—SR³ or —C(O)—N(R³)₂, and R³, each independently, is —R^{β}, and —R^{β} is a C₁-C₄ alkyl group, more preferably, R¹ is —C(O)—OR³ and R³ is —R^{β}, and —R^{β} is a C₁-C₄ alkyl group;
—R⁶ is selected from —OR² or —SR², and —R² is selected from —R^{α}—OR^{β} or —R^{α}—SR^{β}, and —R^{β} is a saccharidyl group;
—R^{α}— is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S;
R⁹ is hydrogen or methyl (preferably —R⁹ is hydrogen); and
M²⁺ is a metal cation.

In a particularly preferred embodiment the first or second aspect of the present invention provides a compound of formula (I) or a complex of formula (II):

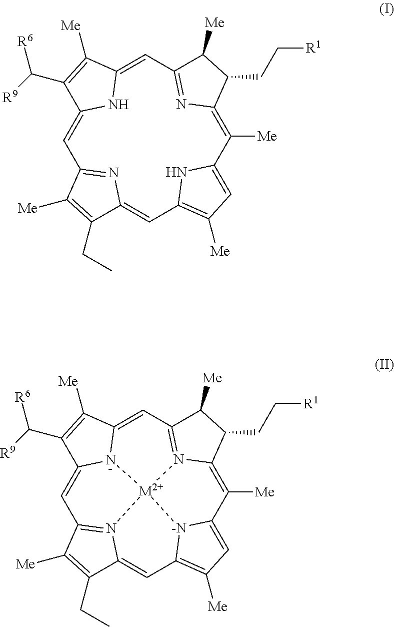

or a pharmaceutically acceptable salt thereof, wherein:
—R¹ is selected from —C(O)—OR³, —C(O)—SR³ or —C(O)—N(R³)(R^{3′}), R³ is selected from —R^{α}—OR^{β} or —R^{α}—SR^{β} and —R^{β} is a saccharidyl group, and R^{3′} is H or C₁-C₄ alkyl;
—R⁶ is selected from —OR² or —SR², and —R² is selected from —R^{α}—OR^{β} or —R^{α}—SR^{β}, and —R^{β} is a saccharidyl group;
—R^{α}— is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S;
—R⁹ is hydrogen or methyl (preferably —R⁹ is hydrogen); and
M²⁺ is a metal cation.

In a particularly preferred embodiment, the first or second aspect of the present invention provides a compound of formula (I) or a complex of formula (II):

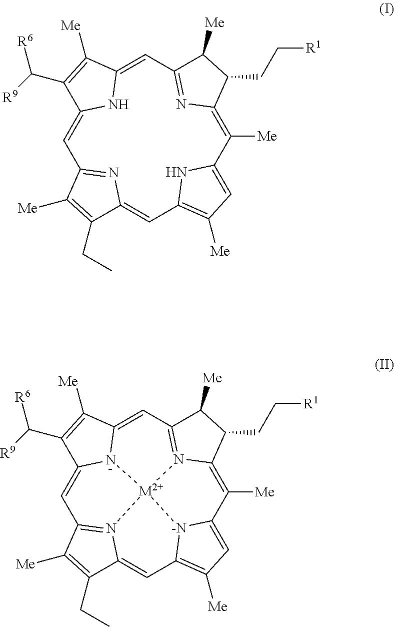

or a pharmaceutically acceptable salt thereof, wherein:
—R¹ is selected from —CH₂OR², —CH₂SR², —CH₂S(O)R², —CH₂S(O)₂R², —CH₂N(R²)(R^{2′}), —R², —C(O)—OR³, —C(O)—SR³, —C(O)—N(R³)(R^{3′}), —C(S)—OR³, —C(S)—SR³ or —C(S)—N(R³)(R^{3′}) [preferably —R¹ is selected from —C(O)—OR³, —C(O)—SR³, —C(O)—N(R³)(R^{3′}), —C(S)—OR³, —C(S)—SR³ or —C(S)—N(R³)(R^{3′}); more preferably —R¹ is —C(O)—N(R³)(R^{3′})];
—R², each independently, is selected from —H, —C(O)R⁴, —C(O)—OR⁴, —C(O)—SR⁴, —C(O)—N(R⁴)(R^{4′}), —C(S)—OR⁴, —C(S)—SR⁴, —C(S)—N(R⁴)(R^{4′}), —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(RP) 2, —R^{α}—X, —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, [(CH₂)ₚQ]ᵣ—(CH₂)ₛ—[R⁷]Y, —[(CH₂)ₚQ]ᵣ—(CH₂)ₛ—[N(R⁵)₂ (R^{5′})], [(CH₂)ₚQ]ᵣ—(CH₂)ₛ—[P(R⁵)₂ (R^{5′})] or —[(CH₂)ₚQ]ᵣ—(CH₂)ₛ—[R⁷];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, [(CH₂)ₚQ]ᵣ—(CH₂)ₛ—[R⁷]Y, —[(CH₂)ₚQ]ᵣ—(CH₂)ₛ—[N(R⁵)₂ (R^{5′})], [(CH₂)ₚQ]ᵣ—(CH₂)ₛ—[P(R⁵)₂ (R^{5′})] or —[(CH₂)ₚQ]ᵣ—(CH₂)ₛ—[R⁷];
wherein at least one of —R², —R³ and —R⁴ is selected from [(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]—(CH₂)ₛ—[R⁷]Y, [(CH₂)ₚQ]ᵣ—(CH₂)ₛ—[N(R⁵)₂ (R^{5′})], —[(CH₂)ₚQ]ᵣ—(CH₂)ₛ—[P(R⁵)₂ (R^{5′})] or [(CH₂)ₚQ]ᵣ—(CH₂)ₛ—[R⁷];
—R^{2′}, —R^{3′} and —R^{4′}, each independently, is selected from hydrogen or C₁-C₆ alkyl [preferably —R^{2′}, —R^{3′} and —R^{4′}, each independently, is selected from hydrogen or C₁-C₃ alkyl; more preferably —R^{2′}, —R^{3′} and —R^{4′}, each independently, is selected from hydrogen or methyl];
—R^{α}—, each independently, is selected from a C₁-C₄₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O), —H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂, wherein the phenyl or C₅-C₆ heteroaryl may optionally be further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from —OR², —N(R²)₂, —SR², —S(O)R², —S(O)₂R², or —X [preferably —R⁶ is selected from —OR², —SR², —S(O)R², —S(O)₂R², or —X];
—R⁷ is —[NC₅H₅] optionally substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁷ is —[NC₅H₅] substituted with —CO₂ and optionally further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O), —CH₃ groups;
—R⁹ is hydrogen or methyl [preferably —R⁹ is hydrogen];
Q is O, S, NH or NMe [preferably Q is O];
X is a halo group;
Y is a counter anion;
Z is a counter cation;
M²⁺ is a metal cation;
n is 1, 2, 3, 4, 5 or 6;
p is 0, 1, 2, 3 or 4;
r is 0, 1, 2, 3, 4, 5 or 6; and
s is 0, 1, 2, 3, 4, 5, 6, 7, 8, 9, 10, 11 or 12.

In a particularly preferred embodiment, the first or second aspect of the present invention provides a compound of formula (I) or a complex of formula (II):

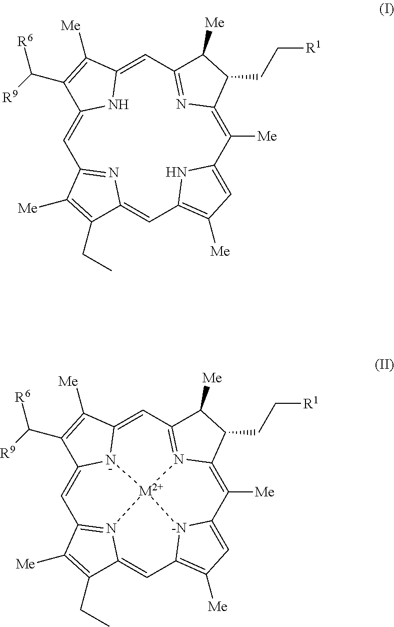

or a pharmaceutically acceptable salt thereof, wherein:
—R¹ is selected from —C(O)—OR³, —C(O)—SR³, —C(O)—N(R³)(R^{3′}), —C(S)—OR³, —C(S)—SR³ or —C(S)—N(R³)(R^{3′}) [preferably —R¹ is —C(O)—N(R³)(R^{3′})];
—R² is selected from —H, —C(O)R⁴, —C(O)—OR⁴, —C(O)—SR⁴, —C(O)—N(R⁴)(R^{4′}), —C(S)—OR⁴, —C(S)—SR⁴, —C(S)—N(R⁴)(R^{4′}), —R^{α}—H, —RP, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]—(CH₂)ₛ—[R⁷]Y, —[(CH₂)ₚQ]ᵣ—(CH₂)ₛ—[N(R⁵)₂ (R^{5′})], —[(CH₂)ₚQ]ᵣ—(CH₂)ₛ—[P(R⁵)₂ (R^{5′})] or —[(CH₂)ₚQ]ᵣ—(CH₂)ₛ—[R⁷];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁷]Y, —[(CH₂)ₚQ]ᵣ—(CH₂)ₛ—[N(R⁵)₂ (R^{5′})], —[(CH₂)ₚQ]ᵣ—(CH₂)ₛ—[P(R⁵)₂ (R^{5′})] or —[(CH₂)ₚQ]ᵣ—(CH₂)ₛ—[R⁷];
wherein at least one of —R², —R³ and —R⁴ is selected from —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁷]Y, —[(CH₂)ₚQ]ᵣ—(CH₂)ₛ—[N(R⁵)₂ (R^{5′})], —[(CH₂)ₚQ]ᵣ—(CH₂)ₛ—[P(R⁵)₂ (R^{5′})] or —[(CH₂)ₚQ]ᵣ—(CH₂)ₛ—[R⁷];
—R^{3′} and —R^{4′}, each independently, is selected from hydrogen or C₁-C₃ alkyl [preferably —R^{3′} and —R^{4′}, each independently, is selected from hydrogen or methyl];
—R^{α}—, each independently, is selected from a C₁-C₄₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R⁵, each independently, is selected from C₁-C₃ alkyl or phenyl, wherein the phenyl may optionally be substituted with one, two, three, four or five substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R^{5′} is selected from C₁-C₃ alkyl substituted with —CO₂ or phenyl substituted with —CO₂, wherein the phenyl may optionally be further substituted with one, two, three or four substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R⁶ is selected from —OR², —N(R²)₂, —SR², —S(O)R², —S(O)₂R², or —X [preferably —R⁶ is selected from —OR², —SR², —S(O)R², —S(O)₂R², or —X];
—R⁷ is —[NC₅H₅] optionally substituted with one, two, three, four or five substituents independently selected from C₁-C₆ alkyl, —O(C₁-C₆ alkyl), —CO₂H, —CO₂Z, —CO₂NH₂, —O—(CH₂CH₂O), —H or —O—(CH₂CH₂O)ₙ—CH₃;
—R^{7′} is —[NC₅H₅] substituted with —CO₂ and optionally further substituted with one, two, three or four substituents independently selected from C₁-C₆ alkyl, —O(C₁-C₆ alkyl), —CO₂H, —CO₂Z, —CO₂NH₂, —O—(CH₂CH₂O)—H or —O—(CH₂CH₂O)ₙ—CH₃;
—R⁹ is hydrogen or methyl [preferably —R⁹ is hydrogen];
Q is O, S, NH or NMe [preferably Q is O];
X is a halo group;
Y is a counter anion;
Z is a counter cation;
M²⁺ is a metal cation;
n is 1, 2, 3, 4, 5 or 6;
p is 0, 1, 2, 3 or 4;
r is 0, 1, 2, 3, 4, 5 or 6; and
s is 0, 1, 2, 3, 4, 5, 6, 7, 8, 9, 10, 11 or 12.

In these two preferred embodiments of the preceding paragraphs, each —R⁵ may be the same or different; preferably each —R⁵ is the same.

In another preferred embodiment of the first or second aspect of the present invention, the compound is a compound of formula (IA), (IB), (IC), (ID), (IE), (IF), (IG), (IH), (IJ), (IK), (IL), (IM), (IN) or (IO):

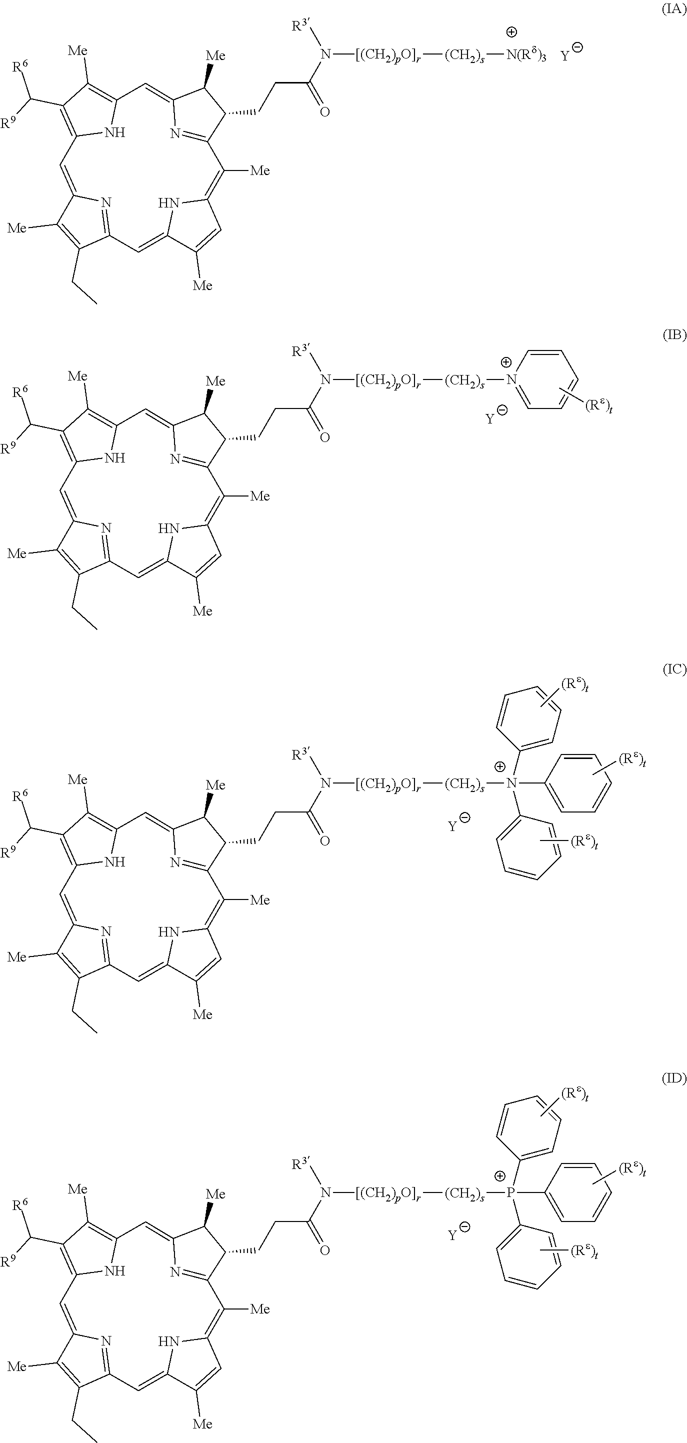

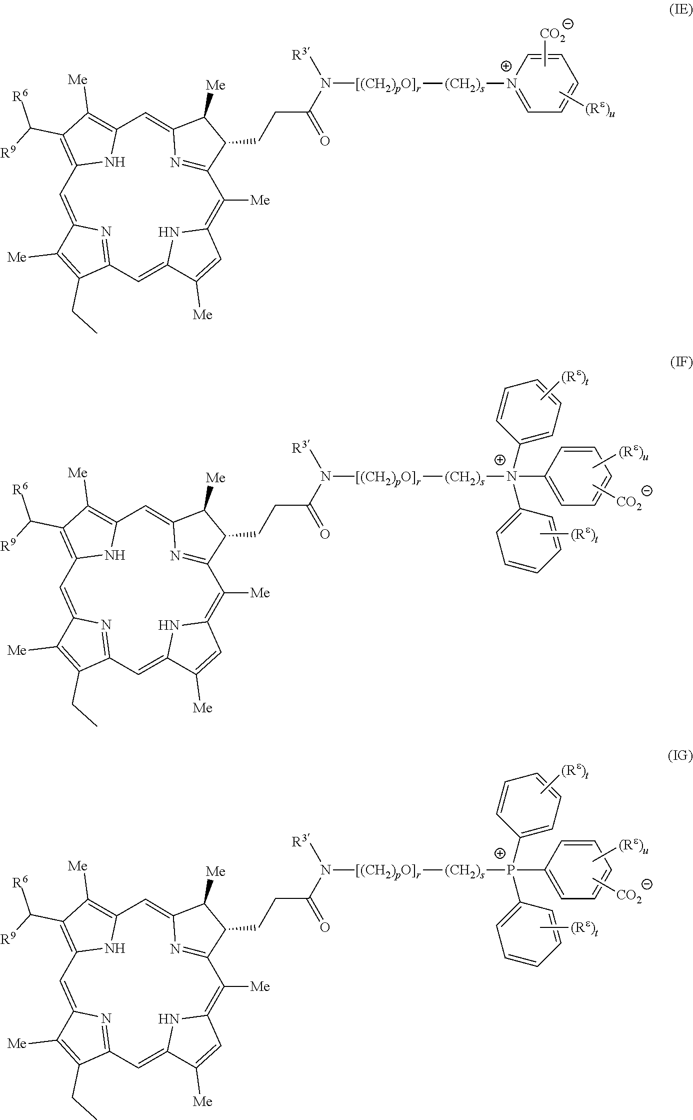

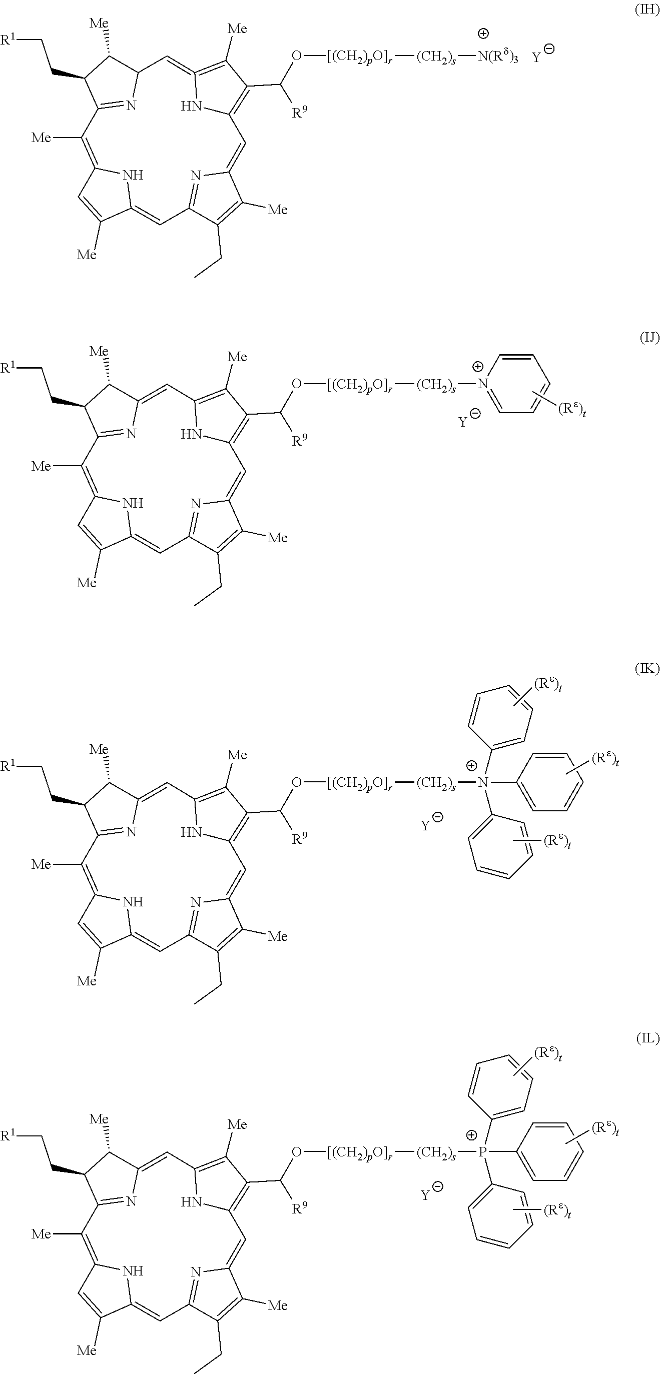

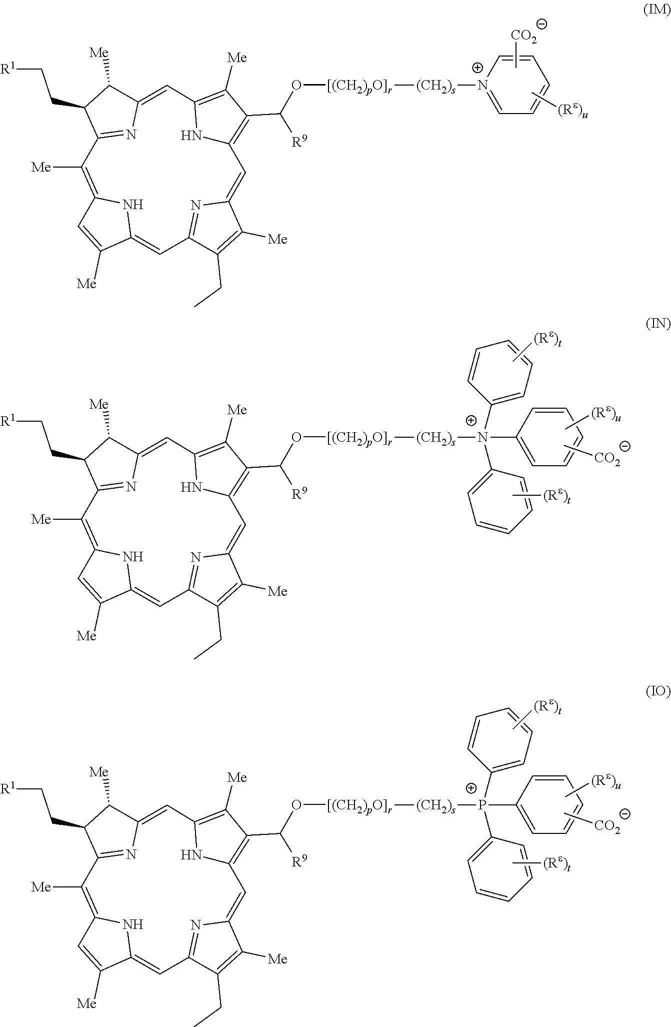

or a metal cation complex thereof, or a pharmaceutically acceptable salt thereof, wherein:
—R¹ is selected from —C(O)—OR³, —C(O)—SR³, —C(O)—N(R³)(R³), —C(S)—OR³, —C(S)—SR³ or —C(S)—N(R³)(R^{3′});
—R² is selected from —H, —C(O)R⁴, —C(O)—OR⁴, —C(O)—SR⁴, —C(O)—N(R⁴)(R^{4′}), —C(S)—OR⁴, —C(S)—SR⁴, —C(S)—N(R⁴)(R^{4′}), —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, or —R^{α}—X;
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, or —R^{α}—X;
—R^{3′} and —R^{4′}, each independently, is selected from hydrogen or C₁-C₃ alkyl [preferably —R^{3′} and —R^{4′}, each independently, is selected from hydrogen or methyl];
—R⁶ is selected from —OR², —N(R²)₂, —SR², —S(O)R², —S(O)₂R², or —X [preferably —R⁶ is selected from —OR², —SR², —S(O)R², —S(O)₂R², or —X];
R⁹ is hydrogen or methyl [preferably —R⁹ is hydrogen];
—R^{α}—, each independently, is selected from a C₁-C₁₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R^{δ} is selected from C₁-C₃ alkyl;
—R^{ε} is selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
X is a halo group;
Y is a counter anion;
Z is a counter cation;
n is 1, 2, 3 or 4;
p is 0, 1, 2, 3 or 4;
r is 0, 1, 2, 3, 4, 5 or 6;
s is 0, 1, 2, 3, 4, 5, 6, 7, 8, 9, 10, 11 or 12;
tis 0, 1, 2, 3, 4 or 5; and
u is 0, 1, 2, 3 and 4.

The compounds of formula (IA), (IB), (IC), (ID), (IE), (IF), (IG), (IH), (IJ), (IK), (IL), (IM), (IN), (IO) and complexes and salts thereof according to the first and second aspect of the present invention comprise a moiety —[(CH₂)ₚO]ᵣ—(CH₂)ₛ—, wherein:
p is 0, 1, 2, 3 or 4;
r is 0, 1, 2, 3, 4, 5 or 6; and
s is 0, 1, 2, 3, 4, 5, 6, 7, 8, 9, 10, 11 or 12.

In one embodiment, p is 2, 3 or 4; r is 1; and s is 2, 3 or 4. In a preferred embodiment, p is 3; r is 1; and s is 3; such that —[(CH₂)ₚO]ᵣ—(CH₂)ₛ— is —(CH₂)₃—O—(CH₂)₃—.

In another embodiment, p is 2 or 3; r is 2 or 3; and s is 2 or 3. In a preferred embodiment, p is 2; r is 2; and s is 2; such that —[(CH₂)ₚO], —(CH₂)ₛ— is —(CH₂CH₂O)₂—(CH₂)₂—.

In yet another embodiment, r is 0; and s is 1, 2, 3, 4, 5, 6, 7, 8, 9, 10, 11 or 12; such that —[(CH₂)ₚO]ᵣ—(CH₂)ₛ— is —(CH₂)₁₋₁₂—.

In another preferred embodiment, the first or second aspect of the present invention provides a compound of formula (I′) or a complex of formula (II′):

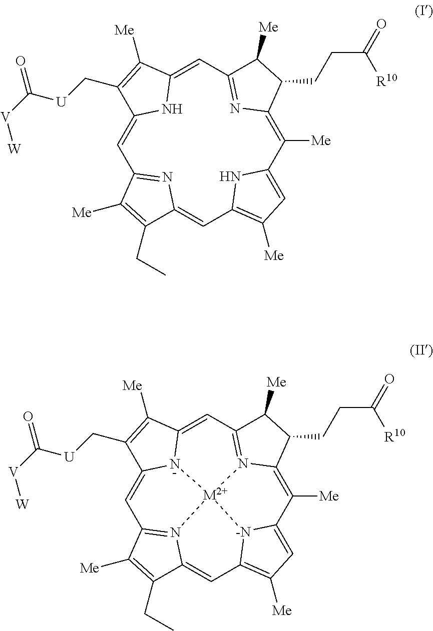

or a pharmaceutically acceptable salt thereof, wherein:
-U- is —O—, —N(R^{u})— or —S—;
-V- is —CH₂—, —O—, —N(R^{v})— or —S—;
—W— is —R^{α}—[N(R⁵)₃]Y, —R^{α}—[P(R⁵)₃]Y, —R^{α}—[R⁷]Y, —R^{α}—[N(R⁵)₂ (R^{5′})], —R^{α}—[P(R⁵)₂ (R^{5′})] or —R^{α}—[R⁷];
—R¹⁰ is selected from —OH or —O—(C₁-C₄ alkyl);
—R^{α}— is selected from a C₁-C₁₂ alkylene group, wherein the alkylene group may optionally be substituted with one or more (such as one, two, three or four) C₁-C₄ alkyl, C₁-C₄ haloalkyl or halo groups, and wherein one or more (such as one, two, three or four) carbon atoms in the backbone of the alkylene group may optionally be replaced by a heteroatom or group independently selected from O, S, NH or NMe;
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂, wherein the phenyl or C₅-C₆ heteroaryl may optionally be further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O), —H or —O—(CH₂CH₂O)ₙ—CH₃ groups;
—R⁷ is —[NC₅H₅] optionally substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{7′} is —[NC₅H₅] substituted with —CO₂ and optionally further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O), —H or —O—(CH₂CH₂O)ₙ—CH₃ groups;
—R^{u} is hydrogen or C₁-C₄ alkyl;
—R^{v} is hydrogen or C₁-C₄ alkyl;
n is 1, 2, 3, 4, 5 or 6;
Y is a counter anion;
Z is a counter cation; and
M²⁺ is a metal cation.

In another preferred embodiment, the first or second aspect of the present invention provides a compound of formula (I′) or a complex of formula (II′):

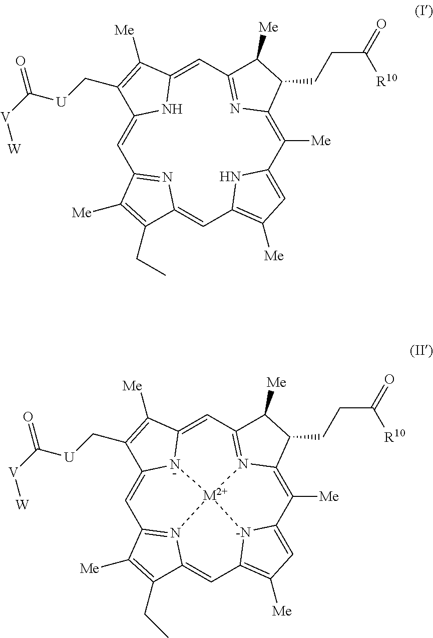

or a pharmaceutically acceptable salt thereof, wherein:
-U- is —O—, —N(R^{u})— or —S—;
-V- is —CH₂—, —O—, —N(R^{v})— or —S—;
—W— is —R^{α}—[N(R⁵)₃]Y, —R^{α}—[P(R⁵)₃]Y or —R^{α}—[R⁷]Y;
—R¹⁰ is selected from —OH or —O—(C₁-C₄ alkyl);
—R^{α}— is selected from a C₁-C₁₂ alkylene group (preferably a C₁-C₉ alkylene group, preferably a C₂-C₆ alkylene group), wherein one or more (such as one, two, three or four, preferably one or two) carbon atoms in the backbone of the alkylene group may optionally be replaced by a heteroatom or group independently selected from O, S, NH or NMe (preferably O, NH or NMe, preferably O);
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl) or —O(C₁-C₄ haloalkyl);
—R⁷ is —[NC₅H₅] optionally substituted with one or more C₁-C₄ alkyl, C₁-C₄ haloalkyl, —O(C₁-C₄ alkyl) or —O(C₁-C₄ haloalkyl);
—R^{u} is hydrogen or C₁-C₄ alkyl;
—R^{v} is hydrogen or C₁-C₄ alkyl;
n is 1, 2, 3, 4, 5 or 6;
Y is a counter anion; and
M²⁺ is a metal cation.

Preferably in the compound or complex according to the first or second aspect of the present invention, the compound or complex is:

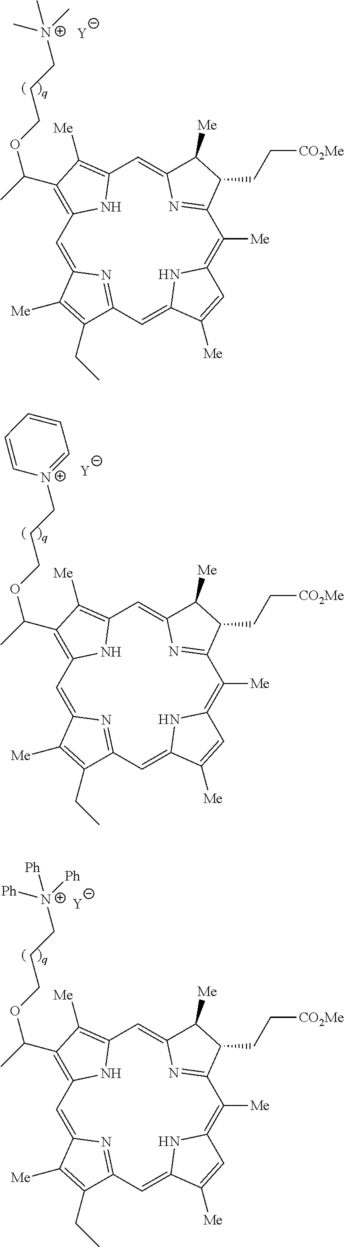

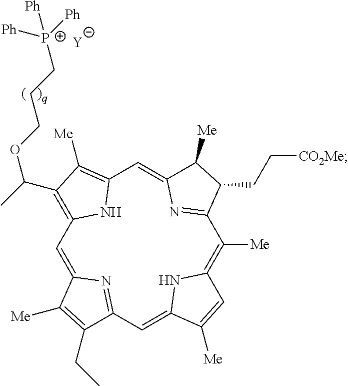

wherein Y is a counter anion, and q is 0, 1, 2, 3 or 4 (preferably q is 1);
or a metal cation complex thereof, or a pharmaceutically acceptable salt thereof.

Preferably in the compound or complex according to the first or second aspect of the present invention, the compound or complex is:

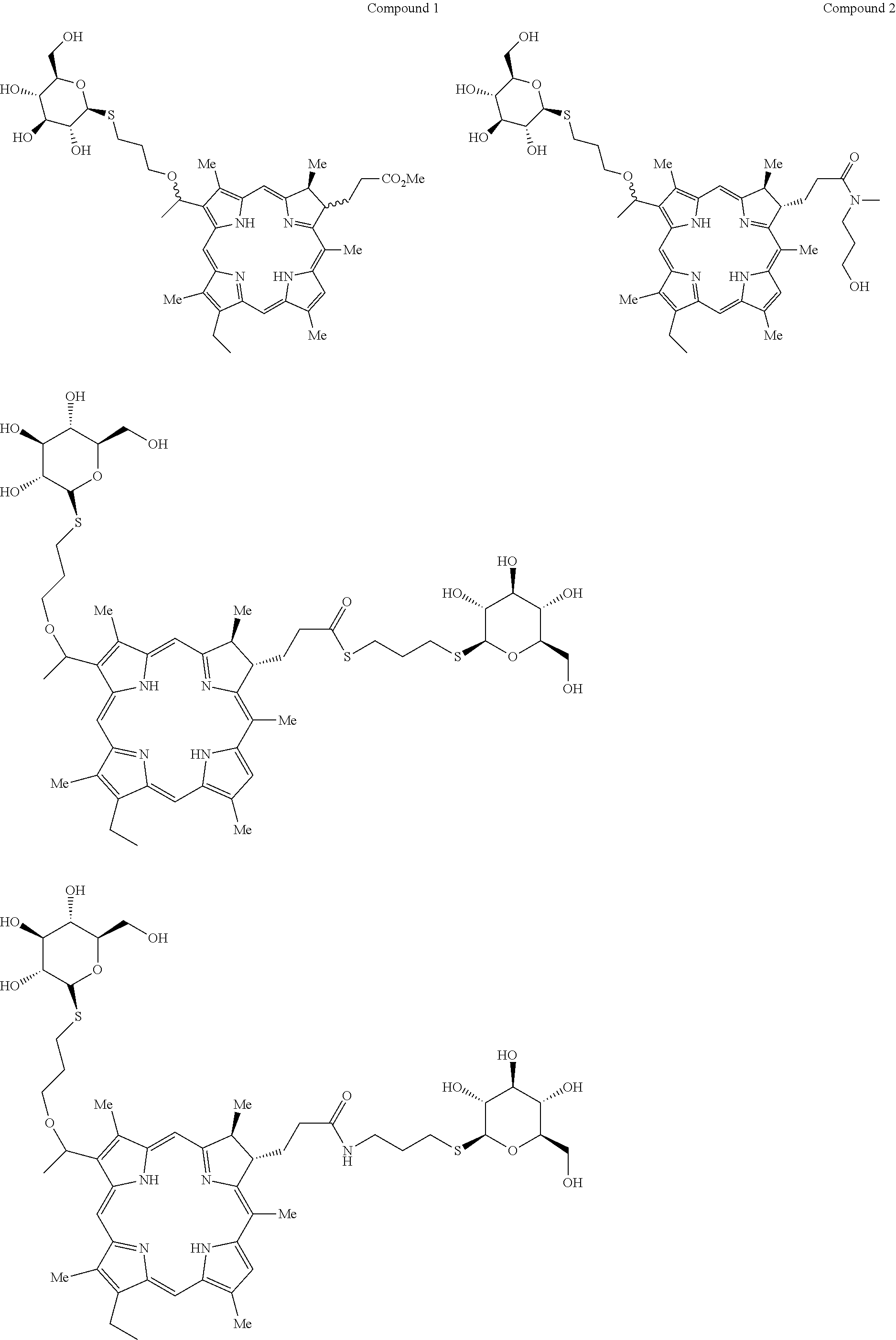

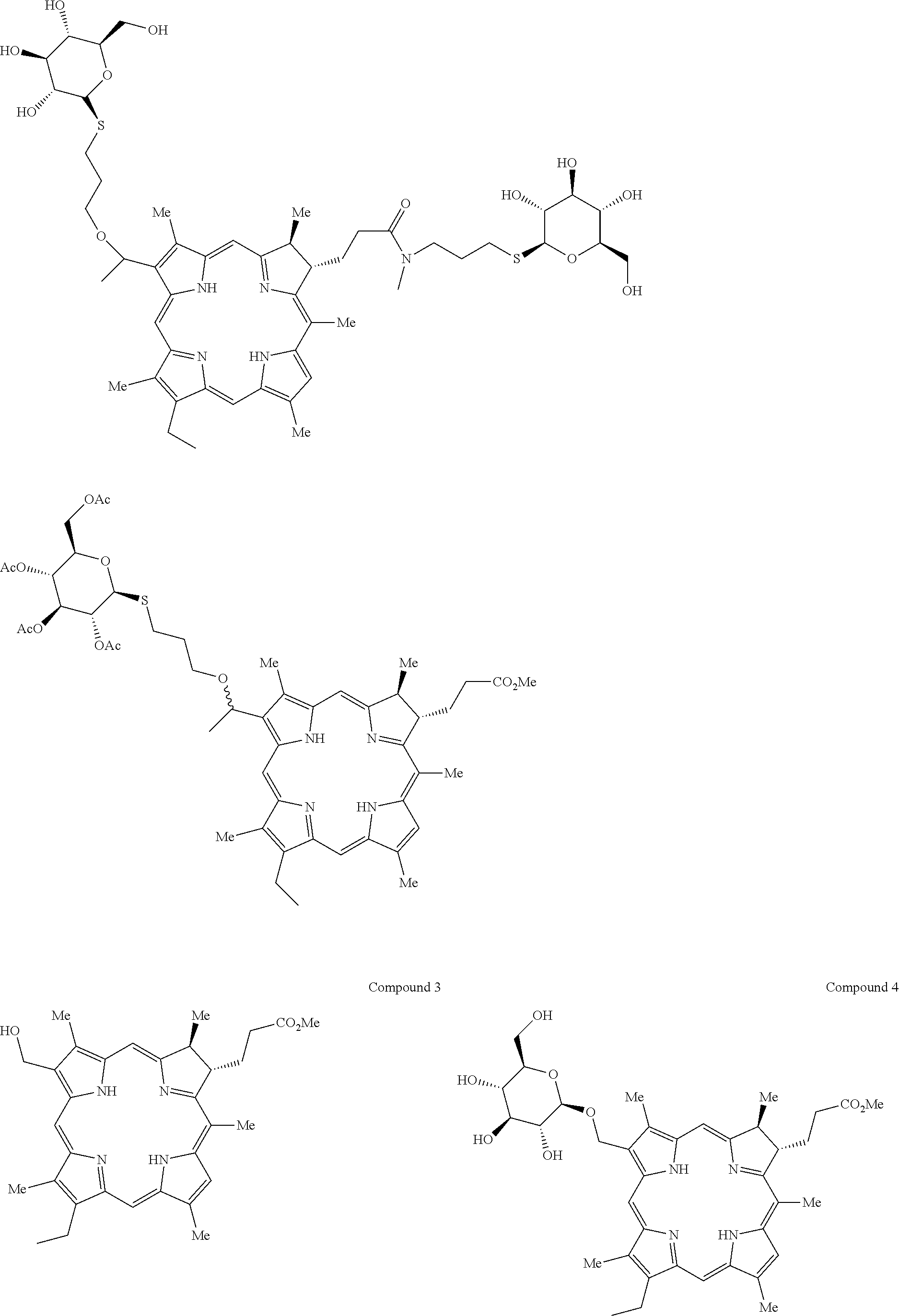

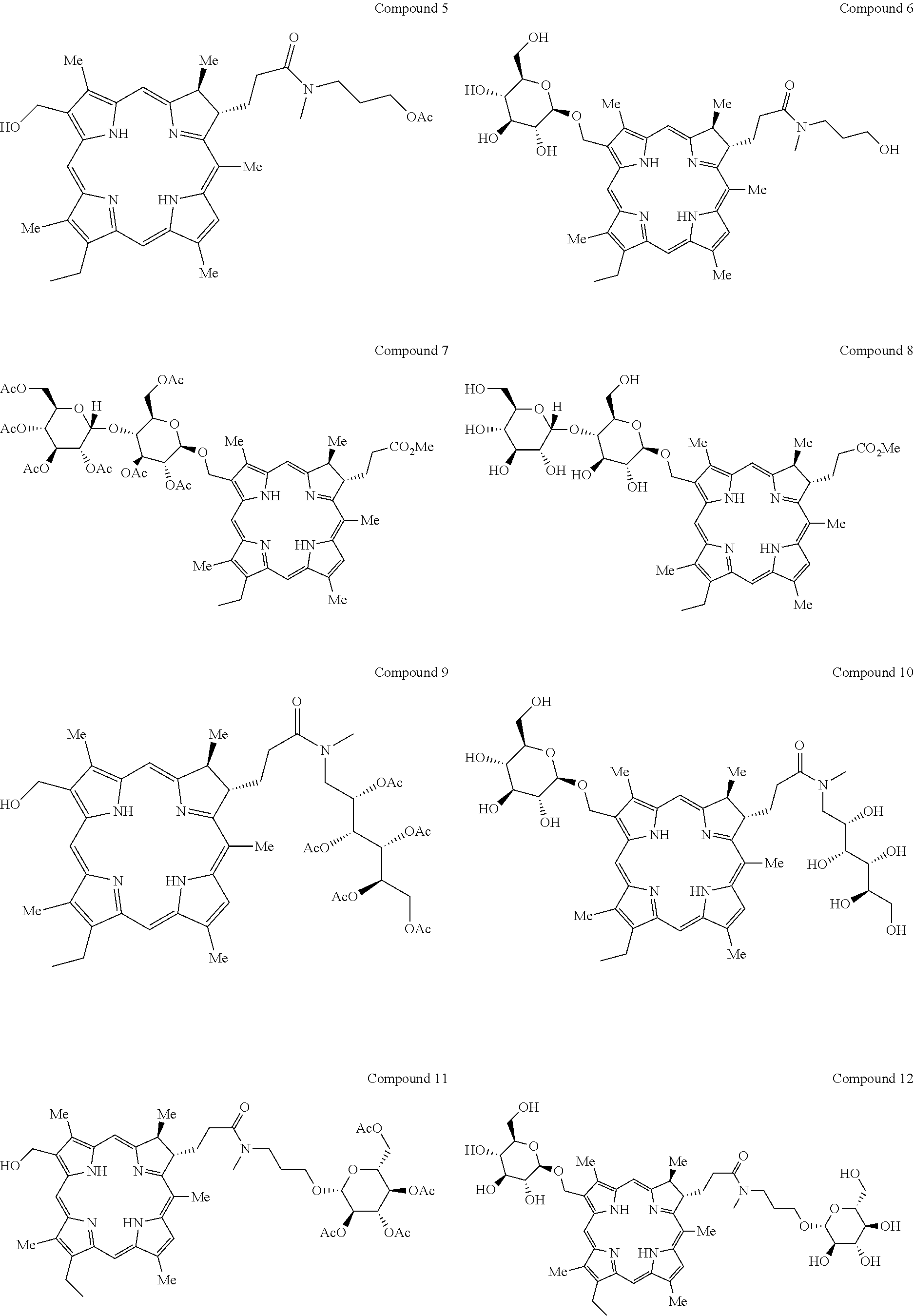

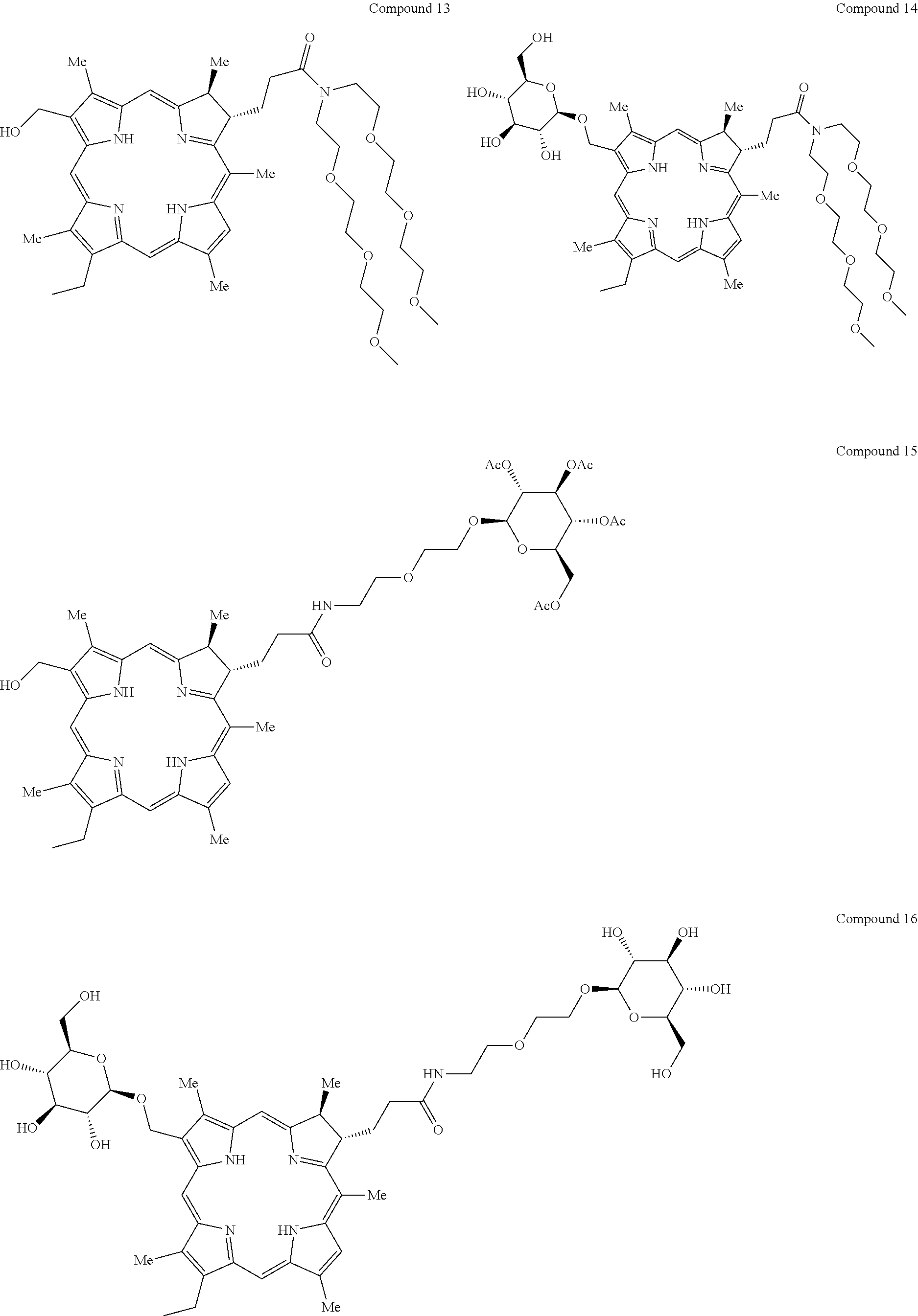

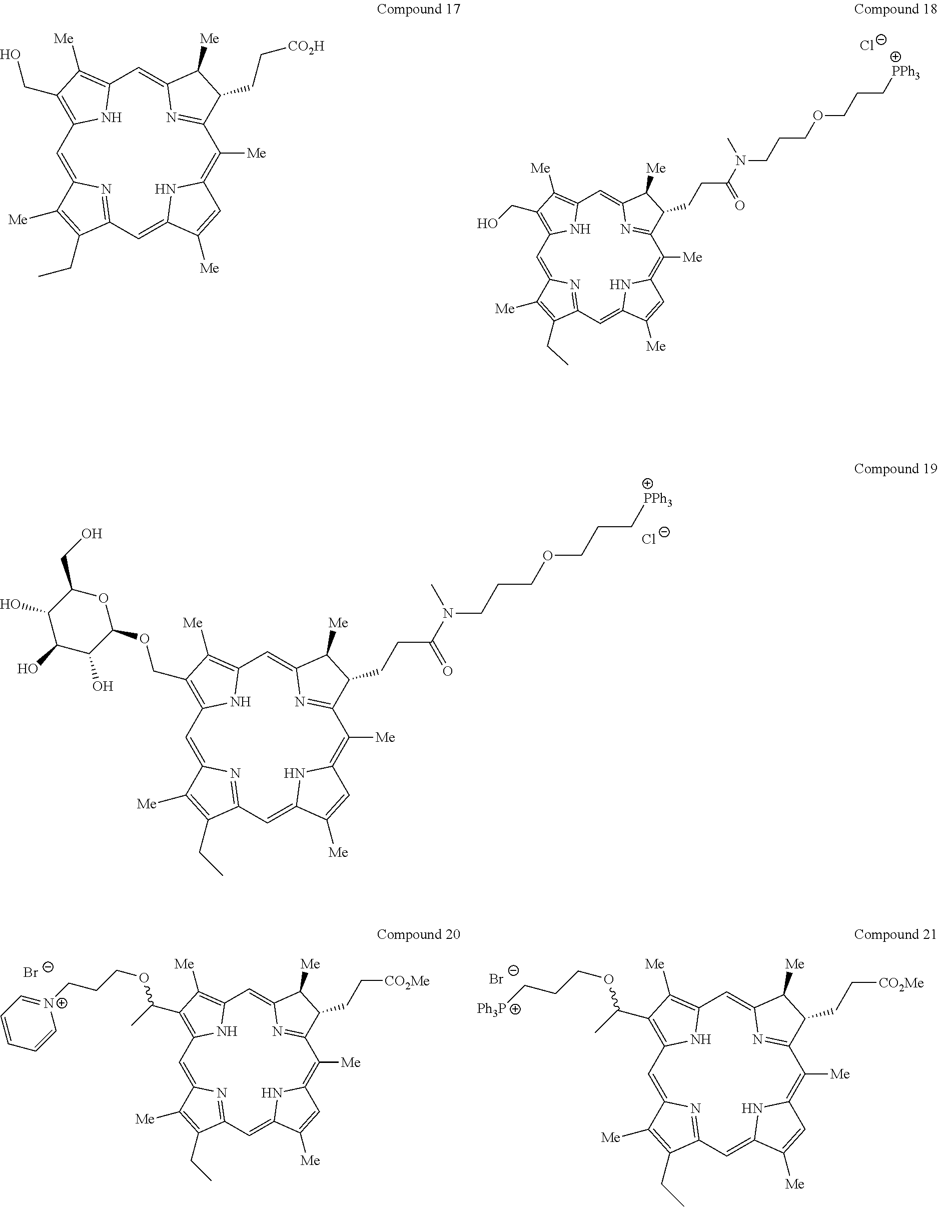

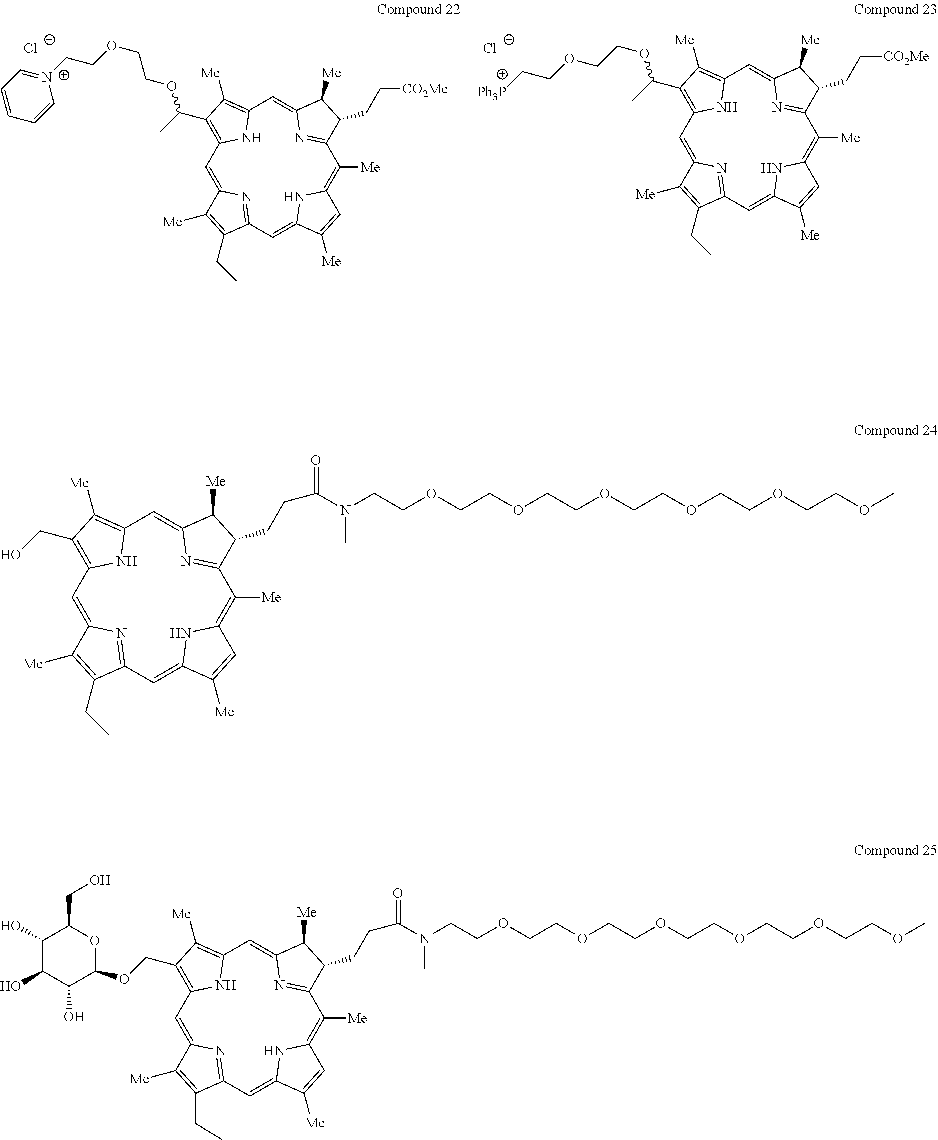

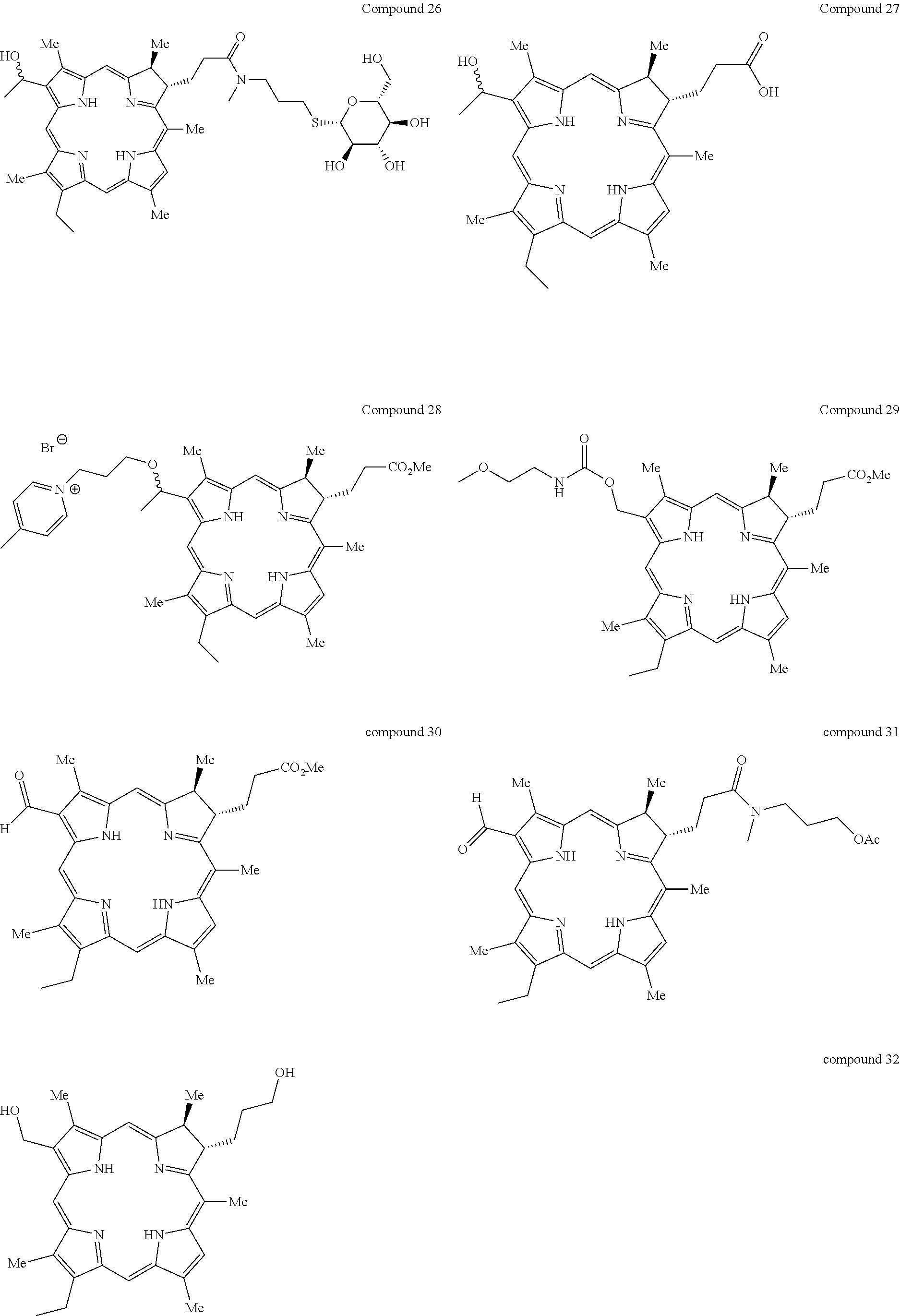

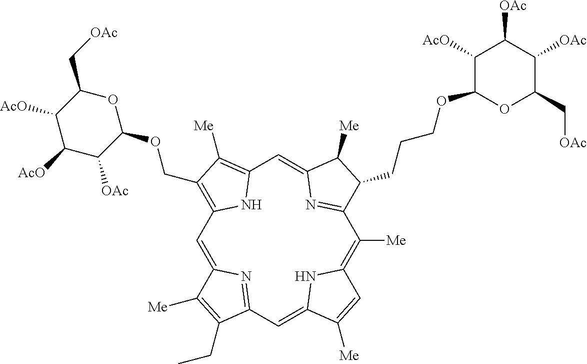 [SMILES: CCC1=C(C)c2cc3[nH]c(cc4nc(c(C)c5cc(C)c(cc1n2)[nH]5)[C@@H](CCCO[C@@H]1O[C@H](COC(C)=O)[C@@H](OC(C)=O)[C@H](OC(C)=O)[C@H]1OC(C)=O)[C@@H]4C)c(C)c3CO[C@@H]1O[C@H](COC(C)=O)[C@@H](OC(C)=O)[C@H](OC(C)=O)[C@H]1OC(C)=O]

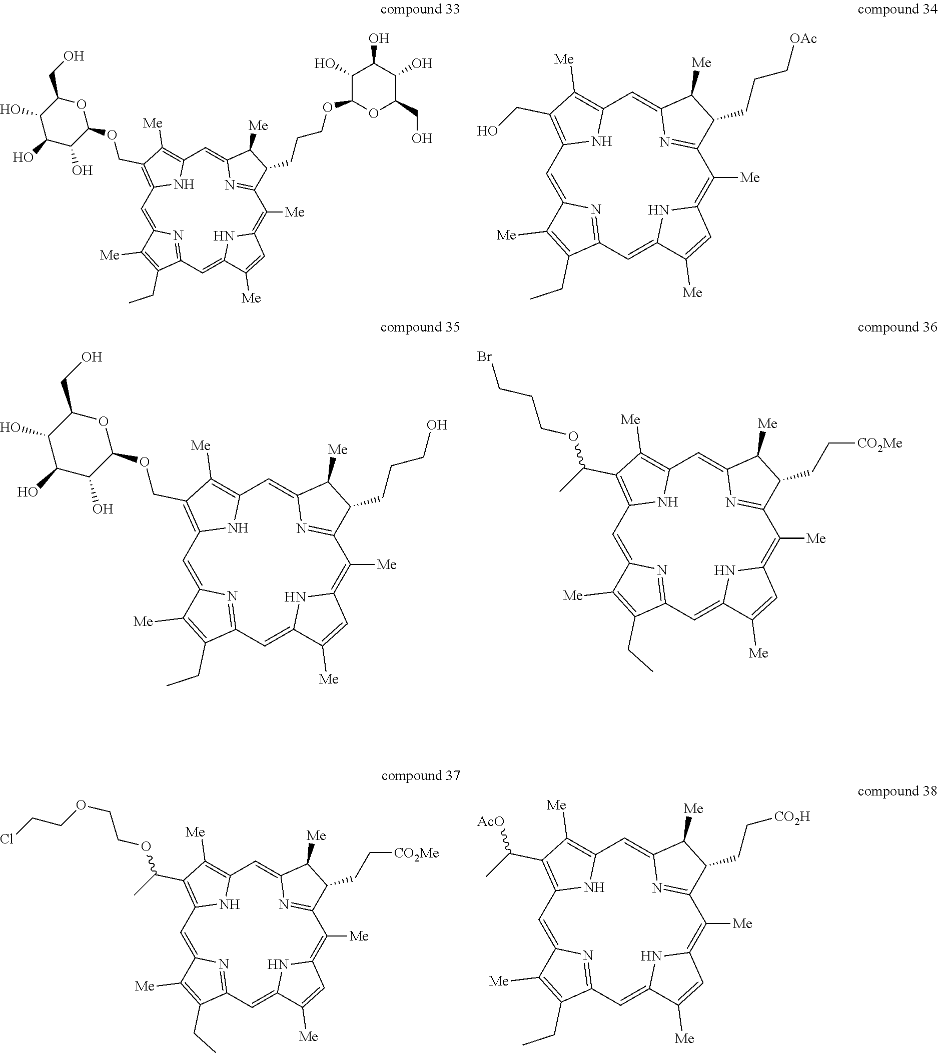

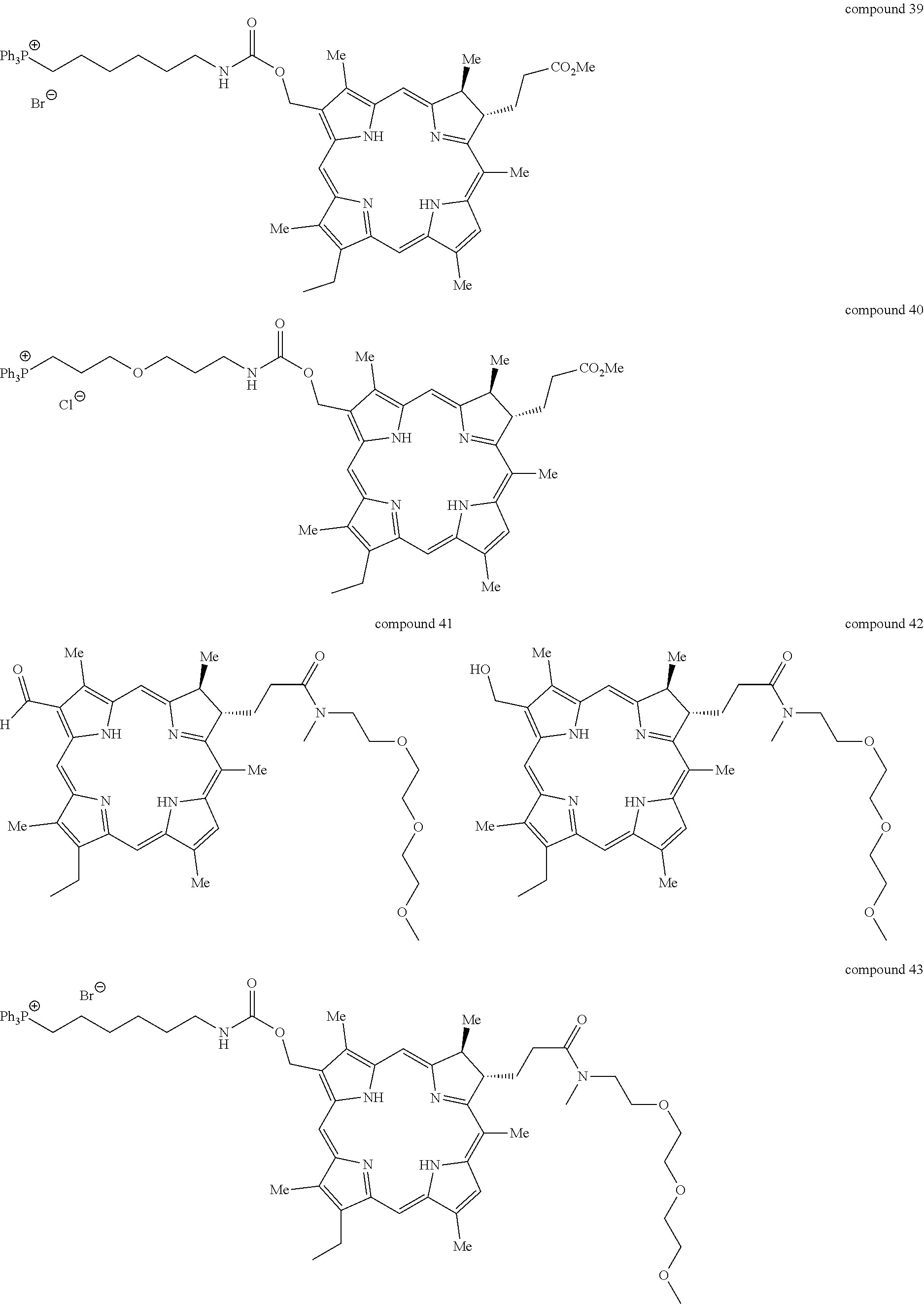

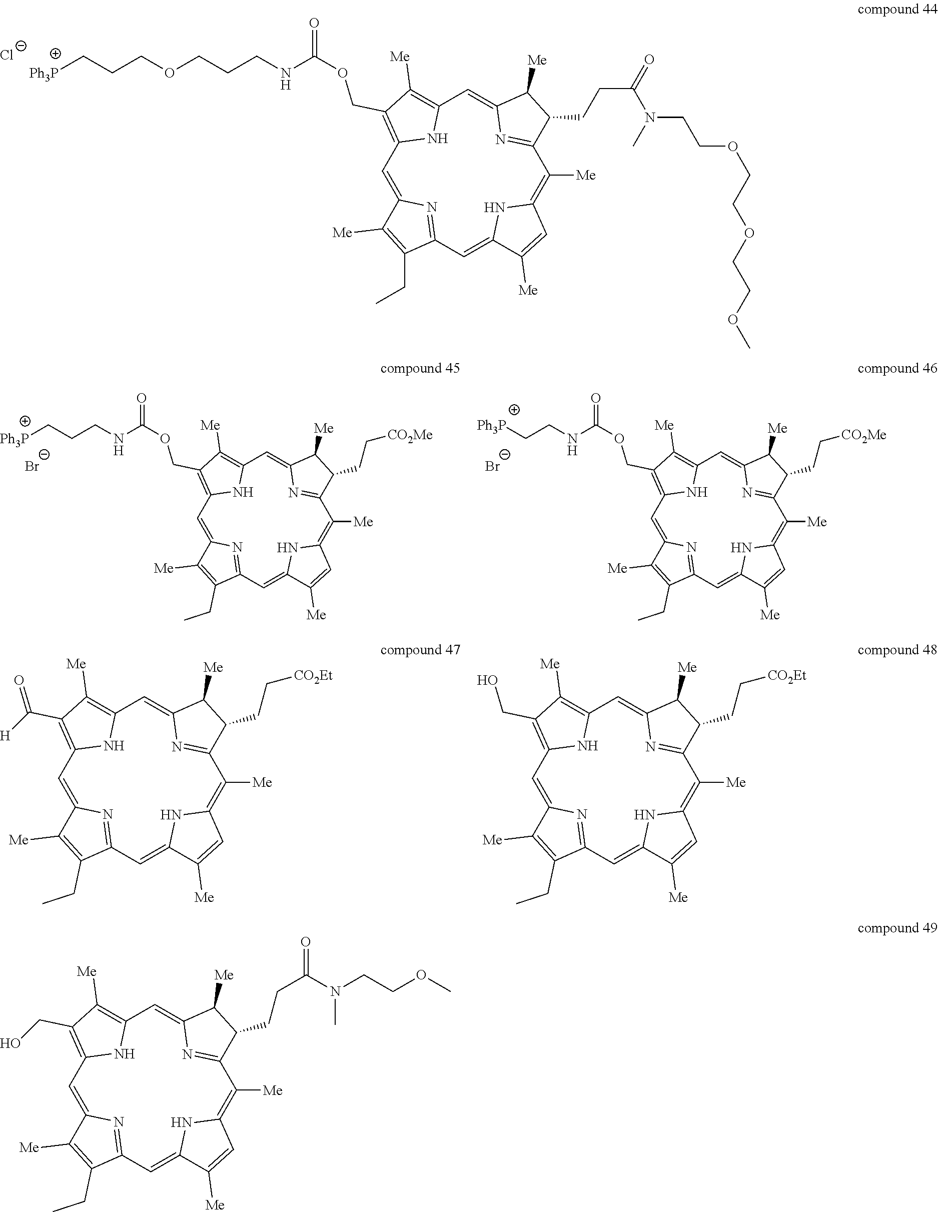

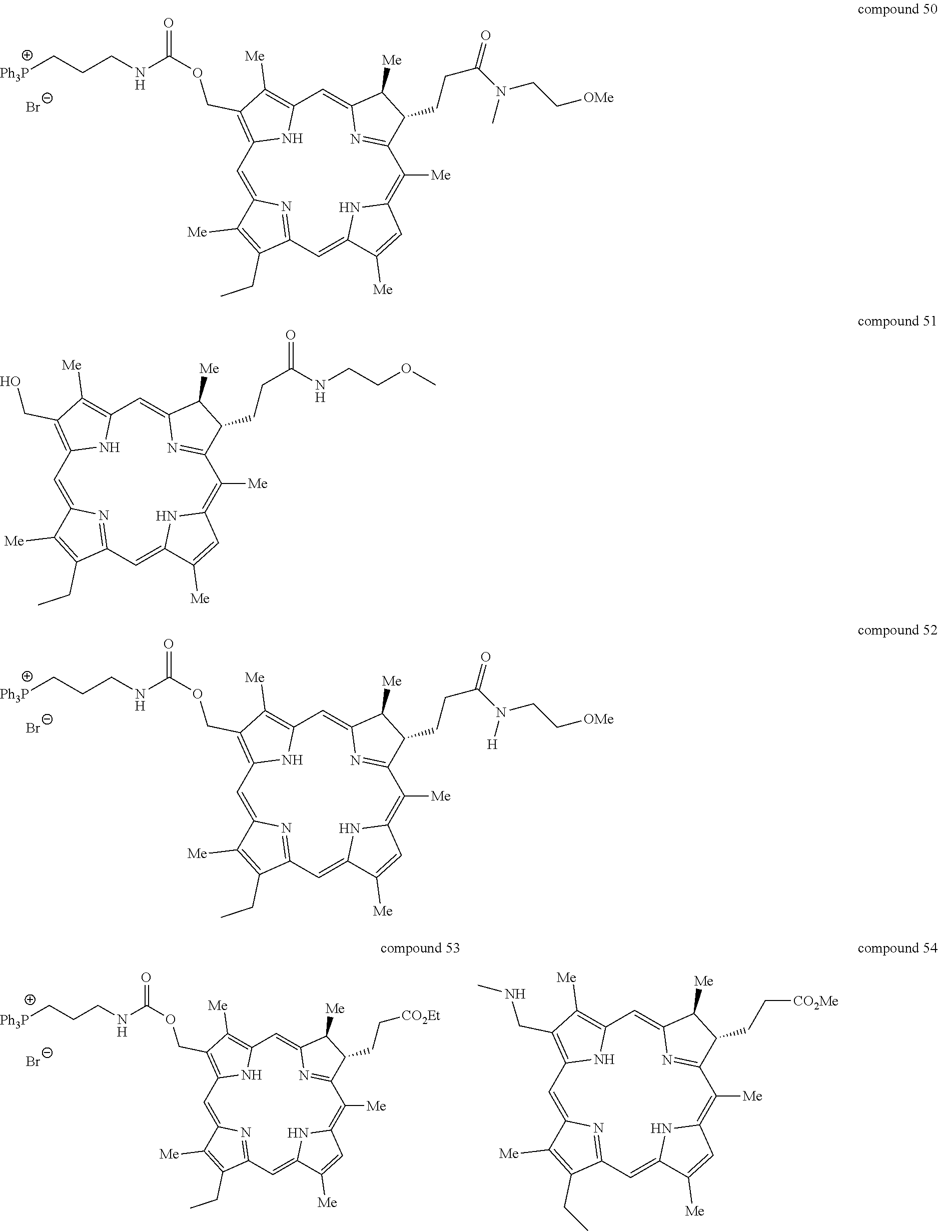

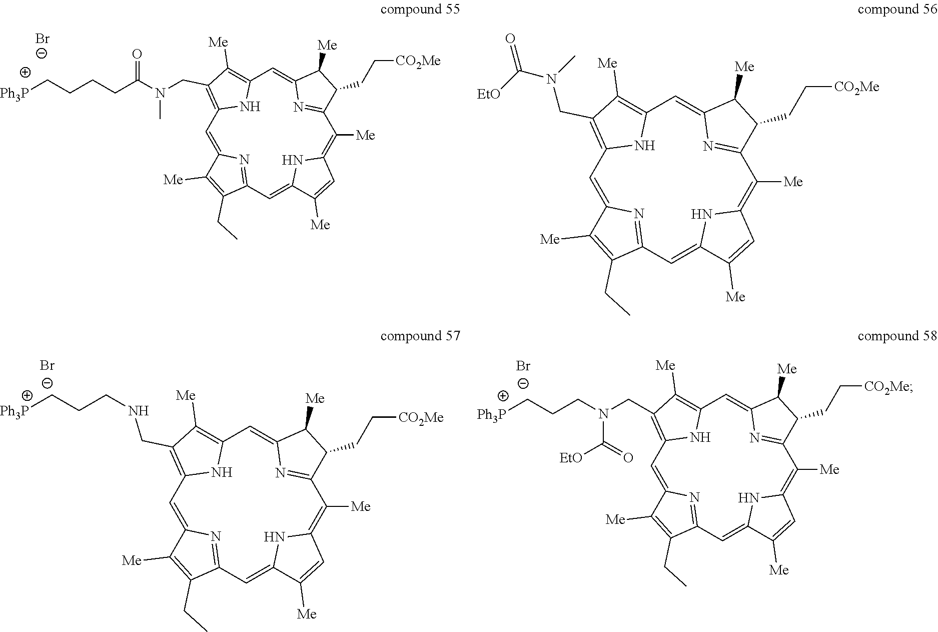

or a metal cation complex thereof, or a pharmaceutically acceptable salt thereof.

In one embodiment, the compound or complex according to the first or second aspect of the invention is in the form of a pharmaceutically acceptable salt. In one embodiment, the compound or complex is in the form of an inorganic salt such as a lithium, sodium, potassium, magnesium, calcium or ammonium salt. In one embodiment, the compound or complex is in the form of a sodium or potassium salt. In one embodiment, the compound is in the form of a sodium salt. In another embodiment, the compound or complex is in the form of an organic salt such as an amine salt (for example a choline or meglumine salt) or an amino acid salt (for example an arginine salt).

The compound or complex according to the first or second aspect of the invention has at least two chiral centres. The compound or complex of the first or second aspect of the invention is preferably substantially enantiomerically pure, which means that the compound or complex comprises less than 10% of other stereoisomers, preferably less than 5%, preferably less than 3%, preferably less than 2%, preferably less than 1%, preferably less than 0.5%, all by weight, as measured by XRPD or SFC.

Preferably, the compound or complex according to the first or second aspect of the invention has a HPLC purity of more than 97%, more preferably more than 98%, more preferably more than 99%, more preferably more than 99.5%, more preferably more than 99.8%, and most preferably more than 99.9%. As used herein the percentage HPLC purity is measured by the area normalisation method.

A third aspect of the invention provides a composition comprising a compound or complex according to the first or second aspect of the invention and a pharmaceutically acceptable carrier or diluent.

In one embodiment, the composition according to the third aspect of the invention further comprises polyvinylpyrrolidone (PVP). In one embodiment, the composition comprises 0.01-10% w/w PVP as percentage of the total weight of the composition, preferably 0.1-5% w/w PVP as a percentage of the total weight of the composition, preferably 0.5-5% w/w PVP as a percentage of the total weight of the composition. In one embodiment, the PVP is K30.

In one embodiment, the composition according to the third aspect of the invention further comprises dimethylsulfoxide (DMSO). In one embodiment, the composition comprises 0.01-99% w/w DMSO as percentage of the total weight of the composition, preferably 40-99% w/w DMSO as a percentage of the total weight of the composition, preferably 65-99% w/w DMSO as a percentage of the total weight of the composition.

In one embodiment, the composition according to the third aspect of the invention further comprises an immune checkpoint inhibitor. In one embodiment, the immune checkpoint inhibitor is an inhibitor of PD-1 (programmed cell death protein 1), PD-L1 (programmed death ligand 1) or CTLA4 (cytotoxic T-lymphocyte associated protein 4). In one embodiment, the immune checkpoint inhibitor is selected from Pembrolizumab, Nivolumab, Cemiplimab, Atezolizumab, Avelumab, Durvalumab or Ipilimumab.

Preferably the compound or complex according to the first or second aspect of the present invention and the pharmaceutical composition according to the third aspect of the present invention are suitable for use in photodynamic therapy or cytoluminescent therapy.

Preferably the compound or complex according to the first or second aspect of the present invention and the pharmaceutical composition according to the third aspect of the present invention are suitable for the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the compound or complex according to the first or second aspect of the present invention and the pharmaceutical composition according to the third aspect of the present invention are suitable for the treatment of a disease characterised by benign or malignant cellular hyperproliferation or by areas of neovascularisation.

Preferably the compound or complex according to the first or second aspect of the present invention and the pharmaceutical composition according to the third aspect of the present invention are suitable for the treatment of a benign or malignant tumour.

Preferably the compound or complex according to the first or second aspect of the present invention and the pharmaceutical composition according to the third aspect of the present invention are suitable for the treatment of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the compound or complex according to the first or second aspect of the present invention and the pharmaceutical composition according to the third aspect of the present invention are suitable for use in photodynamic diagnosis.

Preferably the compound or complex according to the first or second aspect of the present invention and the pharmaceutical composition according to the third aspect of the present invention are suitable for the detection of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the compound or complex according to the first or second aspect of the present invention and the pharmaceutical composition according to the third aspect of the present invention are suitable for the detection of an area that is affected by benign or malignant cellular hyperproliferation or by neovascularisation.

Preferably the compound or complex according to the first or second aspect of the present invention and the pharmaceutical composition according to the third aspect of the present invention are suitable for the detection of a benign or malignant tumour.

Preferably the compound or complex according to the first or second aspect of the present invention and the pharmaceutical composition according to the third aspect of the present invention are suitable for the detection of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the compound or complex according to the first or second aspect of the present invention and the pharmaceutical composition according to the third aspect of the present invention are suitable for the fluorescent or phosphorescent detection of the diseases listed above, preferably for the fluorescent or phosphorescent detection and quantification of the said diseases.

Preferably the compound or complex according to the first or second aspect of the present invention and the pharmaceutical composition according to the third aspect of the present invention are adapted for administration simultaneous with or prior to administration of irradiation or sound, preferably for administration prior to administration of irradiation.

If the compound or complex according to the first or second aspect of the present invention or the pharmaceutical composition according to the third aspect of the present invention are for use in photodynamic therapy or cytoluminescent therapy, then they are preferably adapted for administration 5 to 100 hours before the irradiation, preferably 6 to 72 hours before the irradiation, preferably 24 to 48 hours before the irradiation.

If the compound or complex according to the first or second aspect of the present invention or the pharmaceutical composition according to the third aspect of the present invention are for use in photodynamic diagnosis, then they are preferably adapted for administration 3 to 60 hours before the irradiation, preferably 8 to 40 hours before the irradiation.

Preferably the irradiation used in the photodynamic therapy, cytoluminescent therapy or photodynamic diagnosis is electromagnetic radiation with a wavelength in the range of from 500 nm to 1000 nm, preferably from 550 nm to 750 nm, preferably from 600 nm to 700 nm, preferably from 640 nm to 670 nm. The electromagnetic radiation may be administered for about 5-60 minutes, preferably for about 15-20 minutes, at about 0.1-5 W, preferably at about 1 W. In one embodiment of the present invention, two sources of electromagnetic radiation are used (for example a laser light and an LED light), both sources adapted to provide irradiation with a wavelength in the range of from 550 nm to 750 nm, preferably from 600 nm to 700 nm, preferably from 640 nm to 670 nm. In another embodiment of the present invention, the irradiation may be provided by a prostate, anal, vaginal, mouth and nasal device for insertion into a body cavity. In another embodiment of the present invention, the irradiation may be provided by interstitial light activation, for example, using a fine needle to insert an optical fibre laser into the lung, liver, lymph nodes or breast. In another embodiment of the present invention, the irradiation may be provided by endoscopic light activation, for example, for delivering light to the lung, stomach, colon, bladder or neck.

The pharmaceutical composition according to the third aspect of the present invention may be in a form suitable for oral, parenteral (including intravenous, subcutaneous, intramuscular, intradermal, intratracheal, intraperitoneal, intratumoral, intraarticular, intraabdominal, intracranial and epidural), transdermal, airway (aerosol), rectal, vaginal or topical (including buccal, mucosal and sublingual) administration. The pharmaceutical composition may also be in a form suitable for administration by enema or for administration by injection into a tumour. Preferably the pharmaceutical composition is in a form suitable for oral, parenteral (such as intravenous, intraperitoneal, and intratumoral) or airway administration, preferably in a form suitable for oral or parenteral administration, preferably in a form suitable for oral administration.

In one preferred embodiment, the pharmaceutical composition is in a form suitable for oral administration. Preferably the pharmaceutical composition is provided in the form of a tablet, capsule, hard or soft gelatine capsule, caplet, troche or lozenge, as a powder or granules, or as an aqueous solution, suspension or dispersion. More preferably the pharmaceutical composition is provided in the form of an aqueous solution, suspension or dispersion for oral administration, or alternatively in the form of a freeze-dried powder which can be mixed with water before administration to provide an aqueous solution, suspension or dispersion for oral administration. Preferably the pharmaceutical composition is in a form suitable for providing 0.01 to 10 mg/kg/day of the compound or complex according to the first or second aspect of the invention, preferably 0.1 to 2 mg/kg/day, preferably about 1 mg/kg/day.

In another preferred embodiment, the pharmaceutical composition is in a form suitable for parenteral administration. Preferably the pharmaceutical composition is in a form suitable for intravenous administration. Preferably the pharmaceutical composition is provided in the form of an aqueous solution for parenteral administration, or alternatively in the form of a freeze-dried powder which can be mixed with water before administration to provide an aqueous solution for parenteral administration. Preferably the pharmaceutical composition is an aqueous solution or suspension having a pH of from 6 to 8.5. Preferably the pharmaceutical composition is in a form suitable for providing 0.01 to 10 mg/kg/day of the compound or complex according to the first or second aspect of the invention, preferably 0.1 to 2 mg/kg/day, preferably about 1 mg/kg/day.

In another preferred embodiment, the pharmaceutical composition is in a form suitable for airway administration. Preferably the pharmaceutical composition is provided in the form of an aqueous solution, suspension or dispersion for airway administration, or alternatively in the form of a freeze-dried powder which can be mixed with water before administration to provide an aqueous solution, suspension or dispersion for airway administration. Preferably the pharmaceutical composition is in a form suitable for providing 0.01 to 10 mg/kg/day of the compound or complex according to the first or second aspect of the invention, preferably 0.1 to 2 mg/kg/day, preferably about 1 mg/kg/day.

A fourth aspect of the present invention provides use of a compound or complex according to the first or second aspect of the present invention in the manufacture of a medicament for the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-COV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

The fourth aspect of the present invention also provides use of a compound or complex according to the first or second aspect of the present invention in the manufacture of a phototherapeutic agent for use in photodynamic therapy or cytoluminescent therapy. Preferably the phototherapeutic agent is suitable for the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-COV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the medicament or the phototherapeutic agent of the fourth aspect of the present invention is suitable for the treatment of a disease characterised by benign or malignant cellular hyperproliferation or by areas of neovascularisation.

Preferably the medicament or the phototherapeutic agent of the fourth aspect of the present invention is suitable for the treatment of a benign or malignant tumour.

Preferably the medicament or the phototherapeutic agent of the fourth aspect of the present invention is suitable for the treatment of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

The fourth aspect of the present invention also provides use of a compound or complex according to the first or second aspect of the present invention in the manufacture of a photodiagnostic agent for use in photodynamic diagnosis.

Preferably the photodiagnostic agent of the fourth aspect of the present invention is suitable for the detection of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-COV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the photodiagnostic agent of the fourth aspect of the present invention is suitable for the detection of an area that is affected by benign or malignant cellular hyperproliferation or by neovascularisation.

Preferably the photodiagnostic agent of the fourth aspect of the present invention is suitable for the detection of a benign or malignant tumour.

Preferably the photodiagnostic agent of the fourth aspect of the present invention is suitable for the detection of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the photodiagnostic agent of the fourth aspect of the present invention is suitable for the fluorescent or phosphorescent detection of the said diseases, preferably the fluorescent or phosphorescent detection and quantification of the said diseases.

Preferably the medicament, the phototherapeutic agent or the photodiagnostic agent is adapted for administration simultaneous with or prior to administration of irradiation or sound, preferably for administration prior to administration of irradiation.

If the medicament or the phototherapeutic agent is for use in photodynamic therapy or cytoluminescent therapy, then it is preferably adapted for administration 5 to 100 hours before the irradiation, preferably 6 to 72 hours before the irradiation, preferably 24 to 48 hours before the irradiation.

If the photodiagnostic agent is for use in photodynamic diagnosis, then it is preferably adapted for administration 3 to 60 hours before the irradiation, preferably 8 to 40 hours before the irradiation.

Preferably the irradiation used in the photodynamic therapy, cytoluminescent therapy or photodynamic diagnosis is electromagnetic radiation with a wavelength in the range of from 500 nm to 1000 nm, preferably from 550 nm to 750 nm, preferably from 600 nm to 700 nm, preferably from 640 nm to 670 nm. The electromagnetic radiation may be administered for about 5-60 minutes, preferably for about 15-20 minutes, at about 0.1-5 W, preferably at about 1 W. In one embodiment of the present invention, two sources of electromagnetic radiation are used (for example a laser light and an LED light), both sources adapted to provide irradiation with a wavelength in the range of from 550 nm to 750 nm, preferably from 600 nm to 700 nm, preferably from 640 nm to 670 nm. In another embodiment of the present invention, the irradiation may be provided by a prostate, anal, vaginal, mouth and nasal device for insertion into a body cavity. In another embodiment of the present invention, the irradiation may be provided by interstitial light activation, for example, using a fine needle to insert an optical fibre laser into the lung, liver, lymph nodes or breast. In another embodiment of the present invention, the irradiation may be provided by endoscopic light activation, for example, for delivering light to the lung, stomach, colon, bladder or neck.

A fifth aspect of the present invention provides a method of treating atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-COV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas; the method comprising administering a therapeutically effective amount of a compound or complex according to the first or second aspect of the present invention to a human or animal in need thereof.

The fifth aspect of the present invention also provides a method of photodynamic therapy or cytoluminescent therapy of a human or animal disease, the method comprising administering a therapeutically effective amount of a compound or complex according to the first or second aspect of the present invention to a human or animal in need thereof. Preferably the human or animal disease is atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the method of the fifth aspect of the present invention is a method of treating benign or malignant cellular hyperproliferation or areas of neovascularisation.

Preferably the method of the fifth aspect of the present invention is a method of treating a benign or malignant tumour.

Preferably the method of the fifth aspect of the present invention is a method of treating early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

The fifth aspect of the present invention also provides a method of photodynamic diagnosis of a human or animal disease, the method comprising administering a diagnostically effective amount of a compound or complex according to the first or second aspect of the present invention to a human or animal. Preferably the human or animal disease is atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-COV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas. Preferably the human or animal disease is characterised by benign or malignant cellular hyperproliferation or by areas of neovascularisation. Preferably the human or animal disease is a benign or malignant tumour. Preferably the human or animal disease is early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas. Preferably the method of photodynamic diagnosis is suitable for the fluorescent or phosphorescent detection of the said diseases, preferably for the fluorescent or phosphorescent detection and quantification of the said diseases.

In any of the methods of the fifth aspect of the present invention, the human or animal is preferably further subjected to irradiation or sound simultaneous with or after the administration of the compound or complex according to the first or second aspect of the invention. Preferably the human or animal is subjected to irradiation after the administration of the compound or complex according to the first or second aspect of the invention.

If the method is a method of photodynamic therapy or cytoluminescent therapy, then the human or animal is preferably subjected to irradiation 5 to 100 hours after administration of the compound or complex according to the first or second aspect of the invention, preferably 6 to 72 hours after administration, preferably 24 to 48 hours after administration.

If the method is a method of photodynamic diagnosis, then the human or animal is preferably subjected to irradiation 3 to 60 hours after administration of the compound or complex according to the first or second aspect of the invention, preferably 8 to 40 hours after administration.

Preferably the irradiation is electromagnetic radiation with a wavelength in the range of from 500 nm to 1000 nm, preferably from 550 nm to 750 nm, preferably from 600 nm to 700 nm, preferably from 640 nm to 670 nm. The electromagnetic radiation may be administered for about 5-60 minutes, preferably for about 15-20 minutes, at about 0.1-5 W, preferably at about 1 W. In one embodiment of the present invention, two sources of electromagnetic radiation are used (for example a laser light and an LED light), both sources adapted to provide irradiation with a wavelength in the range of from 550 nm to 750 nm, preferably from 600 nm to 700 nm, preferably from 640 nm to 670 nm. In another embodiment of the present invention, the irradiation may be provided by a prostate, anal, vaginal, mouth and nasal device for insertion into a body cavity. In another embodiment of the present invention, the irradiation may be provided by interstitial light activation, for example, using a fine needle to insert an optical fibre laser into the lung, liver, lymph nodes or breast. In another embodiment of the present invention, the irradiation may be provided by endoscopic light activation, for example, for delivering light to the lung, stomach, colon, bladder or neck.

In any of the methods of the fifth aspect of the present invention, preferably the human or animal is a human.

A sixth aspect of the present invention provides a pharmaceutical combination or kit comprising:
(a) a compound or complex according to the first or second aspect of the present invention; and
(b) an immune checkpoint inhibitor.

In one embodiment, the immune checkpoint inhibitor is an inhibitor of PD-1 (programmed cell death protein 1), PD-L1 (programmed death ligand 1) or CTLA4 (cytotoxic T-lymphocyte associated protein 4). In one embodiment, the immune checkpoint inhibitor is selected from Pembrolizumab, Nivolumab, Cemiplimab, Atezolizumab, Avelumab, Durvalumab or Ipilimumab.

Preferably, the combination or kit of the sixth aspect is for use in the treatment of a disease, disorder or condition, wherein the disease, disorder or condition is responsive to PD-1, PD-L1 or CTLA4 inhibition. Preferably, the combination or kit of the sixth aspect is for use in the treatment of cancer. In one embodiment, the cancer is melanoma, lung cancer (e.g. non small cell lung cancer), kidney cancer, bladder cancer, head and neck cancer, or Hodgkin's lymphoma.

The sixth aspect also provides a use of the combination or kit of the sixth aspect of the invention in the manufacture of a medicament for the treatment of a disease, disorder or condition which is responsive to PD-1, PD-L1 or CTLA4 inhibition. The sixth aspect also provides a use of the combination or kit of the sixth aspect of the invention in the manufacture of a medicament for the treatment of cancer. In one embodiment, the cancer is melanoma, lung cancer (e.g. non small cell lung cancer), kidney cancer, bladder cancer, head and neck cancer, or Hodgkin's lymphoma.

The sixth aspect of the invention also provides a method of treating a disease, disorder or condition which is responsive to PD-1, PD-L1 or CTLA4 inhibition, the method comprising administering a therapeutically effective amount of the combination or kit of the sixth aspect of the present invention to a human or animal in need thereof. The sixth aspect of the invention also provides a method of treating cancer, the method comprising administering a therapeutically effective amount of the combination or kit of the sixth aspect of the present invention to a human or animal in need thereof. In one embodiment, the cancer is melanoma, lung cancer (e.g. non small cell lung cancer), kidney cancer, bladder cancer, head and neck cancer, or Hodgkin's lymphoma.

For the combination or kit of the sixth aspect of the invention, the compound or complex according to the first or second aspect of the invention, and the immune checkpoint inhibitor may be provided together in one pharmaceutical composition or separately in two pharmaceutical compositions. If provided in two pharmaceutical compositions, these may be administered at the same time or at different times.

Preferably the combination or kit of the sixth aspect is adapted for administration simultaneous with or prior to administration of irradiation or sound, preferably for administration prior to administration of irradiation. In one embodiment, the combination or kit of the sixth aspect is adapted for administration 5 to 100 hours before the irradiation, preferably 6 to 72 hours before the irradiation, preferably 24 to 48 hours before the irradiation.

Preferably the irradiation used in the photodynamic therapy or cytoluminescent therapy is electromagnetic radiation with a wavelength in the range of from 500 nm to 1000 nm, preferably from 550 nm to 750 nm, preferably from 600 nm to 700 nm, preferably from 640 nm to 670 nm. The electromagnetic radiation may be administered for about 5-60 minutes, preferably for about 15-20 minutes, at about 0.1-5 W, preferably at about 1 W. In one embodiment of the present invention, two sources of electromagnetic radiation are used (for example a laser light and an LED light), both sources adapted to provide irradiation with a wavelength in the range of from 550 nm to 750 nm, preferably from 600 nm to 700 nm, preferably from 640 nm to 670 nm. In another embodiment of the present invention, the irradiation may be provided by a prostate, anal, vaginal, mouth and nasal device for insertion into a body cavity. In another embodiment of the present invention, the irradiation may be provided by interstitial light activation, for example, using a fine needle to insert an optical fibre laser into the lung, liver, lymph nodes or breast. In another embodiment of the present invention, the irradiation may be provided by endoscopic light activation, for example, for delivering light to the lung, stomach, colon, bladder or neck.

For the avoidance of doubt, insofar as is practicable any embodiment of a given aspect of the present invention may occur in combination with any other embodiment of the same aspect of the present invention. In addition, insofar as is practicable it is to be understood that any preferred or optional embodiment of any aspect of the present invention should also be considered as a preferred or optional embodiment of any other aspect of the present invention.

### SYNTHETIC EXPERIMENTAL DETAILS

### Synthesis Example 1—synthesis of methyl 3-((7S,8S)-18-ethyl-2,5,8,12,17-pentamethyl-13-(1-(3-(((2S,3R,4S,5S,6R)-3,4,5-trihydroxy-6-(hydroxymethyl)tetrahydro-2H-pyran-2-yl)thio) propoxy)ethyl)-7H,8H-porphyrin-7-yl) propanoate (compound 1)

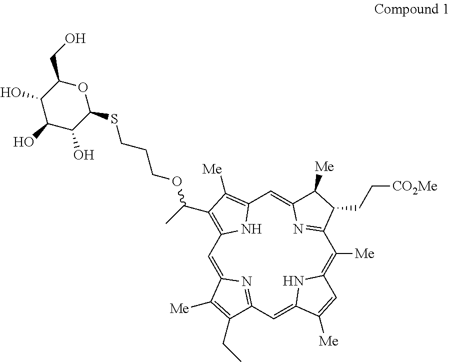 [SMILES: CCC1=C(C)c2cc3[nH]c(cc4nc(c(C)c5cc(C)c(cc1n2)[nH]5)[C@@H](CCC(=O)OC)[C@@H]4C)c(C)c3C(C)OCCCS[C@@H]1O[C@H](CO)[C@@H](O)[C@H](O)[C@H]1O]

### Synthesis of (2R,3R,4S,5R,6S)-2-(acetoxymethyl)-6-((3-hydroxypropyl)thio) tetrahydro-2H-pyran-3,4,5-triyl triacetate

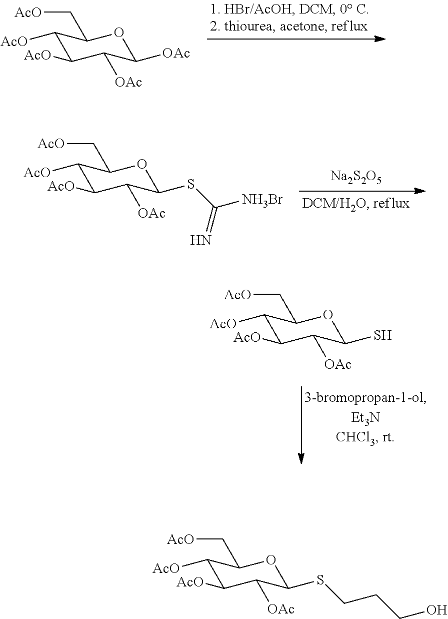

Step 1: A single-neck 100 mL RBF fitted with a 50 mL dropping funnel and nitrogen inlet was charged with 1,2,3,4,6-penta-O-acetyl-β-D-glucose (2.00 g, 5.12 mmol, 1 eq), a stirrer bar and dry DCM (20 mL). The resultant solution was cooled while stirring (420 rpm) under N₂ in an ice-water bath for 10 minutes, before charging the dropping funnel with 33% w/w HBr/AcOH (8.2 mL, 47 mmol, 9.1 eq), and adding it dropwise to the solution over the course of 5 minutes. Following complete addition, the reaction mixture was stirred while being kept in the ice-water bath for 1 hour. The mixture was diluted with Et₂O (100 mL) and washed with H₂O (3×100 mL), then saturated aqueous NaHCO₃ (2×100 mL), before being dried (MgSO₄) and concentrated to give crude 2,3,4,6-tetra-O-acetyl-α-D-glucopyranosyl bromide as a yellow syrup that was used without further purification (2.08 g, 99%).

¹H NMR (400 MHz, CDCl₃) δ 6.61 (d, J=4.1 Hz, 1H), 5.56 (dd, J=9.7, 9.7 Hz, 1H), 5.16 (dd, J=10.1, 9.3 Hz, 1H), 4.83 (dd, J=10.0, 4.1 Hz, 1H), 4.37-4.22 (m, 2H), 4.17-4.08 (m, 1H), 2.10 (s, 3H), 2.10 (s, 3H), 2.05 (s, 3H), 2.03 (s, 3H).

Step 2: The crude glucopyranosyl bromide was dissolved in acetone (20 mL) and added to a 100 mL RBF containing thiourea (0.507 g, 6.66 mmol, 1.3 eq) and 3 A molecular sieves (2.0 g). The resultant mixture was refluxed (external temperature=80° C.) while stirring (420 rpm) under N₂ overnight. The product precipitated as a colourless solid. The reaction mixture was diluted with ether (20 mL) and filtered on a glass sinter funnel and washed briefly with ether. The product was separated from the molecular sieves by washing it from the sinter with MeOH and concentrating the filtrate to give pure 2,3,4,6-tetra-O-acetyl-β-D-glucospyranoyl-1-S-isothiouronium bromide as a colourless solid (1.51 g, 60%).

¹H NMR (400 MHz, DMSO-d₆) δ 9.16 (br s, 4H), 5.69 (d, J=10.0 Hz, 1H), 5.32 (dd, J=9.4, 9.4 Hz, 1H), 5.16-5.05 (m, 2H), 4.25-4.14 (m, 2H), 4.13-4.03 (m, 1H), 2.06 (s, 3H), 2.02 (s, 3H), 2.00 (s, 3H), 1.97 (s, 3H).

Step 3: To a 100 mL RBF containing 2,3,4,6-tetra-O-acetyl-β-D-glucospyranoyl-1-S— isothiouronium bromide (1.51 g, 3.10 mmol, 1 eq) was added DCM (20 mL) (only partially dissolved), then a solution of Na₂S₂O₅ (2.00 g, 10.5 mmol, 3.4 eq) in water (20 mL). The heterogeneous mixture was refluxed (external temperature=70° C.) for 1 hour, during which time all material dissolved. A concentrated aliquot of the organic layer showed clean conversion to the thiol. The organic phase was separated and washed with H₂O (20 mL), then brine (20 mL), before being dried (MgSO₄) and concentrated by rotary evaporation to give (2R,3R,4S,5R,6S)-2-(acetoxymethyl)-6-mercaptotetrahydro-2H-pyran-3,4,5-triyl triacetate as a colourless oil that solidified upon standing (1.09 g, 96%).

¹H NMR (400 MHZ, CDCl₃) δ 5.19 (dd, J=9.9, 9.9 Hz, 1H), 5.09 (dd, J=9.9, 9.9 Hz, 1H), 4.97 (dd, J=9.9, 9.9 Hz, 1H), 4.54 (dd, J=9.9, 9.9 Hz, 1H), 4.25 (dd, J=12.5, 4.8 Hz, 1H), 4.12 (dd, J=12.5, 2.3 Hz, 1H), 3.72 (ddd, J=9.9, 4.8, 2.3 Hz, 1H), 2.31 (d, J=9.9 Hz, 1H), 2.09 (s, 3H), 2.08 (s, 3H), 2.02 (s, 3H), 2.01 (s, 3H).

Step 4: To a single-neck 100 mL RBF were added a small stirrer bar, (2R,3R,4S,5R,6S)-2-(acetoxymethyl)-6-mercaptotetrahydro-2H-pyran-3,4,5-triyl triacetate (3.78 g, 9.54 mmol, 1 eq), CHCl₃ (15 mL) and triethylamine (2.65 mL, 19.1 mmol, 2 eq). To the resultant solution was added 3-bromopropan-1-ol (1.06 mL, 11.7 mmol, 1.2 eq), and the mixture was stirred (420 rpm) under N₂. After 4 hours, only minute traces of starting material remained by TLC (50% EtOAc/hexanes, R_{f} (thioglucose)=0.60, R_{f} (bromide)=0.55, R_{f} (product)=0.2). The reaction was diluted with DCM (20 mL) and water (20 mL), and the organic layer collected and washed with brine (20 mL). The organic phase was dried (MgSO₄) and concentrated by rotary evaporation to give the crude coupled product as a light yellow syrup (5.08 g). The crude product was purified by column chromatography (70% EtOAc/hexanes) to give (2R,3R,4S,5R,6S)-2-(acetoxymethyl)-6-((3-hydroxypropyl)thio)tetrahydro-2H-pyran-3,4,5-triyl triacetate as a very pale yellow oil that solidified to give a grey wax on standing (3.79 g, 94%).

¹H NMR (400 MHZ, CDCl₃) δ 5.22 (dd, J=9.4, 9.4 Hz, 1H), 5.12-4.98 (m, 2H), 4.48 (d, J=10.1 Hz, 1H), 4.23 (dd, J=12.4, 4.9 Hz, 1H), 4.15 (dd, J=12.4, 2.4 Hz, 1H), 3.77-3.67 (m, 3H), 2.89-2.70 (m, 2H), 2.08 (s, 3H), 2.06 (s, 3H), 2.02 (s, 2H), 2.00 (s, 3H), 1.90-1.75 (m, 3H).

### Synthesis of phyllochlorin propyloxythio glucose: methyl 3-((7S,8S)-18-ethyl-2,5,8,12,17-pentamethyl-13-(1-(3-(((2S,3R,4S,5S,6R)-3,4,5-trihydroxy-6-(hydroxymethyl)tetrahydro-2H-pyran-2-yl)thio) propoxy)ethyl)-7H,8H-porphyrin-7-yl) propanoate (compound 1)

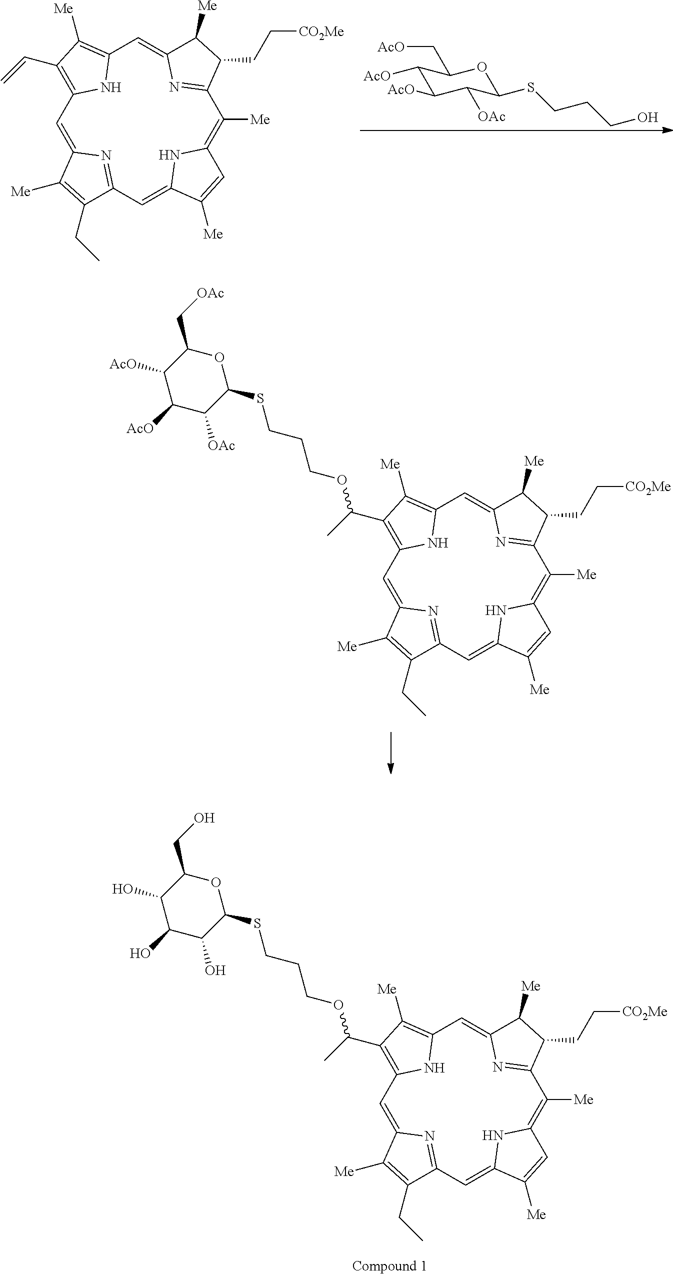 [SMILES: C=Cc1c(C)c2cc3nc(c(C)c4cc(C)c(cc5nc(cc1[nH]2)C(C)=C5CC)[nH]4)[C@@H](CCC(=O)OC)[C@@H]3C ; CC(=O)OCC1OC(SCCCO)C(OC(C)=O)C(OC(C)=O)C1OC(C)=O ; CCC1=C(C)c2cc3[nH]c(cc4nc(c(C)c5cc(C)c(cc1n2)[nH]5)[C@@H](CCC(=O)OC)[C@@H]4C)c(C)c3C(C)OCCCS[C@@H]1O[C@H](COC(C)=O)[C@@H](OC(C)=O)[C@H](OC(C)=O)[C@H]1OC(C)=O ; CCC1=C(C)c2cc3[nH]c(cc4nc(c(C)c5cc(C)c(cc1n2)[nH]5)[C@@H](CCC(=O)OC)[C@@H]4C)c(C)c3C(C)OCCCS[C@@H]1O[C@H](CO)[C@@H](O)[C@H](O)[C@H]1O]

Step 1: To a 100 mL RBF containing phyllochlorin methyl ester (0.500 g, 0.957 mmol, 1 eq) and a stirrer bar was added HBr/AcOH (33% w/w, 9 mL), and the dark blue mixture was stirred (420 rpm) at 30° C. (external temperature) for 2 hours under N₂. A stream of N₂ was passed over the sample for a few minutes to remove some of the HBr before concentrating the bulk by rotary evaporation. The mixture was then further dried under high vacuum (0.2 mbar) at an external temperature of 40° C. for 30 minutes. The residue was reconstituted in DCM (20 mL) before K₂CO₃ (1.32 g, 9.57 mmol, 10 eq), then (2R,3R,4S,5R,6S)-2-(acetoxymethyl)-6-((3-hydroxypropyl)thio)tetrahydro-2H-pyran-3,4,5-triyl triacetate (1.21 g, 2.87 mmol, 3 eq) were added. The dark solution was stirred for 1 hour at 30° C. (external temperature), at which point a concentrated aliquot of the reaction mixture showed the disappearance of the bromide resonances. The reaction mixture was transferred to a separatory funnel and washed with H₂O (20 mL), then brine (20 mL) before being dried (Na₂SO₄) and concentrated to give the crude coupled product as a dark green film (1.78 g). The residue was purified by Biotage autocolumn chromatography (MeOH/DCM gradient), with the sample loaded onto the cartridge as a solution in DCM. The compound had an R_{f} of 0.25 in 1% MeOH/DCM. Phyllochlorin methyl ester propyloxythio-D-glucose peracetate was obtained as a dark green film.

Step 2: To a solution of phyllochlorin methyl ester propyloxythio-D-glucose peracetate (0.400 g, 0.419 mmol, 1 eq) in a mixture of DCM (5 mL) and MeOH (5 mL) was added a 4.6 M solution of NaOMe in MeOH (0.46 mL, 2.10 mmol, 5 eq) dropwise. The dark green solution was stirred at ambient temperature for 20 minutes. The reaction was quenched with AcOH (10 drops) and concentrated by rotary evaporation. The residue was partitioned between DCM (20 mL) and H₂O (20 mL). The organic phase was collected and the aqueous phase re-extracted with DCM (20 mL). The combined organic phases were dried (Na₂SO₄) and concentrated by rotary evaporation to give the crude deacetylated product as a dark green film (334 mg). The product was purified by Biotage autocolumn chromatography, with the sample loaded on the cartridge as a solution in 5% MeOH/DCM. The product, compound 1, had an R_{f} of 0.4 in 10% MeOH/DCM.

### Synthesis Example 2—synthesis of phyllochlorin N-3-hydroxypropyl-N-methyl propylamide propyloxythio-D-glucose (compound 2)

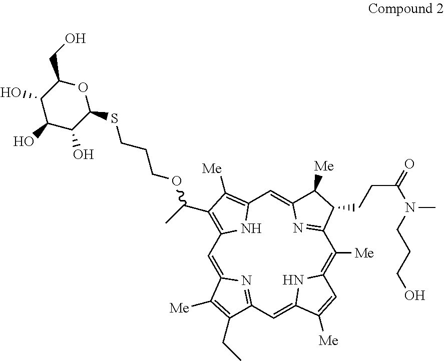 [SMILES: CCC1=C(C)c2cc3[nH]c(cc4nc(c(C)c5cc(C)c(cc1n2)[nH]5)[C@@H](CCC(=O)N(C)CCCO)[C@@H]4C)c(C)c3C(C)OCCCS[C@@H]1O[C@H](CO)[C@@H](O)[C@H](O)[C@H]1O]

### Synthesis of 3-((7S,8S)-18-ethyl-2,5,8,12,17-pentamethyl-13-vinyl-7H,8H-porphyrin-7-yl)-N-(3-hydroxypropyl)-N-methylpropanamide (phyllochlorin N-3-hydroxypropyl-N-methyl propylamide)

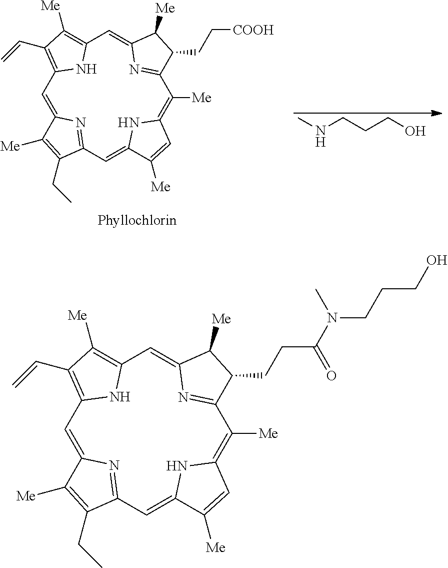

Into a 100 mL RBF fitted with a nitrogen inlet and containing a small stirrer bar was added phyllochlorin (2.00 g, 3.93 mmol, 1 eq), dichloromethane (50 mL), PyBOP (2.26 mg, 1.1 eq), triethylamine (1.64 mL, 3 eq) and 3-(methylamino)-propanol (0.42 g, 1.2 eq). The mixture was stirred at room temperature for 3 hours. Analysis by HPLC showed the reaction to be complete. The reaction mixture was transferred to a separatory funnel and washed with water (2×30 mL). The organic layer was dried (Na₂SO₄) and concentrated by rotary evaporation to give the crude product as a blue/brown film (5.1 g). The crude mixture was loaded directly onto a silica column and eluted with 1.5-2% MeOH/DCM. Pure fractions containing a green/blue spot by TLC with R_{f} 0.30 (5% MeOH/DCM) were combined to give phyllochlorin N-3-hydroxypropyl-N-methyl propylamide (1.29 g, 57%).

¹H NMR (400 MHZ, CDCl₃) δ 9.75-9.70 (m, 2H), 8.85 (brs, 1H), 8.16 (dd, 1H), 6.38 (dd, 1H), 6.15 (dd, 1H), 4.70-4.65 (m, 1H), 4.58-4.50 (m, 1H), 4.00 (s, 3H), 3.89-3.82 (m, 3H), 3.65 (s, 3H), 3.53 (s, 3H), 3.37 (s, 3H), 3.31-3.15 (m, 4H), 2.65-2.53 (m, 2H), 2.37-2.22 (m, 3H), 2.16 (3, 3H), 1.80-1.70 (m, 7H), 1.48-1.40 (m, 2H), −2.10 (s, 1H), −2.22 (m, 1H).

### Acetylation of 3-((7S,8S)-18-ethyl-2,5,8,12,17-pentamethyl-13-vinyl-7H,8H-porphyrin-7-yl)-N-(3-hydroxypropyl)-N-methylpropanamide (phyllochlorin N-3-hydroxypropyl-N-methyl propylamide)

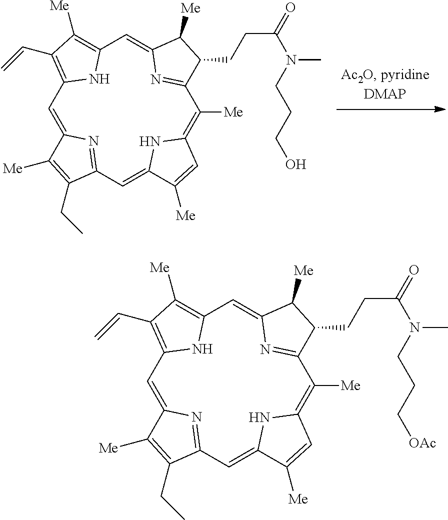

Into a 1 neck 50 mL RBF was added phyllochlorin N-3-hydroxypropyl-N-methyl propylamide (1.20 mg, 2.07 mmol, 1 eq), pyridine (10 mL), acetic anhydride (2.0 mL, 21.2 mmol, 10 eq) and DMAP (5 mg). The solution was stirred at 30° C. (external heat block) for 1 hour. Analysis by TLC indicated the reaction was complete. Ethyl acetate (10 mL) and water (15 mL) were added and the mixture was stirred vigorously for 10 minutes. The layers were separated and the ethyl acetate layer washed with 0.5 M HCl (3×15 mL), saturated NaHCO₃ (3×15 mL), dried (Na₂SO₄) and concentrated to give the crude product as a dark solid. The residue was purified by silica column chromatography (4×18 cm) using a graduated solvent system of 1-2.5% MeOH/DCM. Phyllochlorin N-3-acetoxypropyl-N-methyl propylamide was isolated as a dark green-blue flaky solid (0.97 g, 78%).

¹H NMR (400 MHZ, CDCl₃) δ 9.72 (brs, 2H), 8.86 (m, 2H), 8.16 (dd, 1H), 6.48 (d, 1H), 6.16 (d, 1H), 4.69 (m, 1H), 4.52 (m, 1H), 4.02 (m, 3H), 3.93 (t, 1H), 3.85 (q, 2H), 3.65 (s, 3H), 3.53 (s, 3H), 3.37 (s, 3H), 3.20 (t, 1H), 2.65 (s, 1H), 2.62-2.50 (m, 2H), 2.35-2.20 (m, 5H), 1.96 (s, 2H), 1.81-1.70 (m, 7H), 1.68-1.49 (m, 2H), 1.30-1.20 (m, 1H), −2.13 (br, 1H), −2.22 (br, 1H).

### Bromination/Thio-D-Glucose Substitution

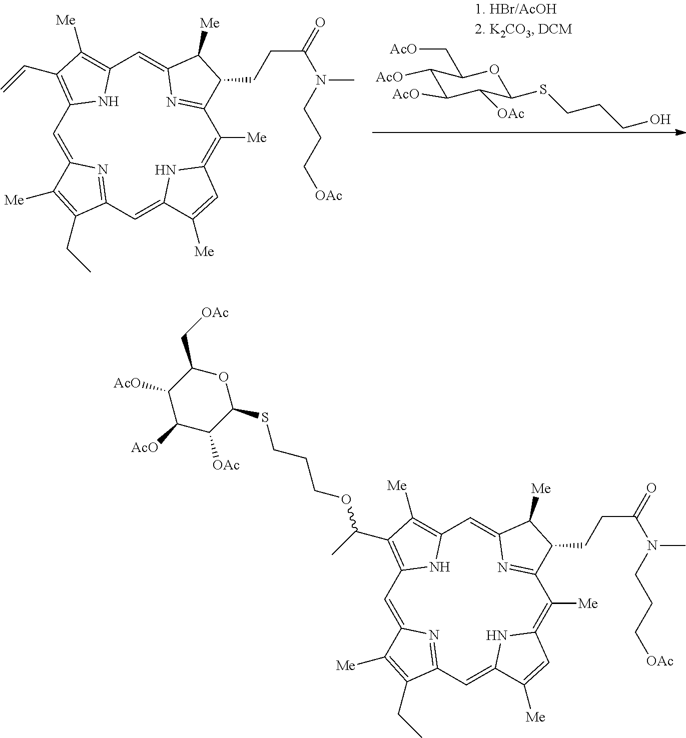

To a 250 mL RBF containing phyllochlorin N-3-acetoxypropyl-N-methyl propylamide (0.82 g, 1.32 mmol, 1 eq) and a stirrer bar was added HBr/AcOH (33% w/w, 14 mL) and the dark blue mixture was stirred (420 rpm) at 30° C. (external) for 2 hours under N₂. ¹H NMR analysis of a concentrated aliquot at this point showed clean conversion to the bromide. A stream of N₂ was passed over the sample for a few minutes to remove some of the HBr before concentrating the bulk by rotary evaporation. The mixture was then further dried under high vacuum (˜0.4 mbar) at an external temperature of 40° C. for 30 minutes. The residue was reconstituted in DCM (20 mL) before K₂CO₃ (1.82 g, 13.19 mmol, 10 eq), then 2,3,4,6-tetra-O-acetyl-1-(3′-hyroxypropyl)thio-D-glucopyranoside (1.53 g, 3.63 mmol, 2.75 eq) were added. The dark solution was stirred overnight at 30° C. (external), at which point a concentrated aliquot of the reaction mixture showed the disappearance of the bromide resonances. The reaction mixture was transferred to a separatory funnel and washed with H₂O (20 mL), then brine (20 mL) before being dried (Na₂SO₄) and concentrated to give the crude coupled product as a dark green film (2.4 g). The residue was purified by silica column chromatography (3×21 cm) using a graduated solvent system of 1-2% MeOH/DCM. Fractions containing the product (R^{β} of 0.3 in 2% MeOH/DCM) were combined to give the crude product as a dark green film (1.75 g) which was used without further purification.

### Global Deacetylation

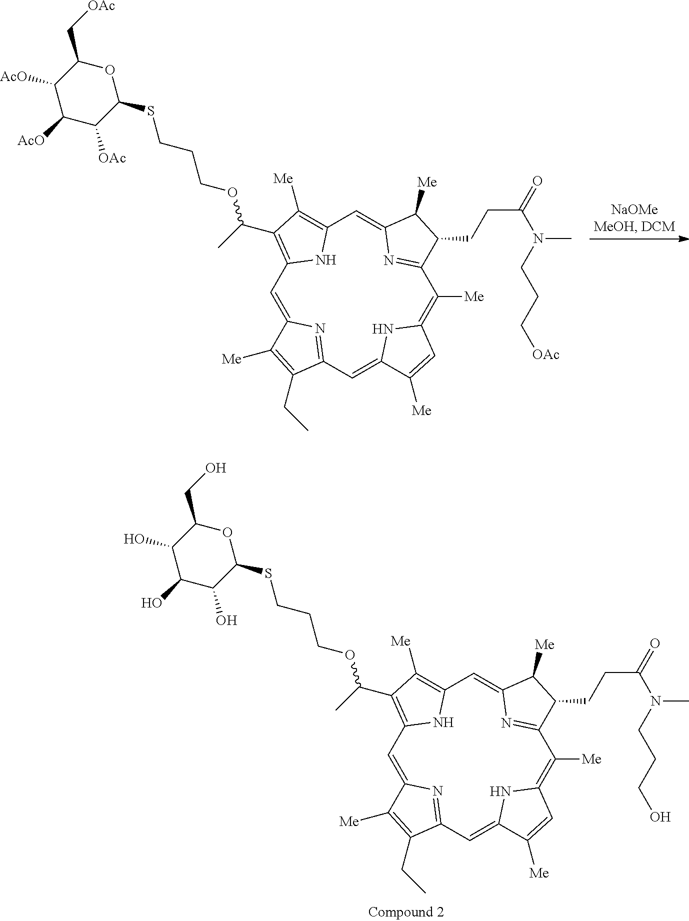 [SMILES: CCC1=C(C)c2cc3[nH]c(cc4nc(c(C)c5cc(C)c(cc1n2)[nH]5)[C@@H](CCC(=O)N(C)CCCOC(C)=O)[C@@H]4C)c(C)c3C(C)OCCCS[C@@H]1O[C@H](COC(C)=O)[C@@H](OC(C)=O)[C@H](OC(C)=O)[C@H]1OC(C)=O ; CCC1=C(C)c2cc3[nH]c(cc4nc(c(C)c5cc(C)c(cc1n2)[nH]5)[C@@H](CCC(=O)N(C)CCCO)[C@@H]4C)c(C)c3C(C)OCCCS[C@@H]1O[C@H](CO)[C@@H](O)[C@H](O)[C@H]1O]

To a solution of phyllochlorin N-3-acetoxypropyl-N-methyl propylamide propyloxythio-D-glucose peracetate (1.75 g, 1.68 mmol, 1 eq) in a mixture of DCM (10 mL) and MeOH (20 mL) was added a 4.6 M solution of NaOMe in MeOH (1.80 mL, 8.38 mmol, 5 eq) dropwise. The dark green solution was stirred at ambient temperature for 90 minutes. The reaction was quenched with AcOH (0.55 g) and concentrated by rotary evaporation to give the deacetylated product as a dark green film (1.75 g). The crude product was purified by silica column chromatography (4×20 cm, 5-12% MeOH/DCM). The pure fractions (TLC R^{β} of 0.2 in 10% MeOH/DCM) were combined to give the product, compound 2, as a dark blue-green flaky solid (0.48 g, 44% over 2 steps) (HPLC purity: 97.3%).

¹H NMR (400 MHZ, CDCl₃) δ 10.20-9.87 (m, 1H), 9.74 (brs, 1H), 8.97-8.80 (brs, 2H), 6.10-5.85 (m, 1H), 4.72-4.47 (m, 2H), 3.97 (s, 3H), 3.90-3.74 (m, 3H), 3.70-3.60 (m, 4H), 3.55-3.40 (m, 4H), 3.40-3.35 (s, 3H), 3.28-3.05 (m, 5H), 2.80-2.52 (m, 5H), 2.50-2.40 (m, 3H), 2.38-2.25 (m, 5H), 2.20-2.10 (m, 6H), 2.08-1.97 (m, 4H), 1.95-1.80 (m, 3H), 1.80-1.67 (m, 8H), 1.45-1.33 (m, 2H), −2.40 (brs, 1H).

### Synthesis Example 3—synthesis of phyllochlorin 13-hydroxymethyl methyl ester (compound 3)

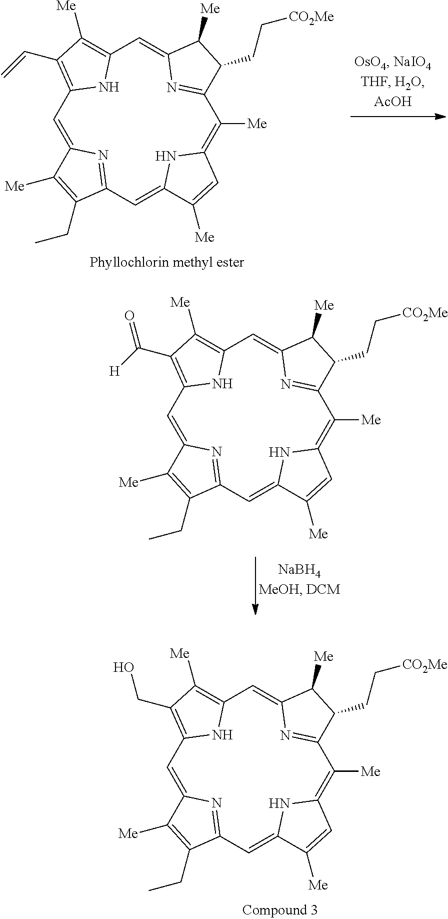 [SMILES: C=Cc1c(C)c2cc3nc(c(C)c4cc(C)c(cc5nc(cc1[nH]2)C(C)=C5CC)[nH]4)[C@@H](CCC(=O)OC)[C@@H]3C ; CCC1=C(C)c2cc3[nH]c(cc4nc(c(C)c5cc(C)c(cc1n2)[nH]5)[C@@H](CCC(=O)OC)[C@@H]4C)c(C)c3C=O ; CCC1=C(C)c2cc3[nH]c(cc4nc(c(C)c5cc(C)c(cc1n2)[nH]5)[C@@H](CCC(=O)OC)[C@@H]4C)c(C)c3CO]

Step 1: To a 250 mL RBF was added phyllochlorin methyl ester (2.00 g, 3.826 mmol, 1 eq), THF (75 mL), osmium tetroxide (5 mg, 0.038 mmol, 0.01 eq), deionized water (5 mL), AcOH (5 mL) and sodium periodate (1.80 g, 8.418 mmol, 2.2 eq). The resultant mixture was stirred (420 rpm) under nitrogen in the dark at ambient temperature for 3 hours. A further portion of sodium periodate (0.35 g, 0.4 eq) was added and the solution stirred overnight. The reaction mixture was concentrated using a rotary evaporator to remove the THF and then re-dissolved in DCM (60 mL), transferred to a separatory funnel and washed with brine (40 mL), saturated NaHCO₃ (40 mL), water (40 mL), dried (Na₂SO₄) and concentrated by rotary evaporation to give a red-brown powdery solid (˜2.5 g). The residue was subjected to column chromatography (4×15 cm) using a gradient of 0-2% MeOH/DCM. Fractions containing the major dark green spot (R_{f}=0.7 in 5% MeOH/DCM) were combined to give the intermediate aldehyde, phyllochlorin 13-formyl methyl ester (1.65 g, 82%) (HPLC purity: 95%).

Step 2: To a 250 mL RBF was added phyllochlorin 13-formyl methyl ester (1.00 g, 1.906 mmol, 1 eq), MeOH (20 mL), DCM (10 mL) and sodium borohydride (145 mg, 3.812 mmol, 2 eq). The resultant mixture was stirred (300 rpm) under nitrogen at ambient temperature for 1 hour. The reaction mixture was diluted with water (40 mL) and stirred for 10 minutes. The mixture was then diluted with DCM (40 mL) and brine (30 mL). The DCM layer was collected and the aqueous was further extracted with DCM (2×20 mL). The combined DCM layers were washed with brine (30 mL), dried (Na₂SO₄) and concentrated by rotary evaporation to give a dark green solid (˜1.2 g). The residue was subjected to column chromatography (3×17 cm) eluting using a gradient of 1-2% MeOH/DCM. Fractions containing the major dark green spot (R_{f}=0.7 in 5% MeOH/DCM) were combined to give compound 3 (0.82 g, 82%) (HPLC purity: 99.5%).

¹H NMR (400 MHZ, CDCl₃) δ 9.72 (s, 1H), 9.63 (m, 1H), 8.86 (m, 2H), 5.88 (m, 2H), 4.58 (m, 1H), 4.51 (m, 1H), 4.00 (s, 3H), 3.84 (q, 2H), 3.63 (s, 3H), 3.58 (s, 3H), 3.48 (m, 3H), 3.35 (s, 3H), 2.63-2.48 (m, 2H), 2.20-2.00 (m, 2H), 1.98-1.90 (m, 1H), 1.79 (d, 3H), 1.75 (d, 3H), −2.21 (brs, 1H), −2.37 (brs, 1H).

### Synthesis Example 4—synthesis of phyllochlorin 13-hydroxymethyl-β-D-glucoside ether methyl ester (compound 4)

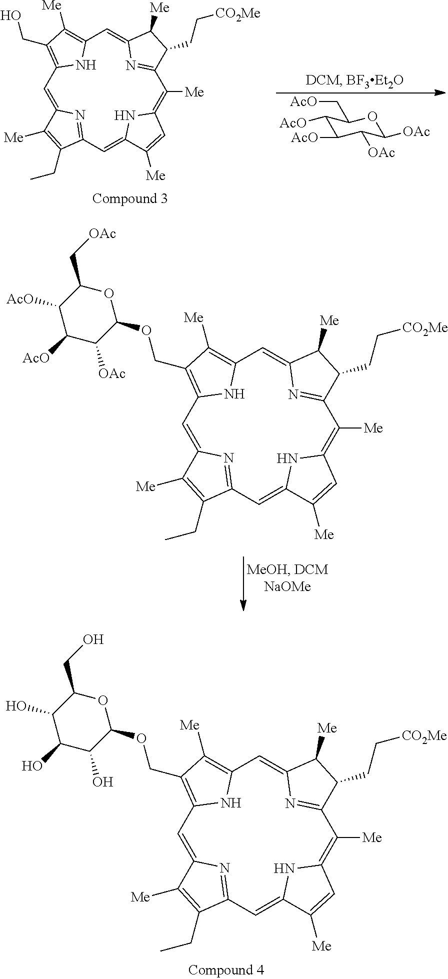 [SMILES: CCC1=C(C)c2cc3[nH]c(cc4nc(c(C)c5cc(C)c(cc1n2)[nH]5)[C@@H](CCC(=O)OC)[C@@H]4C)c(C)c3CO ; CC(=O)OCC1OC(OC(C)=O)C(OC(C)=O)C(OC(C)=O)C1OC(C)=O ; CCC1=C(C)c2cc3[nH]c(cc4nc(c(C)c5cc(C)c(cc1n2)[nH]5)[C@@H](CCC(=O)OC)[C@@H]4C)c(C)c3CO[C@@H]1O[C@H](COC(C)=O)[C@@H](OC(C)=O)[C@H](OC(C)=O)[C@H]1OC(C)=O ; CCC1=C(C)c2cc3[nH]c(cc4nc(c(C)c5cc(C)c(cc1n2)[nH]5)[C@@H](CCC(=O)OC)[C@@H]4C)c(C)c3CO[C@@H]1O[C@H](CO)[C@@H](O)[C@H](O)[C@H]1O]

Step 1: To a 25 mL RBF was added phyllochlorin 13-hydroxymethyl methyl ester (compound 3) (100 mg, 0.190 mmol, 1 eq), glucose pentaacetate (89 mg, 0.228 mmol, 1.2 eq) and DCM (4 mL). The resultant mixture was stirred (420 rpm) under nitrogen with cooling to <5° C. Boron trifluoride diethyl etherate (5 drops via a 1 mL disposable syringe) was added and stirring was continued allowing the solution to warm slowly to ˜10° C. over 1 hour. Further boron trifluoride diethyl etherate (8 drops via a 1 mL disposable syringe) was added and the mixture allowed to warm slowly to room temperature. After 5 hours the reaction mixture was diluted with DCM (20 mL), transferred to a separatory funnel and washed with saturated NaHCO₃ (10 mL), then water (20 mL). The organic phase was dried (Na₂SO₄) and concentrated by rotary evaporation to give a blue-black film (225 mg). The residue was subjected to column chromatography (3×16 cm) eluting using a gradient of 0.5-1.5% MeOH/DCM. The partially purified phyllochlorin 13-hydroxymethyl-β-glucoside ether methyl ester peracetate (180 mg) was used directly in the next step.

Step 2: To a solution of phyllochlorin 13-hydroxymethyl-β-glucoside ether methyl ester peracetate (180 mg, 0.190 mmol, 1 eq) in MeOH (2 mL) and DCM (2 mL) was added NaOMe (4.6 M in MeOH, 0.05 mL, 0.23 mmol, 1.2 eq), and the mixture stirred (420 rpm) under N₂ for 1 hour monitoring by HPLC. The reaction was concentrated by rotary evaporation to give a black film. The residue was purified by column chromatography (3×15 cm) eluting using a gradient of 5-10% MeOH/DCM. Fractions containing a green spot (R_{f}=0.1 in 5% MeOH/DCM) were combined to give compound 4 (38 mg, 29% over 2 steps) (HPLC purity: 99.6%).

¹H NMR (400 MHZ, DMSO-d₆) δ 9.85 (s, 1H), 9.76 (s, 1H), 9.08 (m, 2H), 6.15 (d, 1H), 6.00 (d, 1H), 5.11 (d, 1H), 4.95 (t, 2H), 4.83 (t, 1H), 4.65-4.58 (m, 2H), 4.55 (d, 1H), 4.11 (q, 1H), 3.95 (s, 3H), 3.90 (m, 1H), 3.82 (q, 2H), 3.72-3.65 (m, 1H), 3.62 (s, 3H), 3.53 (s, 3H), 3.51 (s, 3H), 3.26 (m, 1H), 3.21-3.15 (m, 3H), 3.12 (m, 1H), 2.80-2.70 (m, 1H), 2.45-2.35 (m, 2H), 1.85-1.75 (m, 1H), 1.75-1.67 (m, 6H), −2.46 (s, 1H), −2.58 (s, 1H).

### Synthesis Example 5—synthesis of phyllochlorin 13-hydroxymethyl N-3-hydroxypropyl-N-methyl-propylamide acetate (compound 5)

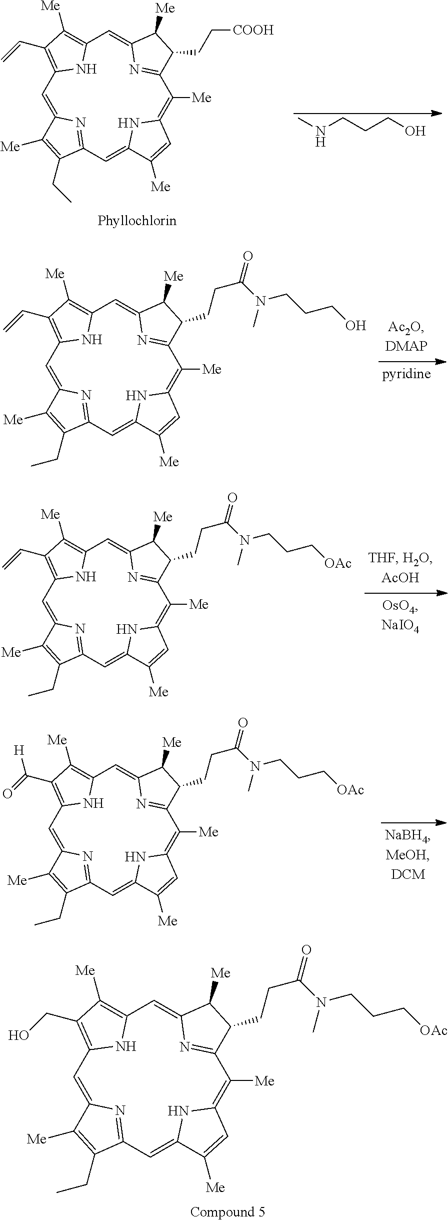

Step 1: Into a 100 mL RBF fitted with a nitrogen inlet and containing a small stirrer bar was added phyllochlorin (2.00 g, 3.93 mmol, 1 eq), dichloromethane (50 mL), PyBOP (2.26 mg, 1.1 eq), triethylamine (1.64 mL, 3 eq) and 3-(methylamino)-propanol (0.42 g, 1.2 eq). The mixture was stirred at room temperature for 3 hours. Analysis by HPLC showed the reaction to be complete. The reaction mixture was transferred to a separatory funnel and washed with water (2×30 mL). The organic layer was dried (Na₂SO₄) and concentrated by rotary evaporation to give the crude product as a blue/brown film (5.1 g). The crude mixture was loaded directly onto a silica column and eluted with 1.5-2% MeOH/DCM. Pure fractions containing a green/blue spot by TLC with R_{f} 0.30 (5% MeOH/DCM) were combined to give phyllochlorin N-3-hydroxypropyl-N-methyl-propylamide (1.29 g, 57%).

¹H NMR (400 MHZ, CDCl₃) δ 9.75-9.70 (m, 2H), 8.85 (br s, 1H), 8.16 (dd, 1H), 6.38 (dd, 1H), 6.15 (dd, 1H), 4.70-4.65 (m, 1H), 4.58-4.50 (m, 1H), 4.00 (s, 3H), 3.89-3.82 (m, 3H), 3.65 (s, 3H), 3.53 (s, 3H), 3.37 (s, 3H), 3.31-3.15 (m, 4H), 2.65-2.53 (m, 2H), 2.37-2.22 (m, 3H), 2.16 (3, 3H), 1.80-1.70 (m, 7H), 1.48-1.40 (m, 2H), −2.10 (s, 1H), −2.22 (m, 1H).

Step 2: Into a 1-necked 25 mL RBF was added phyllochlorin N-3-hydroxypropyl-N-methyl-propylamide (300 mg, 0.517 mmol, 1 eq), pyridine (2 mL), acetic anhydride (0.49 mL, 5.17 mmol, 10 eq) and DMAP (1 mg). The solution was stirred at 30° C. for 1 hour. Analysis by TLC indicated the reaction was complete. Ethyl acetate (15 mL) and water (10 mL) were added and the mixture stirred vigorously for 10 minutes. The layers were separated and the ethyl acetate layer was washed with 0.5 M HCl (2×10 mL), saturated NaHCO₃ (2×10 mL), before being dried (Na₂SO₄) and concentrated to give phyllochlorin N-3-hydroxypropyl-N-methyl-propylamide acetate as a dark green solid that was not purified further (288 mg, 89%) (HPLC purity: 95.6%).

Step 3: To a 100 mL RBF was added phyllochlorin N-3-hydroxypropyl-N-methyl-propylamide acetate (280 mg, 0.450 mmol, 1 eq), THF (15 mL), osmium tetroxide (1 mg, 0.005 mmol, 0.01 eq), deionized water (1 mL), AcOH (1 mL) and sodium periodate (202 mg, 0.946 mmol, 2.1 eq). The resultant mixture was stirred (420 rpm) under nitrogen in the dark at ambient temperature overnight. The reaction mixture was concentrated using a rotary evaporator to remove the THF and then re-dissolved in DCM (75 mL), transferred to a separatory funnel and washed with brine (30 mL), saturated NaHCO₃ (30 mL) and water (40 mL) before being dried (Na₂SO₄) and concentrated by rotary evaporation to give a red-brown powdery solid (˜0.28 g). The residue was subjected to column chromatography (3×15 cm) eluting using a gradient of 1-2.5% MeOH/DCM. Fractions containing the major dark green spot (R_{f}=0.65 in 5% MeOH/DCM) were combined to give the intermediate aldehyde, phyllochlorin 13-formyl N-3-hydroxypropyl-N-methyl-propylamide acetate (200 mg, 71%).

Step 4: To a 50 mL RBF was added phyllochlorin 13-formyl N-3-hydroxypropyl-N-methyl-propylamide acetate (190 mg, 0.305 mmol, 1 eq), MeOH (10 mL), DCM (5 mL) and sodium borohydride (23 mg, 0.609 mmol, 2 eq). The resultant mixture was stirred (420 rpm) under nitrogen at ambient temperature for 30 minutes. The reaction mixture was diluted with water (20 mL) and stirred for 10 minutes. The mixture was then extracted with chloroform (3×10 mL). The combined chloroform layers were washed with water (20 mL), dried (Na₂SO₄) and concentrated by rotary evaporation to give a dark green oil. The residue was subjected to column chromatography (3×12 cm) eluting using a gradient of 2-3% MeOH/DCM. Fractions containing the major dark green spot (R_{f}=0.4 in 5% MeOH/DCM) were combined to give compound 5 (173 mg, 91%).

¹H NMR (400 MHZ, CDCl₃) δ 9.70 (m, 2H), 8.85 (m, 2H), 6.05-5.90 (m, 2H), 4.70-4.65 (m, 1H), 4.55-4.48 (m, 1H), 4.10 (t, 1H), 4.01 (m, 3H), 3.95-3.88 (m, 4H), 3.83 (q, 2H), 3.70 (m, 1H), 3.62 (s, 3H), 3.50 (s, 3H), 3.35 (m, 3H), 3.18 (t, 1H), 3.15 (s, 1H), 2.70-2.65 (m, 1H), 2.60-2.50 (m, 1H), 2.35-2.20 (m, 4H), 2.05 (s, 1H), 2.00-1.95 (m, 2H), 1.80-1.70 (m, 7H), 1.60 (m, 2H), −2.24 (brs, 1H), −2.40 (brs, 1H).

### Synthesis Example 6—synthesis of phyllochlorin N-3-hydroxypropyl-N-methyl-propylamide 13-hydroxymethyl-β-D-glucoside ether (compound 6)

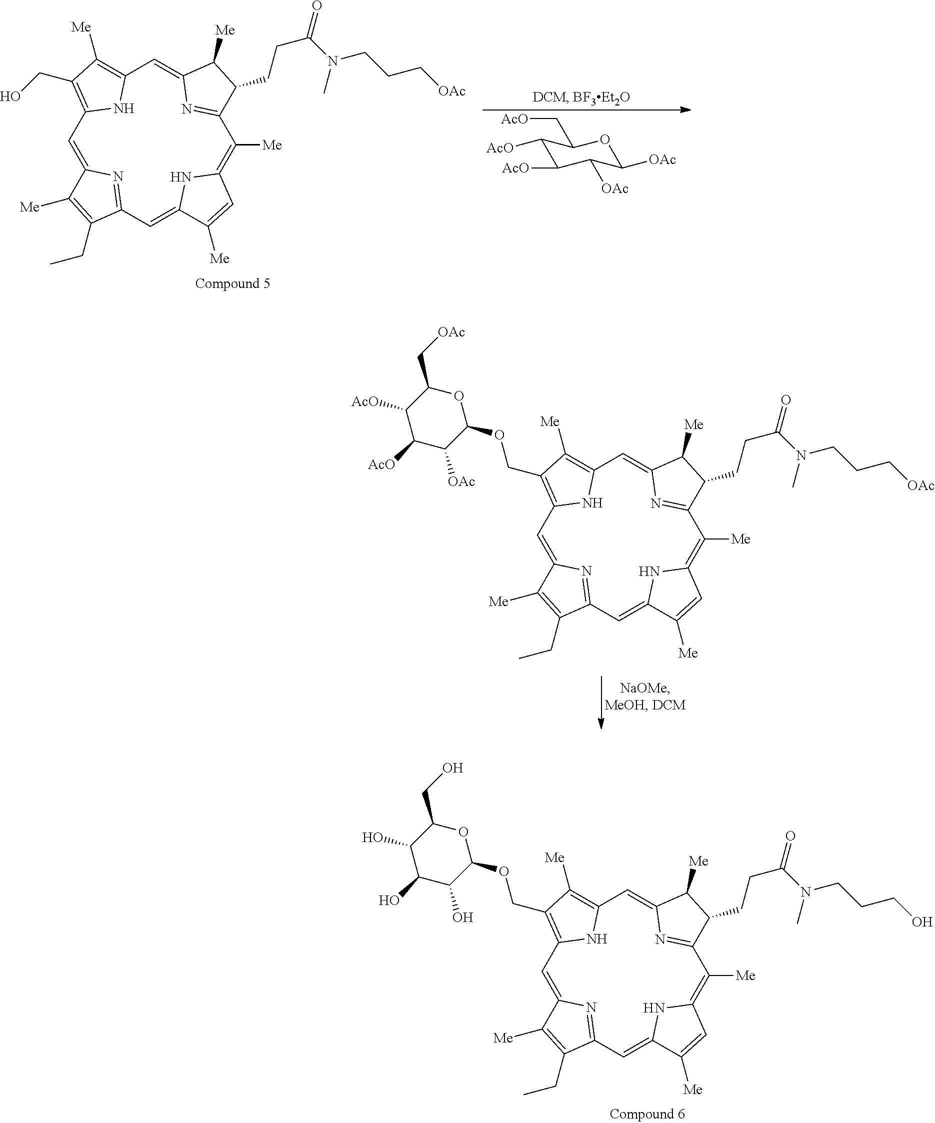 [SMILES: CCC1=C(C)c2cc3[nH]c(cc4nc(c(C)c5cc(C)c(cc1n2)[nH]5)[C@@H](CCC(=O)N(C)CCCOC(C)=O)[C@@H]4C)c(C)c3CO ; CC(=O)OCC1OC(OC(C)=O)C(OC(C)=O)C(OC(C)=O)C1OC(C)=O ; CCC1=C(C)c2cc3[nH]c(cc4nc(c(C)c5cc(C)c(cc1n2)[nH]5)[C@@H](CCC(=O)N(C)CCCOC(C)=O)[C@@H]4C)c(C)c3CO[C@@H]1O[C@H](COC(C)=O)[C@@H](OC(C)=O)[C@H](OC(C)=O)[C@H]1OC(C)=O ; CCC1=C(C)c2cc3[nH]c(cc4nc(c(C)c5cc(C)c(cc1n2)[nH]5)[C@@H](CCC(=O)N(C)CCCO)[C@@H]4C)c(C)c3CO[C@@H]1O[C@H](CO)[C@@H](O)[C@H](O)[C@H]1O]

Step 1: To a 50 mL RBF was added phyllochlorin 13-hydroxymethyl N-3-hydroxypropyl-N-methyl-propylamide acetate (compound 5) (160 mg, 0.256 mmol, 1 eq), glucose pentaacetate (150 mg, 0.384 mmol, 1.5 eq) and DCM (10 mL). The resultant mixture was stirred (420 rpm) under nitrogen cooling to ˜0° C. Boron trifluoride diethyl etherate (0.2 mL) was added and stirring was continued allowing the solution to warm slowly to room temperature over 2 hours and then stirred at room temperature in the dark overnight. The reaction was monitored by HPLC. The reaction mixture was diluted with DCM (20 mL), transferred to a separatory funnel and washed with saturated NaHCO₃ (15 mL), then water (20 mL). The organic phase was dried (Na₂SO₄) and concentrated by rotary evaporation to give a blue-black film (350 mg). The residue was subjected to column chromatography (3×16 cm) eluting using a gradient of 1-2% MeOH/DCM. The partially purified phyllochlorin 13-hydroxymethyl-β-glucoside ether N-3-hydroxypropyl-N-methyl-propylamide peracetate (260 mg) was used directly in the next step.

Step 2: To a solution of phyllochlorin 13-hydroxymethyl-β-glucoside ether N-3-hydroxypropyl-N-methyl-propylamide peracetate (250 mg, 0.255 mmol, 1 eq) in MeOH (2 mL) and DCM (2 mL) was added NaOMe (4.6 M in MeOH, 0.06 mL, 0.23 mmol, 1.2 eq), and the mixture stirred (420 rpm) under N₂ for 1 hour. The reaction was concentrated by rotary evaporation to give a black film. The residue was purified by column chromatography (3×16 cm) eluting using a gradient of 5-12% MeOH/DCM. Fractions containing the major dark green spot (R_{f}<0.1 in 5% MeOH/DCM) were combined to give compound 6 as a dark green solid (75 mg, 39% over 2 steps) (HPLC purity: 95.5%).

¹H NMR (400 MHZ, DMSO-d₆) δ 9.85 (s, 1H), 9.76 (s, 1H), 9.07 (m, 2H), 6.16 (d, 1H), 6.00 (d, 1H), 5.12 (d, 1H), 4.96 (t, 2H), 4.85 (t, 1H), 4.68-4.53 (m, 4H), 4.44 (t, 1H), 3.98 (m, 3H), 3.90 (m, 1H), 3.83 (q, 2H), 3.72-3.65 (m, 1H), 3.63 (s, 3H), 3.50 (s, 3H), 3.22-3.15 (m, 2H), 3.15-3.10 (m, 1H), 2.90 (s, 2H), 2.80 (s, 2H), 2.45-2.35 (m, 1H), 1.75-1.60 (m, 8H), 1.60-1.55 (m, 1H), −2.35 (s, 1H), −2.53 (s, 1H).

### Synthesis Example 7—synthesis of phyllochlorin 13-hydroxymethyl-β-D-maltose ether methyl ester heptaacetate (compound 7)

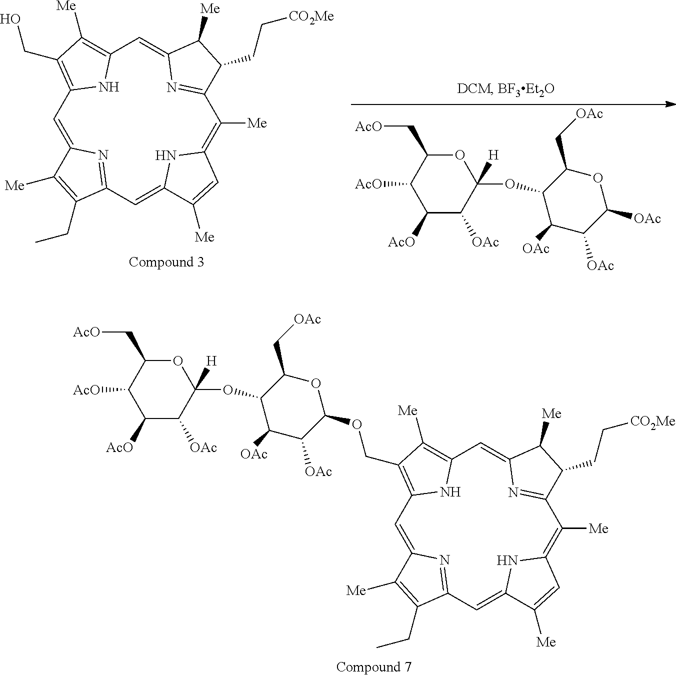 [SMILES: CCC1=C(C)c2cc3[nH]c(cc4nc(c(C)c5cc(C)c(cc1n2)[nH]5)[C@@H](CCC(=O)OC)[C@@H]4C)c(C)c3CO ; CC(=O)OC[C@H]1O[C@H](O[C@H]2[C@H](OC(C)=O)[C@@H](OC(C)=O)[C@H](OC(C)=O)O[C@@H]2COC(C)=O)[C@H](OC(C)=O)[C@@H](OC(C)=O)[C@@H]1OC(C)=O ; CCC1=C(C)c2cc3[nH]c(cc4nc(c(C)c5cc(C)c(cc1n2)[nH]5)[C@@H](CCC(=O)OC)[C@@H]4C)c(C)c3CO[C@@H]1O[C@H](COC(C)=O)[C@@H](O[C@H]2O[C@H](COC(C)=O)[C@@H](OC(C)=O)[C@H](OC(C)=O)[C@H]2OC(C)=O)[C@H](OC(C)=O)[C@H]1OC(C)=O]

To a 50 mL RBF was added phyllochlorin 13-hydroxymethyl methyl ester (compound 3) (100 mg, 0.190 mmol, 1 eq), maltose octaacetate (193 mg, 0.285 mmol, 1.5 eq) and DCM (10 mL). The resultant mixture was stirred (420 rpm) under nitrogen cooling to ˜0° C. Boron trifluoride diethyl etherate (0.2 mL) was added and stirring was continued allowing the solution to warm slowly to room temperature over 2 hours and then stirred at room temperature in the dark overnight. The reaction was monitored by HPLC. The reaction mixture was diluted with DCM (20 mL), transferred to a separatory funnel and washed with saturated NaHCO₃ (15 mL), then water (20 mL). The organic phase was dried (Na₂SO₄) and concentrated by rotary evaporation to give a blue-black film (500 mg). The residue was subjected to column chromatography (3×12 cm) eluting using a gradient of 1-2% MeOH/DCM and the major dark band collected (R_{f}=0.80 in 5% MeOH/DCM) to give compound 7 (265 mg, quantitative) (HPLC purity: 94.7%) as a dark green fluffy solid contaminated with excess maltose octaacetate.

¹H NMR (400 MHZ, CDCl₃) δ 9.74 (s, 1H), 9.63 (s, 1H), 8.89 (m, 2H), 6.20 (d, 1H), 6.05 (d, 1H), 5.32 (d, 1H), 5.38 (dd, 1H), 5.00 (m, 2H), 4.90 (m, 1H), 4.75 (m, 2H), 4.65 (m, 1H), 4.60 (m, 1H), 4.52 (m, 1H), 4.35 (dd, 1H), 4.28 (m, 2H), 4.05 (m, 2H), 4.01 (s, 3H), 3.95 (m, 1H), 3.86 (m, 2H), 3.67 (s, 3H), 3.60 (s, 3H), 3.51 (s, 3H), 3.37 (m, 3H), 2.65-2.50 (m, 2H), 2.30 (s, 3H), 2.10 (m, 5H), 2.02 (m, 7H), 1.92 (s, 3H), 1.83 (s, 3H), 1.80 (m, 5H), 1.75 (t, 3H), −2.20 (s, 1H), −2.41 (s, 1H).

### Synthesis Example 8—synthesis of phyllochlorin 13-hydroxymethyl-β-D-maltose ether methyl ester (compound 8)

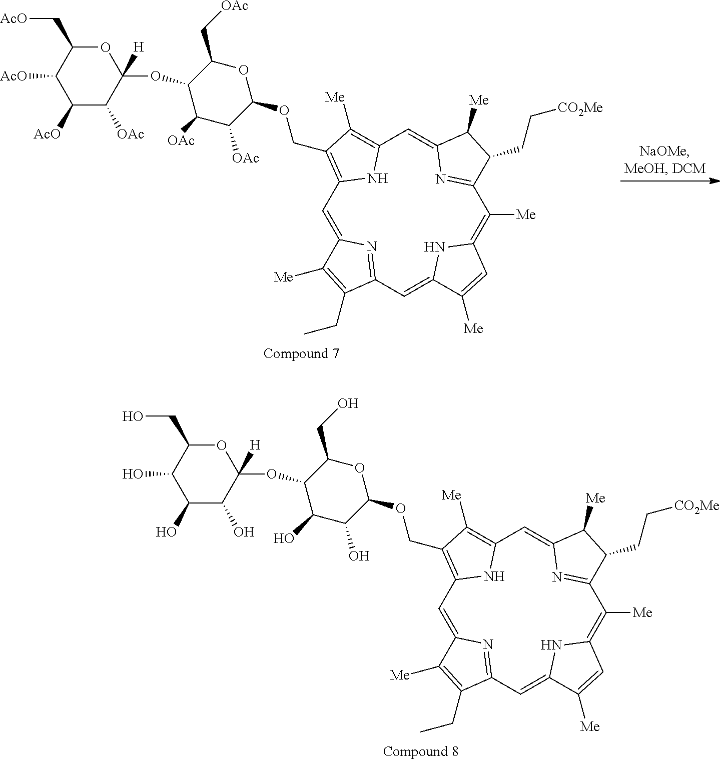 [SMILES: CCC1=C(C)c2cc3[nH]c(cc4nc(c(C)c5cc(C)c(cc1n2)[nH]5)[C@@H](CCC(=O)OC)[C@@H]4C)c(C)c3CO[C@@H]1O[C@H](COC(C)=O)[C@@H](O[C@H]2O[C@H](COC(C)=O)[C@@H](OC(C)=O)[C@H](OC(C)=O)[C@H]2OC(C)=O)[C@H](OC(C)=O)[C@H]1OC(C)=O ; CCC1=C(C)c2cc3[nH]c(cc4nc(c(C)c5cc(C)c(cc1n2)[nH]5)[C@@H](CCC(=O)OC)[C@@H]4C)c(C)c3CO[C@@H]1O[C@H](CO)[C@@H](O[C@H]2O[C@H](CO)[C@@H](O)[C@H](O)[C@H]2O)[C@H](O)[C@H]1O]

To a solution of phyllochlorin 13-hydroxymethyl-β-D-maltose ether methyl ester heptaacetate (compound 7) (230 mg, 0.164 mmol, 1 eq) in MeOH (3 mL) and DCM (3 mL) was added NaOMe (4.6 M in MeOH, 0.04 mL, 0.164 mmol, 1 eq), and the mixture stirred (420 rpm) under N₂ for 2 hours. HPLC analysis after 2 hours showed conversion to the deacetylated product. The reaction was concentrated by rotary evaporation to give a black film. The residue was purified by column chromatography (3×12 cm) eluting using a gradient of 8-12% MeOH/DCM. Fractions containing the product (R_{f}=0.1 in 10% MeOH/DCM) were combined to give compound 8 as blue-black solid (80 mg, 47%) (HPLC purity: 97.8%).

¹H NMR (400 MHZ, DMSO-d₆) δ 9.81 (s, 1H), 9.76 (s, 1H), 9.08 (s, 1H), 9.05 (s, 1H), 6.10 (d, 1H), 5.92 (d, 1H), 5.54 (d, 1H), 5.45 (d, 1H), 5.26 (d, 1H), 5.08 (d, 1H), 4.98-4.90 (m, 2H), 4.86 (t, 1H), 4.65-4.58 (m, 3H), 4.55 (d, 1H), 3.95 (m, 4H), 3.81 (m, 3H), 3.71-3.65 (m, 1H), 3.61 (s, 3H), 3.55-3.45 (m, 9H), 3.35 (s, 3H), 3.26 (m, 2H), 3.14-3.06 (m, 1H), 2.80-2.70 (m, 1H), 2.45-2.35 (m, 2H), 1.85-1.75 (m, 1H), 1.75-1.67 (m, 6H), −2.36 (s, 1H), −2.53 (s, 1H).

### Synthesis Example 9—synthesis of phyllochlorin 13-hydroxymethyl-N-meglumine-propylamide pentaacetate (compound 9)

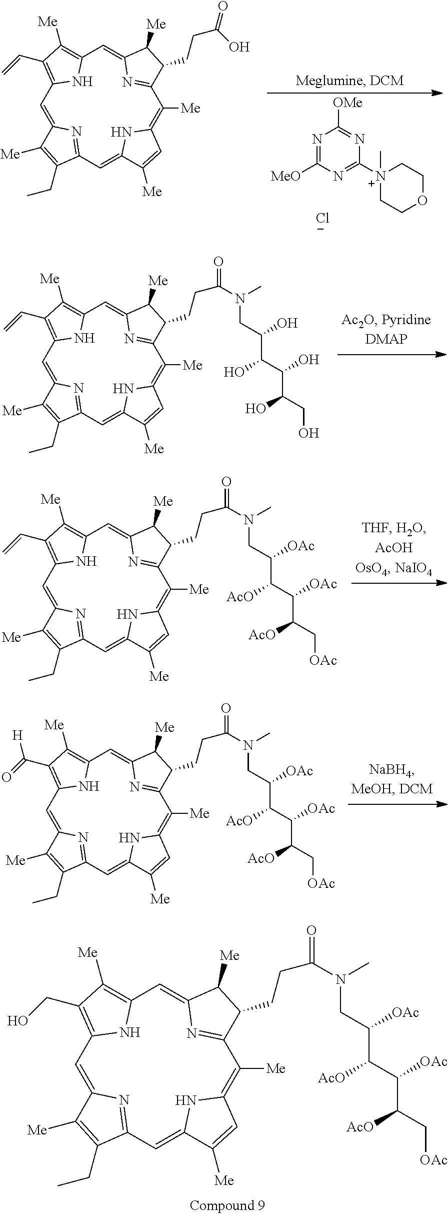

Step 1: To a 50 mL RBF was added phyllochlorin (0.50 g, 0.983 mmol, 1 eq), DMTMM (0.30 g, 1.081 mmol, 1.1 eq), DCM (15 mL) and meglumine (0.23 g, 1.179 mmol, 1.2 eq). The resultant mixture was stirred under nitrogen at ambient temperature for 1 hour. A further portion of meglumine (0.10 g, 0.51 mmol, 0.5 eq) was added and the solution stirred for a further 3 hours. The reaction mixture was transferred to a separatory funnel, diluted with chloroform (30 mL) and washed with 0.5M HCl (50 mL). The aqueous layer was re-extracted with chloroform and the combined organics washed with pH 7 buffer. The organic phase was dried (Na₂SO₄) and concentrated by rotary evaporation to give a blue/black film, which was purified by column chromatography (silica) using a gradient of 5% MeOH/DCM (50 mL), then 7% MeOH/DCM (100 mL), then 9% MeOH/DCM (100 mL), and then 10% MeOH/DCM (200 mL) to give phyllochlorin N-meglumine-propylamide as a dark blue/green solid (432 mg, 64%).

¹H NMR (400 MHZ, d₆-DMSO) δ 9.74 (s, 1H), 9.72 (s, 1H), 9.11 (s, 1H), 9.07 (s, 1H), 8.34 (dd, 1H), 6.44 (d, 1H), 6.17 (d, 1H), 5.10 & 4.75 (2×d, 1H), 4.65-4.40 (m, 5H), 4.30 (m, 1H), 4.00 (m, 3H), 3.90 (m, 1H), 3.80 (m, 2H), 3.68 (m, 1H), 3.62 (s, 3H), 3.60-3.50 (m, 5H), 3.50-3.40 (m, 2H), 3.30-3.08 (m, 1H), 3.00 (s, 1H), 2.90 (s, 2H), 2.86-2.60 (m, 1H), 2.45-2.35 (m, 1H), 1.75-1.65 (m, 6H), 1.75-1.50 (m, 1H), −2.26 (s, 1H), −2.42 (s, 1H).

Step 2: Into a 1 neck 25 mL RBF was added phyllochlorin N-meglumine-propylamide (500 mg, 0.729 mmol, 1 eq), pyridine (3 mL), acetic anhydride (1.38 mL, 14.58 mmol, 20 eq) and DMAP (1 mg). The solution was stirred at 30° C. for 1 hour. Ethyl acetate (20 mL) and water (10 mL) were added and the mixture stirred vigorously for 10 minutes. The layers were separated and the ethyl acetate layer washed with 0.5 M HCl (2×10 mL), saturated NaHCO₃ (2×10 mL), dried (Na₂SO₄) and concentrated to give phyllochlorin N-meglumine-propylamide pentaacetate as a dark green solid (660 mg, quantitative) (HPLC purity: 99.1%).

¹H NMR (400 MHZ, CDCl₃) δ 9.72 (m, 2H), 8.85 (m, 2H), 8.17 (dd, 1H), 6.39 (dd, 1H), 6.14 (dd, 1H), 5.41 (dd, 1H), 5.28 (m, 1H), 5.21 (m, 1H), 4.99 (m, 1H), 4.65 (m, 1H), 4.50 (m, 1H), 4.25 (dd, 1H), 4.08 (dd, 1H), 4.02 (s, 3H), 3.85 (q, 2H), 3.65 (s, 3H), 3.53 (s, 3H), 3.47 (dd, 1H), 3.38 (s, 3H), 3.24 (dd, 1H), 2.60-2.50 (m, 1H), 2.45-2.35 (m, 4H), 2.15 (s, 3H), 2.05 (s, 3H), 2.04 (s, 3H), 1.96 (s, 3H), 1.95 (s, 3H), 1.93 (m, 1H), 1.80-1.73 (m, 6H), −2.10 (brs, 1H), −2.23 (brs, 1H).

Step 3: To a 100 mL RBF was added phyllochlorin N-meglumine-propylamide pentaacetate (600 mg, 0.670 mmol, 1 eq), THF (25 mL), osmium tetroxide (1.7 mg, 0.007 mmol, 0.01 eq), deionized water (1.5 mL), AcOH (1.5 mL) and sodium periodate (358 mg, 1.674 mmol, 2.5 eq). The resultant mixture was stirred (420 rpm) under nitrogen in the dark at ambient temperature overnight. The reaction mixture was concentrated using a rotary evaporator to remove the THF and then re-dissolved in DCM (75 mL), transferred to a separatory funnel and washed with brine (30 mL), saturated NaHCO₃ (30 mL) and water (40 mL) before being dried (Na₂SO₄) and concentrated by rotary evaporation to give a red-brown powdery solid (˜0.80 g). The intermediate aldehyde, phyllochlorin 13-formyl-N-meglumine-propylamide pentaacetate (HPLC purity: 97%) was used without further purification.

Step 4: To a 100 mL RBF was added phyllochlorin 13-formyl-N-meglumine-propylamide pentaacetate (600 mg, 0.668 mmol, 1 eq), MeOH (15 mL), DCM (7 mL) and sodium borohydride (51 mg, 1.336 mmol, 2 eq). The resultant mixture was stirred (420 rpm) under nitrogen at ambient temperature for 30 minutes. The reaction mixture was diluted with water (20 mL) and stirred for 20 minutes. The mixture was then extracted with chloroform (3×10 mL). The combined chloroform layers washed with water (20 mL), dried (Na₂SO₄) and concentrated by rotary evaporation to give a dark green oil. The residue was subjected to column chromatography (4×20 cm) eluting using a gradient of 1-3% MeOH/DCM. Fractions containing the major dark green spot (R_{f}=0.5 in 5% MeOH/DCM) were combined to give compound 9 (267 mg, 44% over 2 steps).

¹H NMR (400 MHZ, CDCl₃) δ 9.70 (m, 2H), 8.86 (m, 2H), 6.05-5.90 (m, 2H), 5.40 (m, 1H), 5.20 (m, 2H), 4.98 (m, 1H), 4.72-4.65 (m, 1H), 4.55-4.48 (m, 1H), 4.25 (dd, 1H), 4.10-4.00 (m, 4H), 3.83 (q, 2H), 3.62 (s, 3H), 3.50 (s, 3H), 3-40-3.35 (m, 4H), 3.26 (dd, 1H), 2.60-2.50 (m, 2H), 2.35-2.20 (m, 4H), 2.13 (s, 3H), 2.05 (s, 3H), 2.04 (s, 3H), 1.96 (s, 3H), 1.94 (s, 3H), 1.85 (m, 2H), 1.80-1.70 (m, 7H), −2.23 (brs, 1H), −2.39 (brs, 1H).

### Synthesis Example 10—synthesis of phyllochlorin N-meglumine-propylamide-13-hydroxymethyl-β-D-glucoside ether (compound 10)

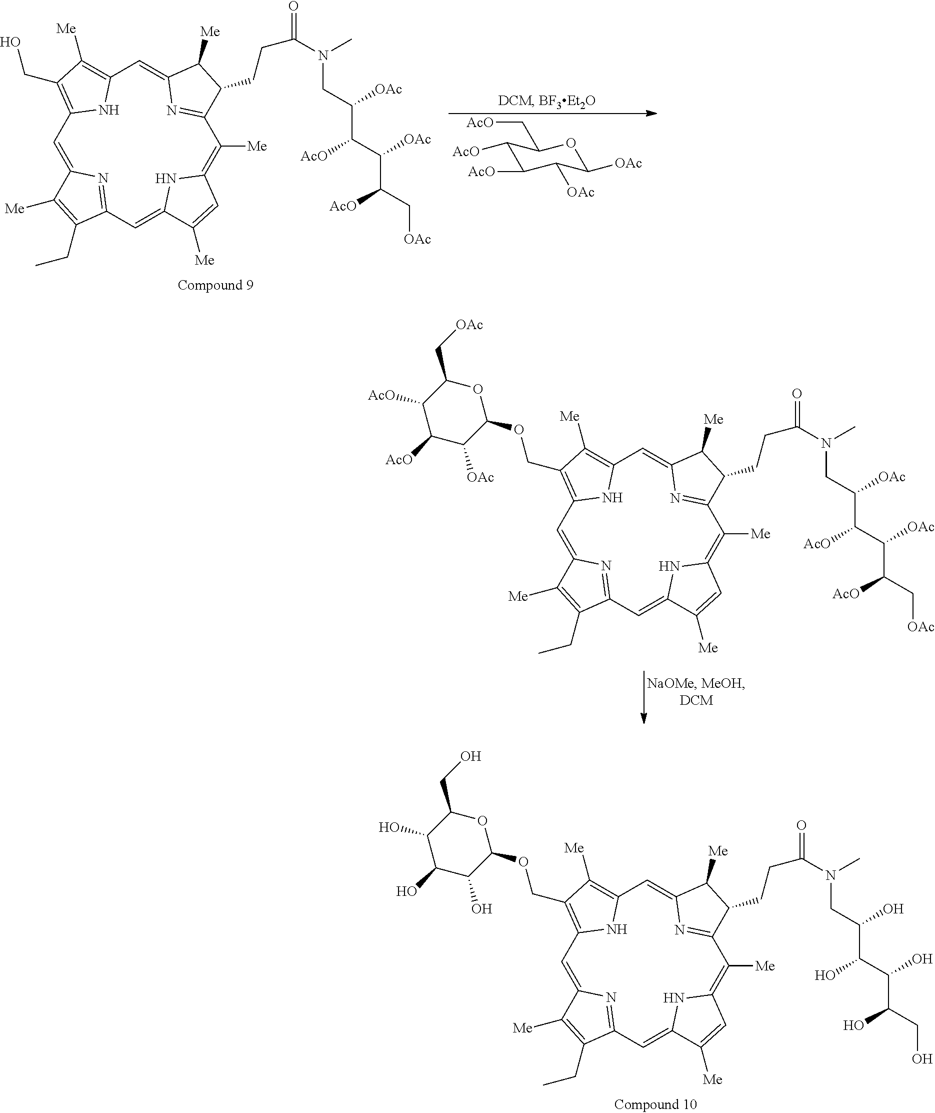

Step 1: To a 50 mL RBF was added phyllochlorin 13-hydroxymethyl-N-meglumine-propylamide pentaacetate (compound 9) (255 mg, 0.283 mmol, 1 eq), glucose pentaacetate (166 mg, 0.425 mmol, 1.5 eq) and DCM (10 mL). The resultant mixture was stirred (420 rpm) under nitrogen cooling to ˜0° C. Boron trifluoride diethyl etherate (0.2 mL) was added and stirring was continued allowing the solution to warm slowly to room temperature over 2 hours and then stirred at room temperature in the dark overnight. The reaction was monitored by HPLC. The reaction mixture was diluted with DCM (20 mL), transferred to a separatory funnel and washed with saturated NaHCO₃ (15 mL), then water (20 mL). The organic phase was dried (Na₂SO₄) and concentrated by rotary evaporation to give a blue-black film (˜0.50 g). The residue was subjected to column chromatography (3×16 cm) eluting using a gradient of 0.5-2% MeOH/DCM. Fractions containing the main dark green band were combined to give a green oil (300 mg). The partially purified phyllochlorin 13-hydroxymethyl-β-glucoside ether-N-meglumine-propylamide peracetate was used directly in the next step.

Step 2: To a solution of phyllochlorin 13-hydroxymethyl-β-glucoside ether-N-meglumine-propylamide peracetate (300 mg, 0.244 mmol, 1 eq) in MeOH (3 mL) and DCM (3 mL) was added NaOMe (4.6 M in MeOH, 0.053 mL, 0.244 mmol, 1 eq), and the mixture stirred (420 rpm) under N₂. After 30 minutes further MeOH (3 mL) was added. HPLC analysis after 1 hour showed conversion to the deacetylated product. AcOH (3 drops) was added and the reaction was concentrated by rotary evaporation to give a black residue. The residue was purified by column chromatography (3×12 cm) eluting using a gradient of 10-20% MeOH/DCM. Fractions containing the main green band (R_{f}=0.1 in 10% MeOH/DCM) were combined to give compound 10 as a dark green solid (70 mg, 29% over 2 steps) (HPLC purity: 94.9%).

¹H NMR (400 MHZ, DMSO-d₆) δ 9.85 (s, 1H), 9.76 (s, 1H), 9.08 (m, 2H), 6.58 (d, 1H), 6.20 (d, 1H), 6.15 (d, 1H), 6.01 (d, 1H), 5.09 (t, 2H), 4.95 (t, 2H), 4.90 (t, 1H), 4.82 (m, 3H), 4.76 (m, 1H), 4.61 (m, 4H), 4.55 (m, 2H), 4.46 (m, 3H), 4.38 (m, 1H), 4.30 (m, 2H), 4.10 (m, 1H), 4.00 (m, 3H), 3.90 (m, 2H), 3.83 (q, 2H), 3.68 (m, 3H), 3.63 (s, 3H), 3.55 (m, 9H), 3.42 (m, 6H), 3.20-3.00 (m, 11H), 2.90 (m, 3H), 1.75-1.67 (m, 7H), 1.60-1.52 (m, 1H), −2.33 (s, 1H), −2.52 (s, 1H).

### Synthesis Example 11—synthesis of phyllochlorin 13-hydroxymethyl-β-D-glucosyl-N-methylpropylamide tetraacetate (compound 11)

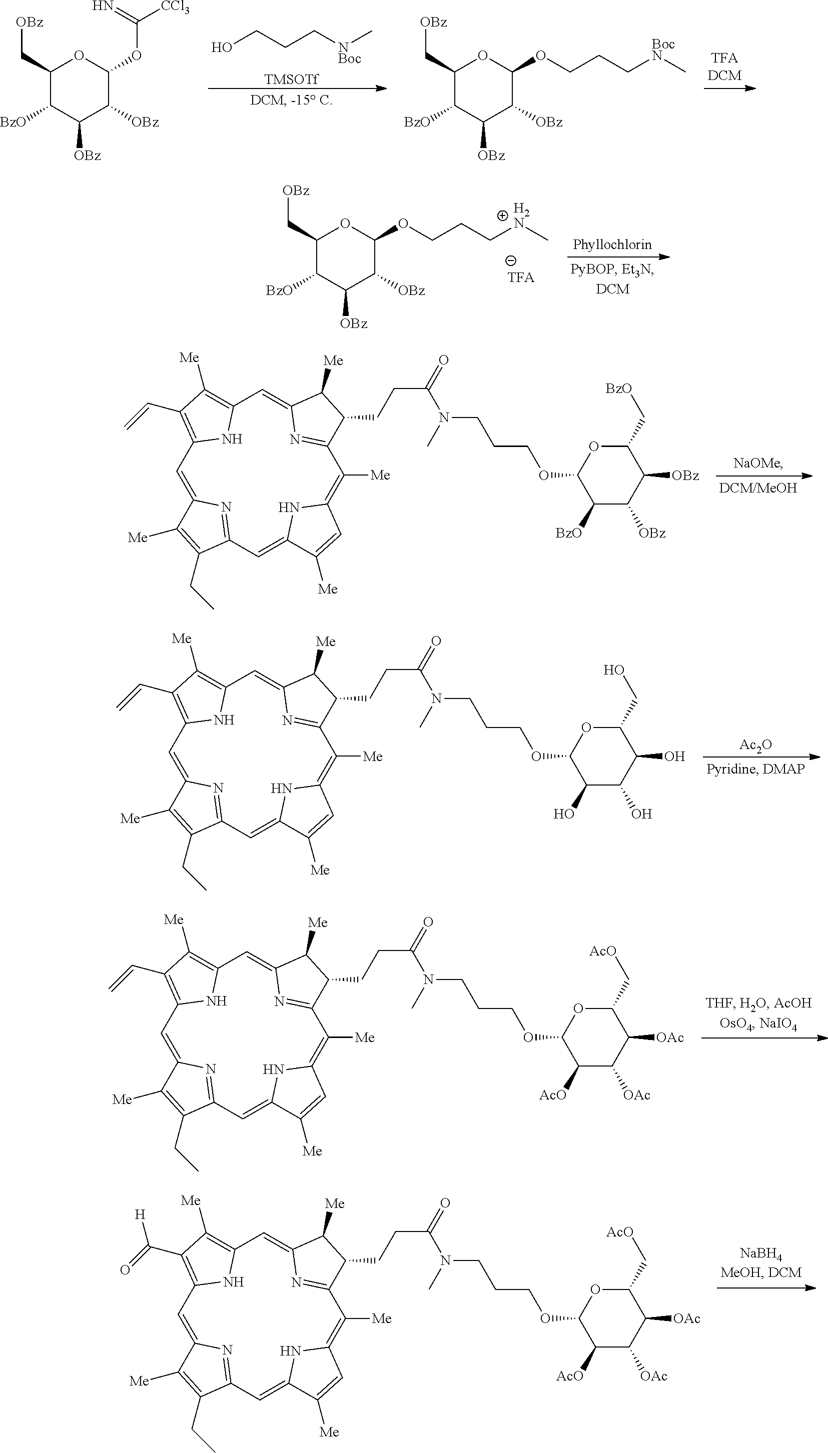

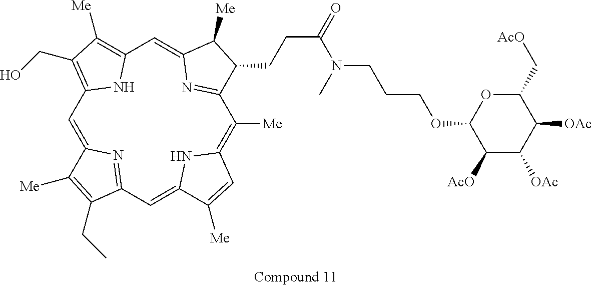 [SMILES: CCC1=C(C)c2cc3[nH]c(cc4nc(c(C)c5cc(C)c(cc1n2)[nH]5)[C@@H](CCC(=O)N(C)CCCO[C@@H]1O[C@H](COC(C)=O)[C@@H](OC(C)=O)[C@H](OC(C)=O)[C@H]1OC(C)=O)[C@@H]4C)c(C)c3CO]

Step 1: A 250 mL 3-neck RBF fitted with an internal thermometer, nitrogen inlet and rubber septum was charged with tert-butyl (3-hydroxypropyl)(methyl) carbamate (2.68 g, 14.17 mmol, 1.05 eq), a stirrer bar, 2,3,4,6-tetra-O-benzoyl-α-D-glucopyranosyl trichloroacetimidate (10.00 g, 13.5 mmol, 1 eq), dry DCM (100 mL) and ground 4 A molecular sieves (5.0 g). The resultant suspension was placed under an atmosphere of nitrogen and stirred (420 rpm) for 0.5 hours before cooling to −15° C. (internal temperature) with the aid of an EtOH/ice/NaCl bath. A solution of TMSOTf (0.2M in DCM, 3.3 mL, 0.67 mmol) was added dropwise over the course of a minute, and stirring continued at low temperature for 0.5 hours, at which point TLC analysis indicated complete reaction (25% EtOAc/hexanes, R_{f} (starting material)=0.4, R_{f} (product)=0.2, visualised by UV). The reaction was quenched with triethylamine (8 drops) and filtered through Celite®, washing through with a further portion of DCM. The filtrate was concentrated to give the crude glycosylated product as a yellow syrup (15.4 g) which was dissolved in minimum DCM and the solution purified by column chromatography (4×22 cm of silica) using a gradient solvent of 25-35% EtOAc in hexane to give 2,3,4,6-tetra-O-benzoyl-β-D-glucopyranose-N-Boc-N-methylproploxyamine as a colourless syrup (7.95 g, 77%) (R_{f} 0.4 in 35% EtOAc in hexane).

¹H NMR (400 MHZ, CDCl₃) δ 8.05-7.99 (m, 2H), 7.98-7.93 (m, 2H), 7.92-7.88 (m, 2H), 7.86-7.79 (m, 2H), 7.59-7.46 (m, 3H), 7.46-7.24 (m, 9H), 5.90 (dd, J=9.7, 9.7 Hz, 1H), 5.68 (dd, J=9.7, 9.7 Hz, 1H), 5.52 (dd, J=9.7, 7.8 Hz, 1H), 4.84 (d, J=7.8 Hz, 1H), 4.64 (dd, J=12.2, 3.2 Hz, 1H), 4.49 (dd, J=12.2, 5.2 Hz, 1H), 4.20-4.13 (m, 1H), 3.95 (ddd, J=9.7, 5.2, 3.2 Hz, 1H), 3.61-3.50 (m, 1H), 3.24-2.99 (m, 2H), 2.66 (s, 3H), 1.82-1.68 (m, 2H), 1.39 (s, 9H).

Step 2: To a 500 mL RBF containing 2,3,4,6-tetra-O-benzoyl-β-D-glucopyranose-N-Boc-N-methylproploxyamine (7.95 g, 10.35 mmol, 1 eq) was added DCM (50 mL) and a stirrer bar. The mixture was stirred (250 rpm) briefly until a solution had formed before TFA (10 mL) was added. The mixture was stirred for 0.5 hours and monitored by TLC (30% EtOAc/hexanes, R_{f} (starting material)=0.4, R_{f} (product)=0), visualised by UV). The reaction was concentrated by rotary evaporation and then reconcentrated from CHCl₃ (2×30 mL) to give 2,3,4,6-tetra-O-benzoyl-β-D-glucopyranose-N-methylpropyloxyammonium trifluoroacetate as lightly coloured syrup (11.0 g) that was used without further purification.

¹H NMR (400 MHZ, CDCl₃) δ 8.69 (br s, 1H), 8.15 (br s, 1H), 8.08-8.02 (m, 2H), 7.99-7.88 (m, 4H), 7.87-7.80 (m, 2H), 7.67 (br s, 1H), 7.63-7.48 (m, 3H), 7.52-7.24 (m, 9H), 5.99 (dd, J=9.8, 9.8 Hz, 1H), 5.71 (dd, J=9.8, 9.8 Hz, 1H), 5.38 (dd, J=9.8, 7.8 Hz, 1H), 4.82 (d, J=7.8 Hz, 1H), 4.74 (dd, J=12.3, 2.8 Hz, 1H), 4.47 (dd, J=12.3, 5.0 Hz, 1H), 4.22-4.06 (m, 2H), 3.73 (app. p, J=5.1 Hz, 1H), 3.31-3.18 (m, 1H), 3.14-3.01 (m, 1H), 2.79 (t, J=5.3 Hz, 3H), 2.04 (app. p, J=5.4 Hz, 2H).

Step 3: A 500 mL RBF was charged with a stirrer bar, phyllochlorin (4.05 g, 7.96 mmol, 1 eq) and DCM (60 mL). 2,3,4,6-Tetra-O-benzoyl-β-D-glucopyranose-N-methylpropyloxyammonium trifluoroacetate (6.91 g, 10.35 mmol, 1.3 eq) was dissolved in DCM (20 mL) and transferred into the RBF. Triethylamine (6.62 mL, 47.8 mmol, 6 eq) was added, followed by PyBOP (4.97 g, 9.55 mmol, 1.2 eq), and the mixture stirred for 0.5 hours. TLC analysis showed consumption of the starting material and the presence of the product (5% MeOH/DCM, R_{f} (phyllochlorin)=0.25, R_{f} (product)=0.95, visualised by UV). The mixture was transferred to a separatory funnel and washed with 1M HCl (2×75 mL), then pH 7 phosphate buffer (100 mL), before being dried (Na₂SO₄) and concentrated by rotary evaporation to give the crude product as a green film (16.3 g). The crude product was purified by Biotage autocolumn chromatography (0-2% MeOH/DCM) to give phyllochlorin β-D-glucosyl-N-methylpropylamide tetrabenzoate as a green film (4.75 g, 52%) (HPLC purity: 95.9%).

Step 4: To a 500 mL RBF containing phyllochlorin-D-glucosyl-N-methylpropylamide tetrabenzoate (4.65 g, 4.01 mmol, 1 eq) was added DCM (50 mL), MeOH (50 mL) and a stirrer bar. The mixture was stirred (300 rpm) briefly until a dark solution had formed, whereupon a solution of NaOMe (4.6M in MeOH, 0.44 mL, 2.01 mmol, 0.5 eq) was added, and the mixture stirred for 2 hours. TLC analysis at this point showed complete reaction (10% MeOH/DCM, R_{f} (starting material)=0.95, R_{f} (product)=0). The reaction mixture was concentrated and the residue purified by Biotage autocolumn chromatography (5-12% MeOH/DCM) to give phyllochlorin β-D-glucosyl-N-methylpropylamide as a blue/green flaky solid (2.34 g, 79%) (HPLC purity: 99.4%).

Step 5: Into a 1-neck 50 mL RBF was added phyllochlorin β-D-glucosyl-N-methylpropylamide (190 mg, 0.256 mmol, 1 eq), pyridine (2 mL), acetic anhydride (0.48 mL, 5.12 mmol, 20 eq) and DMAP (1 mg). The solution was stirred at 30° C. for 1 hour. Ethyl acetate (20 mL) and water (10 mL) were added and the mixture stirred vigorously for 10 minutes. The layers were separated and the ethyl acetate layer washed with 0.5 M HCl (2×10 mL), saturated NaHCO₃ (2×10 mL), dried (Na₂SO₄) and concentrated to give phyllochlorin β-D-glucosyl-N-methylpropylamide tetraacetate as a dark green solid (270 mg, quantitative) (HPLC purity: 99.1%).

¹H NMR (400 MHZ, CDCl₃) δ 9.82-9.72 (m, 2H), 8.88-8.82 (m, 2H), 8.22-8.12 (m, 1H), 6.38 (m, 1H), 6.16 (m, 1H), 5.08-4.92 (m, 1H), 4.80-4.60 (m, 2H), 4.54 (m, 1H), 4.16 (m, 1H), 4.00 (s, 3H), 3.97-3.77 (m, 3H), 3.65 (s, 3H), 3.52 (s, 3H), 3.50-3.42 (m, 1H), 3.40 (s, 1H), 3.38 (s, 2H), 3.10-3.00 (m, 1H), 2.65-2.50 (m, 4H), 2.38-2.20 (m, 2H), 2.14-1.80 (8×s), 1.78-1.70 (m, 6H), −2.10 (brs, 1H), −2.23 (brs, 1H).

Step 6: To a 50 mL RBF was added phyllochlorin β-D-glucosyl-N-methylpropylamide tetraacetate (250 mg, 0.275 mmol, 1 eq), THF (15 mL), osmium tetroxide (0.7 mg, 0.003 mmol, 0.01 eq), deionized water (1 mL), AcOH (1 mL) and sodium periodate (123 mg, 0.577 mmol, 2.1 eq). The resultant mixture was stirred (420 rpm) under nitrogen in the dark at ambient temperature overnight. The reaction mixture was concentrated using a rotary evaporator to remove the THF and then re-dissolved in DCM (50 mL), transferred to a separatory funnel and washed with brine (30 mL), saturated NaHCO₃ (30 mL) and water (40 mL) before being dried (Na₂SO₄) and concentrated by rotary evaporation to give a red-brown powdery solid (220 mg). The intermediate aldehyde, phyllochlorin 13-formyl-β-D-glucosyl-N-methylpropylamide tetraacetate (HPLC purity: 97%) was used without further purification.

Step 7: To a 100 mL RBF was added phyllochlorin 13-formyl-β-D-glucosyl-N-methylpropylamide tetraacetate (210 mg, 0.230 mmol, 1 eq), MeOH (5 mL), DCM (3 mL) and sodium borohydride (17 mg, 0.461 mmol, 2 eq). The resultant mixture was stirred (420 rpm) under nitrogen at ambient temperature for 30 minutes. The reaction mixture was diluted with water (10 mL) and stirred for 10 minutes. The mixture was then extracted with chloroform (3×10 mL). The combined chloroform layers were washed with water (30 mL), dried (Na₂SO₄) and concentrated by rotary evaporation to give a dark green oil (260 mg). The residue was subjected to column chromatography (3×16 cm) eluting using a gradient of 1-3% MeOH/DCM. Fractions containing the major dark green spot (R_{f}=0.45 in 5% MeOH/DCM) were combined to give compound 11 (95 mg, 38% over 2 steps).

¹H NMR (400 MHZ, CDCl₃) δ 9.90-9.72 (m, 2H), 9.0-8.87 (m, 2H), 6.10-5.83 (m, 2H), 4.92 (m, 1H), 4.78 (brs, 1H), 4.70-4.52 (m, 2H), 4.15 (dd, 1H), 4.07-4.02 (m, 3H), 3.96-3.75 (m, 3H), 3.70-3.60 (m, 3H), 3.40-3.35 (m, 3H), 3.30-3.20 (m, 1H), 3.0 (m, 1H), 2.90 (m, 1H), 2.75 (m, 1H), 2.60-2.50 (m, 3H), 2.20-2.05 (m, 2H), 2.03-1.78 (7×m, 16H), 1.75-1.68 (m, 6H), −2.35-−2.55 (m, 2H).

### Synthesis Example 12—synthesis of phyllochlorin 13-hydroxymethyl-β-D-glucoside ether-β-D-glucosyl-N-methylpropylamide (compound 12)

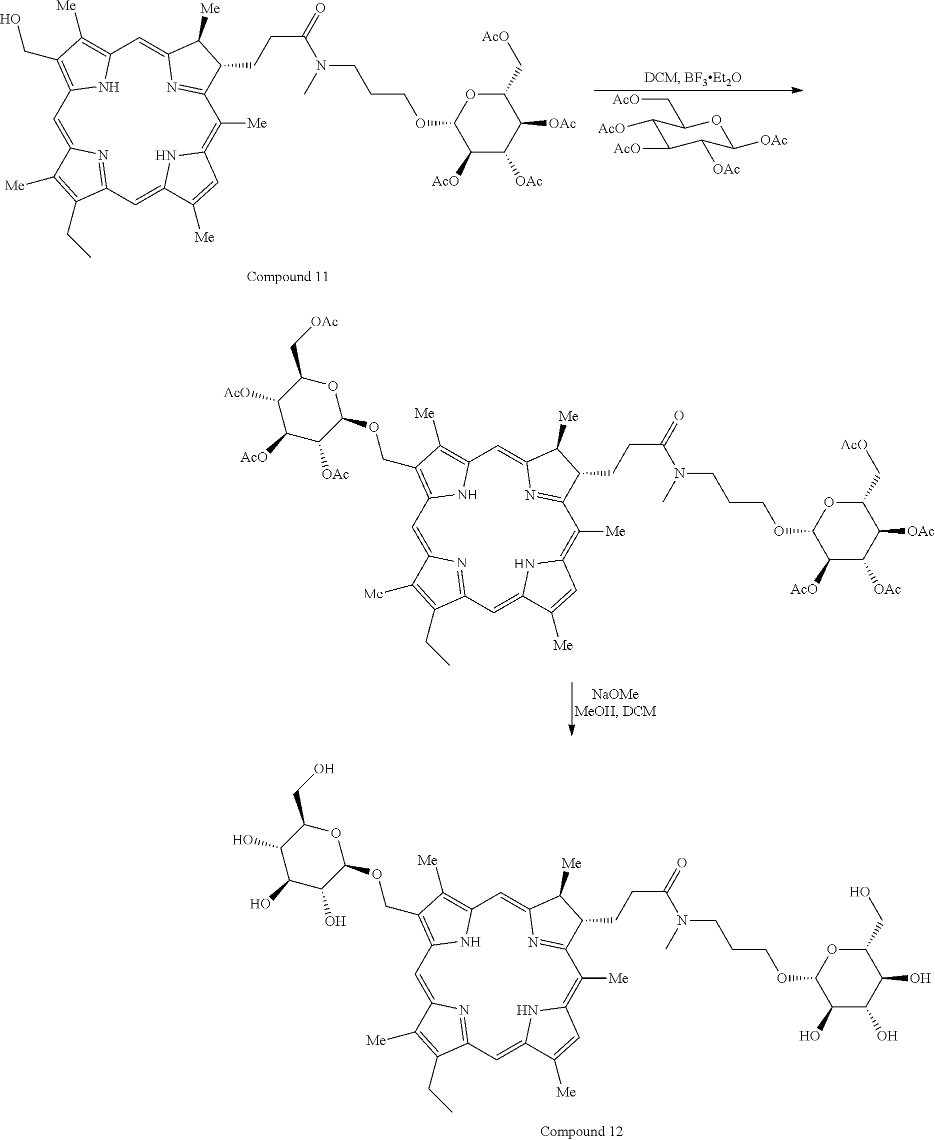

Step 1: To a 50 mL RBF was added phyllochlorin 13-hydroxymethyl-β-D-glucosyl-N-methylpropylamide tetraacetate (compound 11) (90 mg, 0.098 mmol, 1 eq), glucose pentaacetate (58 mg, 0.148 mmol, 1.5 eq) and DCM (5 mL). The resultant mixture was stirred (420 rpm) under nitrogen with cooling to ˜0° C. Boron trifluoride diethyl etherate (0.2 mL) was added and stirring was continued allowing the solution to warm slowly to room temperature over 2 hours and then stirred at room temperature in the dark overnight. The reaction was monitored by HPLC. The reaction mixture was diluted with DCM (20 mL), transferred to a separatory funnel and washed with saturated NaHCO₃ (20 mL), then water (20 mL). The organic phase was dried (Na₂SO₄) and concentrated by rotary evaporation to give a blue-black film (200 mg). The residue was subjected to column chromatography (3×16 cm) eluting using a gradient of 1-3% MeOH/DCM to elute the coupled intermediate (R_{f}=0.6 in 5% MeOH/DCM). The partially purified phyllochlorin 13-hydroxymethyl-β-glucoside ether-β-D-glucosyl-N-methylpropylamide tetraacetate (91 mg) was used directly in the next step.

Step 2: To a solution of phyllochlorin 13-hydroxymethyl-β-glucoside ether-β-D-glucosyl-N-methylpropylamide tetraacetate (91 mg, 0.073 mmol, 1 eq) in MeOH (4 mL) and DCM (2 mL) was added NaOMe (4.6 M in MeOH, 0.015 mL, 0.073 mmol, 1 eq) and the mixture stirred (420 rpm) under N₂ for 1 hour monitoring by HPLC. Acetic acid (2 drops) was added and the reaction concentrated by rotary evaporation to give a black oil. The residue was purified by column chromatography (3×12 cm) eluting using a gradient of 10-20% MeOH/DCM. Fractions containing the main green band (R_{f}<0.1 in 5% MeOH/DCM) were combined to give compound 12 as a dark green solid (31 mg, 35% over 2 steps) (HPLC purity: 97.1%).

¹H NMR (400 MHZ, DMSO-d₆) δ 9.84 (s, 1H), 9.75 (s, 1H), 9.07 (m, 2H), 6.59 (brs, 1H), 6.21 (brs, 1H), 6.15 (d, 1H), 6.00 (d, 1H), 5.10-4.70 (brm, 12H), 4.70-4.35 (brm, 8H), 4.27 (m, 1H), 4.18 (d, 1H), 4.10 (d, 1H), 3.98 (m, 3H), 3.90 (m, 1H), 3.83 (q, 2H), 3.78-3.60 (m, 8H), 3.51 (m, 4H), 3.22-3.00 (m, 14H), 2.92 (m, 4H), 2.8m (m, 2H), 2.60 (m, 1H), 2.40 (m, 1H), 1.80-1.65 (m, 9H), −2.34 (brs, 1H), −2.52 (brs, 1H).

### Synthesis Example 13—synthesis of phyllochlorin 13-hydroxymethyl-N,N-bis-PEG₃OMe propylamide (compound 13)

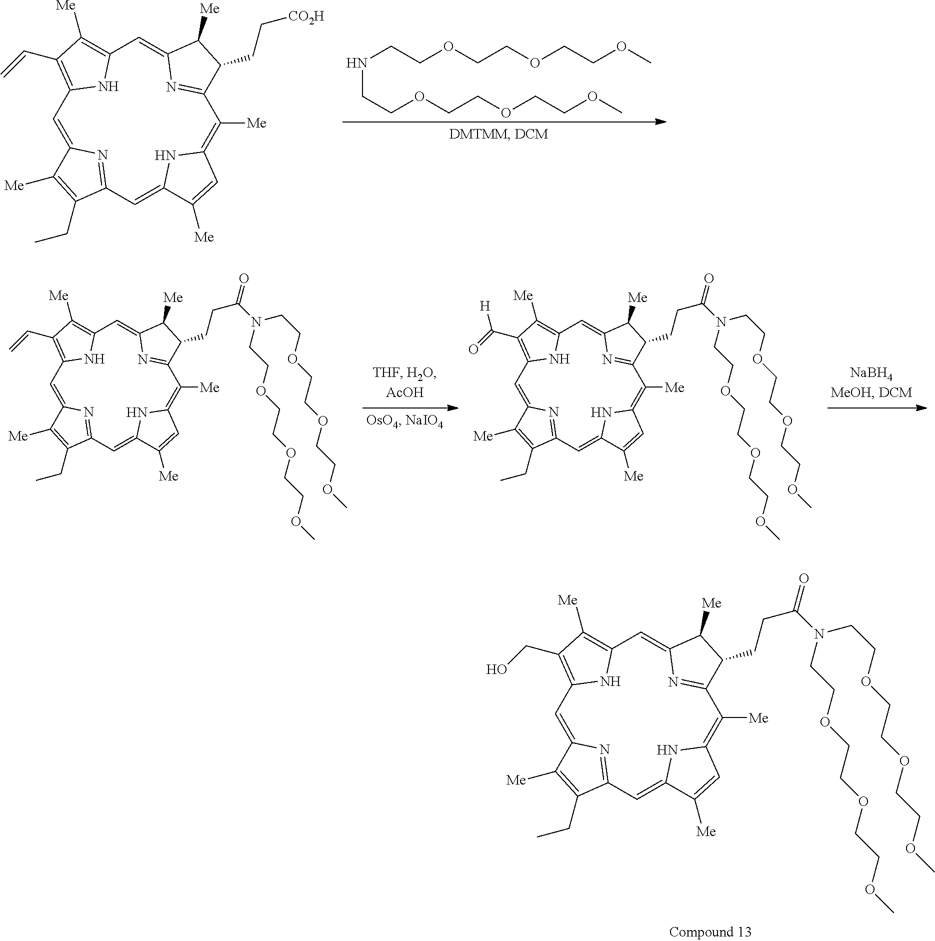 [SMILES: C=Cc1c(C)c2cc3nc(c(C)c4cc(C)c(cc5nc(cc1[nH]2)C(C)=C5CC)[nH]4)[C@@H](CCC(=O)O)[C@@H]3C ; COCCOCCOCCNCCOCCOCCOC ; C=Cc1c(C)c2cc3nc(c(C)c4cc(C)c(cc5nc(cc1[nH]2)C(C)=C5CC)[nH]4)[C@@H](CCC(=O)N(CCOCCOCCOC)CCOCCOCCOC)[C@@H]3C ; CCC1=C(C)c2cc3[nH]c(cc4nc(c(C)c5cc(C)c(cc1n2)[nH]5)[C@@H](CCC(=O)N(CCOCCOCCOC)CCOCCOCCOC)[C@@H]4C)c(C)c3C=O ; CCC1=C(C)c2cc3[nH]c(cc4nc(c(C)c5cc(C)c(cc1n2)[nH]5)[C@@H](CCC(=O)N(CCOCCOCCOC)CCOCCOCCOC)[C@@H]4C)c(C)c3CO]

Step 1: To a 100 mL RBF was added phyllochlorin (0.50 g, 0.983 mmol, 1 eq), DMTMM (0.408 g, 1.474 mmol, 1.1 eq), DCM (15 mL) and bis(2-(2-(2-methoxyethoxy) ethoxy)ethyl)amine (0.456 g, 1.474 mmol, 1.5 eq). The resultant mixture was stirred (420 rpm) under nitrogen at ambient temperature for 2 hours. The reaction mixture was transferred to a separatory funnel, diluted with DCM (20 mL) and washed with 0.5 M HCl (10 mL). The aqueous layer was re-extracted with DCM (2×10 mL) and the combined organics washed with pH 7 buffer (20 mL). The organic phase was dried (Na₂SO₄) and concentrated by rotary evaporation to give a blue-black film (0.95 g). The residue was purified by column chromatography (4×20 cm) using 2-3% MeOH/DCM. Fractions containing the major dark green spot (R_{f}=0.3 in 5% MeOH/DCM) were combined to give phyllochlorin N,N-bis-PEG₃OMe propylamide as a dark green viscous oil (762 mg, 97%) (HPLC purity: 96.5%).

¹H NMR (400 MHZ, CDCl₃) δ 9.71 (m, 2H), 8.86 (m, 2H), 8.16 (dd, 1H), 6.38 (dd, 1H), 6.15 (dd, 1H), 4.62 (m, 1H), 4.54 (m, 1H), 4.05 (s, 1H), 4.01 (s, 3H), 3.85 (q, 2H), 3.64 (s, 3H), 3.53 (s, 3H), 3.50-3.42 (m, 7H), 3.41-3.36 (m, 9H), 3.30-3.25 (m, 5H), 3.25 (s, 3H), 3.20 (m, 2H), 3.00 (m, 2H), 2.97-2.92 (m, 4H), 2.81 (m, 2H), 2.61-2.42 (m, 2H), 2.21-2.11 (m, 1H), 2.08-2.00 (m, 1H), 1.79-1.72 (m, 6H), −2.11 (brs, 1H), −2.23 (brs, 1H).

Step 2: To a 250 mL RBF was added phyllochlorin N,N-bis-PEG₃OMe propylamide (680 mg, 0.850 mmol, 1 eq), THF (30 mL), osmium tetroxide (2 mg, 0.008 mmol, 0.01 eq), deionized water (2 mL), AcOH (2 mL) and sodium periodate (454 mg, 2.125 mmol, 2.5 eq). The resultant mixture was stirred (300 rpm) under nitrogen in the dark at ambient temperature overnight. The reaction mixture was concentrated using a rotary evaporator to remove the THF and then re-dissolved in DCM (100 mL), transferred to a separatory funnel and washed with brine (30 mL), saturated NaHCO₃ (30 mL) and water (40 mL) before being dried (Na₂SO₄) and concentrated by rotary evaporation to give a red-brown oil (760 mg). The intermediate aldehyde, phyllochlorin 13-formyl-N,N-bis-PEG₃OMe-propylamide (HPLC purity: 97%) was used without further purification.

Step 3: To a 100 mL RBF was added phyllochlorin 13-formyl-N,N-bis-PEG₃OMe-propylamide (682 mg, 0.850 mmol, 1 eq), MeOH (15 mL), DCM (10 mL) and sodium borohydride (64 mg, 1.701 mmol, 2 eq). The resultant mixture was stirred (300 rpm) under nitrogen at ambient temperature for 1 hour. The reaction mixture was diluted with water (25 mL) and stirred for 10 minutes. The mixture was then diluted with chloroform (20 mL), washed with brine (20 mL), dried (Na₂SO₄) and concentrated by rotary evaporation to give a dark green oil (790 mg). The residue was subjected to column chromatography (4×18 cm) eluting using a gradient of 1-6% MeOH/DCM. Fractions containing the major dark green spot (R_{f}=0.20 in 5% MeOH/DCM) were combined to give compound 13 (496 mg, 73% over 2 steps) (HPLC purity: 96%).

¹H NMR (400 MHZ, CDCl₃) δ 9.71 (m, 2H), 8.86 (m, 2H), 5.98 (m, 2H), 4.65 (m, 1H), 4.54 (m, 1H), 4.02 (s, 3H), 3.84 (q, 2H), 3.64 (s, 3H), 3.53 (s, 3H), 3.48-3.42 (m, 5H), 3.41-3.36 (m, 10H), 3.27-3.24 (m, 9H), 3.13 (m, 2H), 2.90-2.70 (m, 6H), 2.61-2.52 (m, 3H), 2.50-2.35 (m, 2H), 2.26-2.17 (m, 1H), 1.98-1.90 (m, 1H), 1.78-1.73 (m, 1H), −2.19 (brs, 1H), −2.37 (brs, 1H).

### Synthesis Example 14—synthesis of phyllochlorin 13-hydroxymethyl-β-D-glucoside ether-N,N-bis-PEG₃OMe propylamide (compound 14)

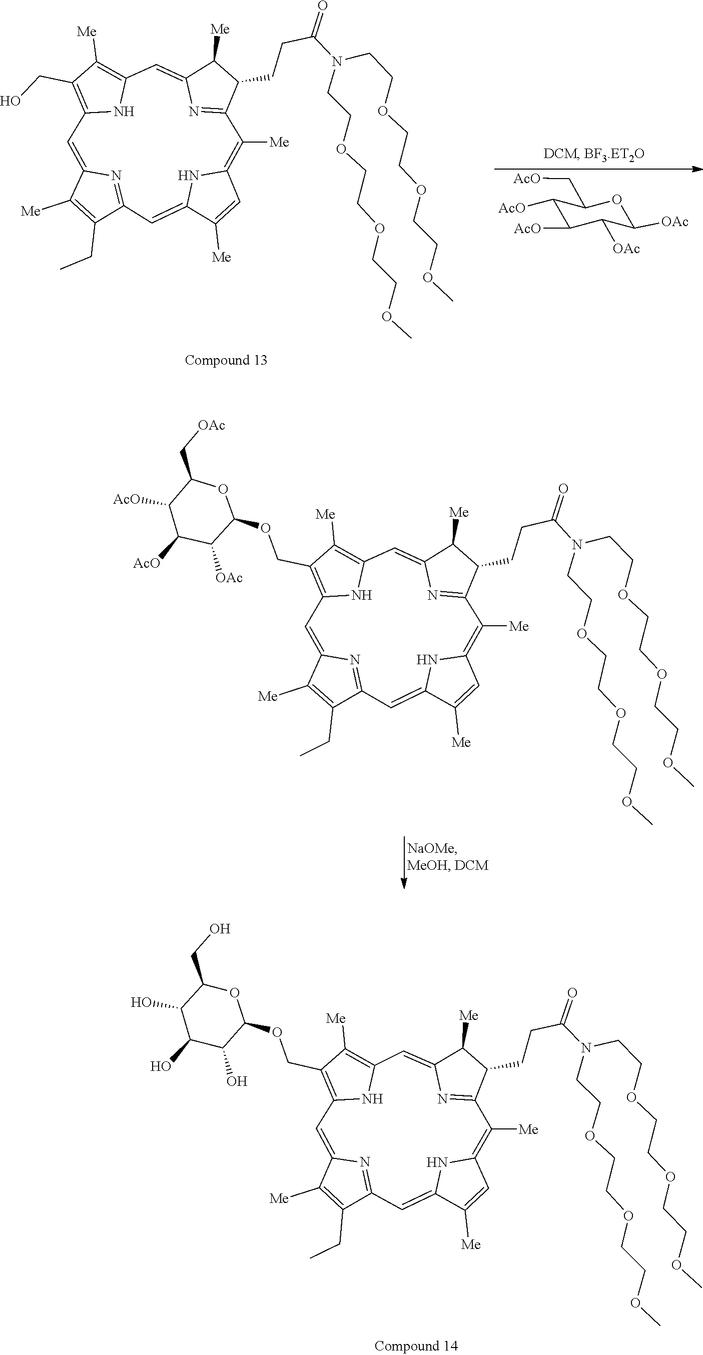 [SMILES: CCC1=C(C)c2cc3[nH]c(cc4nc(c(C)c5cc(C)c(cc1n2)[nH]5)[C@@H](CCC(=O)N(CCOCCOCCOC)CCOCCOCCOC)[C@@H]4C)c(C)c3CO ; CC(=O)OCC1OC(OC(C)=O)C(OC(C)=O)C(OC(C)=O)C1OC(C)=O ; CCC1=C(C)c2cc3[nH]c(cc4nc(c(C)c5cc(C)c(cc1n2)[nH]5)[C@@H](CCC(=O)N(CCOCCOCCOC)CCOCCOCCOC)[C@@H]4C)c(C)c3CO[C@@H]1O[C@H](COC(C)=O)[C@@H](OC(C)=O)[C@H](OC(C)=O)[C@H]1OC(C)=O ; CCC1=C(C)c2cc3[nH]c(cc4nc(c(C)c5cc(C)c(cc1n2)[nH]5)[C@@H](CCC(=O)N(CCOCCOCCOC)CCOCCOCCOC)[C@@H]4C)c(C)c3CO[C@@H]1O[C@H](CO)[C@@H](O)[C@H](O)[C@H]1O]

Step 1: To a 50 mL RBF was added phyllochlorin 13-hydroxymethyl-N,N-bis-PEG₃OMe propylamide (compound 13) (380 mg, 0.473 mmol, 1 eq), glucose pentaacetate (277 mg, 0.709 mmol, 1.5 eq) and DCM (10 mL). The resultant mixture was stirred (300 rpm) under nitrogen cooling to ˜0° C. Boron trifluoride diethyl etherate (0.2 mL) was added and stirring was continued allowing the solution to warm slowly to room temperature over 2 hours and then stirred at room temperature in the dark overnight. The reaction was monitored by HPLC. The reaction mixture was diluted with saturated NaHCO₃ (20 mL) and stirred for 10 minutes. The mixture was transferred to a separatory funnel and extracted with DCM (2×10 mL). The combined DCM layers were washed with water (20 mL), the organic phase was dried (Na₂SO₄) and concentrated by rotary evaporation to give a blue-black film (750 mg). The residue was subjected to column chromatography (3×18 cm) eluting using a gradient of 1-3% MeOH/DCM to elute the product (R_{f}=0.35 in 5% MeOH/DCM). The partially purified phyllochlorin 13-hydroxymethyl-β-glucoside ether N,N-bis-PEG₃OMe propylamide tetraacetate (330 mg) was used directly in the next step.

Step 2: To a solution of phyllochlorin 13-hydroxymethyl-β-glucoside ether N,N-bis-PEG₃OMe propylamide tetraacetate (320 mg, 0.282 mmol, 1 eq) in MeOH (4 mL) and DCM (3 mL) was added NaOMe (4.6 M in MeOH, 0.061 mL, 0.282 mmol, 1 eq) and the mixture stirred (300 rpm) under N₂ overnight. Acetic acid (1 drop) was added and the reaction was concentrated by rotary evaporation to give a black oil. The residue was purified by column chromatography (3×16 cm) eluting using a gradient of 5-10% MeOH/DCM. Fractions containing a green spot (R_{f}=0.1 in 5% MeOH/DCM) were combined to give compound 14 as a dark green solid (130 mg, 28% over 2 steps).

¹H NMR (400 MHZ, DMSO-d₆) δ 9.85 (s, 1H), 9.75 (s, 1H), 9.07 (m, 2H), 6.15 (d, 1H), 6.00 (d, 1H), 5.11 (d, 1H), 4.95 (m, 2H), 4.83 (m, 1H), 4.66-4.54 (m, 3H), 3.98 (s, 3H), 3.90 (m, 1H), 3.83 (q, 2H), 3.72-3.65 (m, 2H), 3.63 (s, 3H), 3.51 (m, 4H), 3.45-3.38 (m, 11H), 3.30-3.25 (m, 6H), 3.22 (m, 5H), 3.13 (s, 3H), 3.10 (s, 3H), 2.88-2.78 (m, 1H), 2.45-2.38 (m, 1H), 1.80-1.65 (m, 7H), −2.34 (brs, 1H), −2.52 (brs, 1H).

### Synthesis Example 15—synthesis of phyllochlorin 13-hydroxymethyl-β-D-1-glucose-2-ethoxy-N-ethanamine tetraacetate (compound 15)

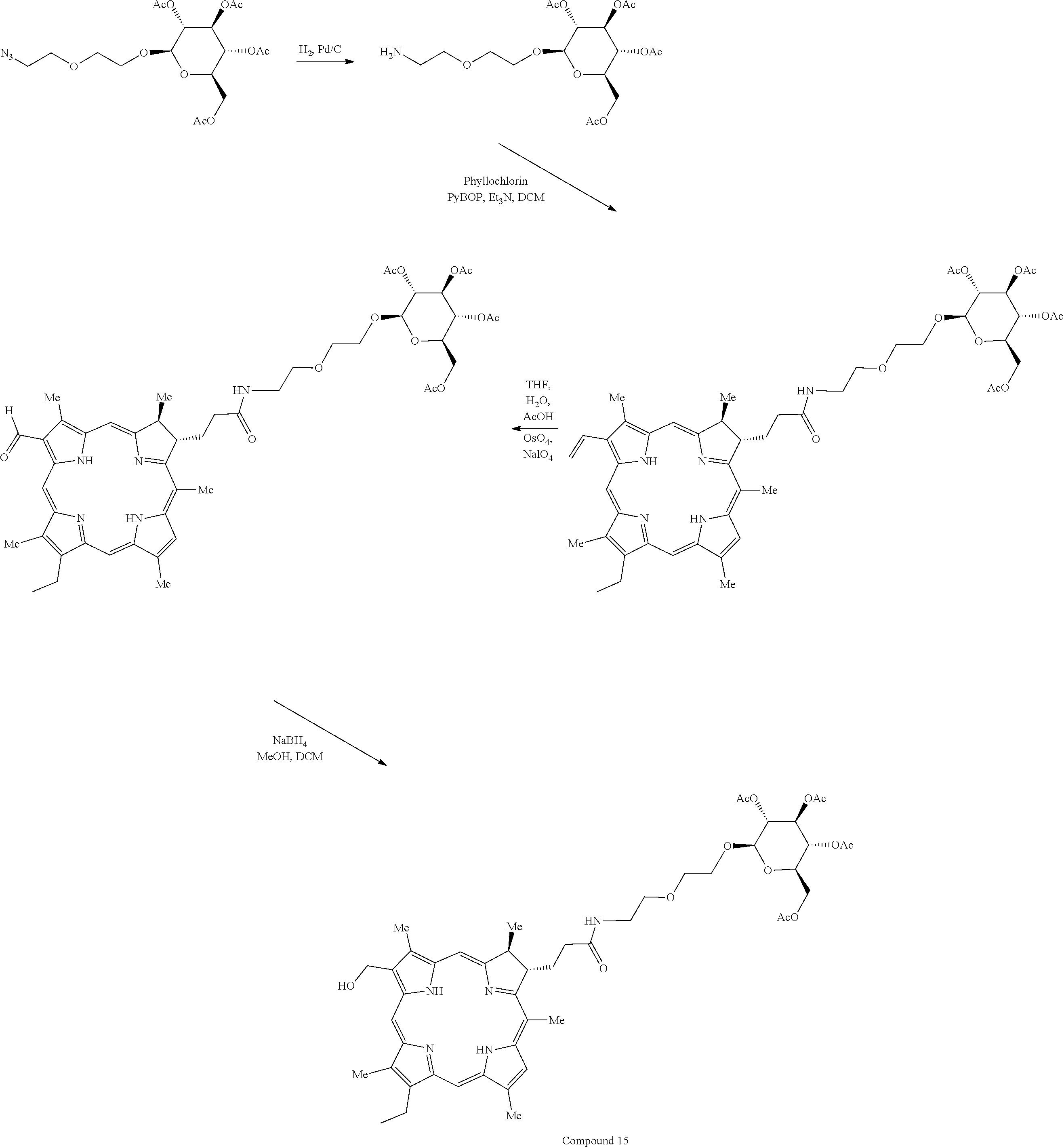

Step 1: A 3-neck 100 mL RBF was charged with (2R,3R,4S,5R,6R)-2-(acetoxymethyl)-6-(2-(2-azidoethoxy) ethoxy)tetrahydro-2H-pyran-3,4,5-triyl triacetate (1.00 g, 2.167 mmol, 1 eq), 10% Pd/C (25 mg), methanol (100 mL) and a stirrer bar. A hydrogen balloon was connected and the flask was evacuated and then re-filled with nitrogen (3 times), evacuated and re-filled with hydrogen (2 times). The resulting solution was then stirred (550 rpm) under the hydrogen atmosphere for 1 hour. The solution was filtered through Celite® (0.5×3 cm), washing with DCM (˜30 mL) and the solvent then removed under reduced pressure to give 1.02 g of crude amine product that was used directly in the next step.

Step 2: To a 50 mL RBF was added phyllochlorin (0.85 g, 1.67 mmol, 1 eq), PyBOP (1.04 g, 2.01 mmol, 1.2 eq), DCM (20 mL) and triethylamine (1.39 mL, 10.03 mmol, 6 eq). The resultant mixture was stirred (250 rpm) under nitrogen at ambient temperature for 15 minutes, and then the amine (1.02 g) in DCM (5 mL) was added in one portion. The resultant mixture was stirred overnight in the dark under nitrogen. The reaction mixture was diluted with DCM (30 mL), transferred to a separatory funnel and washed with 1M HCl (2×75 mL), then pH 7 buffer (1×100 mL). The organic phase was dried (Na₂SO₄) and concentrated by rotary evaporation to give a blue/black oil (˜2.5 g) which was purified by column chromatography (4×25 cm of silica) eluting with 1-2.5% MeOH/DCM. Fractions containing the product (R_{f}˜0.4 in 5% MeOH/DCM) were combined to give phyllochlorin β-D-1-glucose-N-ethoxyethyl amide tetraacetate as a blue/black solid (0.65 g, 42%) which was deprotected without further purification.

Step 3: To a 100 mL RBF was added phyllochlorin β-D-1-glucose-N-ethoxyethyl amide tetraacetate (290 mg, 0.313 mmol, 1 eq), THF (15 mL), osmium tetroxide (1.2 mg, 0.005 mmol, 0.01 eq), deionized water (1 mL), AcOH (1 mL) and sodium periodate (167 mg, 0.782 mmol, 2.5 eq). The resultant mixture was stirred (300 rpm) under nitrogen in the dark at ambient temperature overnight. The reaction mixture was concentrated using a rotary evaporator to remove the THF and then re-dissolved in DCM (50 mL). The mixture was transferred to a separatory funnel and washed with brine (30 mL), saturated NaHCO₃ (30 mL) and water (40 mL) before being dried (Na₂SO₄) and concentrated by rotary evaporation to give phyllochlorin 13-formyl-β-D-1-glucose-2-ethoxy-N-ethanamine tetraacetate as a red-brown oil (270 mg) that was used without further purification.

Step 4: To a 100 mL RBF was added phyllochlorin 13-formyl-β-D-1-glucose-2-ethoxy-N-ethanamine tetraacetate (270 mg, 0.291 mmol, 1 eq), MeOH (6 mL), DCM (4 mL) and sodium borohydride (22 mg, 0.582 mmol, 2 eq). The resultant mixture was stirred (300 rpm) under nitrogen ambient temperature for 30 minutes. The reaction mixture was diluted with water (10 mL) and stirred for 10 minutes. The mixture was then extracted with chloroform (3×10 mL), the combined chloroform layers washed with water (30 mL), dried (Na₂SO₄) and concentrated by rotary evaporation to give a dark green oil (˜500 mg). The residue was subjected to column chromatography (3×16 cm) eluting using a gradient of 1-4% MeOH/DCM. Fractions containing the major dark green spot (R_{f}=0.25 in 5% MeOH/DCM) were combined to give compound 15 (160 mg, 55% over 2 steps).

¹H NMR (400 MHZ, CDCl₃) δ 9.74 (s, 1H), 0.58 (s, 1H), 8.88 (m, 2H), 5.77 (q, 2H), 5.51 (m, 1H), 4.88 (m, 2H), 4.79 (m, 1H), 4.65 (m, 1H), 4.56 (m, 1H), 4.01 (m, 4H), 3.88-3.80 (m, 3H), 3.70 (m, 1H), 3.64 (s, 3H), 3.59 (m, 1H), 3.51-3.44 (m, 4H), 3.30 (s, 3H), 3.20-3.10 (m, 5H), 3.05 (m, 3H), 2.60 (m, 1H), 2.30-2.10 (m, 3H), 1.95 (s, 3H), 1.94 (s, 3H), 1.91 (s, 3H), 1.83 (s, 3H), 1.80 (m, 3H), 1.73 (t, 4H), −2.32 (brs, 1H), −2.41 (brs, 1H).

### Synthesis Example 16—synthesis of phyllochlorin 13-hydroxymethyl-β-D-glucoside ether-β-D-1-glucose-2-ethoxy-N-ethanamine (compound 16)

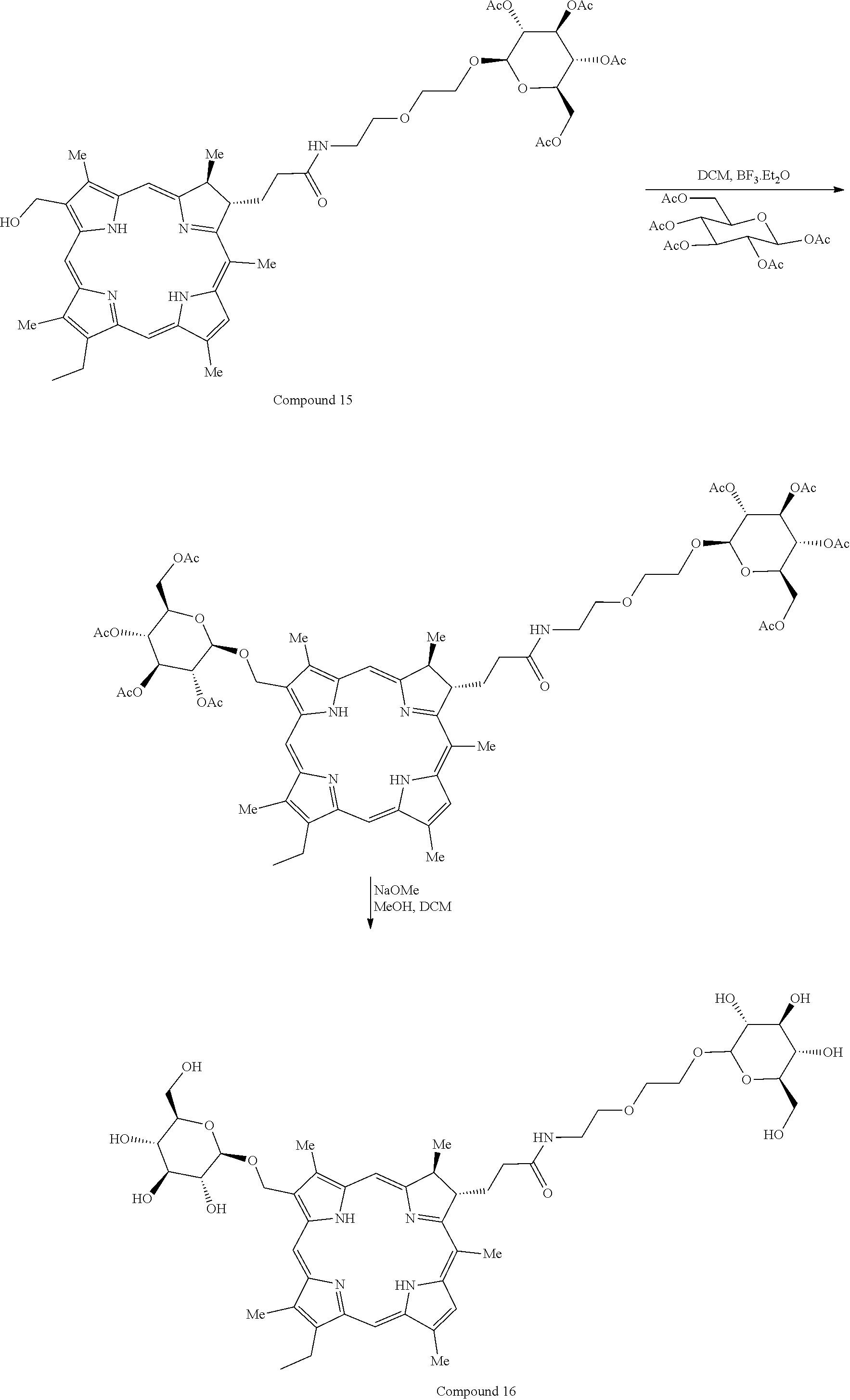

Step 1: To a 50 mL RBF was added phyllochlorin 13-hydroxymethyl-β-D-1-glucose-2-ethoxy-N-ethanamine tetraacetate (compound 15) 150 mg, 0.161 mmol, 1 eq), glucose pentaacetate (94 mg, 0.242 mmol, 1.5 eq) and DCM (8 mL). The resultant mixture was stirred (300 rpm) under nitrogen with cooling to ˜0° C. Boron trifluoride diethyl etherate (0.2 mL) was added and stirring was continued, allowing the solution to warm slowly to room temperature over 2 hours and then stirred at room temperature in the dark overnight. The reaction was monitored by HPLC. The reaction mixture was diluted with saturated NaHCO₃ (20 mL) and stirred for 10 minutes. The mixture was transferred to a separatory funnel and extracted with DCM (3×10 mL) and washed with water (20 mL). The organic phase was dried (Na₂SO₄) and concentrated by rotary evaporation to give a blue-black film (360 mg). The residue was subjected to column chromatography (3×15 cm) eluting using a gradient of 1-3% MeOH/DCM. Fractions containing the intermediate coupled product (R_{f}=0.4 in 5% MeOH/DCM) were combined to give partially purified phyllochlorin 13-hydroxymethyl-β-glucoside ether β-D-1-glucose-2-ethoxy-N-ethanamine peracetate (75 mg) which was used directly in the next step.

Step 2: To a solution of phyllochlorin 13-hydroxymethyl-β-glucoside ether β-D-1-glucose-2-ethoxy-N-ethanamine peracetate (75 mg, 0.060 mmol, 1 eq) in MeOH (3 mL) and DCM (1.5 mL) was added NaOMe (4.6 M in MeOH, 0.012 mL, 0.060 mmol, 1 eq) and the mixture stirred (300 rpm) under N₂ for 2 hours. Acetic acid (1 drop) was added and the reaction was concentrated by rotary evaporation to give a dark green oil. The residue was purified by column chromatography (3×12 cm) eluting using a gradient of 10-20% MeOH/DCM. Fractions containing the major green compound were combined to give compound 16 as a dark green solid (26 mg, 17% over 2 steps).

¹H NMR (400 MHZ, DMSO-d₆) δ 9.84 (s, 1H), 9.74 (s, 1H), 9.08 (m, 2H), 8.01 (m, 1H), 6.59 (m, 1H), 6.22 (m, 1H), 6.16 (d, 1H), 6.00 (d, 1H), 5.11 (m, 1H), 5.05-4.79 (m, 13H), 4.70-4.60 (m, 4H), 4.50 (m, 5H), 4.40 (m, 1H), 4.26 (m, 1H), 4.20-4.10 (m, 4H), 3.94 (m, 5H), 3.83 (m, 4H), 3.70-3.60 (m, 10H), 3.60-3.50 (m, 11H), 3.30-3.10 (m, 19H), 3.05 (m, 6H), 2.98-2.85 (m, 3H), 2.40 (m, 1H), 1.78-1.68 (m, 8H), −2.36 (brs, 1H), −2.54 (brs, 1H).

### Synthesis Example 17—synthesis of phyllochlorin 13-hydroxymethyl (compound 17)

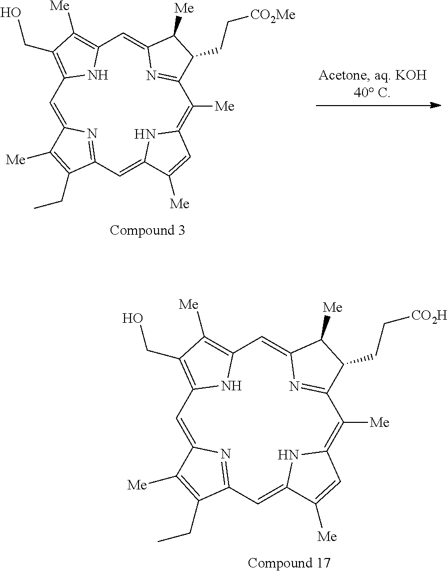 [SMILES: CCC1=C(C)c2cc3[nH]c(cc4nc(c(C)c5cc(C)c(cc1n2)[nH]5)[C@@H](CCC(=O)OC)[C@@H]4C)c(C)c3CO ; CCC1=C(C)c2cc3[nH]c(cc4nc(c(C)c5cc(C)c(cc1n2)[nH]5)[C@@H](CCC(=O)O)[C@@H]4C)c(C)c3CO]

To a 250 mL RBF was added phyllochlorin 13-hydroxymethyl methyl ester (compound 3) (810 mg, 1.538 mmol, 1 eq), acetone (25 mL) and 15% w/v potassium hydroxide (50 mL). The resultant mixture was stirred (300 rpm) under nitrogen at 40° C. for 1 hour. The reaction mixture was concentrated on the rotary evaporator to remove acetone and then DCM (50 mL) was added to the aqueous residue. The mixture was acidified to pH˜5 using 1 M HCl (˜50 mL) and the DCM layer was collected. The aqueous layer was further extracted with DCM (2×20 mL) and the combined DCM layers were washed with water (50 mL) and then pH=7 phosphate buffer (30 mL). The DCM layer was collected and dried (Na₂SO₄) and concentrated by rotary evaporation to give compound 17 as a dark green solid (790 mg, quantitative) (HPLC purity: 99.0%). ¹H NMR (400 MHZ, CDCl₃) δ 9.69 (s, 1H), 9.51 (s, 1H), 8.81 (s, 2H), 5.73 (s, 2H), 4.58 (m, 1H), 4.48 (m, 1H), 3.94 (s, 3H), 3.79 (q, 2H), 3.60 (s, 3H), 3.48 (s, 3H), 3.27 (s, 3H), 2.54-2.42 (m, 2H), 2.17 (m, 1H), 2.14-2.00 (m, 2H), 1.76 (d, 3H), 1.71 (t, 3H), −2.42 (brs, 1H).

### Synthesis Example 18—synthesis of phyllochlorin ((3-(3-(methylamino)propoxy)propyl)triphenylphosphonium chloride propylamide 13-hydroxymethyl (compound 18)

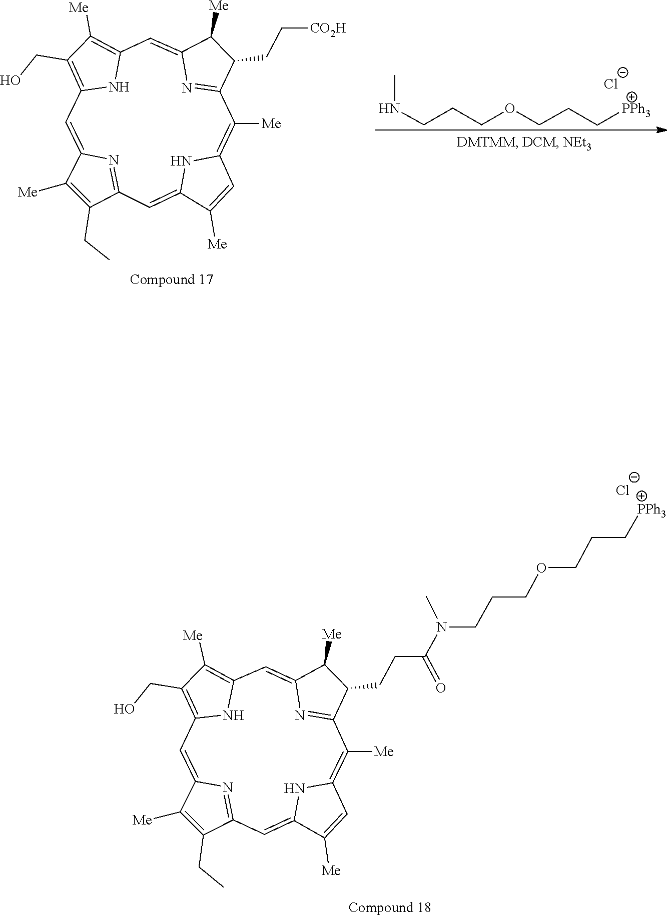

To a 50 mL RBF was added phyllochlorin 13-hydroxymethyl (compound 17) (200 mg, 0.390 mmol, 1 eq), 4-(4,6-dimethoxy-1,3,5-triazin-2-yl)-4-methyl-morpholinium chloride (DMTMM) (140 mg, 0.507 mmol, 1.3 eq), DCM (8 mL) and (3-(3-(methylamino)propoxy)propyl)triphenylphosphonium chloride (270 mg, 0.467 mmol, 1.2 eq) pre-dissolved in DCM (2 mL). The resultant mixture was stirred (300 rpm) under nitrogen at ambient temperature (˜25° C.) for 2 hours. Triethylamine (0.1 mL, 0.721 mmol, 1.8 eq) was added and the mixture was stirred at room temperature overnight. The reaction was monitored by HPLC. The mixture was diluted with DCM (15 mL), washed sequentially with water (15 mL), 0.05 M HCl (15 mL), brine (15 mL) and then dried (Na₂SO₄) and concentrated by rotary evaporation to give a blue-black film (0.6 g). The residue was subjected to column chromatography (3×18 cm) eluting using a gradient of 5-10% MeOH/DCM. Fractions containing a major green spot (R_{f}<0.1 in 5% MeOH/DCM) were combined to give compound 18 (68 mg, 19%) (HPLC purity: 99.7%).

¹H NMR (400 MHZ, CDCl₃) δ 9.88-9.68 (m, 2H), 8.82 (m, 2H), 7.62-7.55 (m, 2H), 7.54-7.50 (m, 2H), 7.45-7.32 (m, 7H), 7.30-7.22 (m, 2H), 6.18-5.90 (m, 2H), 4.71-4.41 (m, 2H), 3.96 (m, 3H), 3.82 (m, 2H), 3.62 (s, 3H), 3.55-3.50 (m, 2H), 3.35-3.25 (m, 2H), 3.20 (s, 2H), 3.18-2.95 (m, 2H), 2.50-2.40 (m, 2H), 2.40-2.35 (m, 1H), 2.28 (s, 2H), 2.18 (s, 1H), 2.08-1.95 (m, 1H), 1.90-1.75 (m, 2H), 1.75-1.60 (m, 13H), 1.40-1.30 (m, 2H), 1.08-0.88 (m, 1H), 0.70-0.45 (m, 2H), −2.16 (brs, 1H), −2.28 (brm, 1H).

### Synthesis Example 19—synthesis of phyllochlorin 13-hydroxymethyl-β-D-glucoside ether ((3-(3-(methylamino)propoxy)propyl)triphenylphosphonium chloride propylamide (compound 19)

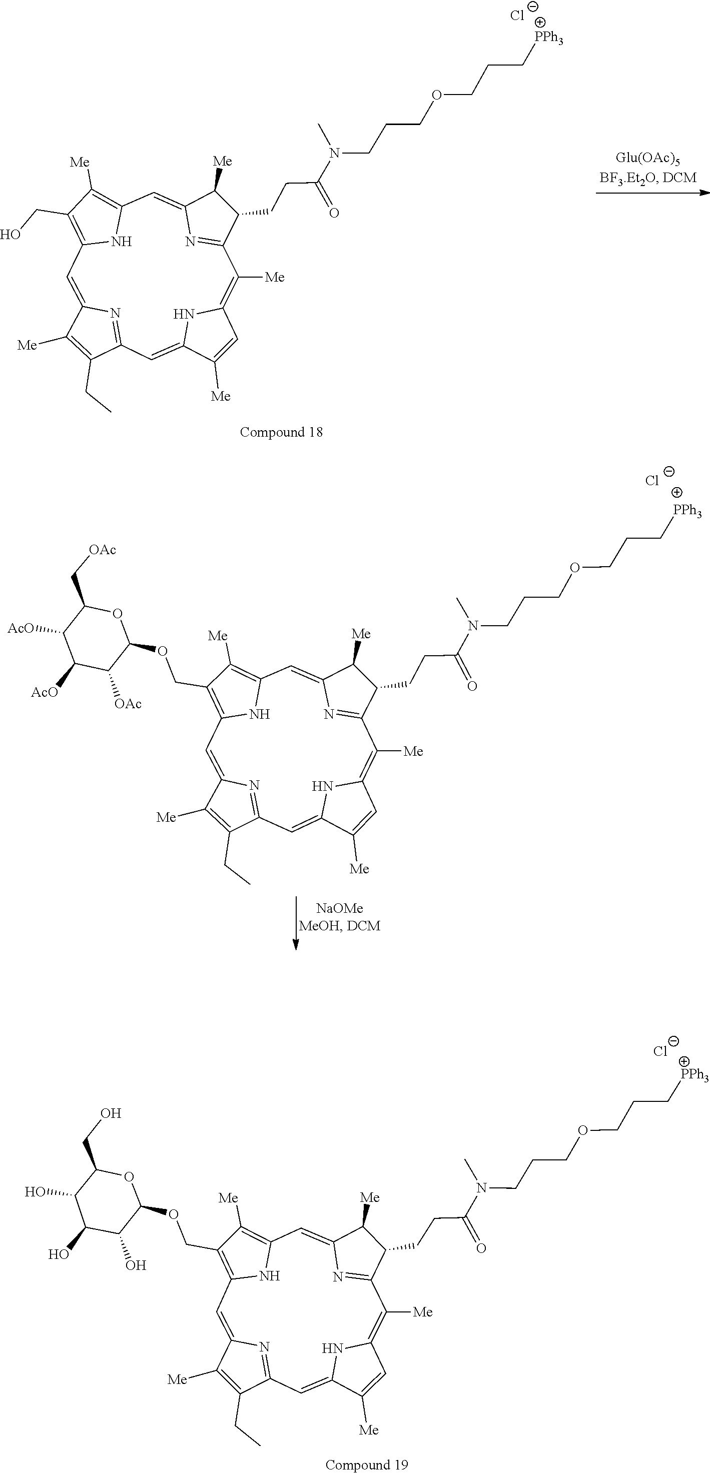

Step 1: To a 50 mL RBF was added phyllochlorin ((3-(3-(methylamino)propoxy)propyl) triphenylphosphonium chloride propylamide 13-hydroxymethyl (compound 18) (68 mg, 0.0737 mmol, 1 eq), glucose pentaacetate (43 mg, 0.111 mmol, 1.5 eq) and DCM (3 mL). The resultant mixture was stirred (420 rpm) under nitrogen with cooling to ˜0° C. Boron trifluoride diethyl etherate (0.1 mL) was added and stirring was continued, allowing the solution to warm slowly to room temperature over 2 hours. The reaction mixture was then stirred at room temperature in the dark overnight. The reaction was monitored by HPLC. The reaction mixture was diluted with DCM (15 mL), water (10 mL), saturated NaHCO₃ (5 mL) and stirred for 10 minutes. After being transferred to a separatory funnel the mixture was extracted with DCM (2×10 mL), washed with water (20 mL) and the organic phase dried (Na₂SO₄) and concentrated by rotary evaporation to give a blue-black film (˜200 mg). The resultant residue was subjected to column chromatography (3×16 cm) eluting using a gradient of 2-3.5% MeOH/DCM. The major green band was collected to give partially purified phyllochlorin-β-glucoside ether tetraacetate (˜80 mg) that was used directly in the next step.

Step 2: To a solution of phyllochlorin-β-glucoside ether tetraacetate (80 mg, 0.0639 mmol, 1 eq) in MeOH (2 mL) and DCM (2 mL) was added NaOMe (4.6 M in MeOH, 0.007 mL, 0.0319 mmol, 0.5 eq), and the mixture stirred (420 rpm) under N₂ for 1 hour. The reaction was concentrated by rotary evaporation to give a black film. The residue was purified by column chromatography (3×16 cm) eluting using a gradient of 8-12% MeOH/DCM. Fractions containing the major green band were combined to give compound 19 as a dark green solid (22 mg, 28% over 2 steps).

¹H NMR (400 MHZ, DMSO-d₆) δ 9.85 (m, 1H), 9.76 (m, 1H), 9.05 (m, 2H), 7.72-7.55 (m, 15H), 6.17 (m, 1H), 6.00 (m, 1H), 5.10 (d, 1H), 4.94 (m, 2H), 4.81 (m, 1H), 4.63-4.53 (m, 3H), 4.10 (q, 2H), 3.95-3.88 (m, 4H), 3.83 (m, 2H), 3.72-3.65 (m, 1H), 3.63 (m, 3H), 3.50 (m, 5H), 3.30-3.20 (m, 4H), 3.18 (m, 8H), 3.12 (m, 2H), 3.05 (m, 1H), 2.86 (s, 2H), 2.75 (s, 1H), 2.40-2.30 (m, 1H), 1.73-1.65 (m, 9H), 1.60-1.50 (m, 3H), −2.33 (s, 1H), −2.51 (s, 1H).

### Synthesis Example 20—synthesis of phyllochlorin methyl ester 3-pyridinium bromide propyl ether (compound 20)

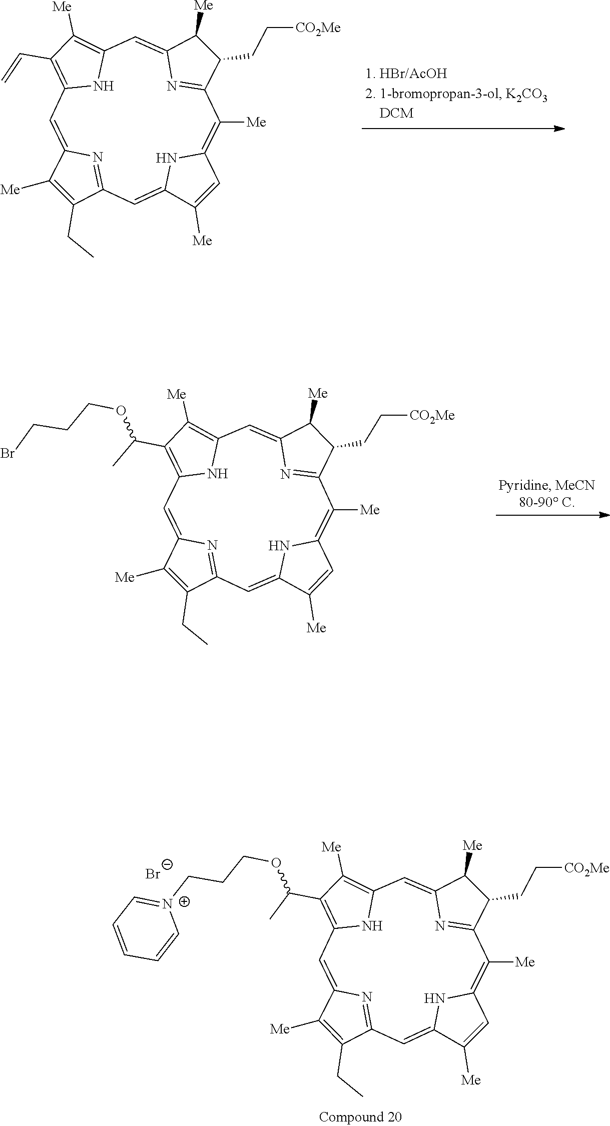 [SMILES: C=Cc1c(C)c2cc3nc(c(C)c4cc(C)c(cc5nc(cc1[nH]2)C(C)=C5CC)[nH]4)[C@@H](CCC(=O)OC)[C@@H]3C ; CCC1=C(C)c2cc3[nH]c(cc4nc(c(C)c5cc(C)c(cc1n2)[nH]5)[C@@H](CCC(=O)OC)[C@@H]4C)c(C)c3C(C)OCCCBr ; CCC1=C(C)c2cc3[nH]c(cc4nc(c(C)c5cc(C)c(cc1n2)[nH]5)[C@@H](CCC(=O)OC)[C@@H]4C)c(C)c3C(C)OCCC[n+]1ccccc1 ; [Br-]]

Step 1: To a 50 mL RBF containing phyllochlorin methyl ester (0.50 g, 0.957 mmol, 1 eq) and a stirrer bar was added HBr/AcOH (33% w/w, 5 mL) and the dark blue mixture stirred (300 rpm) at 30° C. for 2 hours under nitrogen. A stream of nitrogen was passed over the sample for a few minutes to remove some of the HBr before concentrating the bulk by rotary evaporation. The mixture was then further dried under high vacuum (0.6 mbar) with heating at 40° C. for 30 minutes. The residue was reconstituted in DCM (20 mL) before powdered K₂CO₃ (1.32 g, 9.57 mmol, 10 eq), then 3-bromo-propan-1-ol (1.22 mL, 14.35 mmol, 15 eq) were added. The system was flushed with nitrogen, stoppered with a rubber septum and the solution was stirred overnight at 30° C. in the dark under a nitrogen atmosphere. The reaction mixture was transferred to a separatory funnel and washed with H₂O (50 mL), then brine (50 mL), before being dried (Na₂SO₄) and concentrated by rotary evaporation to give the crude ether as a dark green oil (˜2.1 g). The residue was further dried under high vacuum (0.3 mBar) for 30 minutes with heating at 40-50° C. to leave 0.95 g of a dark green residue. The residue was purified by column chromatography (4×18 cm) using 0-2% MeOH/DCM. Fractions containing the major dark green spot (R_{f}=0.6 in 1% MeOH/DCM) were combined to give phyllochlorin methyl ester 3-bromopropyl ether as a dark green solid (305 mg, 51%).

¹H NMR (400 MHZ, CDCl₃) δ 9.95-9.73 (m, 2H), 8.88-8.80 (m, 2H), 6.04 (q, 1H), 4.60-4.47 (m, 2H), 3.99 (s, 3H), 3.90-3.80 (m, 3H), 3.78-3.70 (m, 2H), 3.66 (s, 3H), 3.58 (s, 3H), 3.55-3.50 (m, 4H), 3.39 (s, 3H), 2.60-2.47 (m, 2H), 2.30-2.20 (m, 2H), 2.18-2.14 (m, 3H), 2.14-2.00 (m, 2H), 1.81-1.75 (m, 6H), −2.15 (brs, 1H), −2.27 (brs, 1H).

Step 2: To a sealed tube (2.5×20 cm with Teflon screw cap) containing phyllochlorin methyl ester 3-bromopropyl ether (0.100 g, 0.151 mmol, 1 eq) and a stirrer bar was added MeCN (5 mL) and pyridine (60 mg, 0.756 mmol, 5 eq). After flushing the tube with nitrogen and sealing the tube, the dark green mixture was stirred (300 rpm) at 80° C. for 2 hours and then at 90° C. for a further 17 hours. The mixture was cooled and diluted with DCM (25 mL), washed with 0.05 M HCl (20 mL), dried (Na₂SO₄) and concentrated by rotary evaporation to give the crude product as a dark green oil (113 mg). The residue was purified by column chromatography (3×15 cm) eluting using a gradient of 3-15% MeOH/DCM. Fractions containing the major green band (R_{f}=0.1 in 10% MeOH/DCM) were combined to give compound 20 as a dark green solid (70 mg, 74%) (HPLC purity: 97.8%).

¹H NMR (400 MHZ, CDCl₃) δ 9.75 (m, 2H), 8.85 (m, 2H), 8.37 (dd, 2H), 6.66 (dt, 1H), 6.25 (m, 2H), 5.80 (m, 1H), 4.85 (m, 1H), 4.60-4.47 (m, 3H), 3.99 (s, 3H), 3.90-3.80 (m, 3H), 3.64 (s, 3H), 3.56 (m, 3H), 3.45-3.35 (m, 4H), 3.30 (m, 3H), 2.65-2.45 (m, 2H), 2.30-2.15 (m, 2H), 2.10 (m, 3H), 2.1-1.95 (m, 2H), 1.90-1.70 (m, 10H), −2.32 (brs, 1H), −2.38 (brs, 1H).

### Synthesis Example 21—synthesis of phyllochlorin methyl ester 3-triphenylphosphonium bromide propyl ether (compound 21)

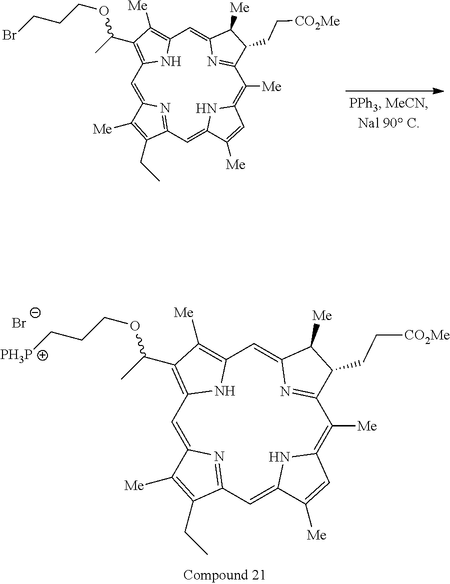

To a sealed tube (2.5×20 cm with Teflon screw cap) containing phyllochlorin methyl ester 3-bromopropyl ether (0.100 g, 0.151 mmol, 1 eq) and a stirrer bar was added MeCN (5 mL), triphenylphosphine (44 mg, 0.166 mmol, 1.1 eq) and sodium iodide (4.5 mg, 0.030 mmol, 0.2 eq). After flushing the tube with nitrogen and sealing the tube, the dark green mixture was stirred (300 rpm) at 90° C. for 22 hours. The mixture was cooled, transferred to a RBF and concentrated by rotary evaporation to give the crude product as a dark green oil. The residue was purified by column chromatography (3×16 cm) eluting using a gradient of 2-5% MeOH/DCM. Fractions containing the major green band (R_{f}=0.15 in 5% MeOH/DCM) were combined to give compound 21 as a dark green solid (102 mg, 86%) (HPLC purity: 98.0%).

¹H NMR (400 MHZ, CDCl₃) δ 9.81 (s, 1H), 9.72 (s, 1H), 8.87 (s, 1H), 8.80 (s, 1H), 7.70-7.18 (m, 15H), 5.98 (q, 1H), 4.60-4.49 (m, 2H), 4.24 (m, 1H), 3.99 (s, 3H), 3.90-3.75 (m, 2H), 3.75-3.65 (m, 5H), 3.63-3.55 (m, 3H), 3.52-3.40 (m, 4H), 3.13 (m, 3H), 2.68-2.45 (m, 2H), 2.20 (m, 3H), 2.18-1.93 (m, 4H), 1.82-1.70 (m, 6H), −2.21 (brm, 1H), −2.28 (brm, 1H).

### Synthesis Example 22—synthesis of phyllochlorin methyl ester 2-(2-pyridiniumethoxy)ethyl ether chloride (compound 22)

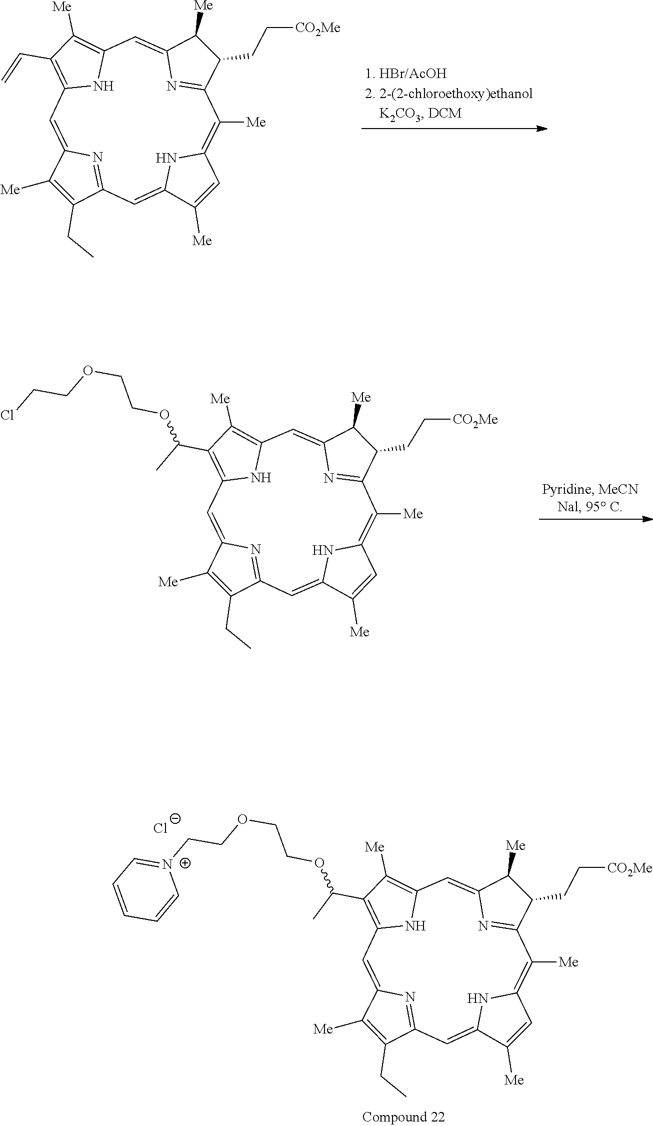 [SMILES: C=Cc1c(C)c2cc3nc(c(C)c4cc(C)c(cc5nc(cc1[nH]2)C(C)=C5CC)[nH]4)[C@@H](CCC(=O)OC)[C@@H]3C ; CCC1=C(C)c2cc3[nH]c(cc4nc(c(C)c5cc(C)c(cc1n2)[nH]5)[C@@H](CCC(=O)OC)[C@@H]4C)c(C)c3C(C)OCCOCCCl ; CCC1=C(C)c2cc3[nH]c(cc4nc(c(C)c5cc(C)c(cc1n2)[nH]5)[C@@H](CCC(=O)OC)[C@@H]4C)c(C)c3C(C)OCCOCC[n+]1ccccc1 ; [Cl-]]

Step 1: To a 50 mL RBF containing phyllochlorin methyl ester (0.50 g, 0.957 mmol, 1 eq) and a stirrer bar was added HBr/AcOH (33% w/w, 5 mL) and the dark blue mixture stirred (300 rpm) at 30° C. for 2 hours under nitrogen. A stream of nitrogen was passed over the sample for a few minutes to remove some of the HBr before concentrating the bulk by rotary evaporation. The mixture was then further dried under high vacuum (0.6 mbar) with heating at 40° C. for 30 minutes. The residue was reconstituted in DCM (20 mL) before powdered K₂CO₃ (1.32 g, 9.57 mmol, 10 eq), then 2-(2-chloroethoxy) ethanol (1.79 g, 14.35 mmol, 15 eq) were added. The system was flushed with nitrogen, stoppered with a rubber septum and the solution was stirred overnight at 30° C. in the dark under a nitrogen atmosphere. The reaction mixture was transferred to a separatory funnel and washed with H₂O (50 mL), then brine (50 mL), before being dried (Na₂SO₄) and concentrated by rotary evaporation (60° C., full vacuum, 1 hr) to give the crude ether as a dark green oil (˜1.1 g). The residue was purified by column chromatography (4×18 cm) using 0-2% MeOH/DCM. Fractions containing the major dark green spot (R_{f}=0.5 in 1% MeOH/DCM) were combined to give phyllochlorin methyl ester 2-(2-chloroethoxy)ethyl ether as a dark green solid (310 mg, 50%).

¹H NMR (400 MHZ, CDCl₃) δ 9.95 (s, 1H), 9.74 (s, 1H), 8.86-8.80 (m, 2H), 6.12 (q, 1H), 4.58 (m, 1H), 4.50 (q, 1H), 3.99 (s, 3H), 3.90-3.70 (m, 9H), 3.66 (s, 3H), 3.65-3.60 (m, 2H), 3.58 (m, 3H), 3.51 (s, 3H), 3.38 (s, 3H), 2.60-2.47 (m, 2H), 2.19 (m, 3H), 2.16-2.00 (m, 2H), 1.80-1.73 (m, 6H), −2.15 (brs, 1H), −2.28 (brs, 1H).

Step 2: To a sealed tube (2.5×20 cm with Teflon screw cap) containing phyllochlorin methyl ester 2-(2-chloroethoxy)ethyl ether (0.110 g, 0.170 mmol, 1 eq) and a stirrer bar was added MeCN (5 mL), pyridine (134 mg, 1.70 mmol, 10 eq) and NaI (5 mg, 0.034 mmol, 0.2 eq). After flushing the tube with nitrogen and sealing the tube, the dark green mixture was stirred (300 rpm) at 95° C. for 16 hours. A further portion of NaI (45 mg, 0.31 mmol, 1.8 eq) was added and the mixture was heated for a further 24 hours. The mixture was cooled and diluted with DCM (25 mL), washed sequentially with 0.05 M HCl (20 mL) and pH=7 phosphate buffer (20 mL), before being dried (Na₂SO₄) and then concentrated by rotary evaporation to give the crude product as a dark green oil. The residue was purified by column chromatography (3×15 cm) eluting using a gradient of 3-8% MeOH/DCM. Fractions containing the major green band (R_{f}<0.1 in 5% MeOH/DCM) were combined to give compound 22 as a dark green solid (48 mg, 39%) (HPLC purity: 98.5%).

¹H NMR (400 MHZ, CDCl₃) δ 10.11 (d, 1H), 9.69 (d, 1H), 8.87-8.80 (m, 2H), 7.12 (d, 1H), 7.05 (d, 1H), 6.31 (m, 1H), 5.81 (q, 1H), 5.23 (m, 2H), 4.64-4.50 (m, 3H), 3.99 (s, 3H), 3.88-3.75 (m, 4H), 3.69 (m, 1H), 3.63 (s, 3H), 3.60-3.55 (m, 3H), 3.51-3.45 (m, 3H), 3.42 (s, 3H), 3.38-3.27 (m, 5H), 2.69-2.45 (m, 2H), 2.26 (m, 3H), 2.22-1.94 (m, 3H), 1.84 (m, 1H), 1.76-1.65 (m, 6H) −2.21 (brm, 1H), −2.34 (brm, 1H).

### Synthesis Example 23—synthesis of phyllochlorin methyl ester 2-(2-triphenylphosphoniumethoxy)ethyl ether chloride (compound 23)

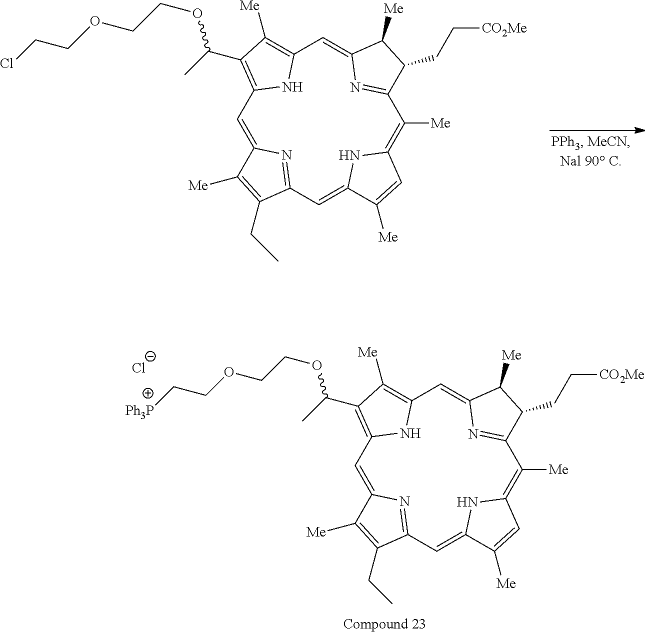

To a sealed tube (2.5×20 cm with Teflon screw cap) containing phyllochlorin methyl ester 2-(2-chloroethoxy)ethyl ether (120 mg, 0.185 mmol, 1 eq) and a stirrer bar was added MeCN (5 mL), triphenylphosphine (53 mg, 0.204 mmol, 1.1 eq) and sodium iodide (42 mg, 0.278 mmol, 1.5 eq). After flushing the tube with nitrogen and sealing the tube, the dark green mixture was stirred (300 rpm) at 90° C. for 72 hours with monitoring by HPLC. The mixture was cooled, transferred to a RBF and concentrated by rotary evaporation to give the crude product as a dark green oil. The residue was purified by column chromatography (3×14 cm) eluting using a gradient of 2-3% MeOH/DCM. Fractions containing the major green band (R_{f}=0.15 in 5% MeOH/DCM) were combined to give compound 23 as a dark green solid (108 mg, 64%) (HPLC purity: 96.4%).

¹H NMR (400 MHZ, CDCl₃) δ 9.88 (s, 1H), 9.70 (s, 1H), 8.86-8.82 (m, 2H), 7.68 (m, 1H), 7.52-7.38 (m, 11H), 7.30-7.23 (m, 8H), 5.80 (q, 1H), 4.60-4.49 (m, 2H), 3.99 (s, 3H), 3.97-3.75 (m, 5H), 3.64 (m, 4H), 3.60-3.55 (m, 3H), 3.50-3.40 (m, 6H), 3.38-3.29 (m, 4H), 2.65-2.55 (m, 2H), 2.20-2.00 (m, 6H), 1.81-1.70 (m, 7H), −2.19 (brs, 1H), −2.31 (brs, 1H).

### Synthesis Example 24—synthesis of phyllochlorin 13-hydroxymethyl N-methyl-N-3,6,9,12,15,18-hexaoxanonadecyl propylamide (compound 24)

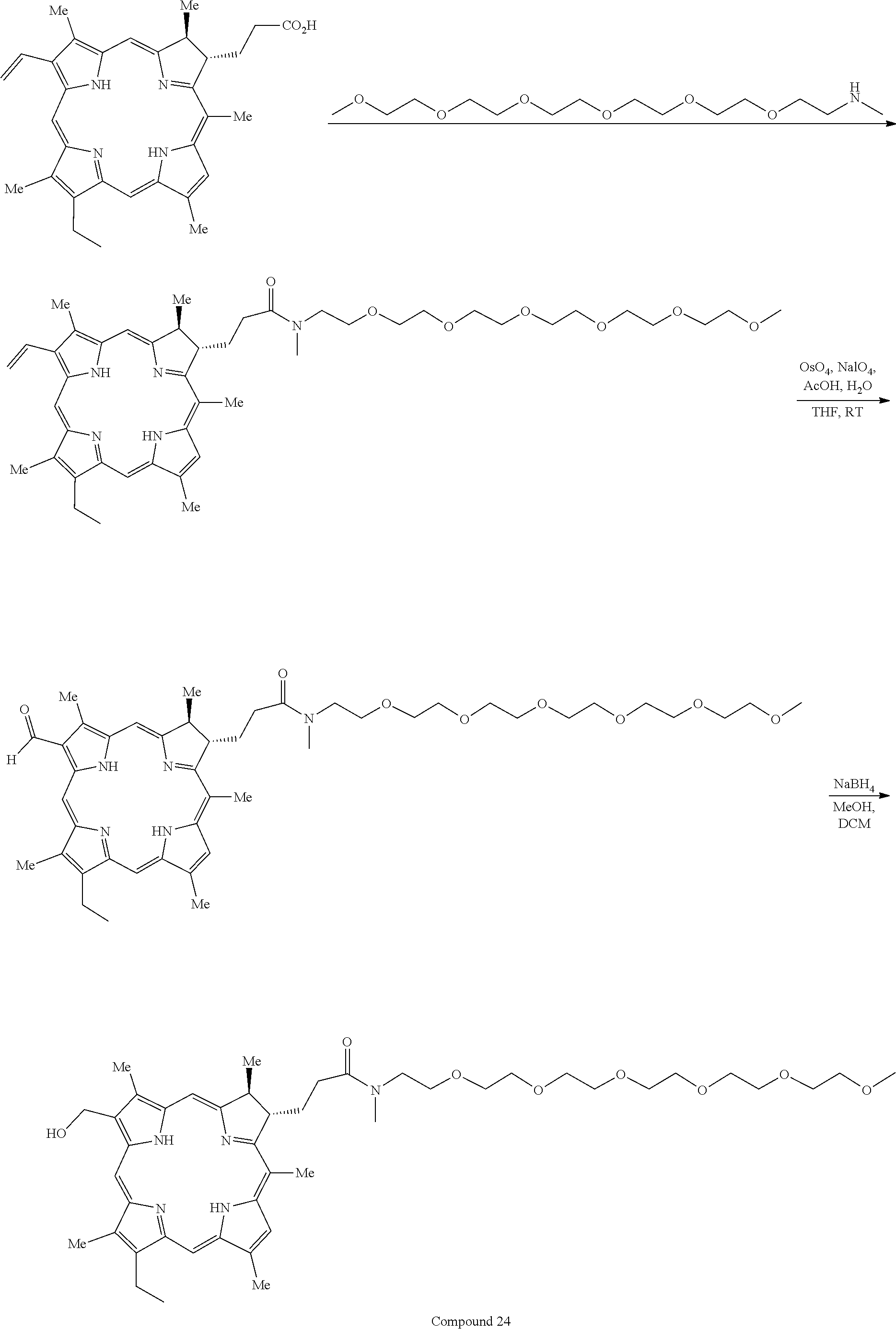

Step 1: Into a 50 mL RBF fitted with a nitrogen inlet was added phyllochlorin (500 mg, 0.983 mmol, 1 eq), dichloromethane (15 mL), DMTMM (354 mg, 1.28 mmol, 1.3 eq) and N-methyl-3,6,9,12,15,18-hexaoxanonadecan-1-amine (365 mg, 1.18 mmol, 1.2 eq). The mixture was stirred at room temperature for 1 hour. The reaction mixture was diluted with DCM (15 mL) and washed with 0.5 M HCl (20 mL) and pH 7 buffer solution (20 mL). The organic layer was then dried (Na₂SO₄), filtered through Celite® and concentrated by rotary evaporation to give the crude product as a dark blue viscous oil (0.86 g). The crude mixture was loaded directly onto a silica column (4×20 cm, pre-equilibrated with 1% MeOH in DCM) and eluted with the same solvent until the first pale-coloured bands eluted and then the solvent was changed incrementally from 1% to 3% MeOH in DCM when the most intense blue/green band began to elute. Fractions containing the product (Major dark green-blue spot, R_{f}=0.35 in 5% MeOH in DCM) were combined and concentrated by rotary evaporation to give phyllochlorin N-methyl-N-3,6,9,12,15,18-hexaoxanonadecyl propylamide as a dark blue viscous oil (750 mg, 95%).

¹H NMR (400 MHZ, CDCl₃) δ 9.76-9.65 (m, 2H), 8.91-8.77 (m, 2H), 8.17 (dd, J=17.8, 11.5 Hz, 1H), 6.38 (dd, J=17.8, 1.6 Hz, 1H), 6.15 (dd, J=11.5, 1.6 Hz, 1H), 4.70-4.62 (m, 1H), 4.54 (q, J=7.2 Hz, 1H), 4.04-3.98 (m, 3H), 3.91-3.77 (m, 2H), 3.67-3.60 (m, 4H), 3.60-3.40 (m, 20H), 3.40-3.35 (m, 5H), 3.34 (s, 3H), 3.26-3.20 (m, 1H), 3.02-2.95 (m, 1H), 2.96-2.89 (m, 1H), 2.86-2.78 (m, 1H), 2.75 (s, 1H), 2.66-2.49 (m, 1H), 2.46 (s, 2H), 2.25-2.12 (m, 1H), 2.06-1.86 (m, 1H), 1.81-1.71 (m, 6H), 1.64 (s, 3H), −2.10 (s, 1H), −2.23 (s, 1H).

Step 2: To a 50 mL RBF was added phyllochlorin N-methyl-N-3,6,9,12,15,18-hexaoxanonadecyl propylamide (660 mg, 0.825 mmol, 1 eq), THF (15 mL), osmium tetroxide (2 mg, 0.008 mmol, 0.01 eq), deionized water (1 mL), AcOH (1 mL) and sodium periodate (441 mg, 2.06 mmol, 2.5 eq). The resultant mixture was stirred (400 rpm) under nitrogen in the dark at ambient temperature overnight. The reaction mixture was concentrated using a rotary evaporator to remove the THF and then re-dissolved in DCM (75 mL), transferred to a separatory funnel and washed with brine (30 mL), saturated NaHCO₃ (30 mL) and water (40 mL) before being dried (Na₂SO₄) and concentrated by rotary evaporation to give phyllochlorin 13-formyl-N-methyl-N-3,6,9,12,15,18-hexaoxanonadecyl propylamide as a red-brown powdery solid.

Step 3: The phyllochlorin 13-formyl-N-methyl-N-3,6,9,12,15,18-hexaoxanonadecyl propylamide (594 mg, 0.741 mmol, 1 eq) was added to a 50 mL RBF, MeOH (10 mL), DCM (5 mL) and sodium borohydride (56.0 mg, 1.48 mmol, 2 eq). The resultant mixture was stirred (420 rpm) under nitrogen ambient temperature for 30 minutes. The reaction mixture was diluted with water (20 mL) and stirred for 10 minutes. The mixture was then extracted with DCM (3×20 mL). The combined DCM layers were washed with water (20 mL), dried (Na₂SO₄) and concentrated by rotary evaporation to give a dark green oil. The dark green oil was purified by silica gel column chromatography using a gradient of 1-7% MeOH/DCM. Fractions containing the product (Major dark green spot, R_{f}=0.28 in 5% MeOH/DCM) were combined to give compound 24 as a dark blue solid (231 mg, 35% over 2 steps).

¹H NMR (400 MHZ, CDCl₃) δ 9.75-9.65 (m, 2H), 8.86 (d, J=3.1 Hz, 2H), 6.01-5.88 (m, 2H), 4.72-4.62 (m, 1H), 4.54 (dd, J=7.5, 3.0 Hz, 1H), 4.02 (d, J=3.5 Hz, 3H), 3.82 (q, J=7.6 Hz, 2H), 3.67-3.61 (m, 3H), 3.60-3.38 (m, 17H), 3.37-3.32 (m, 7H), 3.31-3.27 (m, 1H), 3.15-3.09 (m, 1H), 2.90-2.72 (m, 2H), 2.70 (s, 1H), 2.66 (t, J=5.0 Hz, 1H), 2.63-2.50 (m, 1H), 2.43 (s, 2H), 2.36 (dt, J=15.2, 7.5 Hz, 1H), 2.25-2.13 (m, 1H), 1.97-1.84 (m, 1H), 1.81-1.69 (m, 6H), 1.65 (s, 2H), −2.25 (s, 1H), −2.41 (s, 1H).

### Synthesis Example 25—synthesis of phyllochlorin 13-hydroxymethyl-β-D-glucoside ether-β-D-glucosel-N-methyl-N-3,6,9,12,15,18-hexaoxanonadecyl propylamide (compound 25)

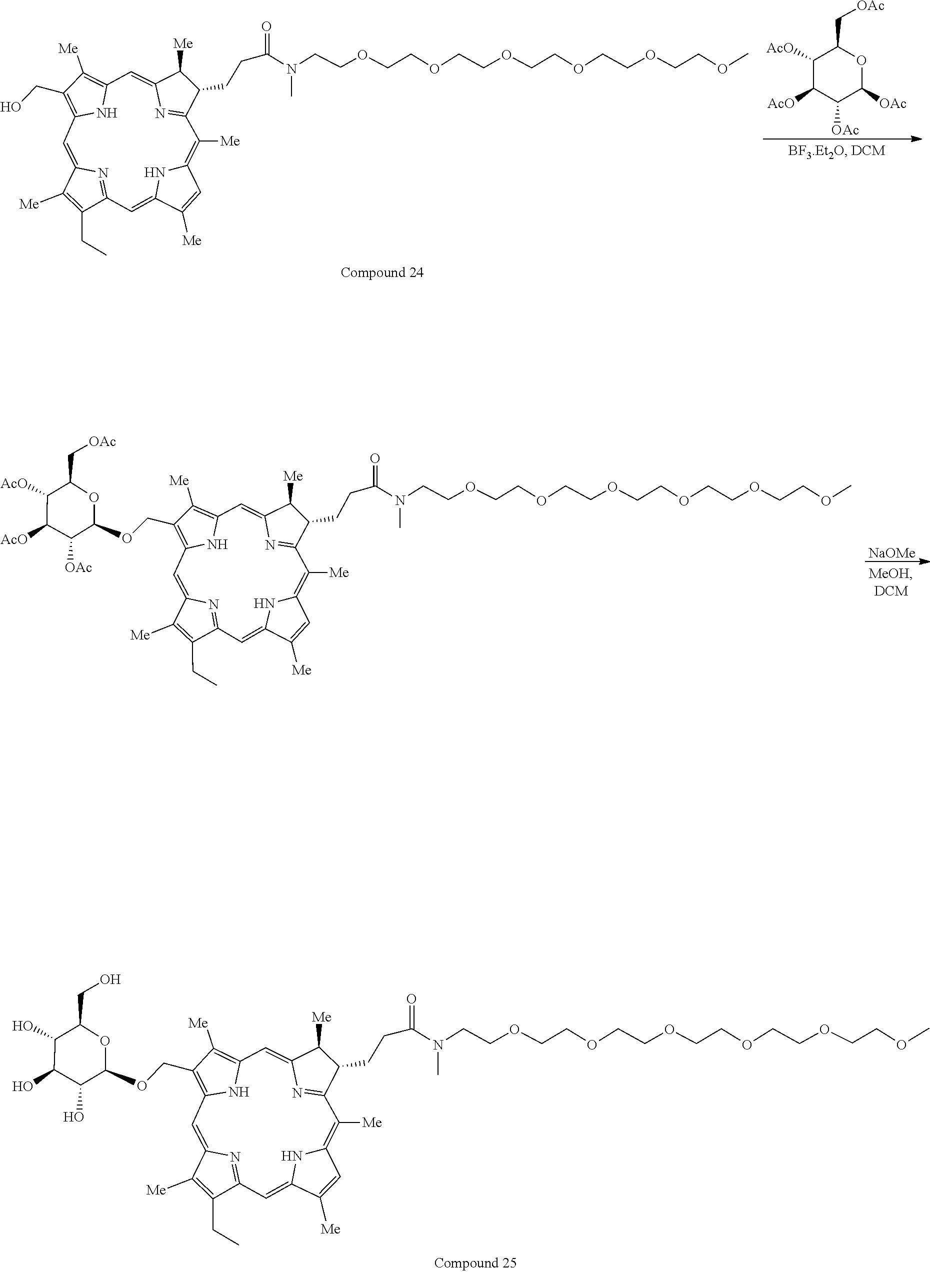

Step 1: To a 25 mL RBF was added phyllochlorin 13-hydroxymethyl N-methyl-N-3,6,9,12,15,18-hexaoxanonadecyl propylamide (compound 24) (200 mg, 0.249 mmol, 1 eq), glucose pentaacetate (146 mg, 0.374 mmol, 1.5 eq) and DCM (10 mL). The resultant mixture was stirred (420 rpm) under nitrogen cooling to ˜0° C. Boron trifluoride diethyl etherate (0.2 mL) was added and stirring was continued allowing the solution to warm slowly to room temperature over 2 hours and then stirred at room temperature in the dark for 18 hours. An additional portion of glucose pentaacetate (48 mg, 0.125 mmol, 0.5 eq) and boron trifluoride diethyl etherate (0.2 mL) were added and the reaction was stirred for an additional 4 hours. The reaction mixture was diluted with DCM (30 mL) and washed with saturated NaHCO₃ (25 mL), then water (20 mL). The organic phase was then dried (Na₂SO₄) and concentrated by rotary evaporation to give a blue-black film. The resultant residue was subjected to column chromatography (3×20 cm silica gel). The crude product was dissolved in DCM and eluted using a gradient of 1-6% MeOH/DCM to give phyllochlorin 13-hydroxymethyl-β-D-glucoside ether tetraacetate-β-D-glucosel-N-methyl-N-3,6,9,12,15,18-hexaoxanonadecyl propylamide tetraacetate as a dark blue-green viscous syrup (300 mg).

Step 2: To a solution of phyllochlorin 13-hydroxymethyl-β-D-glucoside ether tetraacetate-β-D-glucosel-N-methyl-N-3,6,9,12,15,18-hexaoxanonadecyl propylamide tetraacetate (294 mg, 0.249 mmol, 1 eq) in MeOH (4 mL) and DCM (4 mL) was added NaOMe (4.6 M in MeOH, 0.054 mL, 0.249 mmol, 1 eq), and the mixture stirred (420 rpm) under N₂ for 1 hour. The reaction mixture was concentrated by rotary evaporation to give a black film residue. The residue was purified by column chromatography (3×20 cm silica gel) using an eluent gradient of 5-7% MeOH in DCM and then 7%>12% MeOH in DCM. Fractions with R_{f}=0.3 in 10% MeOH/DCM were combined to give compound 25 as a dark green viscous syrup (81 mg, 34% over 2 steps) (HPLC purity: 94.5%).

¹H NMR (400 MHZ, DMSO-d₆) δ 9.86 (s, 1H), 9.76 (s, 1H), 9.07 (s, 2H), 6.08 (dd, J=59.1, 12.2 Hz, 2H), 5.22-4.74 (m, 5H), 4.70-4.50 (m, 3H), 3.99 (d, J=3.7 Hz, 3H), 3.92 (d, J=11.5 Hz, 1H), 3.84 (q, J=7.5 Hz, 2H), 3.70 (dd, J=12.0, 5.7 Hz, 1H), 3.63 (s, 3H), 3.52 (s, 3H), 3.47-3.38 (m, 7H), 3.32-3.18 (m, 7H), 3.15 (d, J=6.9 Hz, 3H), 2.93 (s, 2H), 2.81 (s, 3H), 2.47-2.35 (m, 0H), 1.70 (t, J=7.8 Hz, 7H), −2.36 (s, 1H), −2.53 (s, 1H).

### Synthesis Example 26—synthesis of 13-hydroxyethylphyllochlorin-β-D-1-thioglucose-N-methylpropylamide (compound 26)

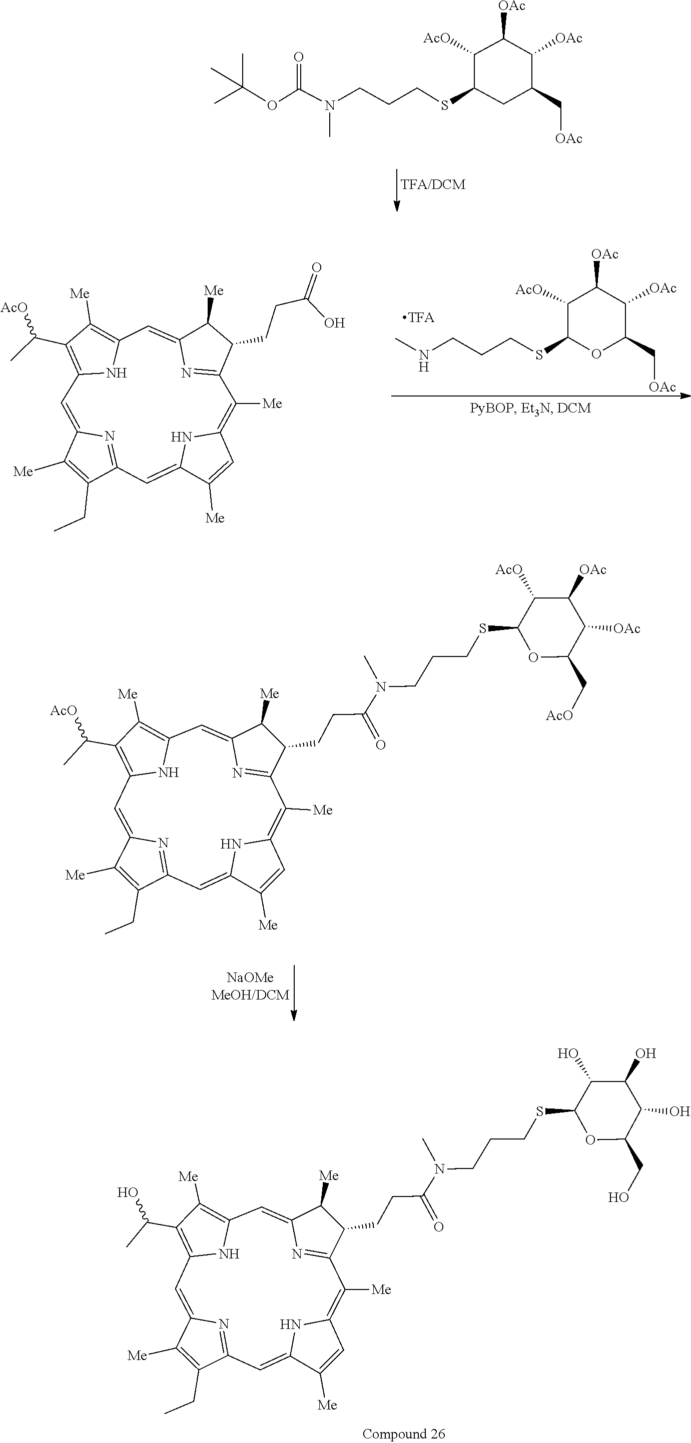

To a solution of (2R,3R,4S,5R,6S)-2-(acetoxymethyl)-6-((3-((tert-butoxycarbonyl) (methyl)amino) propyl)thio)tetrahydro-2H-pyran-3,4,5-triyl triacetate (0.612 g, 1.14 mmol, 1.4 eq) in DCM (5 mL) was added TFA (1 mL). The resultant solution was stirred (420 rpm) for 1 hour at ambient temperature, then concentrated on the rotary evaporator. The residue was resuspended and concentrated twice from chloroform (2×10 mL) to give a viscous oil that was dissolved in DCM (2 mL) for the subsequent coupling reaction. To a 25 mL RBF was added phyllochlorin 13-(ethyl-1-acetate) (0.45 g, 0.791 mmol, 1 eq), PyBOP (0.494 g, 0.749 mmol, 1.2 eq), DCM (6 mL) and triethylamine (0.66 mL, 4.74 mmol, 6 eq). The resultant mixture was stirred (420 rpm) under nitrogen at ambient temperature for 30 minutes, then the prepared solution of the deprotected amine in DCM (2 mL) was added in one portion. The resultant mixture was stirred overnight. The reaction mixture was transferred to a separatory funnel and washed with 1 M HCl (2×10 mL), then pH 7 buffer (1×10 mL). The organic phase was dried (Na₂SO₄) and concentrated by rotary evaporation to give a blue-black film (˜1.15 g). The residue was purified by column chromatography (3×25 cm) using 2-3% MeOH/DCM. Fractions containing the major dark green spot (R_{f}=0.6 in 5% MeOH/DCM) were combined to give compound 26 as a dark blue-black solid (398 mg, 54%). The solid was dissolved in a mixture of MeOH (5 mL) and DCM (5 mL) and NaOMe (4.6 M in MeOH, 0.43 mL, 1.98 mmol, 5 eq) was added. The mixture was stirred (200 rpm) under nitrogen for 90 minutes. The reaction was quenched with AcOH (4.11 mL, 71.81 mmol, 10 eq) and concentrated by rotary evaporation to give a black film. The residue was purified by column chromatography (3×21 cm) using 3-12% MeOH/DCM. Fractions containing the major dark green spot (R_{f}=0.85 in 10% MeOH/DCM) were combined to give compound 26 as a dark blue-black solid (255 mg, 83%).

¹H NMR (400 MHZ, d₆-DMSO) δ 10.05 (m, 1H), 9.74 (s, 1H), 9.05 (s, 1H), 9.00 (d, 1H), 6.41 (m, 1H), 6.10 (s, 1H), 5.20-4.91 (m, 3H), 4.64 (m, 1H), 4.58-4.47 (m, 2H), 4.28 (dd, 1H), 4.11 (q, 1H), 3.98 (d, 3H), 3.82 (q, 2H), 3.63 (brs, 3H), 3.50 (d, 3H), 3.20-3.15 (m, 2H), 3.15-3.05 (m, 2H), 2.90 (s, 2H), 2.81 (s, 1H), 2.70-2.53 (m, 2H), 2.42 (m, 1H), 2.03 (m, 3H), 1.81 (m, 1H), 1.75-1.61 (m, 8H), −2.34 (s, 1H), −2.43 (s, 1H).

### Synthesis Example 27—synthesis of phyllochlorin 13-(ethyl-1-alcohol) (compound 27)

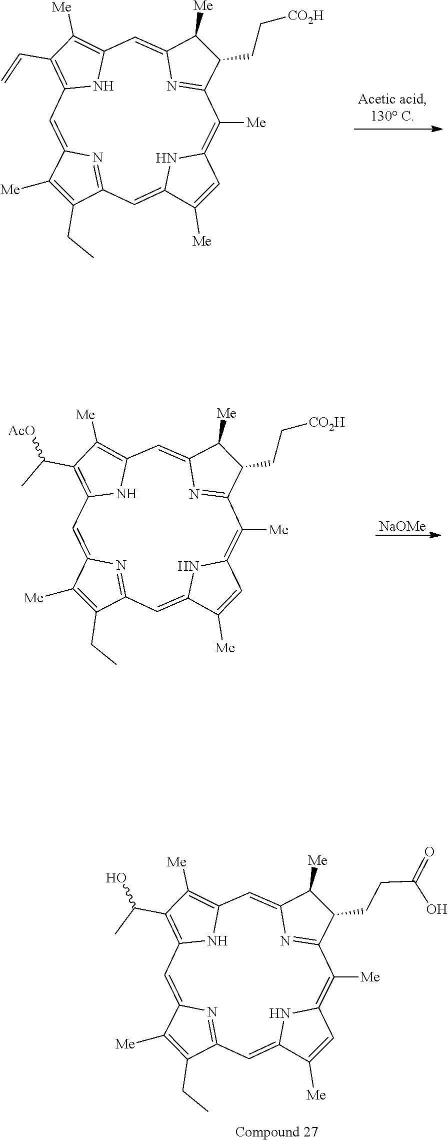 [SMILES: C=Cc1c(C)c2cc3nc(c(C)c4cc(C)c(cc5nc(cc1[nH]2)C(C)=C5CC)[nH]4)[C@@H](CCC(=O)O)[C@@H]3C ; CCC1=C(C)c2cc3[nH]c(cc4nc(c(C)c5cc(C)c(cc1n2)[nH]5)[C@@H](CCC(=O)O)[C@@H]4C)c(C)c3C(C)OC(C)=O ; CCC1=C(C)c2cc3[nH]c(cc4nc(c(C)c5cc(C)c(cc1n2)[nH]5)[C@@H](CCC(=O)O)[C@@H]4C)c(C)c3C(C)O ; "NaOMe"]

Step 1: Into a 50 mL RBF with a stirrer bar and fitted with a condenser with gas inlet/outlet and bubbler was added phyllochlorin (500 mg, 9.87 mmol, 1 eq) and acetic acid (15 mL). The flask was then heated at 130° C. with stirring (400 rpm) in the dark overnight. After 27 hours the reaction was cooled and concentrated on a rotary evaporator to give a blue solid. This material was suspended in DCM (50 mL), washed with water (80 mL), dried (Na₂SO₄), filtered and evaporated to give an intense blue glassy solid (1.1 g). The residue was purified by column chromatography (4×12 cm) using 2% MeOH/DCM. Fractions containing a dark green spot with R_{f}=0.3 in 5% MeOH/DCM were combined to give phyllochlorin 13-(ethyl-1-acetate) as a dark green solid (20 mg, 4%).

¹H NMR (400 MHZ, CDCl₃) δ 9.92 (s, 1H), 9.76 (s, 1H), 8.85 (m, 2H), 7.40 (m, 1H), 4.61-4.55 (m, 1H), 4.53-4.46 m, 1H), 3.96 (s, 3H), 3.87 (q, 2H), 3.64 (s, 3H), 3.55 (s, 3H), 3.40 (s, 3H), 2.56-2.45 (m, 2H), 2.25 (m, 6H), 2.12-1.97 (m, 2H), 1.80-1.71 (m, 7H), −2.38 (brs, 1H).

Step 2: To phyllochlorin 13-(ethyl-1-acetate) (4.14 g, 7.28 mmol, 1 eq) in MeOH (40 mL) and DCM (20 mL) was added NaOMe (4.6 M, 2.37 mL, 1.5 eq) and the solution was stirred at room temperature for 30 minutes with monitoring by HPLC. AcOH (656 mg, 10.92 mmol, 1.5 eq) was added and the solvent removed by rotary evaporation to leave a dark blue solid. The residue was subjected to column chromatography (silica gel, 6×20 cm) using 2-10% MeOH in DCM as eluent. Fractions containing the product (Major dark green spot, R_{f}=0.31 in 5% MeOH in DCM) were combined and concentrated by rotary evaporation. The solid was dried in a vacuum oven at 60° C. overnight to give compound 27 as a dark blue solid (1.37 g, 68%) (HPLC purity: 97.1%).

¹H NMR (400 MHZ, CDCl₃) δ 9.80-9.53 (m, 2H), 8.85-8.76 (m, 2H), 6.07 (p, J=6.7 Hz, 1H), 4.62-4.40 (m, 2H), 3.92 (s, 3H), 3.78 (h, J=8.7, 8.2 Hz, 2H), 3.61-3.53 (m, 3H), 3.32 (d, J=6.3 Hz, 3H), 3.24 (d, J=2.0 Hz, 3H), 2.50-2.23 (m, 1H), 1.94 (t, J=6.7 Hz, 3H), 1.79-1.64 (m, 5H), −2.43 (s, 1H).

### Synthesis Example 28—Synthesis of phyllochlorin methyl ester 3-picolinyl bromide propyl ether (compound 28)

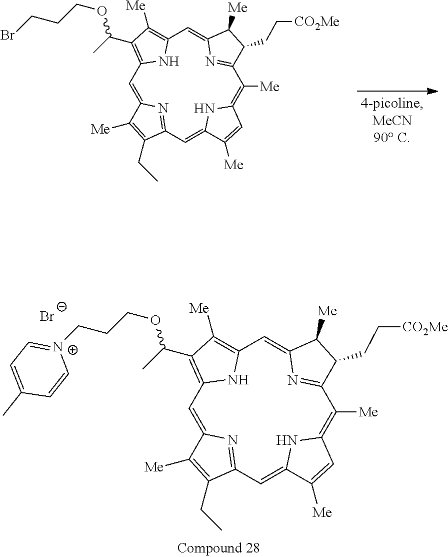 [SMILES: CCC1=C(C)c2cc3[nH]c(cc4nc(c(C)c5cc(C)c(cc1n2)[nH]5)[C@@H](CCC(=O)OC)[C@@H]4C)c(C)c3C(C)OCCCBr ; CCC1=C(C)c2cc3[nH]c(cc4nc(c(C)c5cc(C)c(cc1n2)[nH]5)[C@@H](CCC(=O)OC)[C@@H]4C)c(C)c3C(C)OCCC[n+]1ccc(C)cc1 ; [Br-]]

To a sealed tube (2.5×20 cm with Teflon screw cap) containing phyllochlorin methyl ester 3-bromopropyl ether (70 mg, 0.106 mmol, 1 eq) and a stirrer bar was added MeCN (4 mL) and 4-picoline (49 mg, 0.529 mmol, 5 eq). After flushing the tube with nitrogen and sealing the tube, the dark green mixture was stirred (300 rpm) at 90° C. for 22 hours. The mixture was cooled and diluted with DCM (25 mL), washed with 0.05 M HCl (20 mL), dried (Na₂SO₄) and concentrated by rotary evaporation to give the crude product as a dark green oil (115 mg). The residue was purified by column chromatography (3×12 cm) using 8% MeOH/DCM, loaded as a solution in the eluent, to elute the high R^{β} starting materials. Then a gradient of 8-14% MeOH/DCM was used to elute compound 28 (R_{f}=0.1 in ˜10% MeOH/DCM) as a dark green solid (64 mg, 94%) (HPLC purity: 96.7%-2 isomers, 1.4% DCM w/w).

¹H NMR (400 MHZ, CDCl₃) δ 9.77 (m, 2H), 8.85 (m, 2H), 8.28 (m, 2H), 5.99 (m, 2H), 5.81 (m, 1H), 4.88 (m, 1H), 4.61-4.47 (m, 3H), 4.01-3.94 (m, 4H), 3.86 (m, 2H), 3.66 (s, 3H), 3.59 (m, 3H), 3.51-3.45 (m, 1H), 3.40 (s, 3H), 3.31 (m, 3H), 2.67-2.45 (m, 2H), 2.38-2.18 (m, 3H), 2.11 (m, 3H), 2.07-1.95 (m, 1H), 1.88-1.70 (m, 10H), −2.31-−2.45 (brm, 2H).

### Synthesis Example 29—synthesis of phyllochlorin methyl ester 13-(2-methoxyethyl)carbamate (compound 29)

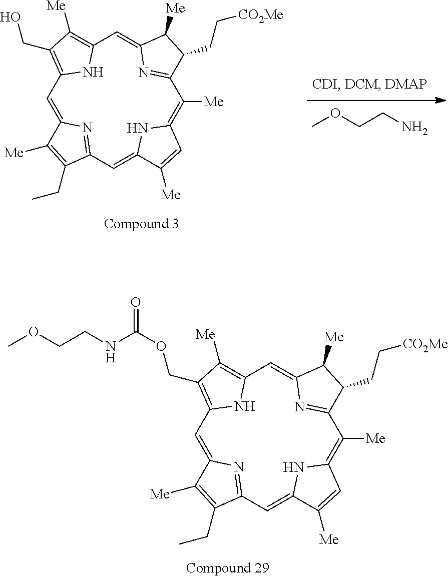

To a 50 mL RBF was added phyllochlorin 13-hydroxymethyl methyl ester (compound 3) (100 mg, 0.190 mmol, 1 eq), carbonyl diimidazole (62 mg, 0.380 mmol, 2 eq), DCM (10 mL) and DMAP (10 mg). The resultant mixture was stirred (300 rpm) under nitrogen for 2 hours. 2-Methoxyethylamine (0.4 mL) was added and stirring was continued for 3 hours. The reaction mixture was diluted with DCM (20 mL), transferred to a separatory funnel and washed with water (2×40 mL), before being dried (Na₂SO₄) and concentrated by rotary evaporation to give a dark green residue (140 mg). The residue was purified by column chromatography (3×12 cm) using 2-5% acetone/DCM. Fractions containing the major dark green spot (R_{f}=0.40 in 5% acetone/DCM) were combined to give compound 29 as a dark green solid (44 mg, 37%).

¹H NMR (400 MHZ, CDCl₃) δ 9.75 (m, 2H), 8.89 (m, 2H), 6.49 (m, 2H), 5.20 (brs, 1H), 4.58 (m, 1H), 4.51 (m, 1H), 4.00 (s, 3H), 3.86 (q, 2H), 3.67 (s, 3H), 3.57 (m, 6H), 3.48 (m, 3H), 3.40 (s, 3H), 3.30 (s, 3H), 2.62-2.48 (m, 2H), 2.18-2.00 (m, 2H), 1.81-1.72 (m, 6H), −2.21 (brs, 1H), −2.38 (brs, 1H).

### Synthesis Example 30—synthesis of phyllochlorin 13-formyl methyl ester (compound 30)

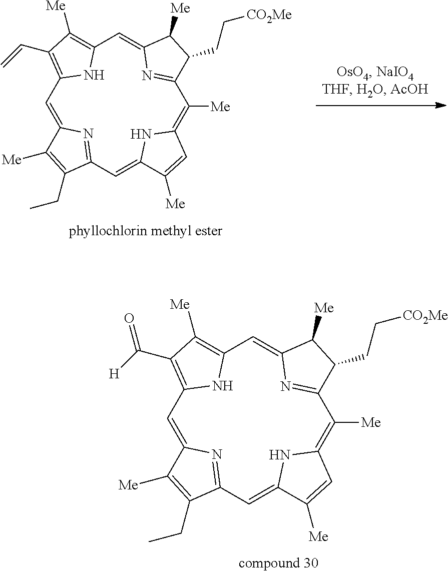 [SMILES: C=Cc1c(C)c2cc3nc(c(C)c4cc(C)c(cc5nc(cc1[nH]2)C(C)=C5CC)[nH]4)[C@@H](CCC(=O)OC)[C@@H]3C ; CCC1=C(C)c2cc3[nH]c(cc4nc(c(C)c5cc(C)c(cc1n2)[nH]5)[C@@H](CCC(=O)OC)[C@@H]4C)c(C)c3C=O]

To a 250 mL RBF was added phyllochlorin methyl ester (2.00 g, 3.826 mmol, 1 eq), THF (75 mL), osmium tetroxide (5 mg, 0.038 mmol, 0.01 eq), deionized water (5 mL), AcOH (5 mL) and sodium periodate (1.80 g, 8.418 mmol, 2.2 eq). The resultant mixture was stirred under nitrogen in the dark at ambient temperature for 3 hours. A further portion of sodium periodate (0.35 g, 0.4 eq) was added and the solution stirred overnight. The reaction mixture was concentrated using a rotary evaporator to remove the THF and then re-dissolved in DCM (60 mL), transferred to a separatory funnel and washed with brine (40 mL), saturated NaHCO₃ (40 mL) and water (40 mL) before being dried (Na₂SO₄) and concentrated by rotary evaporation to give a red-brown powdery solid. The residue was purified by column chromatography using a gradient of 0-2% MeOH/DCM. Fractions containing the major dark green spot (R_{f}=0.7 in 5% MeOH/DCM) were combined to give compound 30 as a red-brown solid (1.65 g, 82%).

¹H NMR (400 MHz, CDCl₃) δ 11.60 (s, 1H), 10.32 (s, 1H), 9.61 (s, 1H), 8.98 (s, 1H), 8.90 (s, 1H), 4.56 (m, 1H), 4.50 (m, 1H), 4.00 (s, 3H), 3.81 (m, 5H), 3.62 (s, 3H), 3.60 (s, 3H), 3.38 (s, 3H), 2.66-2.56 (m, 1H), 2.55-2.47 (m, 1H), 2.25-2.15 (m, 1H), 2.05-1.95 (m, 1H), 1.80-1.71 (m, 6H), −1.80 (s, 1H), −2.25 (s, 1H).

### Synthesis Example 31—synthesis of phyllochlorin 13-formyl N-3-hydroxypropyl-N-methyl propylamide acetate (compound 31)

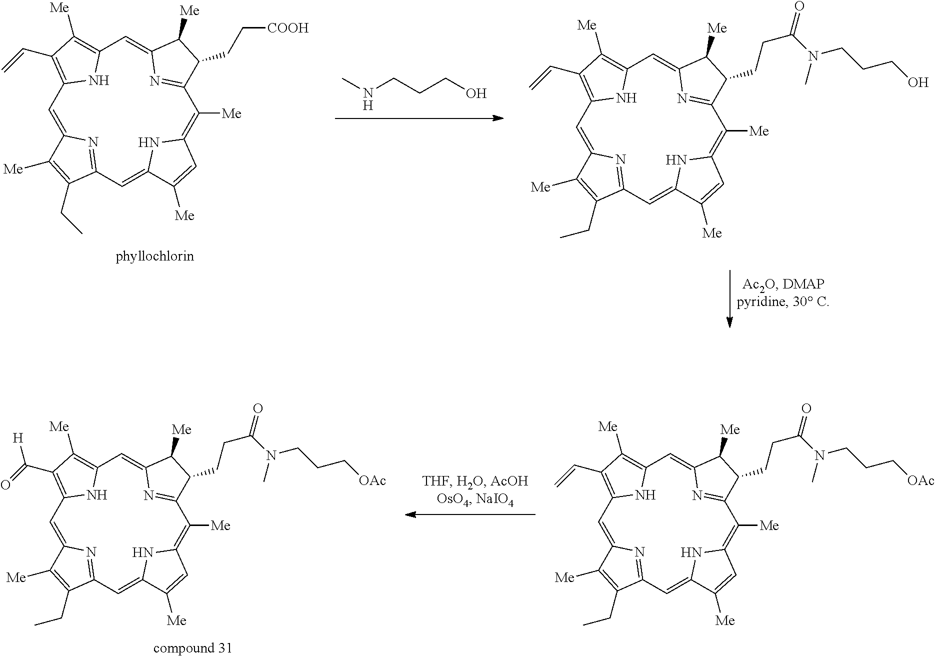

Step 1: Into a 100 mL RBF fitted with a nitrogen inlet and containing a small stirrer bar was added phyllochlorin (2.00 g, 3.93 mmol, 1 eq), dichloromethane (50 mL), PyBOP (2.26 mg, 1.1 eq), triethylamine (1.64 mL, 3 eq) and 3-(methylamino)-propanol (0.42 g, 1.2 eq). The mixture was stirred at room temperature for 3 hours. Analysis by HPLC showed the reaction to be complete. The reaction mixture was transferred to a separatory funnel and washed with water (2×30 mL). The organic layer was dried (Na₂SO₄) and concentrated by rotary evaporation to give the crude product as a blue/brown film (5.1 g). The crude mixture was loaded directly onto a silica column and eluted with 1.5-2% MeOH/DCM. Pure fractions containing a green/blue spot by TLC with R_{f} 0.30 (5% MeOH/DCM) were combined to give phyllochlorin N-3-hydroxypropyl-N-methyl propylamide (1.29 g, 57%).

¹H NMR (400 MHZ, CDCl₃) δ 9.75-9.70 (m, 2H), 8.85 (br s, 1H), 8.16 (dd, 1H), 6.38 (dd, 1H), 6.15 (dd, 1H), 4.70-4.65 (m, 1H), 4.58-4.50 (m, 1H), 4.00 (s, 3H), 3.89-3.82 (m, 3H), 3.65 (s, 3H), 3.53 (s, 3H), 3.37 (s, 3H), 3.31-3.15 (m, 4H), 2.65-2.53 (m, 2H), 2.37-2.22 (m, 3H), 2.16 (3, 3H), 1.80-1.70 (m, 7H), 1.48-1.40 (m, 2H), −2.10 (s, 1H), −2.22 (m, 1H).

Step 2: Into a 25 mL RBF was added phyllochlorin N-3-hydroxypropyl-N-methyl propylamide (300 mg, 0.517 mmol, 1 eq), pyridine (2 mL), acetic anhydride (0.49 mL, 5.17 mmol, 10 eq) and DMAP (1 mg). The solution was stirred at 30° C. for 1 hour. Analysis by TLC indicated the reaction was complete. Ethyl acetate (15 mL) and water (10 mL) were added and the mixture stirred vigorously for 10 minutes. The layers were separated and the ethyl acetate layer was washed with 0.5 M HCl (2×10 mL) and saturated NaHCO₃ (2×10 mL) before being dried (Na₂SO₄) and concentrated to give phyllochlorin N-3-hydroxypropyl-N-methyl propylamide acetate as a dark green solid that was not purified further (288 mg, 89%).

Step 3: To a 100 mL RBF was added phyllochlorin N-3-hydroxypropyl-N-methyl propylamide acetate (280 mg, 0.450 mmol, 1 eq), THF (15 mL), osmium tetroxide (1 mg, 0.005 mmol, 0.01 eq), deionized water (1 mL), AcOH (1 mL) and sodium periodate (202 mg, 0.946 mmol, 2.1 eq). The resultant mixture was stirred under nitrogen in the dark at ambient temperature overnight. The reaction mixture was concentrated using a rotary evaporator to remove the THF and then re-dissolved in DCM (75 mL), transferred to a separatory funnel and washed with brine (30 mL), saturated NaHCO₃ (30 mL) and water (40 mL) before being dried (Na₂SO₄) and concentrated by rotary evaporation to give a red-brown powdery solid. The residue was purified by column chromatography eluting using a gradient of 1-2.5% MeOH/DCM. Fractions containing the major dark green spot (R_{f}=0.65 in 5% MeOH/DCM) were combined to give compound 31 as a red-brown powder (200 mg, 71%).

¹H NMR (400 MHZ, CDCl₃) δ 11.60 (s, 1H), 10.31 (s, 1H), 9.60 (s, 1H), 8.98 (s, 1H), 8.90 (s, 1H), 4.70-4.65 (m, 1H), 4.56-4.50 (m, 1H), 4.10 (t, 1H), 4.02 (m, 3H), 3.98-3.94 (m, 5H), 3.85-3.78 (m, 5H), 3.70 (m, 1H), 3.65-3.56 (m, 4H), 3.36 (s, 3H), 3.25 (t, 1H), 3.15 (s, 3H), 2.75-2.70 (m, 2H), 2.60-2.50 (m, 2H), 2.45-2.38 (m, 3H), 2.20-2.10 (m, 1H), 2.05 (s, 2H), 2.00-1.95 (m, 4H), 1.80-1.70 (m, 7H), 1.68 (m, 2H), −1.75 (s, 1H), −2.25 (s, 1H).

### Synthesis Example 32—synthesis of phyllochlorin 13-hydroxymethyl-3-hydroxypropyl (compound 32)

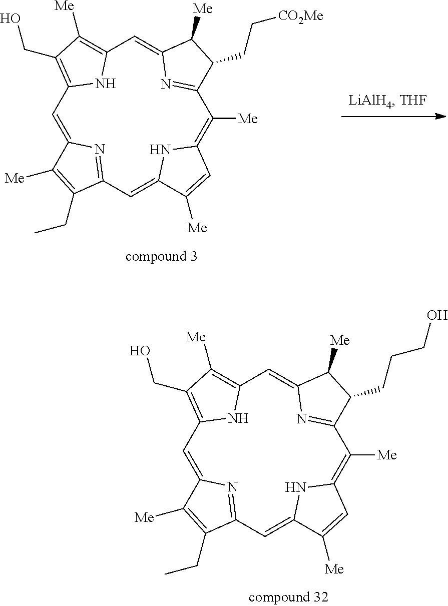 [SMILES: CCC1=C(C)c2cc3[nH]c(cc4nc(c(C)c5cc(C)c(cc1n2)[nH]5)[C@@H](CCC(=O)OC)[C@@H]4C)c(C)c3CO ; CCC1=C(C)c2cc3[nH]c(cc4nc(c(C)c5cc(C)c(cc1n2)[nH]5)[C@@H](CCCO)[C@@H]4C)c(C)c3CO]

Into a 3-neck 100 mL RBF was added lithium aluminium hydride (230 mg, 5.75 mmol, 10 eq) and THF (25 mL) and the solution was stirred and cooled using an ice/water bath for 10 minutes under nitrogen. Phyllochlorin 13-hydroxymethyl methyl ester (compound 3) (300 mg, 0.370 mmol, 1 eq) was added in portions over 2 minutes. After 30 minutes the reaction flask was removed from the cold bath and stirring continued at room temperature. The flask was cooled (ice/water bath) and water (0.15 mL) was added followed by 4M NaOH (0.15 mL). After stirring for 10 minutes further water (0.5 mL) was added and the solution was warmed to room temperature and stirred for 15 minutes. Sodium sulfate (˜5 g) was added and the mixture was stirred for 10 minutes before being filtered, rinsing with DCM until no more colour eluted. The solvent was then removed under reduced pressure to give a residue which was purified by column chromatography eluting using a gradient of 1-5% MeOH/DCM. Fractions containing the product (R_{f}=0.2 in 5% MeOH/DCM) were combined to give compound 32 as a dark green solid (220 mg, 77%).

¹H NMR (400 MHZ, CDCl₃) δ 9.71 (s, 1H), 9.61 (s, 1H), 8.86 (s, 1H), 8.84 (s, 1H), 5.85 (m, 2H), 4.51 (m, 2H), 3.95 (s, 3H), 3.83 (q, 2H), 3.63 (s, 3H), 3.60-3.50 (m, 2H), 3.46 (s, 3H), 3.32 (s, 3H), 2.22-2.15 (m, 1H), 2.05-1.95 (m, 1H), 1.82-1.70 (m, 8H), 1.52-1.42 (m, 2H), 1.20-1.10 (brs, 1H), −2.25 (brs, 1H), −2.39 (brs, 1H).

### Synthesis Example 33—synthesis of phyllochlorin 13-hydroxymethyl-3-hydroxypropyl-bis-β-D-glucoside ether (compound 33)

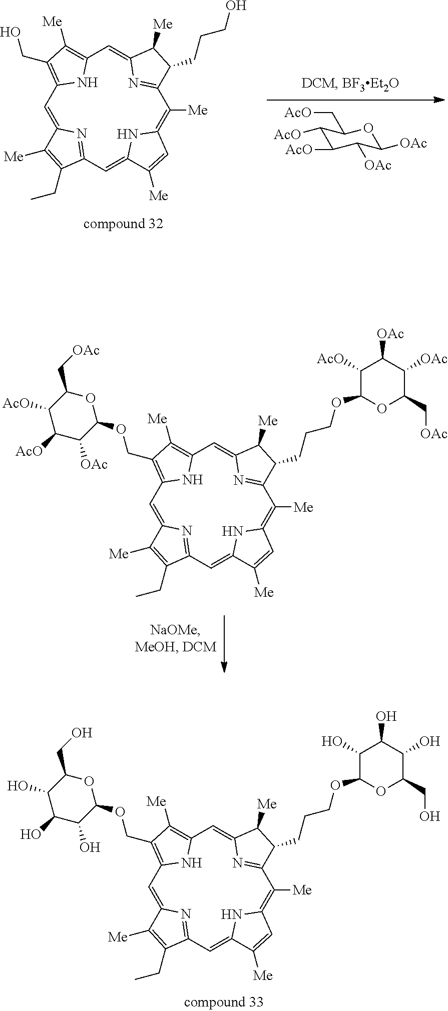

Step 1: To a 50 mL RBF was added phyllochlorin 13-hydroxymethyl-3-hydroxypropyl (compound 32) (120 mg, 0.241 mmol, 1 eq), glucose pentaacetate (282 mg, 0.722 mmol, 3 eq) and DCM (10 mL). The resultant mixture was stirred under nitrogen cooling to ˜0° C. using an ice/water bath. Boron trifluoride diethyl etherate (0.3 mL) was added and stirring was continued allowing the solution to warm slowly to room temperature over 2 hours and then stirred at room temperature in the dark overnight. The reaction mixture was diluted with DCM (20 mL), transferred to a separatory funnel and washed with saturated NaHCO₃ (20 mL) and then water (20 mL). The organic phase was dried (Na₂SO₄) and concentrated by rotary evaporation to give a blue-black film. The residue was purified by column chromatography eluting using a gradient of 0.5-1.5% MeOH/DCM. Fractions containing the product (R_{f}=0.65 in 5% MeOH/DCM) were combined to give phyllochlorin 13-hydroxymethyl-3-hydroxypropyl-bis-β-D-glucoside ether peracetate as dark green solid (170 mg).

Step 2: To a solution of phyllochlorin 13-hydroxymethyl-3-hydroxypropyl-bis-β-D-glucoside ether peracetate (140 mg, 0.121 mmol, 1 eq) in MeOH (3 mL) and DCM (2 mL) was added NaOMe (4.6 M in MeOH, 0.026 mL, 0.121 mmol, 1 eq), and the mixture stirred under N₂ for 1 hour. The reaction was concentrated by rotary evaporation to give a black oil. The residue was purified by column chromatography (3×14 cm) eluting using a gradient of 10-20% MeOH/DCM. Fractions containing the product (R_{f}=0.1 in 15% MeOH/DCM) were combined to give compound 33 as a dark green solid (72 mg, 36% over 2 steps).

¹H NMR (400 MHZ, DMSO-d₆) δ 9.85 (s, 1H), 9.76 (s, 1H), 9.10 (s, 1H), 9.07 (m, 1H), 6.59 (d, 1H), 6.21 (d, 1H), 6.18 (d, 1H), 6.02 (d, 1H), 5.10 (d, 1H), 4.95 (m, 2H), 4.90-4.78 (m, 6H), 4.65-4.55 (m, 4H), 4.50-4.40 (m, 3H), 4.30-4.20 (m, 1H), 4.05 (d, 1H), 3.94 (m, 3H), 3.82 (m, 2H), 3.70-3.60 (m, 7H), 3.51 (m, 4H), 3.22-2.95 (m, 10H), 2.88 (m, 1H), 2.80 (m, 1H), 2.38 (m, 3H), 2.20-2.10 (m, 2H), 1.78-1.68 (m, 8H), −2.34 (brs, 1H), −2.55 (brs, 1H).

### Synthesis Example 34—synthesis of phyllochlorin 3-acetoxypropyl-13-hydroxymethyl (compound 34)

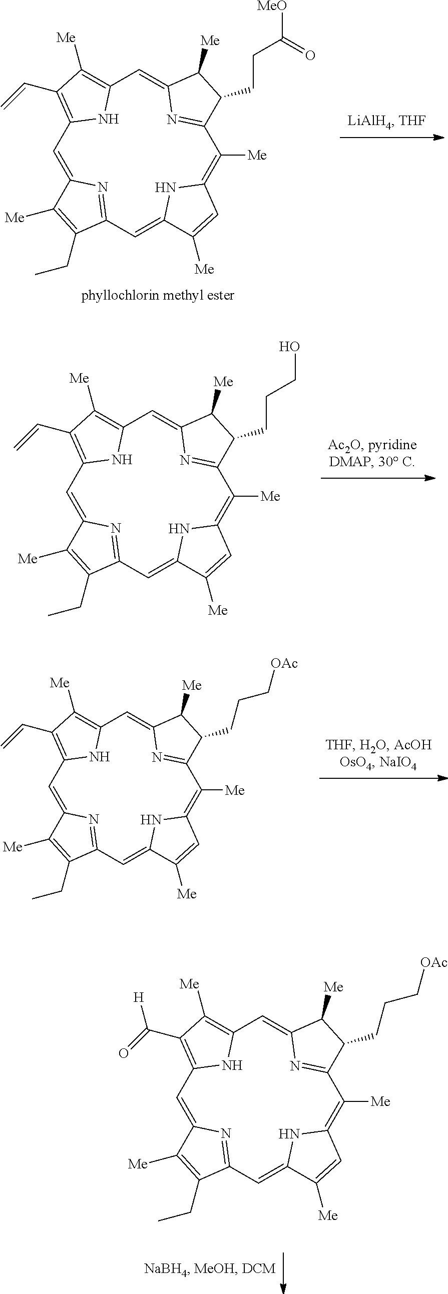 [SMILES: C=Cc1c(C)c2cc3nc(c(C)c4cc(C)c(cc5nc(cc1[nH]2)C(C)=C5CC)[nH]4)[C@@H](CCC(=O)OC)[C@@H]3C ; C=Cc1c(C)c2cc3nc(c(C)c4cc(C)c(cc5nc(cc1[nH]2)C(C)=C5CC)[nH]4)[C@@H](CCCO)[C@@H]3C ; C=Cc1c(C)c2cc3nc(c(C)c4cc(C)c(cc5nc(cc1[nH]2)C(C)=C5CC)[nH]4)[C@@H](CCCOC(C)=O)[C@@H]3C ; CCC1=C(C)c2cc3[nH]c(cc4nc(c(C)c5cc(C)c(cc1n2)[nH]5)[C@@H](CCCOC(C)=O)[C@@H]4C)c(C)c3C=O]

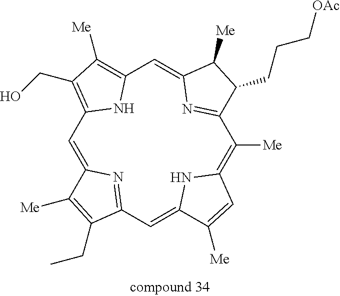 [SMILES: CCC1=C(C)c2cc3[nH]c(cc4nc(c(C)c5cc(C)c(cc1n2)[nH]5)[C@@H](CCCOC(C)=O)[C@@H]4C)c(C)c3CO]

Step 1: Into a 2-neck 500 mL RBF was added lithium aluminium hydride (670 mg, 16.8 mmol, 5 eq) and THF (200 mL) and the solution was stirred and cooled using an ice/water bath for 10 minutes under nitrogen. Phyllochlorin methyl ester (1.75 g, 3.35 mmol, 1 eq) was added in portions over 5 minutes. After 30 minutes further lithium aluminium hydride (670 mg, 16.8 mmol, 5 eq) was added and stirring was continued for 90 minutes during which the reaction was allowed to warm slightly. TLC analysis showed the reaction to be complete. The flask was cooled (ice/water bath) and water (1.3 mL) was added followed by 4M NaOH (1.3 mL). After stirring for 10 minutes further water (4 mL) was added and the solution was warmed to room temperature and stirred for 15 minutes. Sodium sulfate was added and the mixture stirred 10 minutes before being filtered, rinsing with DCM until no more colour eluted. The solvent was then removed under reduced pressure to give the crude product which was purified by column chromatography (4×11 cm) eluting using a gradient of 2-4% MeOH/DCM. Fractions containing the product (R_{f}=0.4 in 5% MeOH/DCM) were combined to give phyllochlorin 3-hydroxypropyl as a dark green solid (1.60 g, 97%).

¹H NMR (400 MHZ, CDCl₃) δ 9.72 (m, 2H), 8.88 (s, 1H), 8.84 (m, 1H), 8.16 (dd, 1H), 6.48 (dd, 1H), 6.14 (dd, 1H), 4.51 (m, 2H), 3.94 (s, 3H), 3.85 (q, 2H), 3.63 (s, 3H), 3.60-3.50 (m, 5H), 3.38 (s, 3H), 2.21-2.612 (m, 1H), 1.85-1.72 (m, 8H), 1.52-1.40 (m, 2H), 1.12-1.05 (m, 1H), −2.10 (br, 1H), −2.22 (br, 1H).

Step 2: Into a 25 mL RBF was added phyllochlorin 3-hydroxypropyl (100 mg, 0.202 mmol, 1 eq), pyridine (0.7 mL), acetic anhydride (0.2 mL, 2.11 mmol, 10 eq) and DMAP (0.5 mg). The solution was stirred at 30° C. for 1 hour. Ethyl acetate (10 mL) and water (5 mL) were added and the mixture stirred vigorously for 10 minutes. The layers were separated and the ethyl acetate layer washed with 0.5 M HCl (3×5 mL), saturated NaHCO₃ (3×5 mL), dried (Na₂SO₄) and concentrated to give phyllochlorin propan-3-ol acetate as a dark green, flaky solid (78 mg, 72%).

¹H NMR (400 MHZ, CDCl₃) δ 9.72 (m, 2H), 8.86 (s, 1H), 8.84 (m, 1H), 8.16 (dd, 1H), 6.48 (dd, 1H), 6.13 (dd, 1H), 4.52 (m, 2H), 4.06 (t, 2H), 3.95 (s, 3H), 3.85 (q, 2H), 3.65 (s, 3H), 3.54 (s, 3H), 3.37 (s, 3H), 2.21-2.13 (m, 1H), 1.96 (s, 3H), 1.95-1.80 (m, 2H), 1.80-1.72 (m, 6H), 1.62-1.50 (m, 2H), −2.10 (br, 1H), −2.22 (br, 1H).

Step 3: To a 100 mL RBF was added phyllochlorin propan-3-ol acetate (340 mg, 0.633 mmol, 1 eq), THF (15 mL), osmium tetroxide (1.6 mg, 0.005 mmol, 0.01 eq), deionized water (1 mL), AcOH (1 mL) and sodium periodate (339 mg, 1.584 mmol, 2.5 eq). The resultant mixture was stirred under nitrogen in the dark at ambient temperature overnight. The reaction mixture was concentrated using a rotary evaporator to remove the THF and then re-dissolved in DCM (75 mL), transferred to a separatory funnel and washed with brine (30 mL), saturated NaHCO₃ (30 mL) and water (40 mL) before being dried (Na₂SO₄) and concentrated by rotary evaporation to give phyllochlorin 3-acetoxypropyl-13-formyl as a red-brown powdery solid (˜0.4 g) which was used directly in the next step.

Step 4: To a 100 mL RBF was added phyllochlorin 3-acetoxypropyl-13-formyl (340 mg, 0.631 mmol, 1 eq), MeOH (10 mL), DCM (5 mL) and sodium borohydride (48 mg, 1.262 mmol, 2 eq). The resultant mixture was stirred under nitrogen ambient temperature for 30 minutes. The reaction mixture was diluted with water (20 mL) and stirred for 10 minutes. The mixture was then extracted with DCM (3×15 mL) and the combined DCM layers washed with water (20 mL) before being dried (Na₂SO₄) and concentrated by rotary evaporation to give a dark green oil. The residue was purified by column chromatography (3×16 cm) eluting using a gradient of 1-2.5% MeOH/DCM. Fractions containing the product (R_{f}=0.5 in 5% MeOH/DCM) were combined to give compound 34 as a dark green solid (160 mg, 47% over 2 steps).

¹H NMR (400 MHZ, CDCl₃) δ 9.70 (s, 1H), 9.65 (m, 1H), 8.87 (s, 1H), 8.84 (s, 1H), 5.90 (m, 2H), 4.57-4.50 (m, 2H), 4.06 (m, 2H), 3.97 (s, 3H), 3.82 (q, 2H), 3.62 (s, 3H), 3.48 (m, 3H), 3.34 (s, 3H), 2.24-2.15 (m, 1H), 2.07-2.00 (m, 1H), 1.97 (s, 3H), 1.95-1.85 (m, 4H), 1.31 (d, 3H), 1.73 (t, 3H), 1.62-1.55 (m, 2H), −2.25 (brs, 1H), −2.40 (brs, 1H).

### Synthesis Example 35—synthesis of phyllochlorin 3-hydroxypropyl-13-hydroxymethyl-β-D-glucoside ether (compound 35)

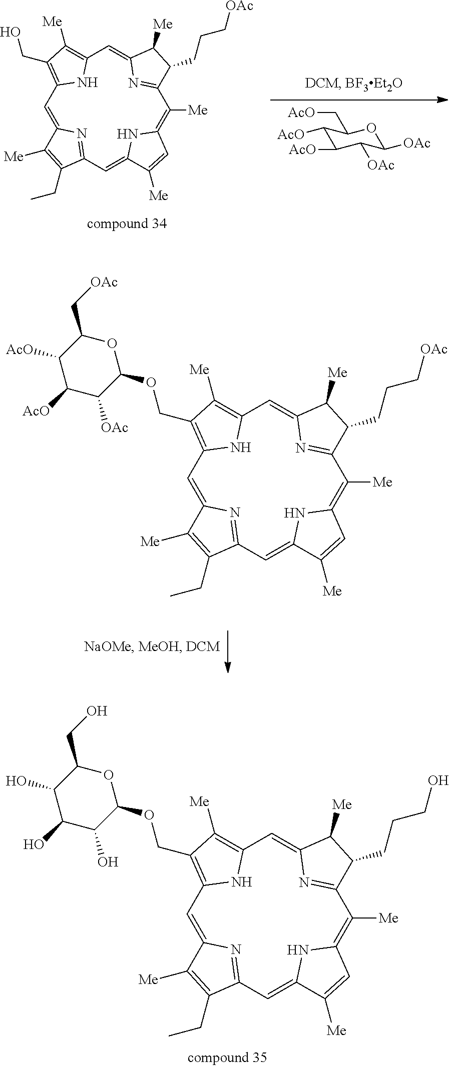 [SMILES: CCC1=C(C)c2cc3[nH]c(cc4nc(c(C)c5cc(C)c(cc1n2)[nH]5)[C@@H](CCCOC(C)=O)[C@@H]4C)c(C)c3CO ; CC(=O)OCC1OC(OC(C)=O)C(OC(C)=O)C(OC(C)=O)C1OC(C)=O ; CCC1=C(C)c2cc3[nH]c(cc4nc(c(C)c5cc(C)c(cc1n2)[nH]5)[C@@H](CCCOC(C)=O)[C@@H]4C)c(C)c3CO[C@@H]1O[C@H](COC(C)=O)[C@@H](OC(C)=O)[C@H](OC(C)=O)[C@H]1OC(C)=O ; CCC1=C(C)c2cc3[nH]c(cc4nc(c(C)c5cc(C)c(cc1n2)[nH]5)[C@@H](CCCO)[C@@H]4C)c(C)c3CO[C@@H]1O[C@H](CO)[C@@H](O)[C@H](O)[C@H]1O]

Step 1: To a 50 mL RBF was added phyllochlorin 3-acetoxypropyl-13-hydroxymethyl (compound 34) (160 mg, 0.296 mmol, 1 eq), glucose pentaacetate (173 mg, 0.443 mmol, 1.5 eq) and DCM (10 mL). The resultant mixture was stirred under nitrogen cooling to ˜0° C. using an ice/water bath. Boron trifluoride diethyl etherate (0.2 mL) was added and stirring was continued allowing the solution to warm slowly to room temperature over 2 hours and then stirred at room temperature in the dark overnight. The reaction mixture was diluted with DCM (30 mL), transferred to a separatory funnel and washed with saturated NaHCO₃ (25 mL) and then water (20 mL). The organic phase was dried (Na₂SO₄) and concentrated by rotary evaporation to give a blue-black film. The residue was purified by column chromatography eluting using a gradient of 0.5-1.5% MeOH/DCM. Fractions containing phyllochlorin 3-acetoxypropyl-13-hydroxymethyl-β-D-glucoside ether tetraacetate were combined to give a dark green oil (260 mg) that was used directly in the next step.

Step 2: To a solution of phyllochlorin 3-acetoxypropyl-13-hydroxymethyl-β-D-glucoside ether tetraacetate (220 mg, 0.253 mmol, 1 eq) in MeOH (3 mL) and DCM (3 mL) was added NaOMe (4.6 M in MeOH, 0.055 mL, 0.253 mmol, 1 eq), and the mixture stirred under N₂ for 1 hour. The reaction was concentrated by rotary evaporation to give a black film. The residue was purified by column chromatography (3×16 cm) eluting using a gradient of 5-12% MeOH/DCM. Fractions containing the product (R_{f}=0.25 in 10% MeOH/DCM) were combined to give compound 35 as a dark green solid (100 mg, 51% over 2 steps).

¹H NMR (400 MHZ, DMSO-d₆) δ 9.85 (s, 1H), 9.76 (s, 1H), 9.09 (m, 2H), 6.16 (d, 1H), 6.01 (d, 1H), 5.12 (d, 1H), 4.94 (t, 2H), 4.82 (t, 1H), 4.63-4.54 (m, 3H), 4.35 (t, 1H), 3.95-3.90 (m, 4H), 3.83 (q, 2H), 3.72-3.65 (m, 1H), 3.63 (s, 3H), 3.52 (s, 3H), 3.45-3.38 (m, 2H), 3.30-3.25 (m, 1H), 3.20-3.15 (m, 2H), 3.15-3.10 (m, 1H), 2.20-2.10 (m, 1H), 1.77-1.68 (m, 8H), 1.47-1.37 (m, 1H), −2.35 (s, 1H), −2.53 (s, 1H).

### Synthesis Example 36—synthesis of phyllochlorin methyl ester 3-bromopropyl ether (compound 36)

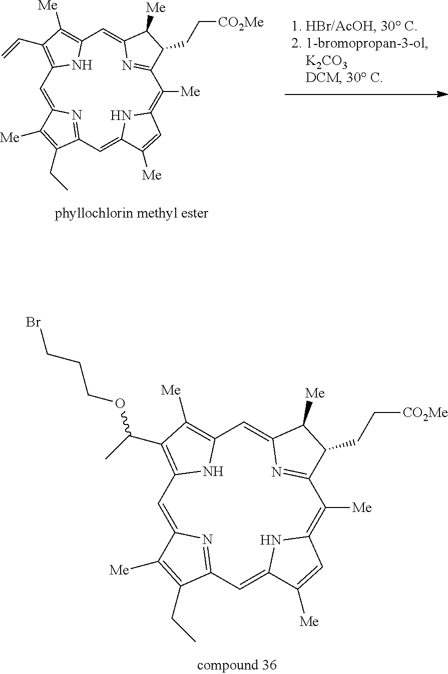 [SMILES: C=Cc1c(C)c2cc3nc(c(C)c4cc(C)c(cc5nc(cc1[nH]2)C(C)=C5CC)[nH]4)[C@@H](CCC(=O)OC)[C@@H]3C ; CCC1=C(C)c2cc3[nH]c(cc4nc(c(C)c5cc(C)c(cc1n2)[nH]5)[C@@H](CCC(=O)OC)[C@@H]4C)c(C)c3C(C)OCCCBr]

To a 50 mL RBF containing phyllochlorin methyl ester (0.50 g, 0.957 mmol, 1 eq) and a stirrer bar was added HBr/AcOH (33% w/w, 5 mL) and the dark blue mixture stir at 30° C. for 2 hours under N₂. A stream of N₂ was passed over the sample for a few minutes to remove some of the HBr before concentrating the bulk by rotary evaporation. The mixture was then further dried under high vacuum (0.6 mbar) at an external temperature of 40° C. for 30 minutes. The residue was reconstituted in DCM (20 mL) before powdered K₂CO₃ (1.32 g, 9.57 mmol, 10 eq), then 3-bromo-propan-1-ol (1.22 mL, 14.35 mmol, 15 eq) were added. The system was flushed with N₂, stoppered with a rubber septum and nitrogen needle and the solution was stirred overnight at 30° C. in the dark. The reaction mixture was transferred to a separatory funnel and washed with H₂O (50 mL), then brine (50 mL), before being dried (Na₂SO₄) and concentrated by rotary evaporation to give the crude ether as a dark green oil that was further dried under high vacuum (0.3 mbar) for 30 minutes at an external temperature of 40-50° C. to give a green oil (0.95 g). The residue was purified by column chromatography eluting using a gradient of 0.5-2% MeOH/DCM. Fractions containing the major green band (R_{f}=0.6 in ˜1% MeOH/DCM) were combined to give compound 36 as a dark green solid (305 mg, 51%).

¹H NMR (400 MHZ, CDCl₃) δ 9.95-9.73 (m, 2H), 8.88-8.80 (m, 2H), 6.04 (q, 1H), 4.60-4.47 (m, 2H), 3.99 (s, 3H), 3.90-3.80 (m, 3H), 3.78-3.70 (m, 2H), 3.66 (s, 3H), 3.58 (s, 3H), 3.55-3.50 (m, 4H), 3.39 (s, 3H), 2.60-2.47 (m, 2H), 2.30-2.20 (m, 2H), 2.18-2.14 (m, 3H), 2.14-2.00 (m, 2H), 1.81-1.75 (m, 6H), −2.15 (brs, 1H), −2.27 (brs, 1H).

### Synthesis Example 37—synthesis of phyllochlorin methyl ester 2-(2-chloroethoxy)ethyl ether (compound 37)

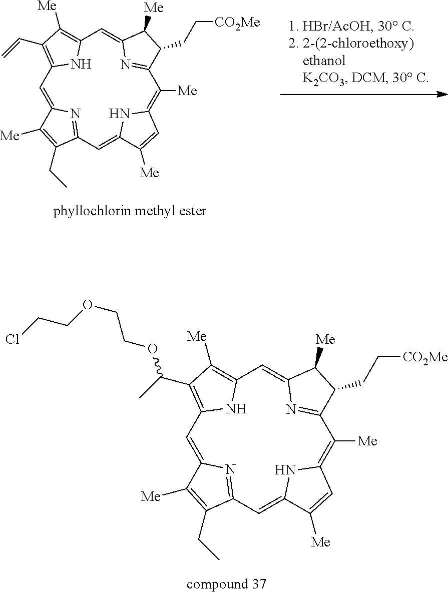 [SMILES: C=Cc1c(C)c2cc3nc(c(C)c4cc(C)c(cc5nc(cc1[nH]2)C(C)=C5CC)[nH]4)[C@@H](CCC(=O)OC)[C@@H]3C ; CCC1=C(C)c2cc3[nH]c(cc4nc(c(C)c5cc(C)c(cc1n2)[nH]5)[C@@H](CCC(=O)OC)[C@@H]4C)c(C)c3C(C)OCCOCCCl]

To a 50 mL RBF containing phyllochlorin methyl ester (0.50 g, 0.957 mmol, 1 eq) and a stirrer bar was added HBr/AcOH (33% w/w, 5 mL) and the dark blue mixture stirred at 30° C. for 2 hours under N₂. A stream of N₂ was passed over the sample for a few minutes to remove some of the HBr before concentrating the bulk by rotary evaporation. The mixture was then further dried under high vacuum (0.6 mbar) at an external temperature of 40° C. for 30 minutes. The residue was reconstituted in DCM (20 mL) before powdered K₂CO₃ (1.32 g, 9.57 mmol, 10 eq), then 2-(2-chloroethoxy) ethanol (1.79 g, 14.35 mmol, 15 eq) were added. The system was flushed with N₂, stoppered with a rubber septum and nitrogen needle and the solution was stirred overnight at 30° C. in the dark. The reaction mixture was transferred to a separatory funnel and washed with H₂O (50 mL), then brine (50 mL), before being dried (Na₂SO₄) and concentrated by rotary evaporation (60° C., full vacuum, 1 hr) to give the crude ether as a dark green oil (˜1.1 g) which was purified by column chromatography eluting using a gradient of 0.5-1% MeOH/DCM. Fractions containing the major green band (R_{f}=0.5 in ˜1% MeOH/DCM) were combined to give compound 37 as a dark green solid (310 mg, 50%).

¹H NMR (400 MHZ, CDCl₃) δ 9.95 (s, 1H), 9.74 (s, 1H), 8.86-8.80 (m, 2H), 6.12 (q, 1H), 4.58 (m, 1H), 4.50 (q, 1H), 3.99 (s, 3H), 3.90-3.70 (m, 9H), 3.66 (s, 3H), 3.65-3.60 (m, 2H), 3.58 (m, 3H), 3.51 (s, 3H), 3.38 (s, 3H), 2.60-2.47 (m, 2H), 2.19 (m, 3H), 2.16-2.00 (m, 2H), 1.80-1.73 (m, 6H), −2.15 (brs, 1H), −2.28 (brs, 1H).

### Synthesis Example 38—synthesis of phyllochlorin 13-(ethyl-1-acetate) (compound 38)

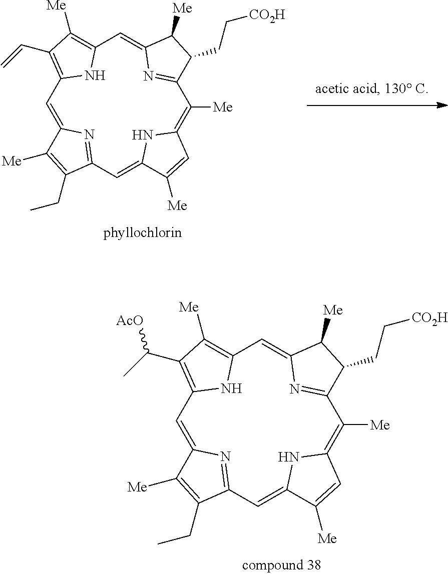 [SMILES: C=Cc1c(C)c2cc3nc(c(C)c4cc(C)c(cc5nc(cc1[nH]2)C(C)=C5CC)[nH]4)[C@@H](CCC(=O)O)[C@@H]3C ; CCC1=C(C)c2cc3[nH]c(cc4nc(c(C)c5cc(C)c(cc1n2)[nH]5)[C@@H](CCC(=O)O)[C@@H]4C)c(C)c3C(C)OC(C)=O]

Into a 50 mL RBF with a stirrer bar and fitted with a condenser with gas inlet/outlet and bubbler was added phyllochlorin (500 mg, 9.87 mmol, 1 eq) and acetic acid (15 mL). The flask was then heated at 130° C. with stirring in the dark overnight. After 27 hours the reaction was cooled and concentrated on a rotary evaporator to give a blue solid. This material was suspended in DCM (50 mL) and washed with water (80 mL) before being dried (Na₂SO₄), filtered and evaporated to give an intense blue glassy solid. The residue was purified by column chromatography (4×12 cm) eluting using 2% MeOH/DCM. Fractions containing the major green band (R_{f}=0.30 in 5% MeOH/DCM) were combined to give compound 38 as a dark green solid (20 mg, 4%).

¹H NMR (400 MHZ, CDCl₃) δ 9.92 (s, 1H), 9.76 (s, 1H), 8.85 (m, 2H), 7.40 (m, 1H), 4.61-4.55 (m, 1H), 4.53-4.46 (m, 1H), 3.96 (s, 3H), 3.87 (q, 2H), 3.64 (s, 3H), 3.55 (s, 3H), 3.40 (s, 3H), 2.56-2.45 (m, 2H), 2.25 (m, 6H), 2.12-1.97 (m, 2H), 1.80-1.71 (m, 7H), −2.38 (brs, 1H).

### Synthesis Example 39—synthesis of phyllochlorin methyl ester 13-(6-(triphenylphosphonium bromide)hexyl)carbamate (compound 39)

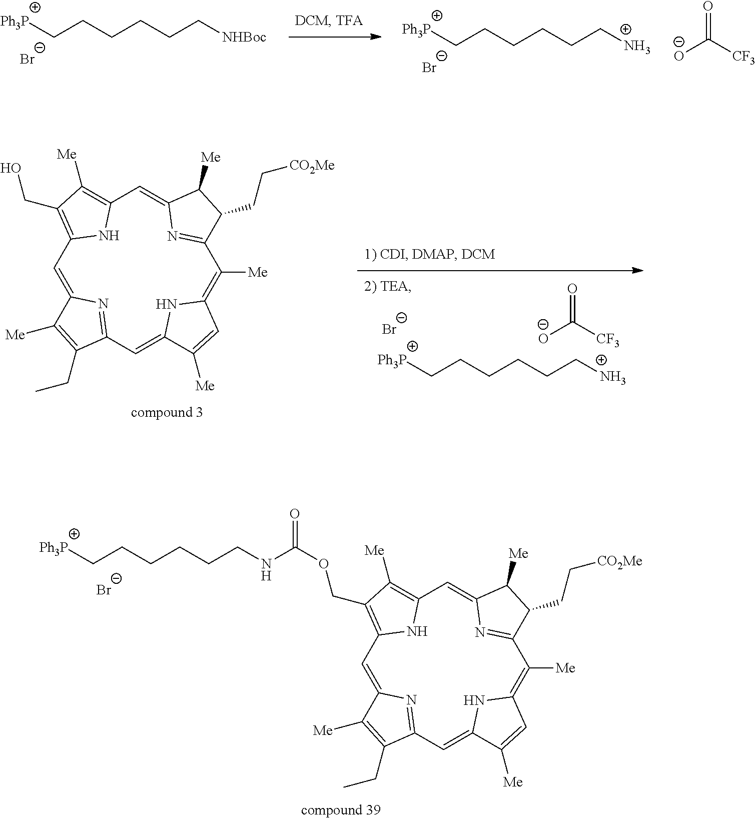

Step 1: To a 50 mL RBF was added (6-((tert-butoxycarbonyl)amino) hexyl) triphenylphosphonium bromide (1.00 g, 1.843 mmol, 9.7 eq), DCM (10 mL) and TFA (2 mL). The resultant solution was stirred for 1 hour at ambient temperature, then concentrated on the rotary evaporator. The residue was resuspended and concentrated twice from chloroform (2×40 mL) to give 6-aminohexyltriphenylphosphonium bromide TFA as a viscous oil (1.50 g) that was dissolved in DCM (1 mL) for the subsequent coupling reaction.

Step 2: To a 50 mL RBF was added phyllochlorin 13-hydroxymethyl methyl ester (compound 3) (100 mg, 0.190 mmol, 1 eq), carbonyl diimidazole (62 mg, 0.380 mmol, 2 eq), DCM (5 mL) and DMAP (10 mg). The resultant mixture was stirred under nitrogen for 3 hours with monitoring by TLC. TEA (810 mg, 8.00 mmol) was added followed by 6-aminohexyltriphenylphosphonium bromide (1.5 g, containing ˜0.69 g TFA, in DCM 1 mL) and stirring was continued for 3 days. The reaction mixture was diluted with DCM (20 mL), transferred to a separatory funnel and washed with 1 M HCl (25 mL), pH=7 phosphate buffer (25 mL) before being dried (Na₂SO₄) and concentrated by rotary evaporation to give a dark green residue. The residue was purified by column chromatography eluting using a gradient of 5-7% MeOH/DCM. Fractions containing the major green band (R_{f}=0.30 in 10% MeOH/DCM) were combined to give compound 39 as a dark green solid (96 mg, 51% over 2 steps).

¹H NMR (400 MHZ, CDCl₃) δ 9.73 (s, 1H), 9.69 (s, 1H), 8.85 (m, 2H), 7.65-7.51 (m, 8H), 7.50-7.35 (m, 7H), 6.44 (m, 2H), 5.60 (brs, 1H), 4.56 (m, 1H), 4.48 (m, 1H), 3.99 (s, 3H), 3.80 (q, 2H), 3.63 (s, 3H), 3.57 (s, 3H), 3.53 (s, 3H), 3.46 (m, 3H), 3.34 (s, 3H), 3.21 (m, 2H), 2.62-2.46 (m, 2H), 2.20-2.10 (m, 1H), 2.06-1.95 (m, 1H), 1.85-1.69 (m, 7H), 1.58-1.38 (m, 6H), 1.37-1.25 (m, 3H), −2.22 (brs, 1H), −2.39 (brs, 1H).

### Synthesis Example 40—synthesis of phyllochlorin methyl ester 13-(N-(3-(3-triphenylphosphoniumpropoxy)propyl)chloride)carbamate (compound 40)

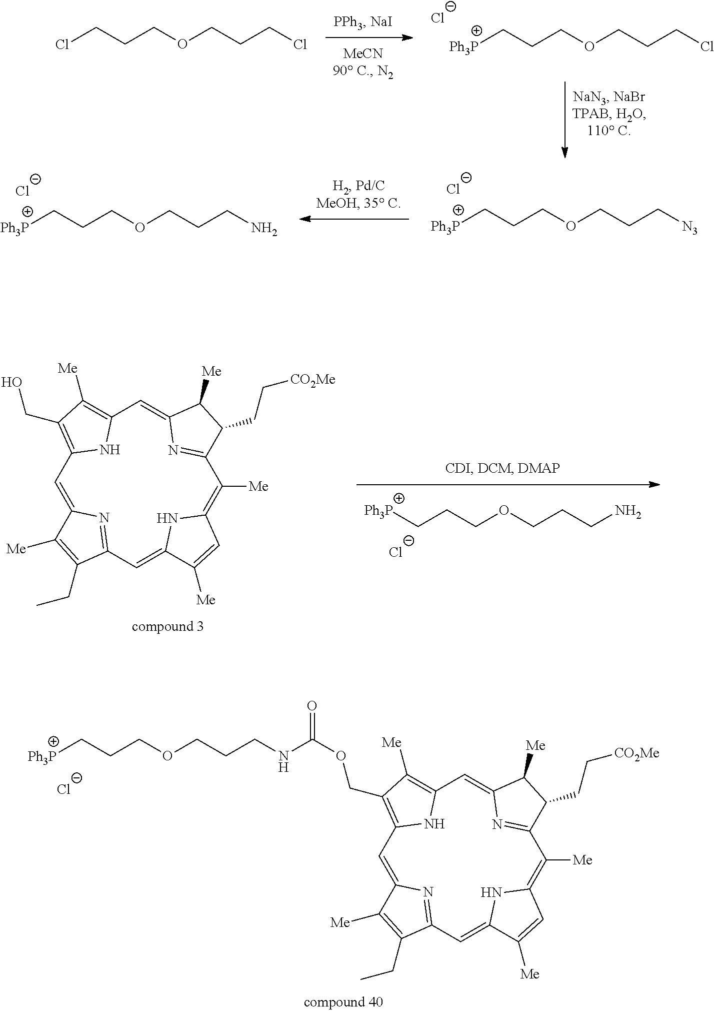

Step 1: A 500 mL 3-neck RBF, equipped with a 3 cm stirrer bar was charged with bis(3-chloropropyl) ether (20.00 g, 70.15 mmol, 1.66 eq), triphenyl phosphine (18.40 g, 70.15 mmol, 1 eq), sodium iodide (7.01 g, 46.77 mmol, 0.66 eq) and acetonitrile (340 mL). The RBF was set over an oil bath and fitted with an air condenser, where stirring (500 rpm) commenced under N₂ at an external temperature of 90° C. The mixture was left to stir for 72 hours. After this time, the reaction flask was cooled to room temperature and the suspension was filtered through a 2 cm plug of Celite®, washing through with acetonitrile (250 mL). The faint yellow solution was then evaporated to dryness to leave a dark yellow oil (44.40 g) which was subject to column chromatography (silica gel, 9×12 cm) using 6% MeOH in DCM as eluent. Fractions with R_{f}=0.35 as visualised by UV in 6% MeOH/DCM were combined and concentrated by rotary evaporation. The resulting residue was purified further by column chromatography (silica gel, 9×7 cm). DCM was used as eluent until no further triphenyl phosphine was present as observed by TLC, then the eluent was changed to 10% MeOH in DCM to remove the product from the column. Fractions with R_{f}=0.35 as visualised by UV in 6% MeOH/DCM were combined and concentrated by rotary evaporation to give (3-(3-chloropropoxy)propyl) triphenylphosphonium chloride as a faint red solid (18.74 g, 62%).

¹H NMR (400 MHZ, CDCl₃) δ 7.87-7.76 (m, 9H), 7.75-7.63 (m, 6H), 3.93-3.80 (m, 2H), 3.75 (td, J=5.7, 1.3 Hz, 2H), 3.58 (q, J=6.3 Hz, 4H), 2.05-1.88 (m, 4H).

Step 2: To a 50 mL RBF was added (3-(3-chloropropoxy)propyl)triphenylphosphonium chloride (4.0 g, 9.23 mmol, 1 eq), NaN₃ (11.08 g, 1.2 eq), NaBr (38 mg, 0.04 eq), tetrapropylammonium bromide (49 mg, 0.02 eq) and water (10 mL). After connecting a water condenser, the flask was heated at 110° C. with stirring for 44 hours. Then the mixture was cooled and EtOAc (50 mL) was added. The mixture was transferred to a separating funnel and washed with water (3×30 mL) and brine (30 mL). The combined aqueous layers were extracted with DCM (3×20 mL). The combined organic layers were then dried (MgSO₄), filtered and concentrated to give (3-(3-azidopropoxy)propyl)triphenylphosphonium chloride as a pale yellow solid (3.20 g, 79%).

¹H NMR (400 MHZ, CDCl₃) δ 7.86-7.77 (m, 9H), 7.73-7.66 (m, 6H), 3.90-3.80 (m, 2H), 3.75 (t, 2H), 3.53 (t, 2H), 3.33 (t, 2H), 1.99-1.89 (m, 2H), 1.82 (p, 2H).

Step 3: A 3-neck 100 mL RBF was charged with (3-(3-azidopropoxy)propyl) triphenylphosphonium chloride (1.00 g, 2.273 mmol, 1 eq), 10% Pd/C (20 mg), methanol (10 mL) and a stirrer bar. A hydrogen balloon was connected to the middle joint of the flask via a short length air condenser and the side-arm was connected to a 3-way tap. The setup was evacuated and then re-filled with nitrogen (3 times), evacuated and re-filled with hydrogen (2 times). The resulting solution was then stirred (550 rpm) under the hydrogen atmosphere for 2 hours at 35° C. Then the solution was filtered through Celite® (0.5×3 cm), washing with chloroform (2×10 mL) and the solvent then removed under reduced pressure to give (3-(3-aminopropoxy)propyl) triphenylphosphonium chloride as a viscous oil that solidified on standing (1.05 g, quantitative).

¹H NMR (400 MHZ, CDCl₃) δ 7.86-7.76 (m, 9H), 7.73-7.66 (m, 6H), 3.88-3.79 (m, 2H), 3.73 (t, 2H), 3.51 (t, 2H), 2.75 (t, 2H), 1.99-1.87 (m, 2H), 1.68 (p, 2H), 1.41 (brs, 2H).

Step 4: To a 25 mL RBF was added phyllochlorin 13-hydroxymethyl methyl ester (compound 3) (50 mg, 0.0949 mmol, 1 eq), carbonyl diimidazole (31 mg, 0.1899 mmol, 2 eq), DCM (3 mL) and DMAP (5 mg). The resultant mixture was stirred under nitrogen for 3 hours with monitoring by TLC. (3-(3-Aminopropoxy)propyl) triphenylphosphonium chloride (196 mg, 0.4747 mmol, 5 eq) dissolved in DCM (1 mL) was added and stirring was continued for 4 days. The reaction mixture was diluted with DCM (20 mL), transferred to a separatory funnel and washed with 1 M HCl (2×20 mL), pH=7 phosphate buffer (30 mL) before being dried (Na₂SO₄) and concentrated by rotary evaporation to give a dark green residue. The residue was purified by column chromatography eluting using a gradient of 3-5% MeOH/DCM. Fractions containing the major green band (R_{f}=0.20 in 5% MeOH/DCM) were combined to give compound 40 as a dark green solid (46 mg, 50%).

¹H NMR (400 MHZ, CDCl₃) δ 9.70 (m, 2H), 8.87 (m, 2H), 7.61-7.50 (m, 6H), 7.49-7.36 (m, 9H), 6.41 (s, 2H), 5.39 (m, 1H), 4.57 (m, 1H), 4.50 (m, 1H), 3.99 (s, 3H), 3.81 (q, 2H), 3.64 (s, 3H), 3.60-3.52 (m, 7H), 3.52-3.46 (m, 5H), 3.40-3.34 (m, 2H), 3.32 (s, 3H), 2.62-2.46 (m, 2H), 2.20-2.12 (m, 1H), 2.07-1.96 (m, 1H), 1.80-1.69 (m, 9H), 1.69-1.55 (m, 4H), 0.90-0.80 (m, 1H), −2.23 (brs, 1H), −2.40 (brs, 1H).

### Synthesis Example 41—synthesis of phyllochlorin 13-formyl N-methyl-N-3,6,9-trioxadecyl propylamide (compound 41)

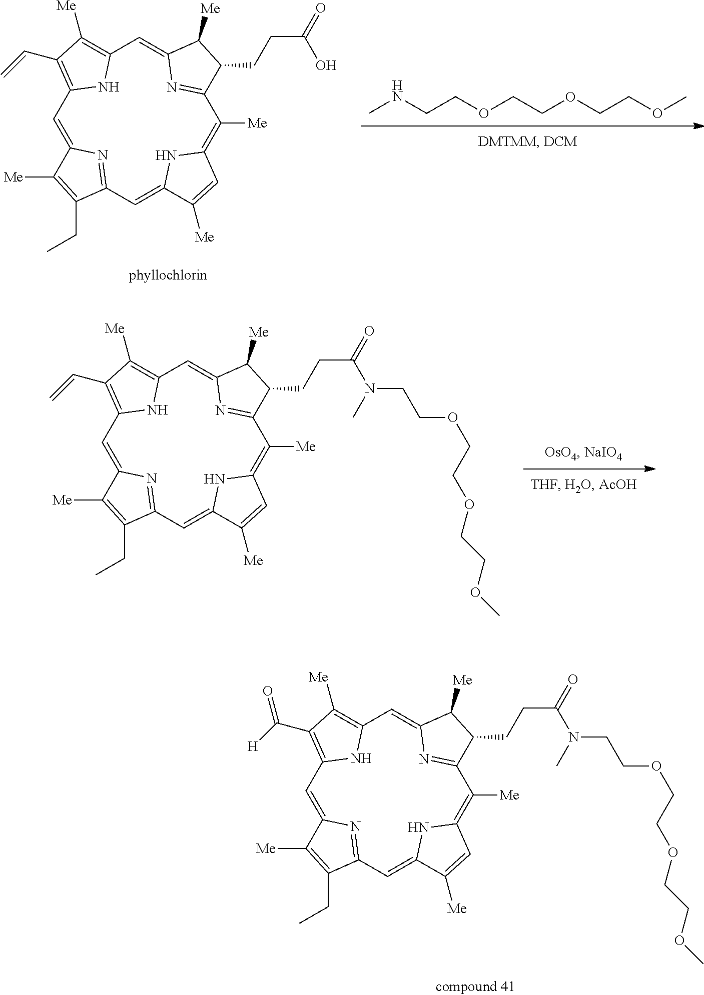

Step 1: To a 250 mL RBF fitted with a nitrogen inlet and containing a small stirrer bar was added phyllochlorin (2.00 g, 3.93 mmol, 1 eq), dichloromethane (50 mL), DMTMM (1.41 g, 5.11 mmol, 1.3 eq), and 2-(2-(2-(methylamino) ethoxy) ethoxy) ethanol (905 mg, 5.11 mmol, 1.3 eq). The mixture was stirred at room temperature for 2 hours. The reaction progress was monitored by HPLC. Then the reaction mixture was transferred to a separatory funnel and washed with 0.5 M HCl (2×50 mL) and pH=7 phosphate buffer (50 mL). The organic layer was dried (Na₂SO₄) and concentrated by rotary evaporation to give a dark green film (2.77 g) which was purified by column chromatography (silica gel, 4×20 cm) using 2-4% MeOH/DCM as eluent gradient. Pure fractions containing a green spot by TLC with R_{f}=0.40 in 5% MeOH/DCM were combined to give phyllochlorin N-methyl-N-3,6,9-trioxadecyl propylamide as a dark blue viscous syrup (2.61 g, quantitative) (HPLC purity: 96.7%).

¹H NMR (400 MHZ, CDCl₃) δ 9.78-9.65 (m, 2H), 8.85 (d, J=2.2 Hz, 2H), 8.17 (dd, J=17.8, 11.5 Hz, 1H), 6.38 (dd, J=17.8, 1.6 Hz, 1H), 6.15 (dd, J=11.5, 1.5 Hz, 1H), 4.72-4.62 (m, 1H), 4.55 (q, J=7.2 Hz, 1H), 4.02 (d, J=4.2 Hz, 3H), 3.90-3.81 (m, 2H), 3.66-3.60 (m, 3H), 3.54 (s, 3H), 3.51-3.41 (m, 4H), 3.41-3.34 (m, 5H), 3.27-3.22 (m, 4H), 3.21-3.15 (m, 1H), 2.97-2.86 (m, 1H), 2.83-2.77 (m, 1H), 2.74 (s, 1H), 2.64-2.51 (m, 1H), 2.46 (s, 2H), 2.44-2.35 (m, 1H), 2.26-2.11 (m, 1H), 2.05-1.86 (m, 1H), 1.81-1.71 (m, 6H), −2.07 (s, 1H), −2.21 (s, 1H).

Step 2: To a 100 mL RBF was added phyllochlorin N-methyl-N-3,6,9-trioxadecyl propylamide (2.46 g, 3.68 mmol, 1 eq), THF (100 mL), osmium tetroxide (9.4 mg, 0.0368 mmol, 0.01 eq), deionized water (8 mL), AcOH (8 mL) and sodium periodate (2.05 g, 9.57 mmol, 2.6 eq). The resultant mixture was stirred under nitrogen in the dark at ambient temperature for 18 hours. The reaction mixture was concentrated using a rotary evaporator to remove the THF and then re-dissolved in DCM (130 mL), transferred to a separatory funnel and washed with brine (70 mL), saturated NaHCO₃ (70 mL) and water (100 mL) before being dried (Na₂SO₄) and concentrated by rotary evaporation to give compound 41 as a dark blue solid (1.77 g, 72%).

¹H NMR (400 MHZ, CDCl₃) δ 11.60 (s, 1H), 10.31 (d, J=2.6 Hz, 1H), 9.60 (d, J=3.7 Hz, 1H), 9.03-8.85 (m, 2H), 4.68-4.58 (m, 1H), 4.56-4.43 (m, 1H), 4.02 (d, J=5.5 Hz, 3H), 3.82 (d, J=2.2 Hz, 3H), 3.80-3.70 (m, 1H), 3.66 (d, J=4.6 Hz, 2H), 3.63 (d, J=4.3 Hz, 4H), 3.58-3.43 (m, 4H), 3.43-3.39 (m, 2H), 3.37 (s, 3H), 3.35-3.28 (m, 2H), 3.26 (d, J=2.4 Hz, 3H), 3.21-2.86 (m, 4H), 2.79 (s, 1H), 2.59 (s, 2H), 2.57-2.41 (m, 2H), 2.20-1.97 (m, 1H), 1.89-1.83 (m, 2H), 1.81-1.62 (m, 6H), −1.78 (s, 1H), −2.25 (s, 1H).

### Synthesis Example 42—synthesis of phyllochlorin 13-hydroxymethyl N-methyl-N-3,6,9-trioxadecyl propylamide (compound 42)

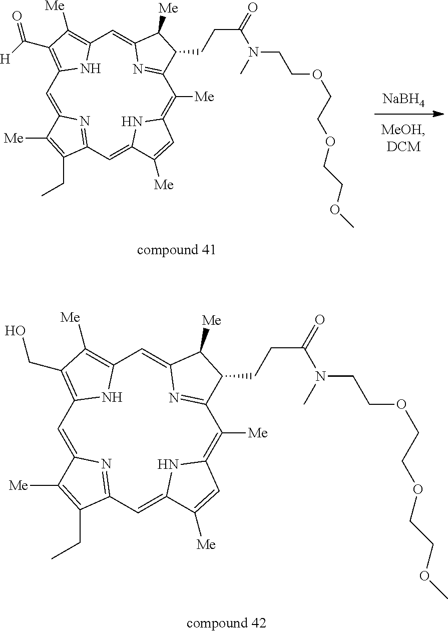 [SMILES: CCC1=C(C)c2cc3[nH]c(cc4nc(c(C)c5cc(C)c(cc1n2)[nH]5)[C@@H](CCC(=O)N(C)CCOCCOCCOC)[C@@H]4C)c(C)c3C=O ; "NaBH4" ; CCC1=C(C)c2cc3[nH]c(cc4nc(c(C)c5cc(C)c(cc1n2)[nH]5)[C@@H](CCC(=O)N(C)CCOCCOCCOC)[C@@H]4C)c(C)c3CO]

To a 250 mL RBF was added phyllochlorin 13-formyl N-methyl-N-3,6,9-trioxadecyl propylamide (compound 41) (1.75 g, 2.61 mmol, 1 eq), MeOH (40 mL), DCM (20 mL) and sodium borohydride (197 mg, 5.22 mmol, 2 eq). The resultant mixture was stirred under nitrogen ambient temperature for 1 hour. The reaction mixture was diluted with water (50 mL) and stirred for 10 minutes. The mixture was then diluted with DCM (20 mL) and brine (20 mL). The DCM layer was collected and the aqueous layer was further extracted with DCM (2×20 mL). The combined DCM layers were washed with brine (20 mL) before being dried (Na₂SO₄) and concentrated by rotary evaporation to give a dark green solid (1.21 g). The residue was purified by column chromatography (4×20 cm). It was dissolved in DCM and eluted using a gradient of 1.5-3.5% MeOH/DCM. Fractions containing the product (Major dark green spot, R_{f}=0.5 in 5% MeOH/DCM) were combined to give compound 42 as a dark blue-green solid (548 mg, 31%).

¹H NMR (400 MHZ, CDCl₃) δ 9.69 (d, J=3.4 Hz, 1H), 9.65 (d, J=4.3 Hz, 1H), 8.84 (d, J=2.7 Hz, 2H), 5.96-5.84 (m, 2H), 4.65 (dt, J=8.0, 3.2 Hz, 1H), 4.56-4.48 (m, 1H), 4.46 (d, J=5.8 Hz, 1H), 4.27 (dd, J=10.0, 6.4 Hz, 1H), 4.01 (d, J=3.3 Hz, 3H), 3.82 (q, J=7.6 Hz, 2H), 3.62 (s, 3H), 3.56 (d, J=1.7 Hz, 1H), 3.51-3.47 (m, 3H), 3.46-3.35 (m, 4H), 3.34 (s, 3H), 3.23 (d, J=6.7 Hz, 4H), 3.12 (td, J=5.1, 4.6, 2.1 Hz, 1H), 2.87-2.73 (m, 2H), 2.68 (s, 1H), 2.64 (t, J=4.8 Hz, 1H), 2.59-2.48 (m, 1H), 2.42 (s, 2H), 2.35 (dt, J=15.5, 6.6 Hz, 1H), 2.17 (tdt, J=13.0, 8.8, 4.7 Hz, 1H), 1.94-1.81 (m, 1H), 1.80-1.69 (m, 6H), 1.61 (s, 2H), −2.23 (s, 1H), −2.39 (s, 1H).

### Synthesis Example 43—synthesis of phyllochlorin 13-(6-(triphenylphosphonium bromide)hexyl)carbamate N-methyl-N-3,6,9-trioxadecyl propylamide (compound 43)

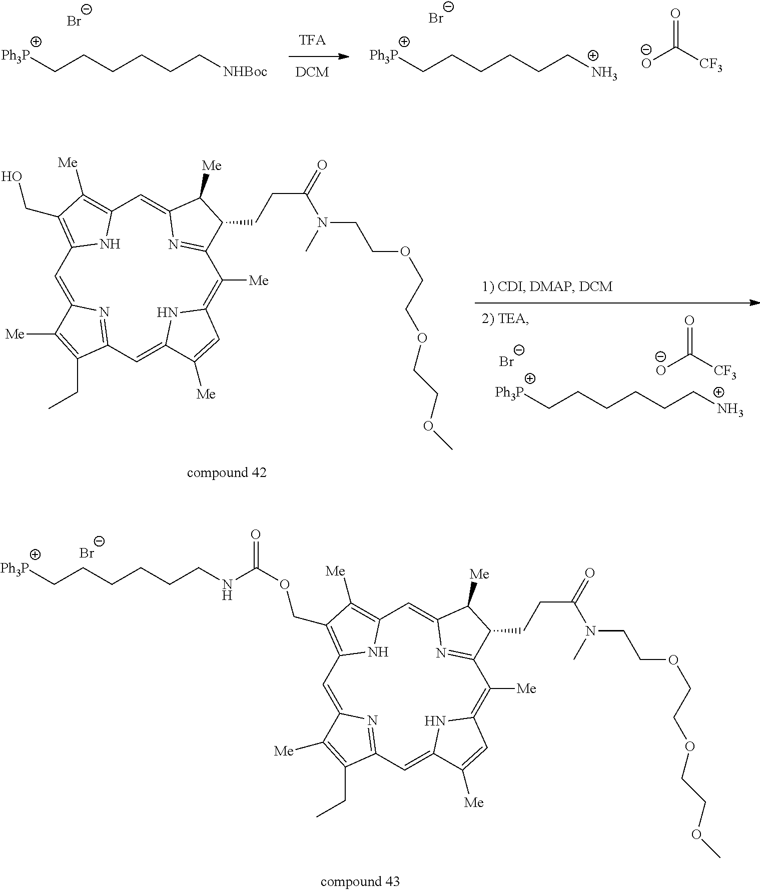

Step 1: To a 50 mL RBF was added (6-((tert-butoxycarbonyl)amino) hexyl) triphenylphosphonium bromide (608 mg, 1.12 mmol, 5 eq), DCM (10 mL) and TFA (1.2 mL). The resultant solution was stirred for 1 hour at ambient temperature, then concentrated on the rotary evaporator. The residue was resuspended and concentrated twice from chloroform (2×40 mL) to give 6-aminohexyltriphenylphosphonium bromide TFA as a viscous oil (1.207 g) that was dissolved in DCM (2 mL) for the subsequent coupling reaction.

Step 2: To a 50 mL RBF was added phyllochlorin 13-hydroxymethyl N-methyl-N-3,6,9-trioxadecyl propylamide (compound 42) (150 mg, 0.224 mmol, 1 eq), carbonyl diimidazole (73 mg, 0.448 mmol, 2 eq), DCM (5 mL) and DMAP (10 mg). The resultant mixture was stirred under nitrogen for 3 hours and the reaction was monitored by TLC. TEA (821 mg, 8.11 mmol) was added followed by 6-aminohexyltriphenylphosphonium bromide (1.207 g, containing ˜711 mg TFA, in DCM 2 mL) and stirring was continued for 3 days. The reaction mixture was diluted with DCM (20 mL), transferred to a separatory funnel and washed with 1 M HCl (2×25 mL) and pH=7 phosphate buffer (25 mL) before being dried (Na₂SO₄) and concentrated by rotary evaporation to give a dark green residue. The residue was purified by column chromatography (4×18 cm) using 5-10% MeOH/DCM, loaded as a solution in the eluent. Fractions containing the major dark green band (R_{f}=0.20 in 5% MeOH/DCM) were concentrated by rotary evaporation to give compound 43 as a dark green solid (45 mg, 18% over 2 steps).

¹H NMR (400 MHZ, CDCl₃) δ 9.71 (dd, J=25.2, 5.0 Hz, 2H), 8.85 (d, J=5.0 Hz, 2H), 7.77-7.34 (m, 16H), 6.43 (s, 2H), 5.77-5.58 (m, 1H), 4.69-4.59 (m, 1H), 4.52 (q, J=7.4 Hz, 1H), 4.01 (d, J=4.2 Hz, 3H), 3.79 (q, J=7.8 Hz, 2H), 3.61 (s, 3H), 3.55-3.30 (m, 11H), 3.22 (d, J=7.3 Hz, 6H), 3.03-2.91 (m, 1H), 2.91-2.81 (m, 1H), 2.74 (s, 1H), 2.50 (s, 2H), 2.19-2.00 (m, 1H), 1.89 (s, 4H), 1.72 (dt, J=23.6, 7.0 Hz, 6H), 1.61-1.37 (m, 5H), 1.37-1.04 (m, 5H), 0.93-0.75 (m, 1H), −2.29 (s, 1H), −2.43 (s, 1H).

### Synthesis Example 44—synthesis of phyllochlorin 13-(N-(3-(3-triphenylphosphoniumpropoxy)propyl)chloride)carbamate N-methyl-N-3,6,9-trioxadecyl propylamide (compound 44)

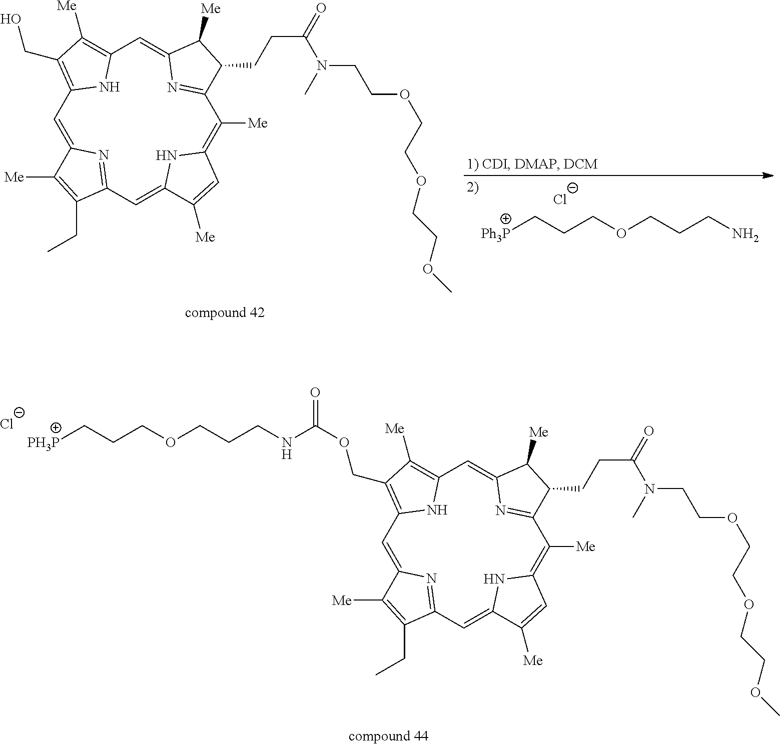

To a 50 mL RBF was added phyllochlorin 13-hydroxymethyl N-methyl-N-3,6,9-trioxadecyl propylamide (compound 42) (73 mg, 0.109 mmol, 1 eq), carbonyl diimidazole (35 mg, 0.218 mmol, 2 eq), DCM (3 mL) and DMAP (5 mg). The resultant mixture was stirred under nitrogen for 3 hours and the reaction progress was monitored by TLC. (3-(3-Aminopropoxy)propyl)triphenylphosphonium chloride (225 mg, 0.544 mmol, 5 eq, dissolved in DCM 2 mL) was added and stirring was continued for 16 hours. The reaction mixture was diluted with DCM (20 mL), transferred to a separatory funnel and washed with 1 M HCl (2×20 mL) and pH=7 phosphate buffer (20 mL) before being dried (Na₂SO₄) and concentrated by rotary evaporation to give a dark green residue. The residue was purified by column chromatography (3×22 cm) using 5% MeOH/DCM, loaded as a solution in the eluent. Fractions containing the major dark green band (R_{f}=0.2 in 5% MeOH/DCM) were concentrated by rotary evaporation to give compound 44 as a dark green solid (35 mg, 29%).

¹H NMR (400 MHZ CDCl₃) δ 9.72 (d, J=4.5 Hz, 2H), 8.88 (d, J=6.2 Hz, 2H), 7.63-7.53 (m, 7H), 7.51-7.35 (m, 10H), 6.43 (s, 2H), 5.50 (d, J=6.0 Hz, 1H), 4.66 (d, J=8.2 Hz, 1H), 4.55 (q, J=7.2 Hz, 1H), 4.04 (d, J=4.3 Hz, 3H), 3.83 (q, J=7.5 Hz, 2H), 3.65 (s, 3H), 3.58 (d, J=5.7 Hz, 1H), 3.54-3.44 (m, 8H), 3.43-3.36 (m, 3H), 3.34 (s, 3H), 3.26 (s, 3H), 3.10-3.01 (m, 1H), 2.99-2.90 (m, 1H), 2.78 (s, 1H), 2.57 (s, 2H), 2.55-2.41 (m, 1H), 2.18-2.06 (m, 2H), 1.83-1.69 (m, 6H), 1.64 (s, 8H), −2.26 (s, 1H), −2.41 (s, 1H).

### Synthesis Example 45—synthesis of phyllochlorin methyl ester 13-(N-(3-triphenylphosphoniumpropyl)bromide)carbamate (compound 45)

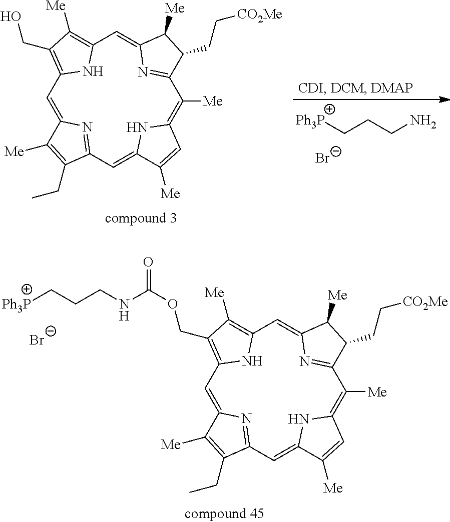

To a 25 mL RBF was added phyllochlorin 13-hydroxymethyl methyl ester (compound 3) (75 mg, 0.142 mmol, 1 eq), carbonyl diimidazole (46 mg, 0.285 mmol, 2 eq), DCM (4 mL) and DMAP (5 mg). The resultant mixture was stirred under nitrogen for 3 hours. (3-Aminopropyl)triphenylphosphonium bromide (285 mg, 0.712 mmol, 5 eq) was added and stirring was continued overnight at ambient temperature. The reaction was then heated at 30° C. (heat block) for a further 6 hours. The reaction mixture was diluted with DCM (15 mL), transferred to a separatory funnel and washed with 1 M HCl (2×10 mL) and pH=7 phosphate buffer (25 mL) before being dried (Na₂SO₄) and concentrated by rotary evaporation to give a dark green residue (˜210 mg). The residue was purified by column chromatography (3×12 cm) eluting using a gradient of 3-9% MeOH/DCM. Fractions containing the major green band (R_{f}=0.15 in 5% MeOH/DCM) were combined to give compound 45 as a dark green solid (27 mg, 20%).

¹H NMR (400 MHZ, CDCl₃) δ 9.87 (s, 1H), 9.70 (s, 1H), 8.84 (m, 2H), 9.68 (t, 1H), 7.44-7.37 (m, 6H), 7.19-7.10 (m, 9H), 6.92-6.85 (m, 1H), 6.70-6.58 (m, 1H), 6.44 (m, 2H), 4.55 (m, 1H), 4.49 (m, 1H), 3.99 (s, 3H), 3.80 (q, 2H), 3.64 (m, 5H), 3.60-3.50 (m, 9H), 3.35 (s, 3H), 2.62-2.46 (m, 2H), 2.20-2.12 (m, 1H), 2.06-1.95 (m, 1H), 1.92-1.84 (m, 4H), 1.80-1.65 (m, 9H), −2.22 (brs, 1H), −2.36 (brs, 1H).

### Synthesis Example 46—synthesis of phyllochlorin methyl ester 13-(N-(2-triphenylphosphoniumethyl)bromide)carbamate (compound 46)

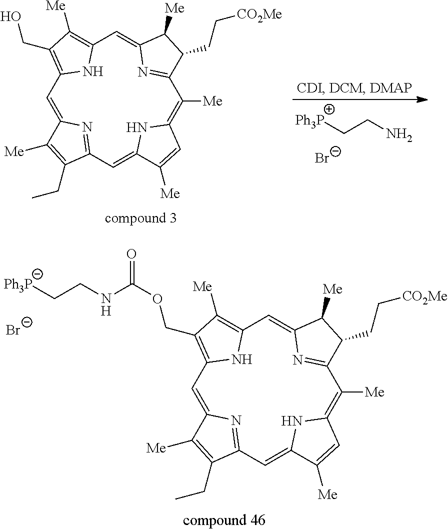

To a 25 mL RBF was added phyllochlorin 13-hydroxymethyl methyl ester (compound 3) (125 mg, 0.237 mmol, 1 eq), carbonyl diimidazole (77 mg, 0.475 mmol, 2 eq), DCM (4 mL) and DMAP (5 mg). The resultant mixture was stirred under nitrogen for 3 hours. (2-Aminoethyl)triphenylphosphonium bromide (458 mg, 1.187 mmol, 5 eq) was added and stirring was continued overnight at ambient temperature. The reaction mixture was diluted with DCM (15 mL), transferred to a separatory funnel and washed with 1 M HCl (2×10 mL) and pH=7 phosphate buffer (25 mL) before being dried (Na₂SO₄) and concentrated by rotary evaporation to give a dark green residue. The residue was purified by column chromatography eluting using a gradient of 4-7% MeOH/DCM. Fractions containing the major green band (R_{f}=0.15 in 10% MeOH/DCM) were combined to give compound 46 as a dark green solid (52 mg, 23%).

¹H NMR (400 MHZ, CDCl₃) δ 9.77 (s, 1H), 9.71 (s, 1H), 8.87 (m, 2H), 7.65-7.54 (m, 6H), 7.46 (t, 1H), 7.40-7.31 (m, 9H), 6.86-6.75 (m, 1H), 6.62-6.50 (m, 1H), 6.32 (m, 2H), 4.61-4.48 (m, 2H), 3.99 (s, 3H), 3.86-3.70 (m, 7H), 3.64 (m, 4H), 3.57 (m, 4H), 3.52 (s, 3H), 3.39 (s, 3H), 2.64-2.46 (m, 2H), 2.20-2.12 (m, 1H), 2.08-1.97 (m, 1H), 1.93-1.81 (m, 3H), 1.80-1.70 (m, 8H), −2.24 (brs, 1H), −2.38 (brs, 1H).

### Synthesis Example 47—synthesis of phyllochlorin 13-formyl ethyl ester (compound 47)

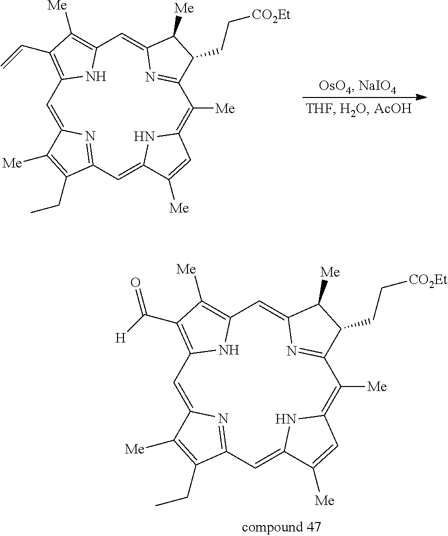 [SMILES: C=Cc1c(C)c2cc3nc(c(C)c4cc(C)c(cc5nc(cc1[nH]2)C(C)=C5CC)[nH]4)[C@@H](CCC(=O)OCC)[C@@H]3C ; CCOC(=O)CC[C@@H]1c2nc(cc3[nH]c(cc4nc(cc5[nH]c(cc5C)c2C)C(CC)=C4C)c(C=O)c3C)[C@H]1C]

To a 250 mL RBF was added phyllochlorin ethyl ester (2.25 mg, 1 eq), THF (75 mL), osmium tetroxide (10 mg, 0.01 eq), deionized water (5 mL), AcOH (5 mL) and sodium periodate (2.33 g, 2.2 eq). The resultant mixture was stirred under nitrogen in the dark at ambient temperature overnight. The reaction mixture was concentrated using a rotary evaporator to remove the THF and then re-dissolved in DCM (60 mL), transferred to a separatory funnel and washed with brine (40 mL), saturated NaHCO₃ (40 mL) and water (40 mL) before being dried (Na₂SO₄) and concentrated by rotary evaporation to give a red-brown powdery solid. The residue was subjected to column chromatography (4×15 cm). It was dissolved in DCM (using minimum MeOH to help solubilize) to load onto the column that had been pre-equilibrated with DCM. The column was eluted using a gradient of 1-2% MeOH/DCM. Fractions containing the product (Major dark green spot, R_{f}=0.7 in 5% MeOH/DCM) were combined to give compound 47 as a dark green solid (1.68 g, 75%).

¹H NMR (400 MHZ, CDCl₃) δ 11.60 (s, 1H), 10.32 (s, 1H), 9.61 (s, 1H), 8.98 (s, 1H), 8.90 (s, 1H), 4.56 (m, 1H), 4.50 (m, 1H), 4.20 (m, 2H), 4.00 (s, 3H), 3.81 (m, 5H), 3.62 (s, 3H), 3.60 (s, 3H), 2.66-2.56 (m, 1H), 2.55-2.47 (m, 1H), 2.25-2.15 (m, 1H), 2.05-1.95 (m, 1H), 1.80-1.71 (m, 6H), 1.12 (t, 3H), −1.80 (s, 1H), −2.25 (s, 1H).

### Synthesis Example 48—synthesis of phyllochlorin 13-hydroxymethyl ethyl ester (compound 48)

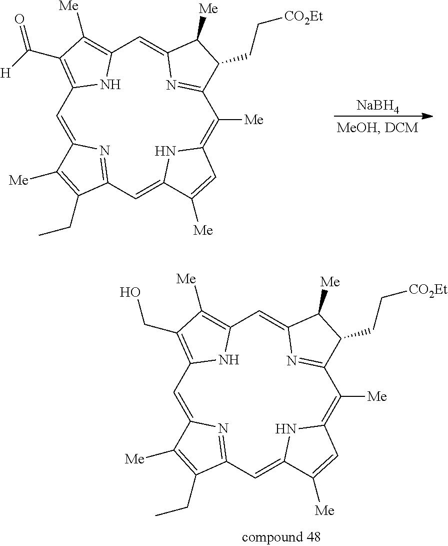 [SMILES: CCOC(=O)CC[C@@H]1c2nc(cc3[nH]c(cc4nc(cc5[nH]c(cc5C)c2C)C(CC)=C4C)c(C=O)c3C)[C@H]1C ; "NaBH4" ; CCOC(=O)CC[C@@H]1c2nc(cc3[nH]c(cc4nc(cc5[nH]c(cc5C)c2C)C(CC)=C4C)c(CO)c3C)[C@H]1C]

To a 250 mL RBF was added phyllochlorin 13-formyl ethyl ester (compound 47) (1.60 g, 1 eq), MeOH (35 mL), DCM (17 mL) and sodium borohydride (224 mg, 2 eq). The resultant mixture was stirred under nitrogen ambient temperature for 1 hour. The reaction mixture was diluted with water (100 mL) and stirred for 10 minutes. The mixture was then diluted with DCM (30 mL) and brine (30 mL). The DCM layer was collected and the aqueous layer was further extracted with DCM (2×20 mL). The combined DCM layers were washed with brine (30 mL), dried (Na₂SO₄) and concentrated by rotary evaporation to give a dark green solid. The residue was subjected to column chromatography (7×15 cm). It was dissolved in DCM and eluted using a gradient of 1-3% MeOH/DCM. Fractions containing the product (R_{f}=0.7 in 5% MeOH/DCM) were combined concentrated by rotary evaporation to give compound 48 as a dark green solid (1.50 g, 93%).

¹H NMR (400 MHZ, CDCl₃) δ 9.72 (s, 1H), 9.67 (m, 1H), 8.86 (m, 2H), 5.88 (m, 2H), 4.58 (m, 1H), 4.51 (m, 1H), 4.06 (m, 2H), 4.00 (s, 3H), 3.84 (q, 2H), 3.63 (s, 3H), 3.58 (s, 3H), 3.48 (m, 3H), 2.63-2.48 (m, 2H), 2.20-1.90 (m, 3H), 1.75 (d, 6H), 1.12 (t, 3H), −2.21 (brs, 1H), −2.37 (brs, 1H).

### Synthesis Example 49—synthesis of phyllochlorin 13-hydroxymethyl-N-methyl-N-(2-methoxy)ethyl propylamide (compound 49)

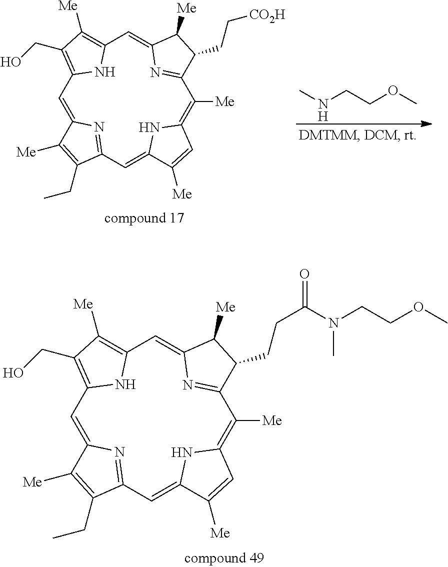

To a 25 mL RBF was added phyllochlorin 13-hydroxymethyl (compound 17) (250 mg, 1 eq), DMTMM (202 mg, 1.5 eq), DCM (10 mL) and 2-methoxy-N-methylethan-1-amine (65 mg, 1.5 eq). The resultant mixture was stirred under nitrogen at ambient temperature for 2 hours. The reaction mixture was transferred to a separatory funnel, diluted with DCM (15 mL) and washed with 0.5 M HCl (20 mL). The aqueous layer was re-extracted with DCM (2×5 mL) and the combined organic layers were washed with pH=7 phosphate buffer (20 mL). The organic phase was dried (Na₂SO₄) and concentrated by rotary evaporation to give a blue-black film. The residue was subjected to column chromatography (3×16 cm). It was dissolved in DCM to load onto the column that had been pre-equilibrated with DCM. The column was eluted using a gradient of 1-6% MeOH/DCM. Fractions containing the product (Major dark green spot, R_{f}=0.5 in 5% MeOH/DCM) were combined to give compound 49 as a dark green solid (270 mg, 95%).

¹H NMR (400 MHZ, CDCl₃) δ 9.72 (m, 2H), 8.85 (m, 2H), 6.01 (s, 2H), 6.14 (dd, 1H), 4.62 (m, 1H), 4.50 (m, 1H), 4.05 (s, 1H), 3.85 (q, 2H), 3.64 (s, 3H), 3.53 (s, 3H), 3.38 (m, 5H), 3.20-3.03 (m, 2H), 3.01 (s, 3H), 2.80 (m, 1H), 2.58-2.48 (m, 3H), 2.22-2.10 (m, 2H), 1.81-1.72 (m, 6H), −2.21 (brs, 1H), −2.40 (brs, 1H).

### Synthesis Example 50—synthesis of phyllochlorin N-methyl-N-(2-methoxy)ethyl propylamide 13-(N-(3-triphenylphosphoniumpropyl)bromide)carbamate (compound 50)

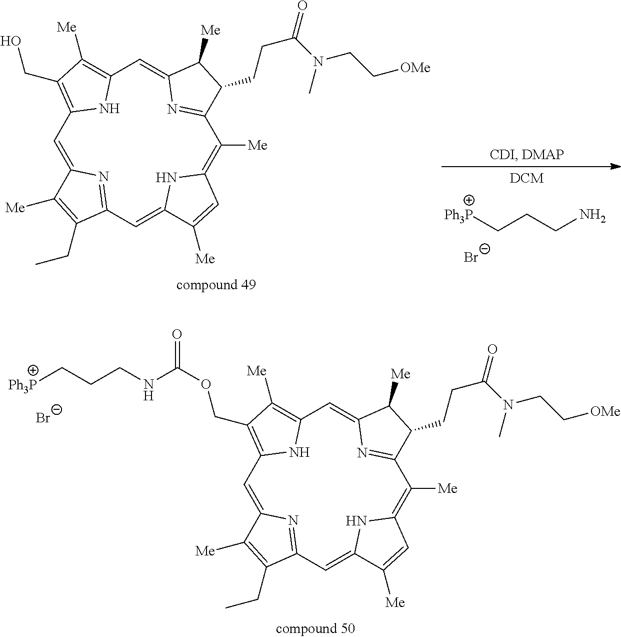

To a 25 mL RBF was added phyllochlorin 13-hydroxymethyl-N-methyl-N-(2-methoxy)ethyl propylamide (compound 49) (150 mg, 0.256 mmol, 1 eq), carbonyl diimidazole (83 mg, 0.513 mmol, 2 eq), DCM (5 mL) and DMAP (5 mg). The resultant mixture was stirred under nitrogen in the dark for 3 hours at 25° C. (3-Aminopropyl)triphenylphosphonium bromide (514 mg, 1.284 mmol, 5 eq) was added and stirring was continued overnight at 25° C. The reaction mixture was diluted with DCM (15 mL), transferred to a separatory funnel and washed with water (10 mL). The aqueous layer was re-extracted with DCM (2×10 mL) and the combined organic layers were dried (Na₂SO₄) and concentrated by rotary evaporation to give a dark green residue. The residue was subjected to column chromatography (3×12 cm). It was dissolved in DCM to load onto the column that had been pre-equilibrated with DCM. The column was eluted using a gradient of 3-9% MeOH/DCM. Fractions containing the product (Major dark green spot, R_{f}=0.05 in 5% MeOH/DCM) were combined to give compound 50 as a dark green solid (145 mg, 56%) (HPLC purity: 99.7%).

¹H NMR (400 MHZ, CDCl₃) δ 9.82 (s, 1H), 9.69 (m, 1H), 8.84 (m, 2H), 7.31-7.35 (m, 7H), 7.22-7.10 (m, 10H), 6.88-6.80 (m, 1H), 6.68-6.55 (m, 1H), 6.42 (m, 2H), 4.62 (m, 1H), 4.50 (m, 1H), 4.01 (m, 4H), 3.80 (q, 2H), 3.64 (m, 6H), 3.57 (m, 6H), 3.45-3.35 (m, 7H), 3.30-3.20 (m, 3H), 3.18 (m, 1H), 2.90 (m, 1H), 2.80 (m, 2H), 2.60-2.40 (m, 4H), 2.20 (m, 1H), 2.01 (m, 1H), 1.70-1.64 (m, 9H), −2.22 (brs, 1H), −2.37 (brs, 1H).

### Synthesis Example 51—synthesis of phyllochlorin 13-hydroxymethyl-N-(2-methoxy)ethyl propylamide (compound 51)

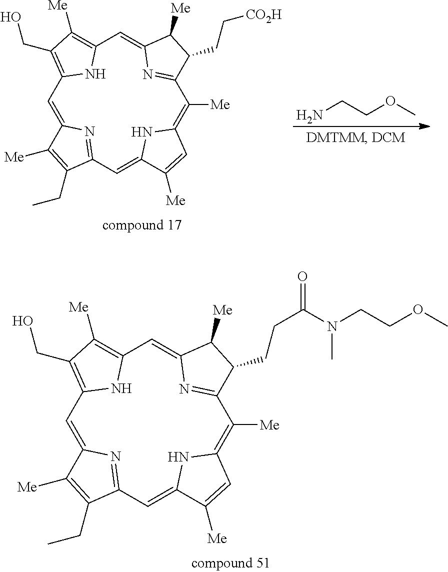

To a 50 mL RBF was added phyllochlorin 13-hydroxymethyl (compound 17) (250 mg, 0.488 mmol, 1 eq), DMTMM (202 mg, 0.731 mmol, 1.5 eq), DCM (8 mL) and 2-methoxyethan-1-amine (55 mg, 0.731 mmol, 1.5 eq). The resultant mixture was stirred under nitrogen at ambient temperature for 3 hours. The reaction mixture was transferred to a separatory funnel, diluted with DCM (15 mL) and washed with 0.5 M HCl (20 mL). The aqueous layer was re-extracted with DCM (2×5 mL) and the combined organic layers were washed with pH=7 phosphate buffer (20 mL), dried (Na₂SO₄) and concentrated by rotary evaporation to give a blue-black film which was purified by column chromatography (3×15 cm) eluting using a gradient of 1-3% MeOH/DCM. Fractions containing the major green band (R_{f}=0.15 in 5% MeOH/DCM) were combined to give compound 51 as a dark green solid (170 mg, 61%).

¹H NMR (400 MHZ, CDCl₃) δ 9.72 (s, 1H), 9.51 (s, 1H), 8.83 (m, 2H), 5.71 (s, 2H), 5.21 (m, 1H), 4.61 (m, 1H), 4.49 (m, 1H), 3.97 (s, 3H), 3.95 (s, 3H), 3.82 (q, 2H), 3.63 (s, 3H), 3.41 (s, 3H), 3.30 (s, 3H), 3.18-2.99 (m, 4H), 2.98 (s, 3H), 2.55-2.47 (m, 1H), 2.20-2.10 (m, 2H), 1.81-1.72 (m, 6H), −2.22 (brs, 1H), −2.36 (brs, 1H).

### Synthesis Example 52—synthesis of phyllochlorin N-(2-methoxy)ethyl propylamide 13-(N-(3-triphenylphosphoniumpropyl)bromide)carbamate (compound 52)

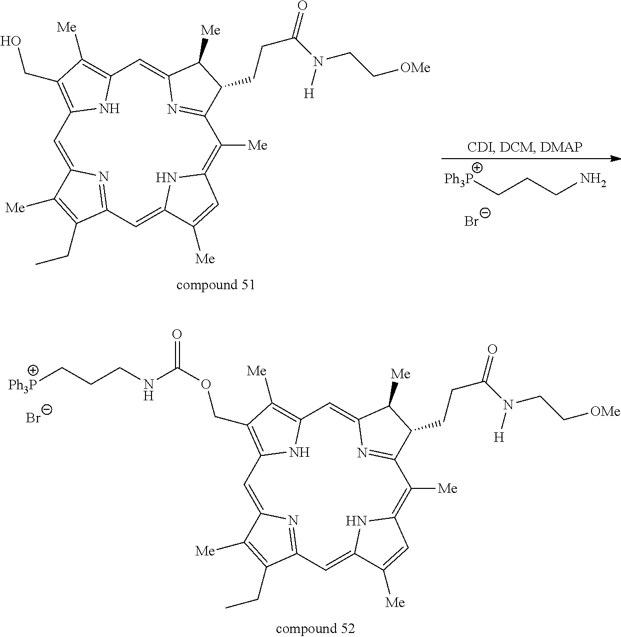

To a 25 mL RBF was added phyllochlorin 13-hydroxymethyl-N-(2-methoxy)ethyl propylamide (compound 51) (150 mg, 0.263 mmol, 1 eq), carbonyl diimidazole (85 mg, 0.527 mmol, 2 eq), DCM (4 mL) and DMAP (5 mg). The resultant mixture was stirred under nitrogen in the dark for 3 hours at 23° C. (3-Aminopropyl)triphenylphosphonium bromide (527 mg, 1.316 mmol, 5 eq) was added and stirring was continued overnight at 23° C. The reaction mixture was diluted with DCM (15 mL), transferred to a separatory funnel and washed with 1 M HCl (10 mL) and pH=7 phosphate buffer (25 mL). The aqueous layer was extracted with DCM (3×5 mL) and the combined organic layers were dried (Na₂SO₄) and concentrated by rotary evaporation to give a dark green residue. The residue was purified by column chromatography (3×12 cm) eluting using a gradient of 3-9% MeOH/DCM. Fractions containing the major green band (R_{f}=0.05 in 5% MeOH/DCM) were combined to give compound 52 as a dark green solid (9 mg, 3%).

¹H NMR (400 MHZ, CDCl₃) δ 9.82 (s, 1H), 9.69 (s, 1H), 8.84 (m, 2H), 7.80-7.50 (brm, 1H), 7.45 (t, 1H), 7.41-7.35 (m, 6H), 7.22-7.10 (m, 10H), 6.88-6.80 (m, 1H), 6.68-6.55 (m, 1H), 6.22 (m, 2H), 5.38 (m, 1H), 4.60 (m, 1H), 4.48 (m, 1H), 3.99 (m, 4H), 3.80 (q, 2H), 3.64 (m, 4H), 3.55 (m, 8H), 3.34 (m, 4H), 3.25-3.05 (m, 6H), 3.04 (s, 3H), 2.51 (m, 1H), 2.30-2.18 (m, 2H), 2.16-2.07 (m, 2H), 1.85-1.65 (m, 21H), −2.22 (brs, 1H), −2.37 (brs, 1H).

### Synthesis Example 53—synthesis of phyllochlorin ethyl ester 13-(N-(3-triphenylphosphoniumpropyl)bromide)carbamate (compound 53)

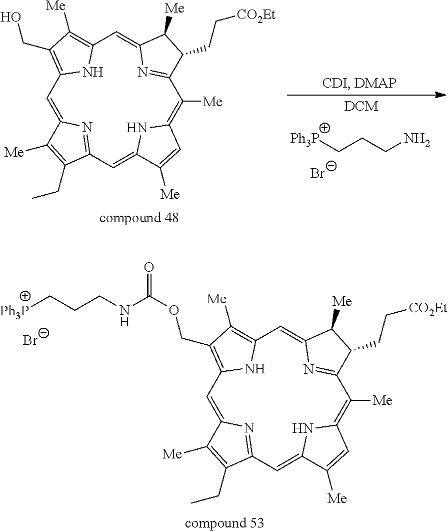

To a 25 mL RBF was added phyllochlorin 13-hydroxymethyl ethyl ester (compound 48) (200 mg, 1 eq), carbonyl diimidazole (119 mg, 2 eq), DCM (4 mL) and DMAP (5 mg). The resultant mixture was stirred under nitrogen for 3 hours. (3-Aminopropyl)triphenylphosphonium bromide (740 mg, 5 eq) was added and stirring was continued overnight at ambient temperature. The reaction mixture was diluted with DCM (20 mL), transferred to a separatory funnel and washed with water (20 mL) before being dried (Na₂SO₄) and concentrated by rotary evaporation to give a dark green residue. The residue was subjected to column chromatography. It was dissolved in DCM to load onto the column that had been pre-equilibrated with DCM. The column was eluted using a gradient of 3-7% MeOH/DCM. Fractions containing the product (Major dark green spot, R_{f}=0.15 in 5% MeOH/DCM) were combined to give compound 53 as a dark green solid (222 mg, 62%).

¹H NMR (400 MHZ, CDCl₃) δ 9.87 (s, 1H), 9.70 (s, 1H), 8.84 (m, 2H), 7.44-7.37 (m, 6H), 7.19-7.10 (m, 9H), 6.92-6.85 (m, 1H), 6.70-6.58 (m, 1H), 6.44 (m, 2H), 4.55 (m, 1H), 4.49 (m, 1H), 3.99 (m, 5H), 3.80 (q, 2H), 3.64 (m, 5H), 3.60-3.50 (m, 9H), 3.35 (s, 3H), 2.62-2.46 (m, 2H), 2.20-2.12 (m, 1H), 2.06-1.95 (m, 1H), 1.92-1.84 (m, 4H), 1.80-1.65 (m, 9H), 1.12 (t, 3H), −2.22 (brs, 1H), −2.38 (brs, 1H).

### Synthesis Example 54—synthesis of phyllochlorin 13-(N-methylamino)methyl methyl ester (compound 54)

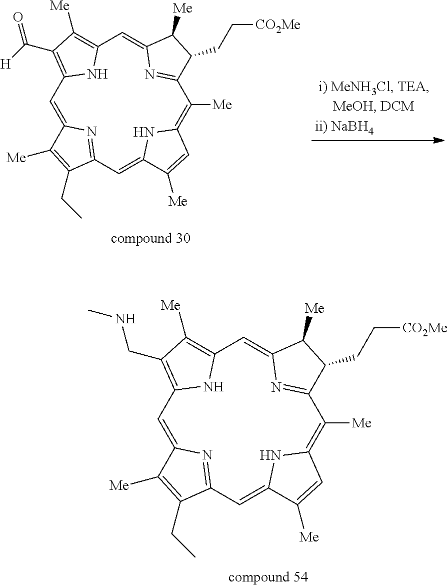 [SMILES: CCC1=C(C)c2cc3[nH]c(cc4nc(c(C)c5cc(C)c(cc1n2)[nH]5)[C@@H](CCC(=O)OC)[C@@H]4C)c(C)c3C=O ; CCC1=C(C)c2cc3[nH]c(cc4nc(c(C)c5cc(C)c(cc1n2)[nH]5)[C@@H](CCC(=O)OC)[C@@H]4C)c(C)c3CNC]

To a 25 mL RBF was added phyllochlorin 13-formyl methyl ester (compound 30) (100 mg, 0.191 mmol, 1 eq), DCM (1 mL), methanol (3 mL), TEA (58 mg, 0.572 mmol, 3 eq) and methylamine hydrochloride (39 mg, 0.572 mmol, 3 eq). The resultant mixture was stirred under nitrogen in the dark for 1 hour and then a further portion of methylamine hydrochloride (39 mg, 0.572 mmol, 3 eq), TEA (58 mg, 0.572 mmol, 3 eq) and 4 Å sieves (100 mg) were added and stirring continued for 3 hours. NaBH₄ (72 mg, 1.906 mmol, 10 eq) was added and stirring was continued for 30 minutes. The reaction was acidified with 2 M HCl (1 mL) and stirred for 10 minutes. Phosphate buffer pH=7 (15 mL) was added and the mixture extracted with DCM (3×5 mL). The combined organic layers were dried (Na₂SO₄) and concentrated by rotary evaporation to give a dark green residue. The residue was purified by column chromatography (3×14 cm) eluting using a gradient of 3-7% MeOH/DCM. Fractions containing the major green band (R_{f}=0.30 in 10% MeOH/DCM) were combined to give compound 54 as a dark green solid (54 mg, 52%).

¹H NMR (400 MHZ, CDCl₃) δ 9.51 (s, 1H), 9.46 (s, 1H), 8.75 (s, 1H), 8.69 (s, 1H), 4.82 (brm, 2H), 4.50 (m, 1H), 4.41 (m, 1H), 3.92 (s, 3H), 3.71 (q, 2H), 3.62-3.51 (m, 4H), 3.42 (s, 3H), 3.36 (s, 3H), 3.22 (s, 3H), 2.59-2.38 (m, 6H), 2.13-2.05 (m, 1H), 1.96-1.86 (m, 1H), 1.78-1.60 (m, 7H), −2.32 (brs, 1H), −2.55 (brs, 1H).

### Synthesis Example 55—synthesis of phyllochlorin 13-(N-methyl-5-triphenylphosphonium bromide pentanamide)methyl ester (compound 55)

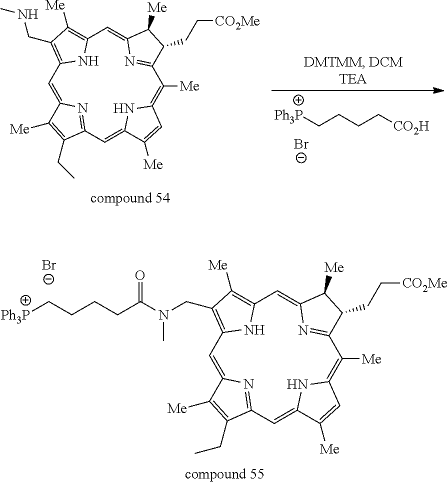

To a 25 mL RBF was added phyllochlorin 13-(N-methylamino)methyl methyl ester (compound 54) (50 mg, 0.0926 mmol, 1 eq), 4-(carboxybutyl)triphenylphosphonium bromide (82 mg, 0.1853 mmol, 2 eq), DCM (2 mL) and DMTMM (55 mg, 0.1853 mmol, 2 eq). The resultant mixture was stirred under nitrogen at ambient temperature in the dark. After 3 hours TEA (5 drops) was added and stirring continued for a further 1 hour. The reaction mixture was transferred to a separatory funnel, diluted with DCM (15 mL) and washed with 0.5 M HCl (10 mL). The aqueous layer was re-extracted with DCM (2×5 mL) and the combined organic layers were washed with pH=7 phosphate buffer (10 mL) followed by 1 M NaHCO₃ (10 mL). The organic phase was dried (Na₂SO₄) and concentrated by rotary evaporation to give a blue-black film. The residue was purified by column chromatography (3×13 cm) eluting using a gradient of 4-7% MeOH/DCM. Fractions containing the major green band (R_{f}=0.30 in 7% MeOH/DCM) were combined to give compound 55 as a dark green solid (51 mg, 57%).

¹H NMR (400 MHZ, CDCl₃) δ 9.81 (s, 1H), 9.68 (s, 1H), 8.86-8.80 (m, 2H), 7.75-7.67 (m, 6H), 7.55-7.45 (m, 9H), 5.91 (brm, 2H), 4.58-4.47 (m, 2H), 3.98 (s, 3H), 3.86-3.77 (m, 4H), 3.63 (s, 3H), 3.58 (s, 3H), 3.45 (s, 3H), 3.26 (s, 3H), 3.09 (s, 3H), 2.73 (t, 2H), 2.51-2.46 (m, 2H), 2.20-2.10 (m, 3H), 2.09-1.98 (m, 1H), 1.90-1.68 (m, 13H), −2.22 (brs, 1H), −2.32 (brs, 1H).

### Synthesis Example 56—synthesis of phyllochlorin 13-(N-methyl-ethylcarbamate) aminomethyl methyl ester (compound 56)

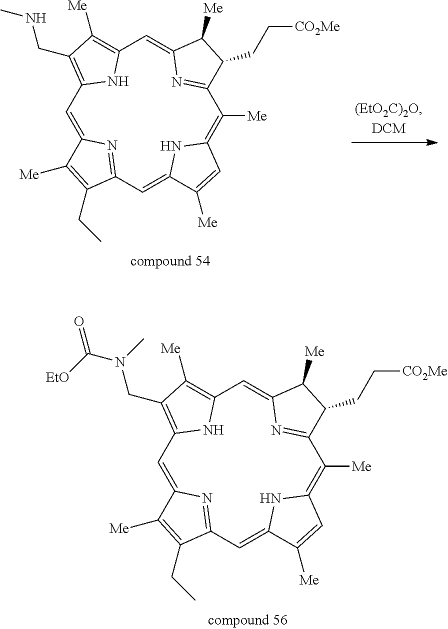 [SMILES: CCC1=C(C)c2cc3[nH]c(cc4nc(c(C)c5cc(C)c(cc1n2)[nH]5)[C@@H](CCC(=O)OC)[C@@H]4C)c(C)c3CNC ; CCOC(=O)N(C)Cc1c(C)c2cc3nc(c(C)c4cc(C)c(cc5nc(cc1[nH]2)C(C)=C5CC)[nH]4)[C@@H](CCC(=O)OC)[C@@H]3C]

To a 25 mL RBF was added phyllochlorin 13-(N-methylamino)methyl methyl ester (compound 54) (50 mg, 0.0926 mmol, 1 eq), DCM (2 mL) and diethyl dicarbonate (75 mg, 0.4632 mmol, 5 eq). The resultant mixture was stirred under nitrogen at ambient temperature. After 30 minutes the reaction mixture was diluted with pH 7 phosphate buffer (10 mL), transferred to a separatory funnel and extracted with DCM (2×5 mL) and the combined organics dried (Na₂SO₄) and concentrated by rotary evaporation to give a dark green oil. The residue was purified by silica gel column chromatography using 0.5-1% MeOH/DCM. Fractions containing the major dark green spot (R_{f}=0.75 in 5% MeOH/DCM) were combined to give compound 56 as a dark green solid (37 mg, 65%).

¹H NMR (400 MHZ, CDCl₃) δ 9.91-9.67 (m, 2H), 8.88-8.82 (m, 2H), 5.90 (brm, 2H), 4.61-4.38 (m, 4H), 4.01 (s, 3H), 3.87 (q, 2H), 3.67 (s, 3H), 3.58 (s, 3H), 3.51 (s, 3H), 3.39 (s, 3H), 2.99-2.89 (m, 3H), 2.62-2.48 (m, 2H), 2.20-2.00 (m, 2H), 1.82-1.74 (m, 6H), 1.65-1.55 (m, 2H), 1.42-1.36 (m, 2H), −2.15 (brs, 1H), −2.30 (brs, 1H).

### Synthesis Example 57—synthesis of phyllochlorin 13-(N-3-triphenylphosphonium bromide)aminomethyl methyl ester (compound 57)

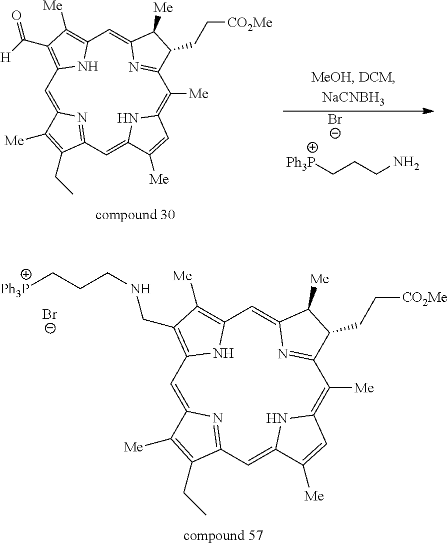

To a 25 mL RBF was added phyllochlorin 13-formyl methyl ester (compound 30) (50 mg, 0.0953 mmol, 1 eq), DCM (1 mL), MeOH (3 mL), (3-aminopropyl) triphenylphosphonium bromide (57 mg, 0.1430 mmol, 1.5 eq), sodium cyanoborohydride (18 mg, 0.2860 mmol, 3 eq) and 4 A sieves (400 mg). The resultant mixture was stirred under nitrogen in the dark at 22° C. After 6 hours (3-aminopropyl)triphenylphosphonium bromide (57 mg, 0.1430 mmol, 1.5 eq) and sodium cyanoborohydride (18 mg, 0.2860 mmol, 3 eq) were added and stirring was continued for 4 days. Water (10 mL) was added and the mixture stirred for 10 minutes then extracted with DCM (3×10 mL). The combined organic extracts were washed with saturated aqueous NaHCO₃ (2×15 mL), then dried (Na₂SO₄) and concentrated by rotary evaporation to give a dark green residue. The residue was purified by column chromatography using 3-5% MeOH/DCM. Fractions containing the major dark green spot (R_{f}=0.20 in 5% MeOH/DCM) were combined to give compound 57 as a dark green solid (18 mg, 21%).

¹H NMR (400 MHZ, CDCl₃) δ 9.82 (s, 1H), 9.70 (s, 1H), 8.86 (s, 1H), 8.78 (s, 1H), 7.31-7.26 (m, 3H), 7.13-7.06 (m, 12H), 5.12 (brm, 2H), 4.59-4.47 (m, 2H), 4.00 (m, 4H), 3.80 (q, 3H), 3.67 (m, 4H), 3.59 (m, 4H), 3.42 (s, 3H), 3.23 (s, 3H), 3.16-3.10 (m, 2H), 2.66-2.48 (m, 3H), 2.24-2.17 (m, 1H), 2.05-1.95 (m, 3H), 1.80-1.60 (m, 1H), −2.28 (brm, 2H).

### Synthesis Example 58—synthesis of phyllochlorin 13-(N-3-triphenylphosphonium bromide ethylcarbamate)aminomethyl methyl ester (compound 58)

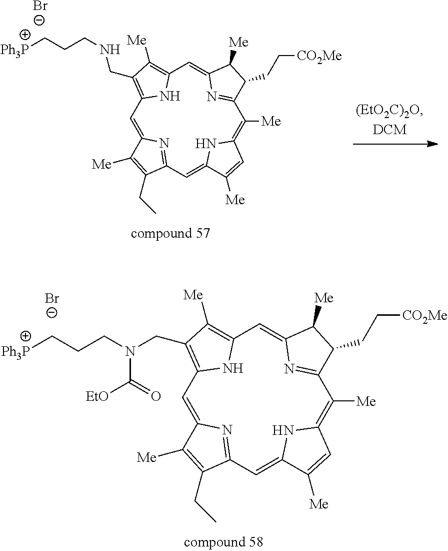

To a 25 mL RBF was added phyllochlorin 13-(N-3-triphenylphosphonium bromide)aminomethyl methyl ester (compound 57) (15 mg, 0.0165 mmol, 1 eq), DCM (2 mL) and diethyl dicarbonate (13 mg, 0.0825 mmol, 5 eq). The resultant mixture was stirred under nitrogen at ambient temperature. After 30 minutes the reaction mixture was diluted with pH 7 phosphate buffer (10 mL), transferred to a separatory funnel and extracted with DCM (3×5 mL) and the combined organics dried (Na₂SO₄) and concentrated by rotary evaporation to give a blue-black oil. The residue was purified by column chromatography using 3-5% MeOH/DCM. Fractions containing the major dark green spot (R_{f}=0.15 in 5% MeOH/DCM) were combined to give compound 58 as a dark green solid (12 mg, 75%).

¹H NMR (400 MHZ, CDCl₃) δ 9.86-9.55 (m, 2H), 8.92-8.85 (m, 2H), 6.70-6.52 (m, 3H), 6.50-6.25 (m, 12H), 5.95 (d, 1H), 5.81 (d, 1H), 4.65-4.52 (m, 3H), 4.31 (m, 1H), 4.05 (s, 3H), 3.81 (m, 4H), 3.68-3.63 (m, 6H), 3.56 (s, 3H), 3.32-3.23 (m, 3H), 2.75-2.65 (m, 1H), 2.60-2.50 (m, 1H), 2.40-2.30 (m, 1H), 2.00-1.91 (m, 1H), 1.83-1.79 (m, 3H), 1.75-1.55 (m, 9H), −2.41 (brm, 2H).

### Biological Experimental Details

### Example 1—Determination of Solubility of Phyllochlorin Analogues

Absorbance maxima were used as a surrogate measure of solubility. The relevant phyllochlorin analogue was diluted to 50 μM in PBS (phosphate buffered saline) solutions containing decreasing amounts of DMSO from 100% to 0%.

Where required, polyvinylpyrrolidone (K30) was added to a final concentration of 1% w/v. Absorbance was measured using a Cytation 3 Multimode Plate Reader (Biotek) in spectral scanning mode, with spectra captured between 500-800 nm in 2 nm increments. Equivalent blank solutions were also measured and subtracted accordingly. Each spectrum was normalized to have a minimum signal of 0, and a maximum signal in pure DMSO solution (the most soluble state) of 100%.

### Results—Solubility and Absorbance Maxima of Phyllochlorin Analogues

The absorbance spectrum of compound 1 was measured in the presence of 1% PVP (w/v). Compound 1 had an absorbance maxima at 650±2 nm.

The solubility of compound 1 was assessed in solutions containing DMSO as an organic solvent. Compound 1 (50 μM final concentration) was resuspended in 100% DMSO to fully dissolve compound 1, in the presence or absence of 1% PVP (w/v). With concentrations of compound 1 maintained at 50 μM the % DMSO was decreased in a stepwise manner from 100% to 0%. All solutions were mixed by vortexing, then centrifuged for 2 mins to pellet any insoluble material. A 150 μl aliquot was transferred to individual wells of a 96-well clear microplate, and absorbance spectra collected between 500-800 nm in 2 nm increments. Equivalent solutions containing decreasing % DMSO were used to control for background.

In concentrations above 70% DMSO compound 1 was completely soluble, however at DMSO concentrations below 40% its apparent solubility was reduced to ˜45%. The addition of PVP 1% improved solubility in aqueous solution (i.e. no DMSO) to ˜75% of maximal. Thus, addition of PVP improved the solubility of compound 1.

### Example 2—Cytotoxicity, Phototoxicity and Therapeutic Index

### Preparation of Photosensitizer Stock Solutions

Photosensitizers (e.g. phyllochlorin analogue, chlorin e4 disodium (provided by Advanced Molecular Technologies, Scoresby) or Talaporfin sodium (purchased from Focus Bioscience cat #HY-16477-5 MG)) were resuspended in 100% dimethylsulfoxide (DMSO) at a concentration of 5.5 mM. Samples were stored at 4° C. protected from light.

### Preparation of Photosensitizers for In Vitro Studies

For in vitro experiments, photosensitizers (stock solution 5.5 mM in 100% DMSO) were diluted 1:100 in concentrated excipient solution (final 55 μM photosensitizer in 10% w/v Kollidon-12, 42.4% w/v polysorbate 80, 0.6% w/v citric acid anhydrous, 40% w/v ethanol, 1.0% DMSO). Serial dilutions were prepared in cell culture media (Dulbecco's Modified Eagle Medium/Nutrient Mixture F-12 (DMEM/F-12)) supplemented with 10% v/v Fetal Bovine Serum, 100 U/mL penicillin, 100 μg/mL streptomycin and the same excipient solution at a constant 1:55 dilution.

### Cell Culture

Human ovarian cancer cell line SKOV3 (ATCC #HTB-77) was maintained in Dulbecco's Modified Eagle Medium/Nutrient Mixture F-12 (DMEM/F-12), supplemented with 10% v/v Fetal Bovine Serum, 100 U/mL penicillin and 100 μg/mL streptomycin. Monolayer cultures were grown in a humidified incubator at 37° C. with 5% CO₂. Once cells had reached ˜80% confluence, spent media was replaced with media containing photosensitizer at the required concentration and cells were incubated for the desired period of time to allow photosensitizer uptake.

### Statistical Analyses

All data were analysed using GraphPad PRISM v8.3.1 (549) (GraphPad Software, CA). Spectral absorbance and viability measurements were normalized in the range 0-100%, with a minimum of 0 and a maximum value determined from the dataset. Dose response was determined using a sigmoidal four-point non-linear regression with variable slope, and IC10 or IC90 calculated for each compound. All data are shown as mean±SD (where appropriate).

### Cytotoxicity

SKOV3 cells were seeded in 96-well black wall plates (Greiner #655090) at a cell density of 5000 cells in 100 μl culture medium per well. On reaching ˜60% confluence, media was aspirated and replaced with fresh media containing the relevant phyllochlorin analogue from 0-100 μM in DMSO. Cells were incubated for a further 24 hours, allowing uptake of phyllochlorin analogues.

To test for inherent cytotoxicity (i.e. “dark toxicity”) of the phyllochlorin analogues, the culture media was replaced after 24 hours with fresh media containing 10% (v/v) AlamarBlue Cell Viability Reagent (ThermoFisher) and cells incubated at 37° C. for 6 hours. Untreated cells were used as a control. Fluorescence (Ex 555 nm/Em 596 nm) was measured using a Cytation 3 Cell Imaging Multi-Mode Reader (Biotek), and cytotoxicity assessed according to the % viable cells remaining. All measurements were made in quadruplicate.

### Phototoxicity

SKOV3 cells were seeded in 96-well black wall plates (Greiner #655090) at a cell density of 5000 cells in 100 μl culture medium per well. On reaching ˜60% confluence, media was aspirated and replaced with fresh media containing the relevant phyllochlorin analogue from 0-100 μM in DMSO. Cells were incubated for a further 24 hours, allowing uptake of phyllochlorin analogues.

To test for phototoxicity, cells incubated with phyllochlorin analogues (0-10 UM in DMSO) had culture media replaced after 24 hours (as above) and were then exposed to a 660 nm laser (Invion) or light-emitting diode (LED) panel (Invion) with optical power density at 50 mW/cm² for 5 mins (total 15 J/cm²). Laser and LED exposure induce an equivalent response in relation to phototoxicity. Following activation, cells were cultured for a further 24 hours. Media was then replaced with fresh media containing AlamarBlue, and % viable cells remaining assessed as above. Controls included cells treated with phyllochlorin analogues but not activated by laser light; cells without phyllochlorin analogue treatment but with laser light; and untreated controls. All measurements were made in quadruplicate.

### Toxicity Profile for Phyllochlorin Analogues

The phototoxicity and inherent cytotoxicity (i.e. “dark toxicity”) of phyllochlorin analogues were assessed as previously using SKOV3 ovarian cancer cells. For comparative purposes, phyllochlorin analogues were compared against chlorin e4 disodium and Talaporfin sodium, a clinically approved photosensitizer used in the photodynamic treatment of lung cancers. Phototoxicity IC90 values and dark toxicity IC10 values were calculated using a log[inhibitor]-vs normalized response dose curve with variable slope, using the formula Y=100/(1+ (IC90/X)\hat{ }HillSlope (phototoxicity IC90)) or Y=100/(1+ (IC10/X)\hat{ }HillSlope (dark toxicity IC10)).

Phototoxicity and dark toxicity values are provided in Table 1. With only three exceptions (compounds 10, 16 and 30) all phyllochlorin analogues had phototoxicity IC90 values in the nM range; with an IC90 for eighteen compounds below 50 nM, and an IC90 for six compounds below 2 nM (Table 1). These were substantially better than chlorin e4 disodium (IC90 21.32 μM) or Talaporfin sodium (IC90 22.83 μM); indeed, the best-performing compound (compound 53) achieved 5 orders of magnitude greater phototoxicity compared to Talaporfin sodium. Thus, phyllochlorin analogues achieved an up to ˜30,000-fold increase in phototoxicity compared to Talaporfin sodium, a clinically approved photosensitizer.

Substantial variation in the dark toxicity of the phyllochlorin analogues of the present invention was observed (Table 1). The greater phototoxicity afforded by the phyllochlorin analogues of the present invention, however, is expected to offset any dark toxicity issue through a decreased dose requirement in use.

### Therapeutic Index for Phyllochlorin Analogues

To evaluate the therapeutic potential of phyllochlorin analogues, the therapeutic index (TI) was calculated. TI provides a quantitative measurement to describe relative drug safety, by comparing the drug concentration required for desirable effects versus the concentration resulting in undesirable off-target toxicity. TI was calculated using phototoxicity IC90 vs dark toxicity IC10.

TI values are provided in Table 1. Talaporfin sodium had a low therapeutic index (TI=0.49) with chlorin e4 disodium only marginally better (TI=1.89), indicating that whilst their relative cytotoxicity is low, the potential therapeutic window for their use is small. Most of the phyllochlorin analogues of the present invention had comparatively significantly improved TIs with substantially greater phototoxicity (Table 1).

Thus, the phyllochlorin analogues of the present invention have a desirable therapeutic index that is better than a clinically applied photosensitizer. Moreover the greater phototoxicity of the phyllochlorin analogues suggests their potential use at a greatly reduced dose in vivo. The phyllochlorin analogues therefore have an acceptable therapeutic profile for clinical application.

TABLE 1
Toxicity profile and therapeutic index for phyllochlorin analogues:

| | Phototoxicity | Dark toxicity | Therapeutic |
|---|---|---|---|
| Species | μM (IC90) | μM (IC10) | Index |
| Compound 1 | 0.060 | 13.500 | 225.00 |
| Compound 2 | 0.030 | 7.640 | 254.67 |
| Compound 3 | 0.308 | 45.720 | 148.44 |
| Compound 4 | 0.099 | 0.775 | 7.83 |
| Compound 6 | 0.084 | 0.528 | 6.29 |
| Compound 8 | 0.665 | 7.442 | 11.19 |
| Compound 10 | 1.496 | 4.732 | 3.16 |
| Compound 12 | 0.353 | 2.466 | 6.99 |
| Compound 13 | 0.421 | 60.750 | 144.30 |
| Compound 14 | 0.186 | 0.626 | 3.37 |
| Compound 16 | 1.824 | 5.929 | 3.25 |
| Compound 17 | 0.891 | 25.000 | 28.06 |
| Compound 18 | 0.007 | 4.393 | 627.57 |
| Compound 19 | 0.018 | 0.090 | 5.00 |
| Compound 20 | 0.003 | 1.998 | 666.00 |
| Compound 21 | 0.012 | 3.801 | 316.75 |
| Compound 22 | 0.004 | 2.228 | 557.00 |
| Compound 23 | 0.012 | 3.086 | 257.17 |
| Compound 24 | 0.503 | 41.190 | 81.89 |
| Compound 25 | 0.146 | 0.279 | 1.91 |
| Compound 26 | 0.248 | 21.550 | 86.90 |
| Compound 27 | 0.397 | 10.520 | 26.50 |
| Compound 28 | 0.003 | 4.877 | 1,625.67 |
| Compound 29 | 0.0867 * | 30.000 | 346.02 |
| Compound 30 | 2.270 | 45.760 | 20.16 |
| Compound 32 | 0.0724 | 30.850 | 426.10 |
| Compound 33 | 0.7649 | 14.080 | 18.41 |
| Compound 35 | 0.0467 | 1.651 | 35.35 |
| Compound 39 | 0.0018 * | 6.403 | 3,557.22 |
| Compound 40 | 0.0008 * | 0.6145 | 768.13 |
| Compound 42 | 0.2450 * | 4.252 | 17.36 |
| Compound 43 | 0.0042 * | 0.5889 | 140.21 |
| Compound 44 | 0.0057 * | 0.5603 | 98.30 |
| Compound 45 | 0.0012 * | 3.977 | 3,314.17 |
| Compound 46 | 0.0014 * | 1.277 | 912.14 |
| Compound 50 | 0.0016 * | 0.470 | 293.75 |
| Compound 52 | 0.0052 * | 1.060 | 203.85 |
| Compound 53 | 0.00077 * | 2.240 | 2,909.09 |
| Chlorin e4 disodium | 21.32 | 40.23 | 1.89 |
| Talaporfin sodium | 22.83 | 11.10 | 0.49 |

* denotes that the phototoxicity was measured by LED

### Example 3—Phyllochlorin Glucose Analogues are Actively Transported into Cancer Cells

### Method

SKOV3 cells were seeded in 96-well black wall plates (Greiner #655090) at a cell density of 5000 cells in 100 μl culture medium per well. On reaching ˜60% confluence, media was aspirated and replaced with fresh media containing compound 1 or chlorin e4 disodium at 5 μM. Cells were incubated for up to 18 hours. At time periods of 10 min, 20 min, 30 min, 60 min, 2 hrs, 4 hrs and 18 hrs, media was removed and the cells lysed in 100 μl of lysis buffer (PBS+1% SDS+1% PVP+Benzonase Nuclease 500 units/mL). Each sample was snap-frozen on dry ice until use.

Eight point standard curves using compound 1 or chlorin e4 disodium were constructed in the range 0-10 μM using serial dilutions in lysis buffer. Cell lysates (100 μl) were thawed, centrifuged at room temperature (21,000 g, 10 mins) and the supernatants transferred to fresh tubes. 100 μl of each supernatant was added neat to 96-well microplates and absorbance measured at 405 nm using a Cytation 3 multimode plate reader. All wells were measured against blank wells containing buffer alone, and a background value measured using untreated cells was subtracted from all samples. The concentration of each chlorin species was determined against the appropriate standard curve in each case.

### Results

Conjugation of a glucose moiety to phyllochlorin was hypothesized to promote active uptake into cancer cells, through interaction with one or more glucose transporters present on the cell surface. To assess the rate of sensitizer uptake in vitro, SKOV3 ovarian cancer cells were incubated in the presence of 5 μM compound 1 for up to 18 hours. Chlorin e4 disodium (which enters cells via a passive diffusion process) was included as a comparator, and untreated cells were used as a control. Cells were harvested at time periods from 10 mins to 18 hours post addition of photosensitizers, cells were lysed and the supernatants measured for uptake of photosensitizers against an appropriate standard curve.

Consistent with its passive uptake, chlorin e4 disodium remained undetectable until after one hour of incubation, and accumulated slowly until reaching a maximum at 18 hours.

Compound 1 on the other hand was observed in cells within 10 minutes of incubation; and the rate of uptake was substantially faster than that of chlorin e4 disodium and this rate was maintained for 2-4 hours after addition of photosensitizer. The amount of compound 1 recovered in cell lysates also reached a comparatively higher concentration, supporting an active uptake process compared to the simple passive equilibrium observed for non-conjugated chlorins.

Thus, phyllochlorin glucose analogues are actively transported into cancer cells and reach higher intracellular concentrations than non-conjugated forms.

### Example 4—Phyllochlorin Glucose Analogues are Absorbed by Bacterial Cells

### Method

E coli DH5α cells were inoculated into LB media and incubated overnight (shaking at 37° C.) in the presence of 50 μM compound 1 or chlorin e4 disodium. Untreated cells acted as the control. The following day cells were harvested by centrifugation (5000 g, 10 mins, room temperature) and lysed by addition of PBS+1% SDS (w/v)+1% PVP-17 (w/v). Insoluble material was pelleted by centrifugation at 21,000 g (10 mins, bench top centrifuge) and the supernatant transferred to a fresh tube. A 100 μl aliquot was added to clear 96-well microtitre plates and absorbance spectra measured between 600-700 nm (Cytation 3 multimode plate reader). The optical density (OD) was plotted against wavelength in each case to determine uptake of photosensitizer into cells.

### Result

Conjugation of a glucose moiety to phyllochlorin analogues promoted active uptake into cancer cells (above); we therefore tested whether this would also promote uptake into bacterial cells, a crucial step required for the application of phyllochlorin analogues as an antibacterial agent. Compound 1 was clearly detectible in bacterial lysates following the incubation period. By comparison, chlorin e4 disodium (which is taken up by passive diffusion in cancer cells) was detected marginally above background. Thus, similar to cancer cells, phyllochlorin glucose analogues are likely to be absorbed by bacteria via an active transport process and have additional potential applications as antibacterial agents.

It will be understood that the present invention has been described above by way of example only. The examples are not intended to limit the scope of the invention. Various modifications and embodiments can be made without departing from the scope and spirit of the invention, which is defined by the following claims only.

1. A compound of formula (I) or a complex of formula (II): 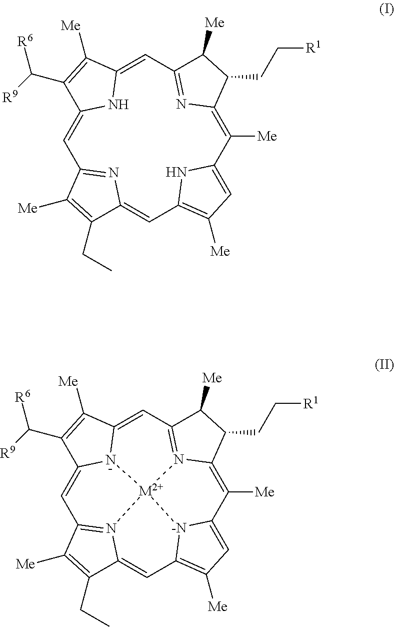
or a pharmaceutically acceptable salt thereof, wherein:
—R¹ is selected from —CH₂OR², —CH₂SR², —CH₂S(O)R², —CH₂S(O)₂R², —CH₂N(R²)₂, —R², —C(O)—OR³, —C(O)—SR³, —C(O)—N(R³)₂, —C(S)—OR³, —C(S)—SR³ or —C(S)—N(R³)₂;
—R², each independently, is selected from —H, —C(O)R⁴, —C(O)—OR⁴, —C(O)—SR⁴, —C(O)—N(R⁴)₂, —C(S)—OR⁴, —C(S)—SR⁴, —C(S)—N(R⁴)₂, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁷]Y, —R^{α}—[N(R⁵)₂ (R^{5′})], —R^{α}—[P(R⁵)₂ (R^{5′})] or —R^{α}—[R⁷];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R) 2, —R^{α}—X, —R^{α}—[N(R⁵)₃]Y, —R^{α}—[P(R⁵)₃]Y, —R^{α}—[R⁷]Y, —R^{α}—[N(R⁵)₂ (R^{5′})], —R^{α}—[P(R⁵)₂ (R^{5′})] or —R^{α}—[R⁷];
—R^{α}—, each independently, is selected from a C₁-C₄₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O, S, P or Se in its carbon skeleton;
—R⁵, each independently, is selected from C₁-C₄ alkyl, C₁-C₄ haloalkyl, —(CH₂CH₂O)ₙ—H,
(CH₂CH₂O)ₙ—CH₃, phenyl or C₅-C₆ heteroaryl, wherein the phenyl or C₅-C₆ heteroaryl may optionally be substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂, wherein the phenyl or C₅-C₆ heteroaryl may optionally be further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from —OR², —N(R²)₂, —SR², —S(O)R², —S(O)₂R², or —X;
—R⁷ is —[NC₅H₅] optionally substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{7′} is —[NC₅H₅] substituted with —CO₂ and optionally further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁹ is hydrogen or methyl; or —R⁶ and —R⁹ together form an oxo (═O) group;
n is 1, 2, 3, 4, 5 or 6;
X is a halo group;
Y is a counter anion;
Z is a counter cation; and
M²⁺ is a metal cation.

2. The compound or complex according to claim 1, wherein each —R^{α}— is independently selected from C₁-C₆ alkylene.

3. The compound or complex according to claim 1, wherein at least one of —R², —R³ and —R⁴ is selected from —R^{α}—OR^{β}, —R^{α}—SR^{β}, —R^{α}—S(O)R^{β} or —R^{α}—S(O)₂R^{β}, and —R^{β} is a saccharidyl group.

4. The compound or complex according to claim 3, wherein —R^{β} is a saccharidyl group selected from: 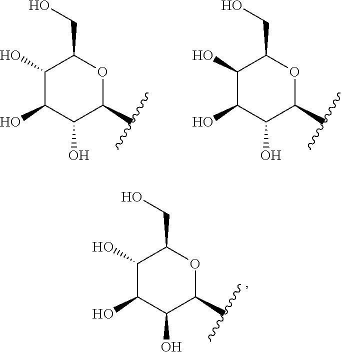 [SMILES: *[C@@H]1O[C@H](CO)[C@@H](O)[C@H](O)[C@H]1O ; *[C@@H]1O[C@H](CO)[C@H](O)[C@H](O)[C@H]1O ; *[C@@H]1O[C@H](CO)[C@@H](O)[C@H](O)[C@@H]1O]
optionally wherein the saccharidyl group is:

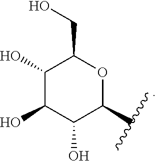 [SMILES: *[C@@H]1O[C@H](CO)[C@@H](O)[C@H](O)[C@H]1O]

5. (canceled)

6. The compound or complex according to claim 3, wherein —R^{β} is a saccharidyl group selected from: 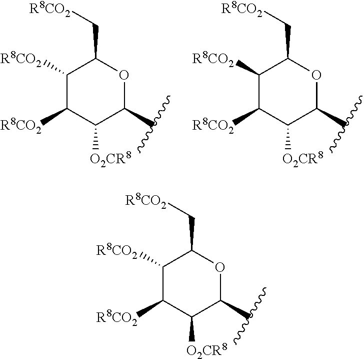
wherein —R⁸ is selected from C₁-C₄ alkyl, optionally wherein R⁸ is methyl.

7. (canceled)

8. The compound or complex according to claim 1, wherein —R¹ is;
(a) —C(O)—OR³, —R³ is —R^{β}, and —R^{β} is a C₁-C₄ alkyl group; or
(b) selected from —C(O)—OR³, —C(O)—SR³ or —C(O)—N(R³)(R^{3′}), wherein —R³ is selected from —R^{α}—OR^{β}, —R^{α}—SR^{β} or —R^{α}—S(O)₂R^{β}, and —R^{β} is a saccharidyl group, and —R^{3′} is H or C₁-C₄ alkyl.

9. (canceled)

10. The compound or complex according to claim 1, wherein —R⁶ is selected from —OR² or —SR², and —R² is selected from —R^{α}—OR^{β}, —R^{α}—SR^{β}, —R^{α}—S(O)R^{β} or —R^{α}—S(O)₂R^{β}, and —R^{β} is a saccharidyl group.

11. The compound or complex according to claim 1, wherein the compound or complex is: 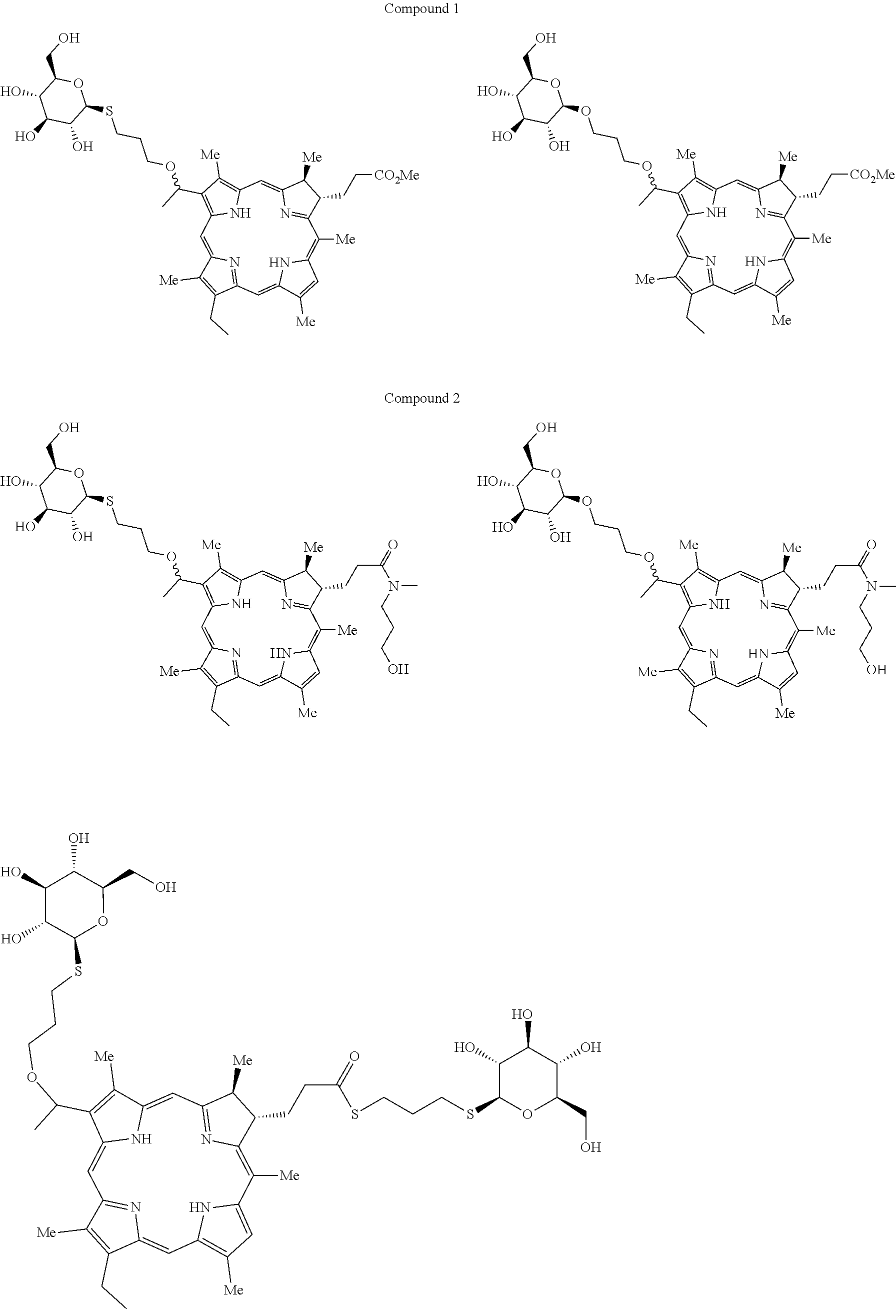 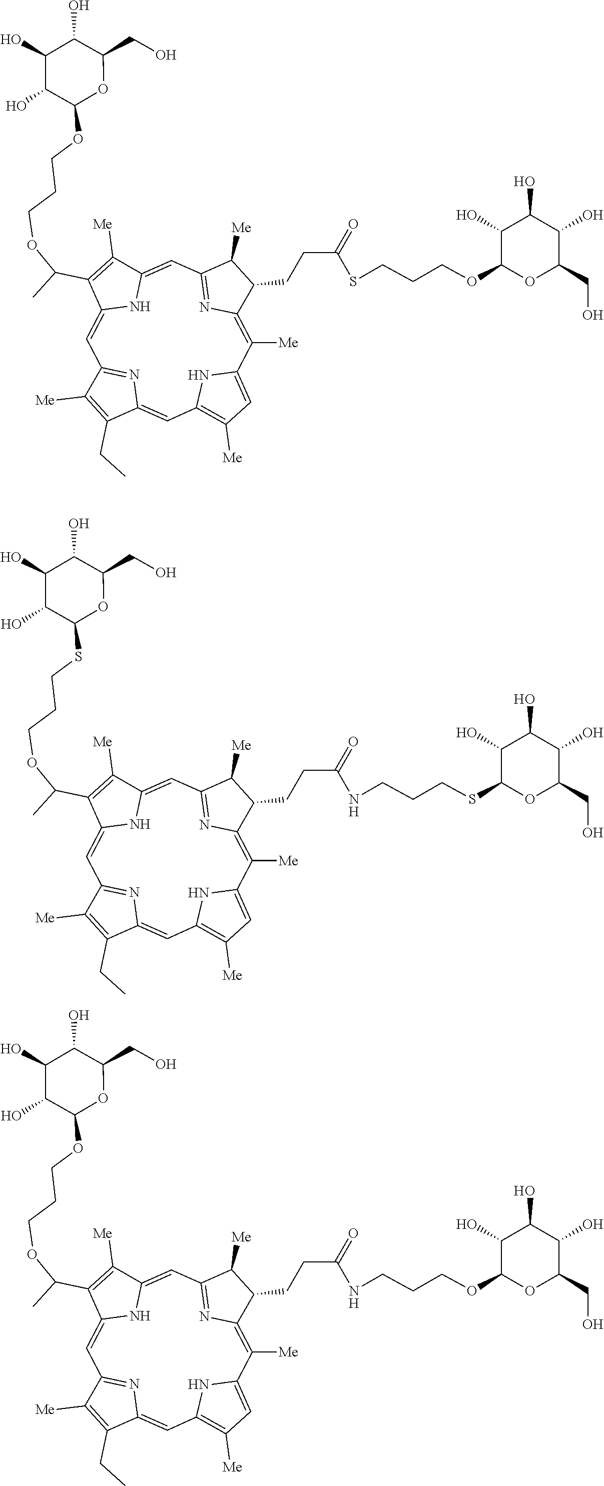 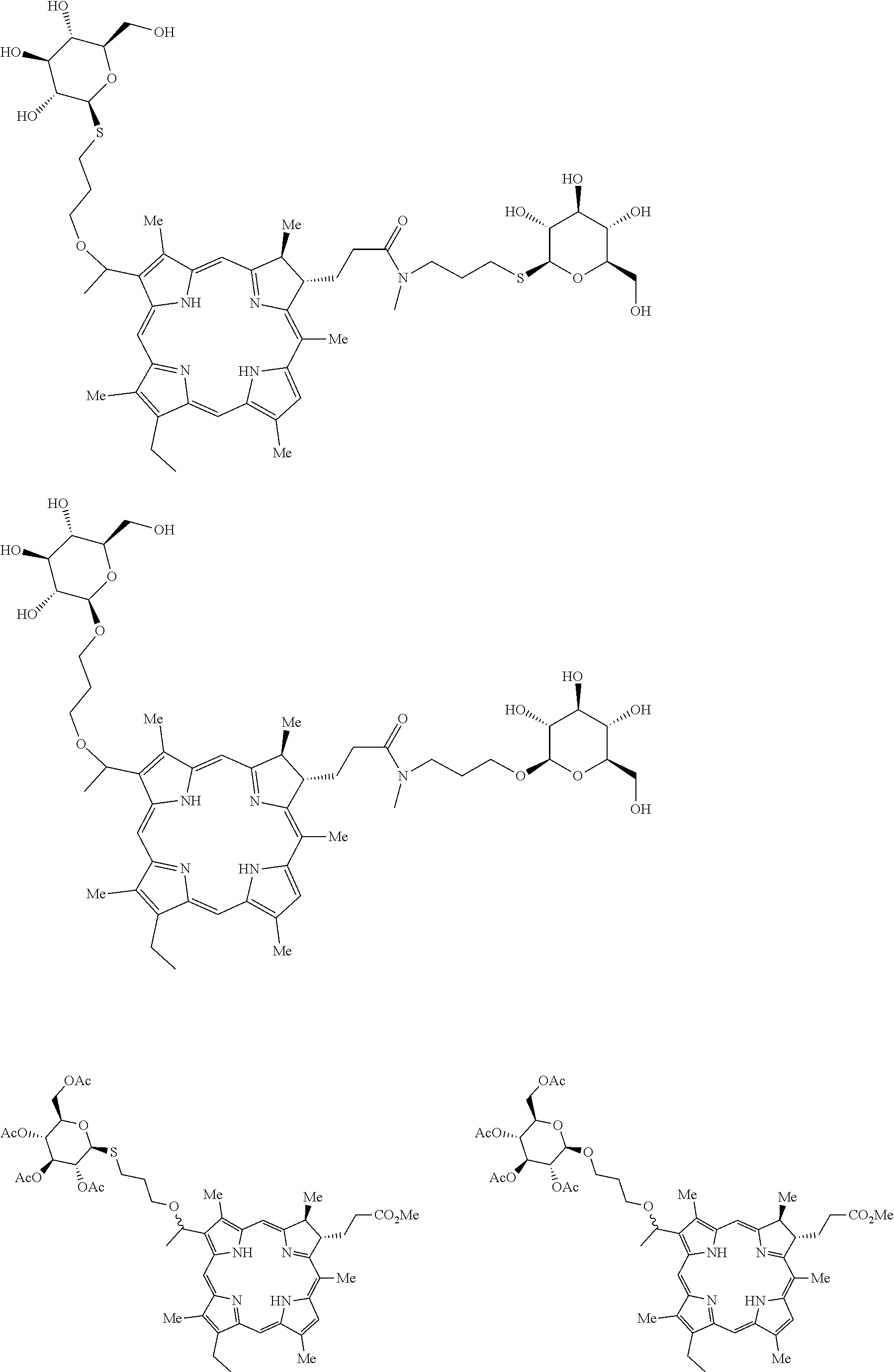 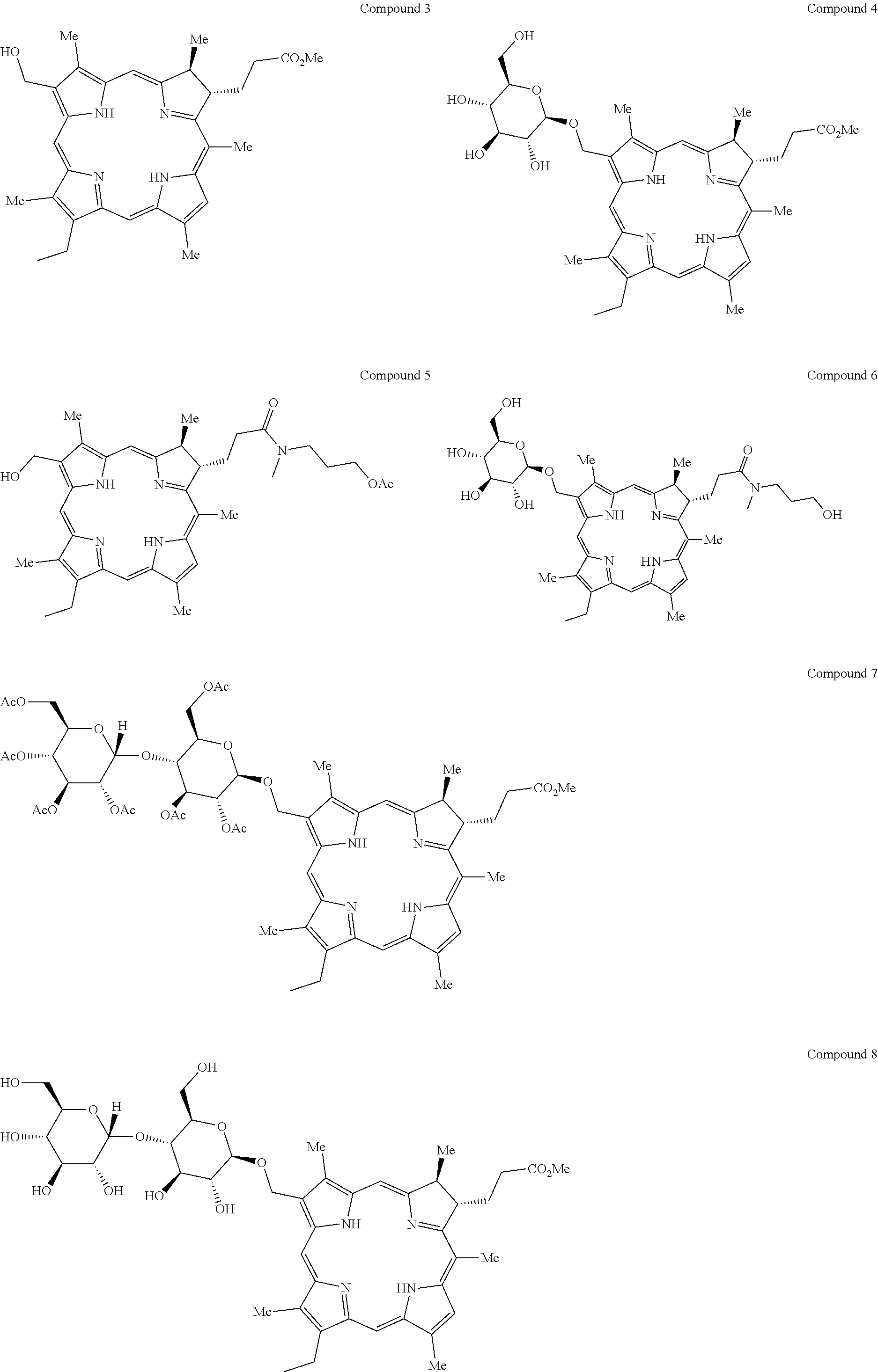 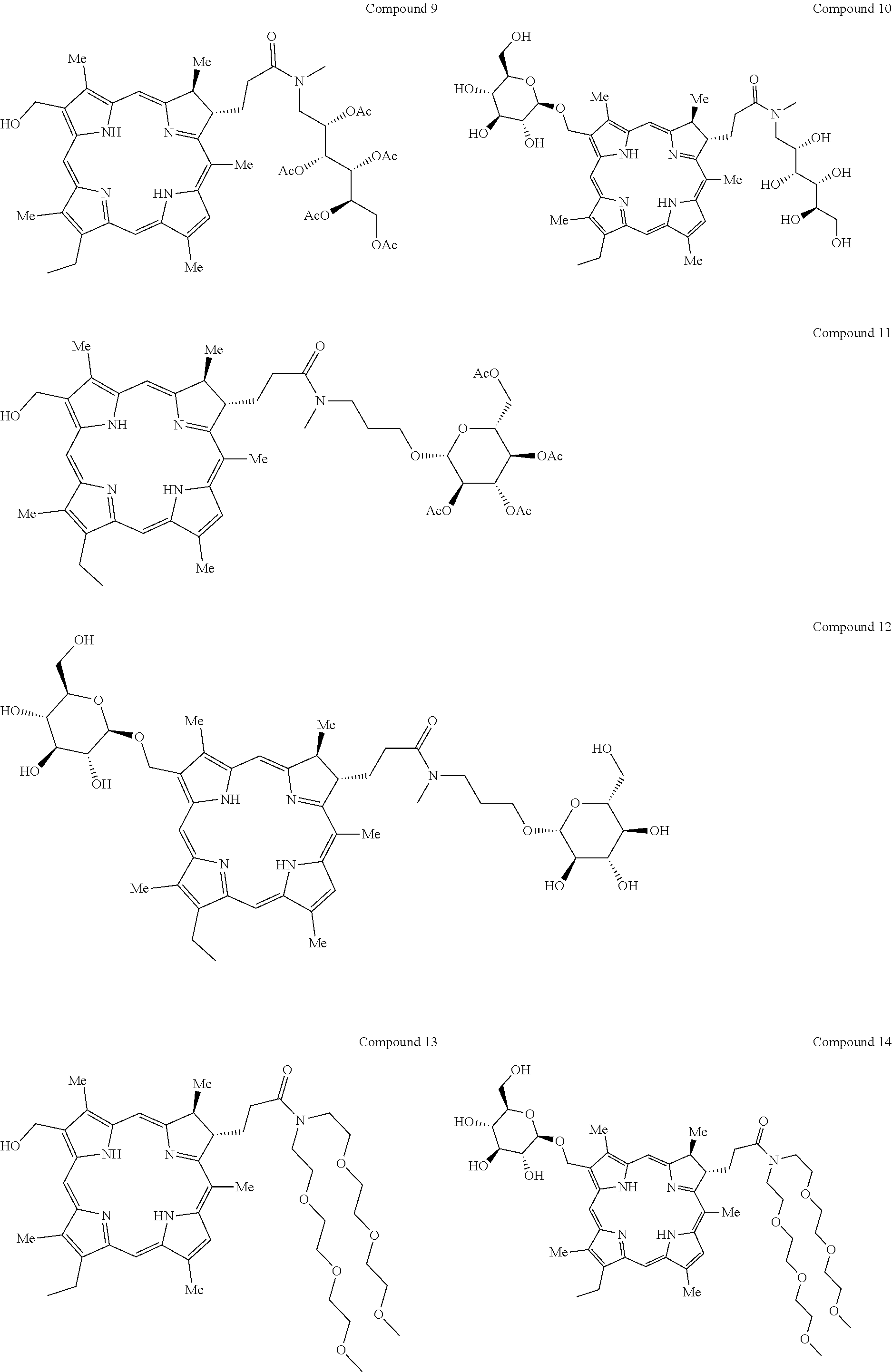 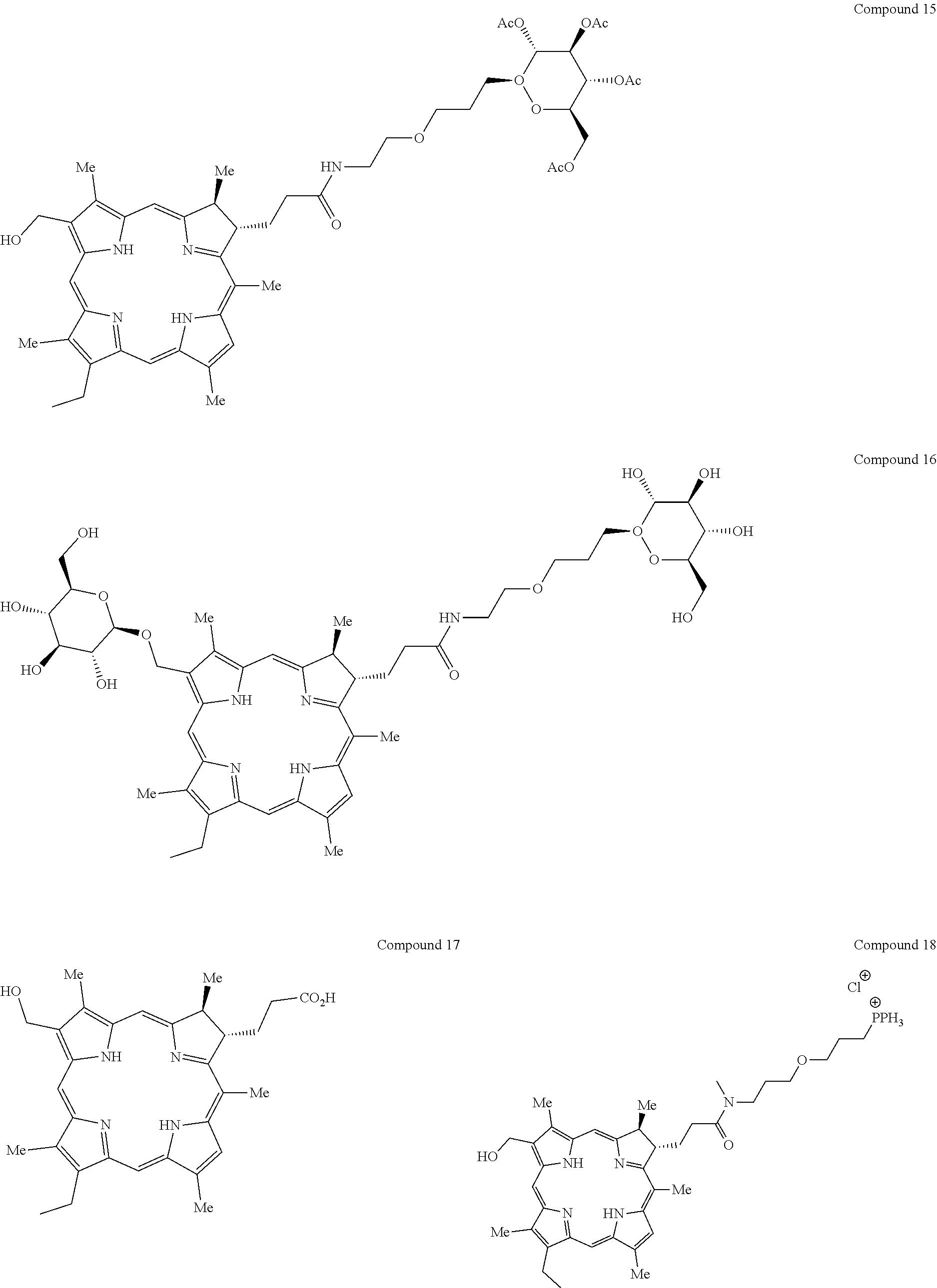 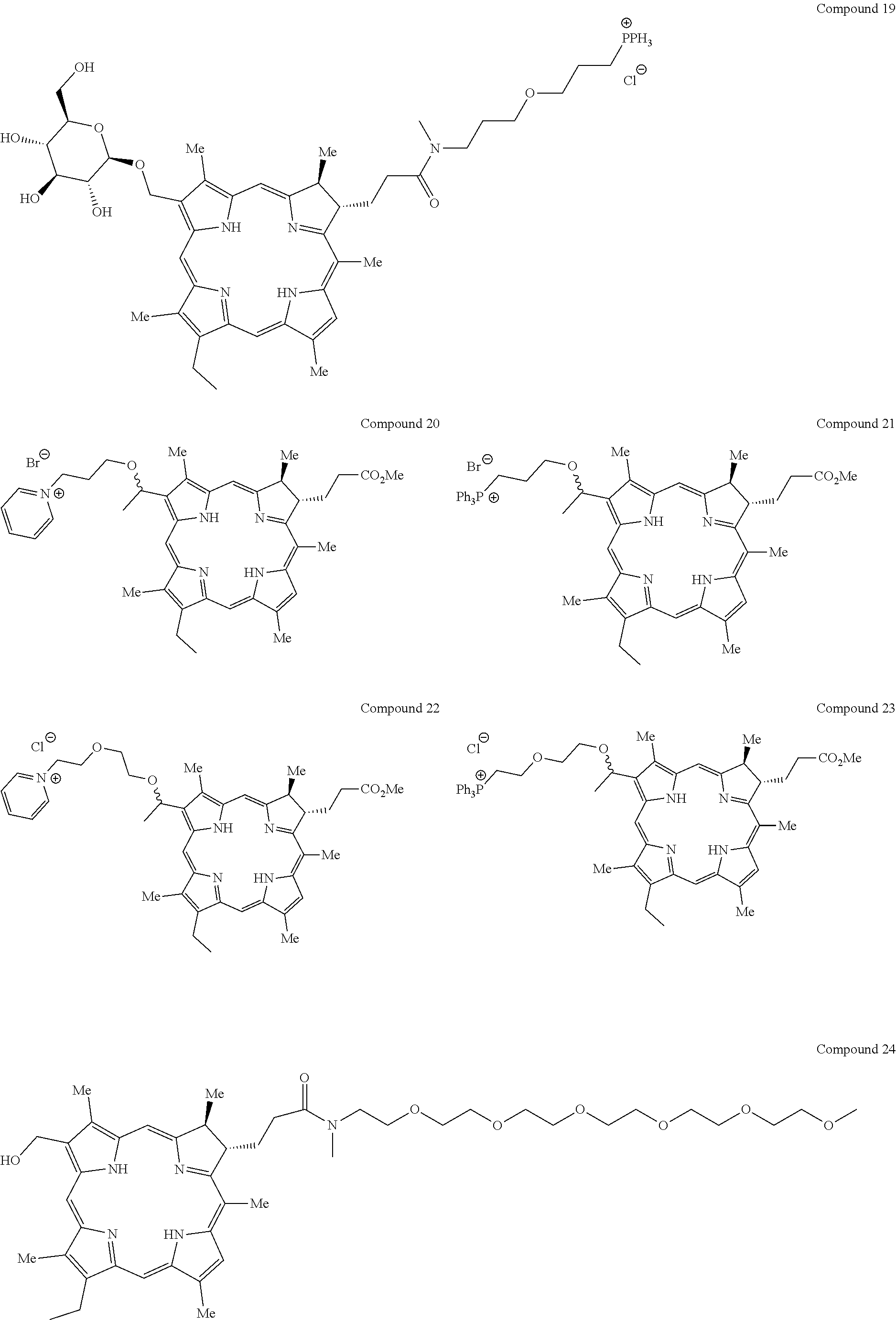 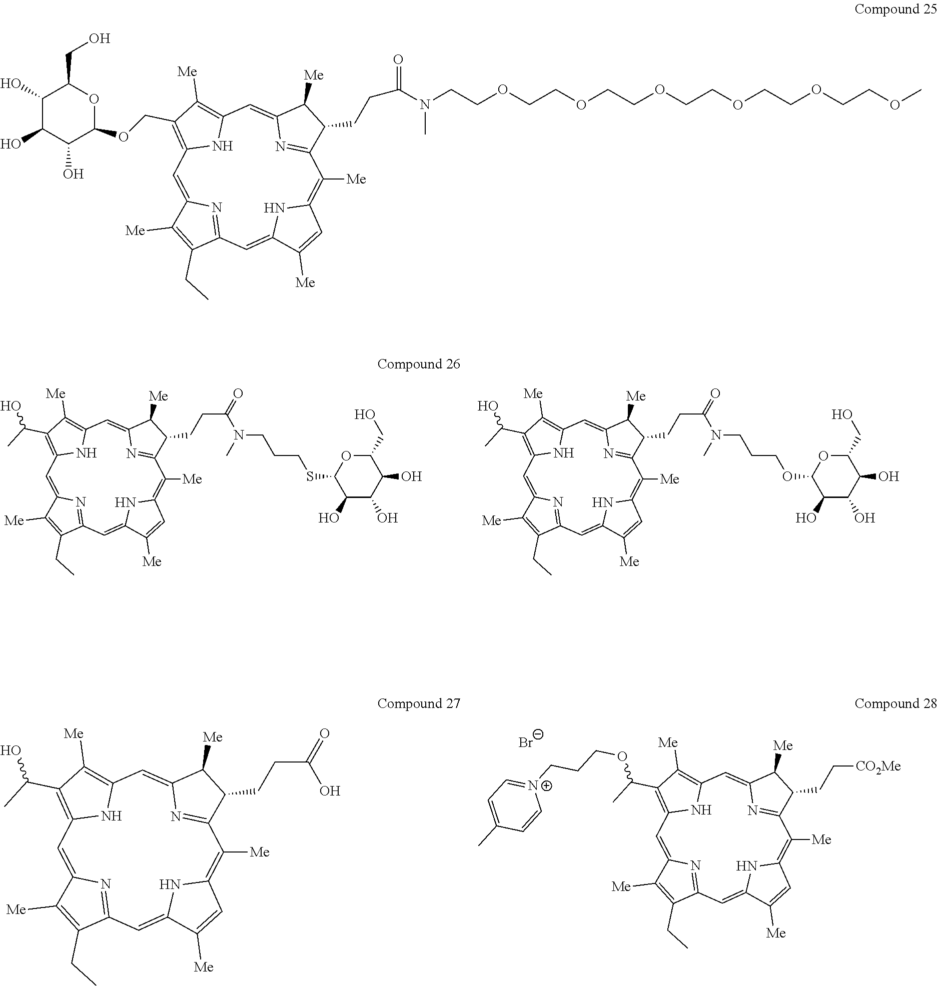 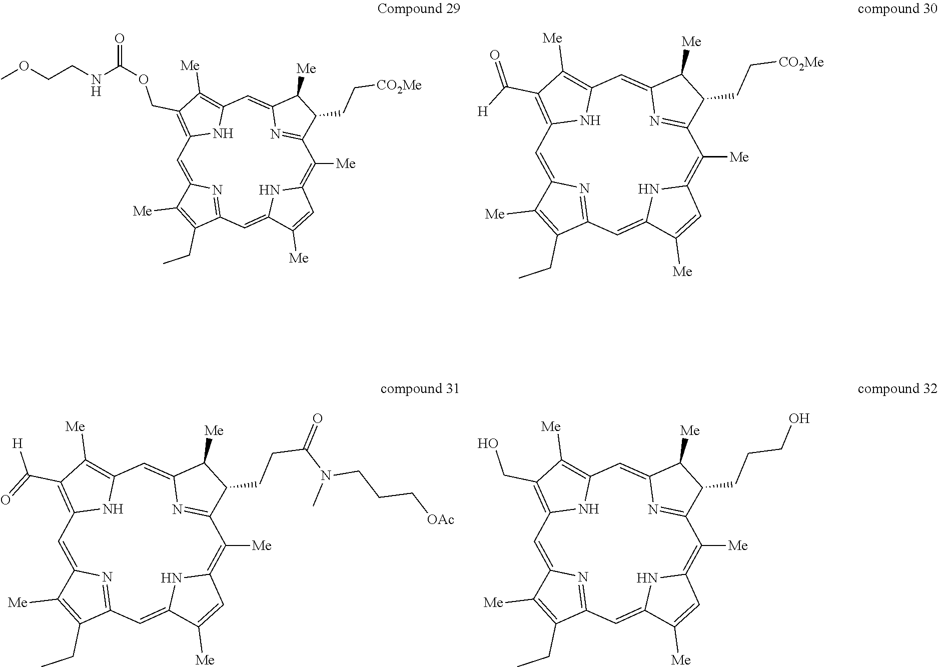 [SMILES: CCC1=C(C)c2cc3[nH]c(cc4nc(c(C)c5cc(C)c(cc1n2)[nH]5)[C@@H](CCC(=O)OC)[C@@H]4C)c(C)c3COC(=O)NCCOC ; CCC1=C(C)c2cc3[nH]c(cc4nc(c(C)c5cc(C)c(cc1n2)[nH]5)[C@@H](CCC(=O)OC)[C@@H]4C)c(C)c3C=O ; CCC1=C(C)c2cc3[nH]c(cc4nc(c(C)c5cc(C)c(cc1n2)[nH]5)[C@@H](CCC(=O)N(C)CCCOC(C)=O)[C@@H]4C)c(C)c3C=O ; CCC1=C(C)c2cc3[nH]c(cc4nc(c(C)c5cc(C)c(cc1n2)[nH]5)[C@@H](CCCO)[C@@H]4C)c(C)c3CO] 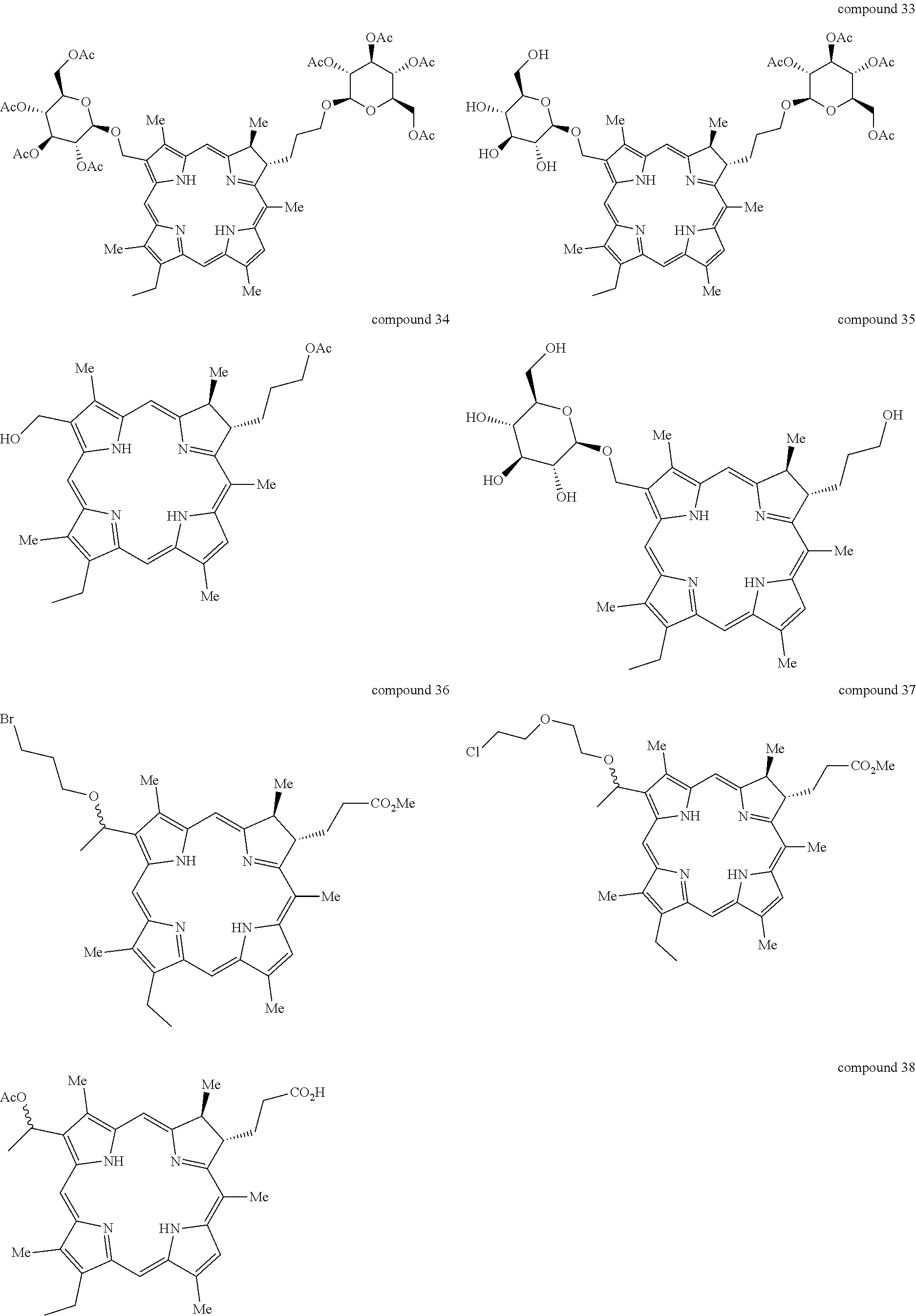 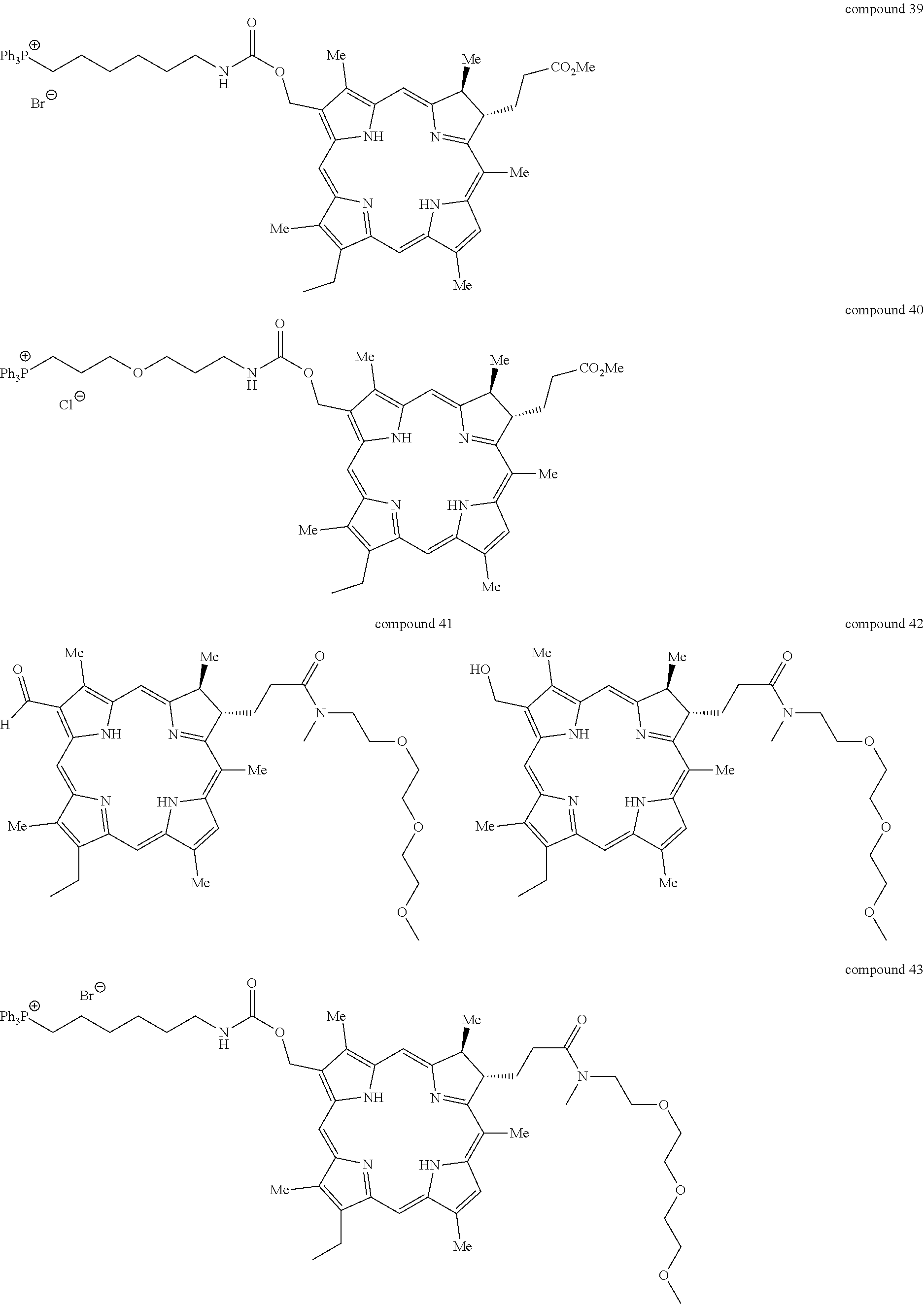 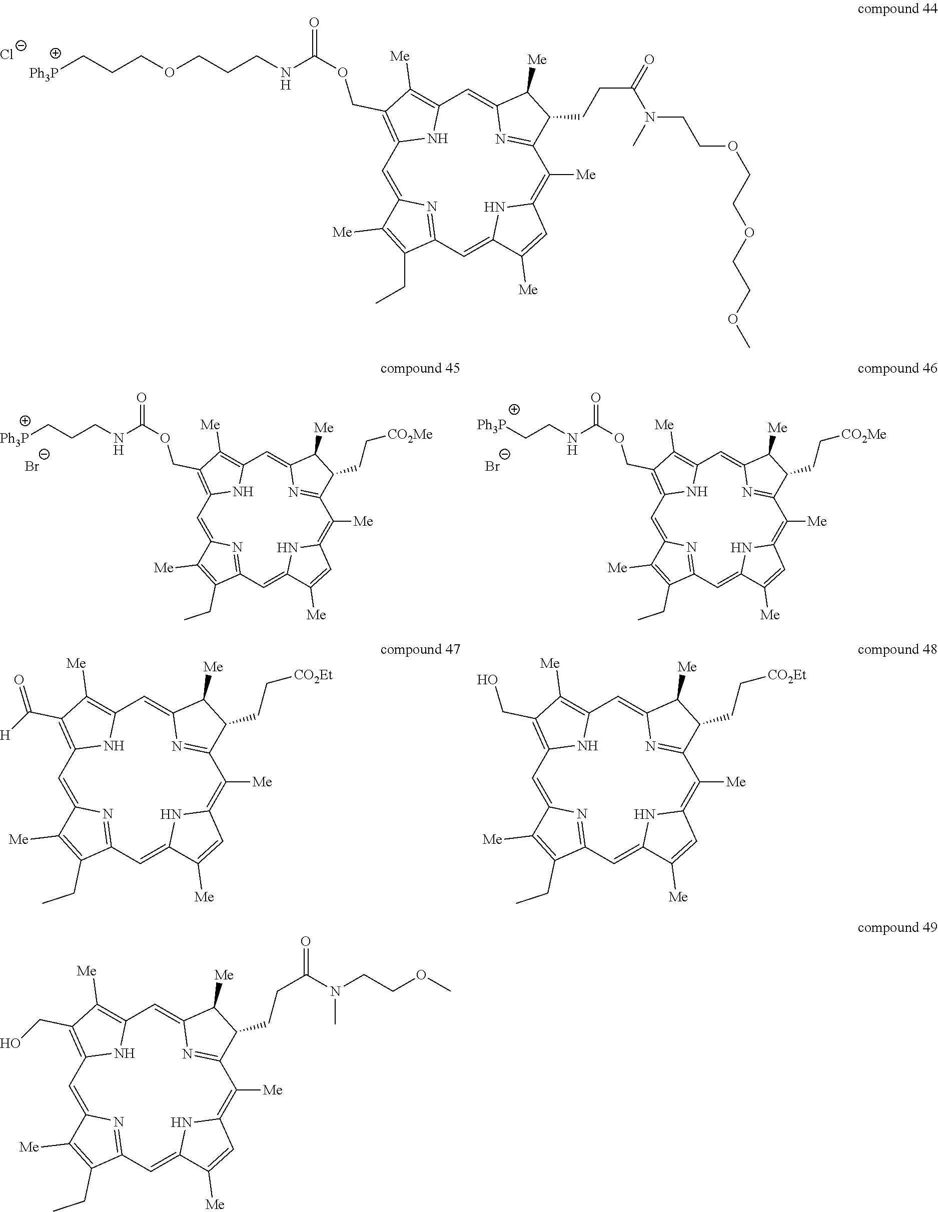 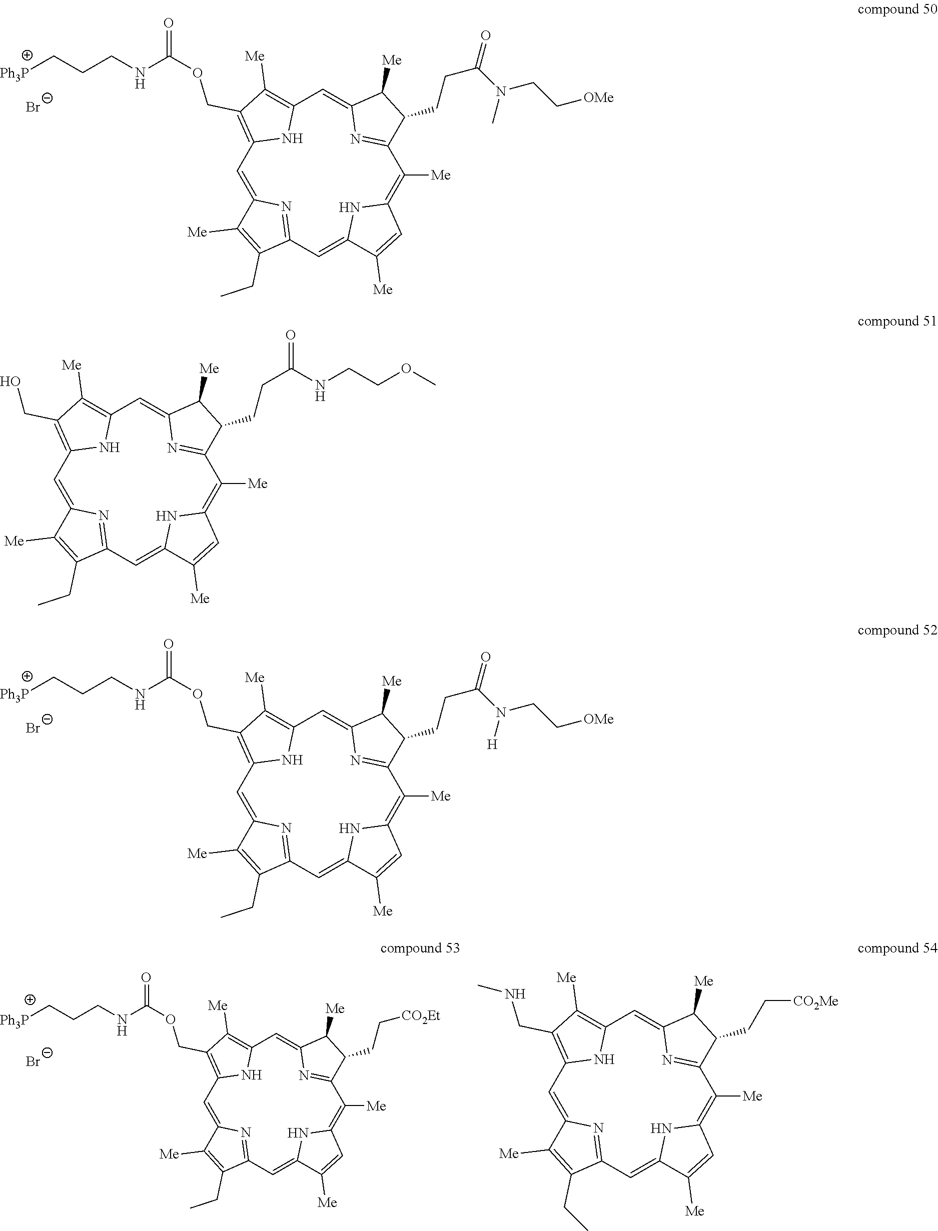 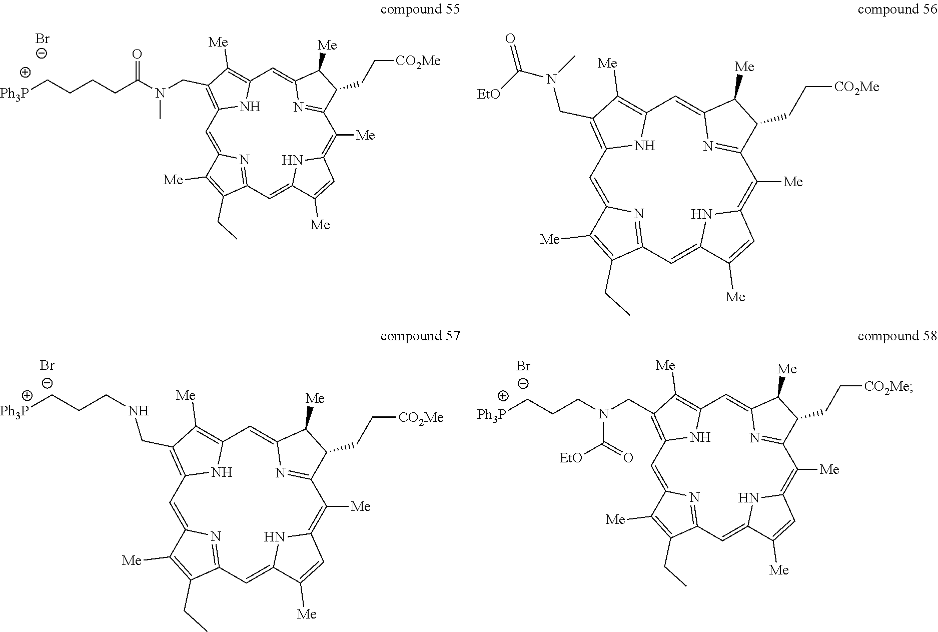
or a metal cation complex thereof, or a pharmaceutically acceptable salt thereof.

12. The compound or complex according to claim 1, for use:
(a) in medicine;
(b) in photodynamic therapy or cytoluminescent therapy;
(c) in the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-COV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas;
(d) in the treatment of a disease characterised by benign or malignant cellular hyperproliferation or by areas of neovascularisation;
(e) in the treatment of a benign or malignant tumour;
(f) in the treatment of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas; and/or
(g) in photodynamic diagnosis.

13. (canceled)

14. (canceled)

15. (canceled)

16. (canceled)

17. (canceled)

18. (canceled)

19. The compound or complex according to claim 12, wherein the compound is adapted for administration prior to administration of irradiation, optionally wherein the irradiation is electromagnetic radiation with a wavelength in the range of from 500 nm to 1000 nm.

20. (canceled)

21. A pharmaceutical composition comprising the compound or complex according to claim 1 and a pharmaceutically acceptable carrier or diluent.

22. The pharmaceutical composition according to claim 21, further comprising:
(a) polyvinylpyrrolidones; or
(b) an immune checkpoint inhibitor; or
(c) an immune checkpoint inhibitor selected from Pembrolizumab, Nivolumab, Cemiplimab, Atezolizumab, Avelumab, Durvalumab or Ipilimumab.

23. (canceled)

24. (canceled)

25. The pharmaceutical composition according to claim 21, for use;
(a) in photodynamic therapy or cytoluminescent therapy;
(b) in the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-COV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas;
(c) in the treatment of a disease characterised by benign or malignant cellular hyperproliferation or by areas of neovascularisation;
(d) in the treatment of a benign or malignant tumour;
(e) in the treatment of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas; and/or
(f) in photodynamic diagnosis.

26. (canceled)

27. (canceled)

28. (canceled)

29. (canceled)

30. (canceled)

31. The pharmaceutical composition according to claim 21, wherein the pharmaceutical composition is:
(a) adapted for administration prior to administration of irradiation; or
(b) adapted for administration prior to administration of electron radiation with a wavelength in the range of from 500 nm to 1000 nm, or
(c) in the form suitable for oral, parenteral (including intravenous, subcutaneous, intramuscular, intradermal, intratracheal, intraperitoneal, intratumoral, intraarticular, intraabdominal, intracranial and epidural), transdermal, airway (aerosol), rectal, vaginal or topical (including buccal, mucosal and sublingual) administration; or
(d) in a form suitable for oral or parenteral administration.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. A method of treating atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-COV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas; the method comprising administering a therapeutically effective amount of the compound or complex according to claim 1 to a human or animal in need thereof.

45. A method of photodynamic therapy or cytoluminescent therapy of a human or animal disease, the method comprising administering a therapeutically effective amount of the compound or complex according to claim 1 to a human or animal in need thereof, optionally wherein the human or animal disease is atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-COV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

46. (canceled)

47. The method according to claim 44, wherein the human or animal disease is:
(a) characterised by benign or malignant cellular hyperproliferation or by areas of neovascularisation;
(b) a benign or malignant tumour;
(c) early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

48. (canceled)

49. (canceled)

50. A method of photodynamic diagnosis of a human or animal disease, the method comprising administering a diagnostically effective amount of the compound or complex according to claim 1 to a human or animal.

51. A method of treating atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-COV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas; the method comprising administering a therapeutically effective amount of the compound or complex according to claim 1 to a human or animal in need thereof, wherein the human or animal is subjected to irradiation after the administration of the compound or complex, optionally wherein the irradiation is electromagnetic radiation with a wavelength in the range of from 500 nm to 1000 nm.

52. (canceled)

53. A pharmaceutical combination or kit comprising:
(a) the compound or complex according to claim 1; and
(b) a co-agent which is an immune checkpoint inhibitor, optionally wherein the immune checkpoint inhibitor is selected from Pembrolizumab, Nivolumab, Cemiplimab, Atezolizumab, Avelumab, Durvalumab or Ipilimumab.

54. (canceled)
